(19) United States
(12) Patent Application Publication
(10) Pub. No.: US 2025/0262291 A1
(43) Pub. Date: Aug. 21, 2025

(54) MICROPARTICLES FROM STREPTOCOCCUS PNEUMONIAE AS VACCINE ANTIGENS

(71) Applicant: ZalVac AB, Stockholm (SE)

(72) Inventors: Birgitta Henriques Normark, Stockholm (SE); Mario Codemo, Stockholm (SE); Federico Iovino, Bromma (SE); Sandra Muschiol, Solna (SE); Staffan Normark, Stockholm (SE); Sun Nyunt Wai, Umea (SE)

(21) Appl. No.: 19/200,040

(22) Filed: May 6, 2025

Related U.S. Application Data
(63) Continuation of application No. 18/212,857, filed on Jun. 22, 2023.
Application No. 18/212,857 is a continuation of application No. 16/474,940, filed on Jun. 28, 2019, now Pat. No. 11,844,829.

(30) Foreign Application Priority Data
Dec. 28, 2016 (SE) 1651746-8

(51) Int. Cl. A61K 39/09; A61K 39/00; C07K 14/315; C07K 16/12

(52) U.S. Cl. CPC A61K 39/092; A61K 39/00; C07K 14/3156; C07K 16/1275; A61K 2039/55555

(57) ABSTRACT

An isolated Streptococcus pneumoniae membrane vesicle microparticle (MP), wherein said MP comprises: the protein Ply at the level of ≥0.070 μg/mg total protein in the MP; and/or the protein LytA at the level of ≥0.070 μg/mg total protein in the MP; and/or the protein PspC at the level of ≥0.130 μg/mg total protein in the MP; and/or the protein RrgB at the level of ≥0.020 μg/mg total protein in the MP. Compositions comprising such MPs. Uses thereof in particular in immunization, as well as methods of manufacture thereof.

### CROSS-REFERENCE TO RELATED APPLICATIONS

This application is a continuation of U.S. application Ser. No. 18/212,857, filed on Jun. 22, 2023, which is a continuation of U.S. application Ser. No. 16/474,940, filed on Jun. 28, 2019, which is a National Stage application under 35 U.S.C. § 371 of International Application No. PCT/SE2017/051323, having an International Filing Date of Dec. 21, 2017, which claims the benefit of SE Application No. 1651746-8 filed Dec. 28, 2016. The contents of the aforementioned applications are hereby incorporated by reference in their entirety.

### TECHNICAL FIELD

The present invention relates to the field of vaccines against Streptococcus pneumoniae.

### BACKGROUND TO THE INVENTION

Streptococcus pneumoniae is a Gram-positive bacterium that is a major contributor to morbidity and mortality worldwide causing about 2 million deaths annually, i e the same range as for tuberculosis. Pneumococci are the major cause of common infections such as sinusitis, otitis, and community-acquired pneumonia (CAP), but also a common cause of severe invasive diseases (IPD) such as sepsis and meningitis. Young children and the elderly are the most susceptible age groups to acquire pneumococcal infections, but other factors such as underlying diseases, splenectomy, immunosuppression (including HIV), diabetes and a prior influenza-virus infection also sensitize for a pneumococcal infection. Pneumococcal infections such as CAP also predispose for cardiovascular diseases such as cardiac infarction, and meningitis patients may get neurological sequelae such as hearing loss and cognitive impairments.

Pneumococci have the human upper airways as their normal ecological niche, and up to 60% of preschool children may be colonized at any given time without symptoms. It is believed that most transmission events to the elderly come from young colonized children. S. pneumoniae is a highly genetically diverse species due to an efficient DNA exchange system resulting in a multitude of clonal lineages that may differ from one another in the presence and absence of genes and gene clusters, as well as in variations in individual genes.

Variations in the capsular locus, the major virulence factor and vaccine target of pneumococci, have resulted in at least 97 known capsular serotypes with different potential of causing disease.

Pneumococcal conjugate vaccines (PCVs), targeting a limited number (7, 10 or 13 in PCV7, PCV10 and PCV13 respectively) of the known 97 capsular serotypes, have been introduced in the childhood vaccination program in many countries. Vaccine introduction has led to a decrease in IPD caused by vaccine-types in vaccinated children, however, non-vaccine type pneumococci have rapidly expanded and replaced vaccine-types in child-hood carriage and in IPD, but also in non-vaccinated population such as the elderly. Furthermore, protection against vaccine types differ, and for serotype 3, a serotype included in PCV13 and with a high mortality rate, there is more or less no evidence for protection against IPD so far. Data from Sweden, where both PCV10 and PCV 13 are used, show that serotype 3 was a dominaiting serotype among IPD cases during 2016, also in counties using only PCV13. Hence, novel vaccine approaches are needed.

In Sweden the total number of patients with IPD has remained almost the same after vaccine introduction. Moreover, in a recent study we found that pneumococcal carriage rates remained around 30% in the youngest children 8 years after vaccine introduction in Stockholm, and non-vaccine types were found in over 90% of the isolates. Antibiotic resistance is emerging among clinical pneumococcal isolates and according to reports from the European Centre for Disease prevention and Control (ECDC), and the European Antimicrobial Resistance Surveillance Network (EARS-Net), reduced susceptibility to penicillin has increased during the last years in Sweden and now exceeds 5% among IPD cases. In addition, the need for multiple antigens covering various capsular serotypes lead to high production costs for the known vaccines.

Opsonophagocytosis is the primary mechanism for clearance of pneumococci from the host, and the measurement of opsonophagocytic antibodies appears to correlate with vaccine-induced protection. However, it has proven difficult to design vaccines that elicit an efficient response in form of opsonophagocytic antibodies.

Taken together, the above issues stress the need for novel vaccine approaches. Thus, an object of the present invention is the provision of improved pneumococcal antigens able to confer serotype-independent immunological responses and/or improved response in terms of opsonophagocitic antibodies and protection in experimental models.

### Background for Pneumococcal Microparticles

Spherical membranous blebs, so called outer membrane vesicles (OMVs), are produced by Gram-negative bacteria. OMVs range in size from 10 to 300 nm and they have been shown to be formed by budding from the bacterial outer membrane, and to have many functions, such as influencing virulence by different mechanisms. In Gram-negative bacteria OMVs have been suggested to act as vehicle to deliver virulence factors to host cells. Only recently, membrane vesicles (MV_{L}) were discovered also in Gram-positive bacteria and their formation and function is poorly understood compared to OMVs. Gram-positive bacteria do not contrain an outer membrane and the the cell wall differs from Gram-negative bacteria.

For Streptococcus pneumoniae MV_{L} (membrane vesicle) were isolated from pneumococci belonging to different serotypes (2, 6B, 8 and 23F) grown in liquid culture medium (1). MV_{L} from serotype 8 were shown to be protective against pneumococcal challenge with the same serotype in mice. Importantly, no cross protection was shown to pneumococci of other serotypes.

### Definitions

The pneumococcal capsule. The pneumococcal capsule, consisting of polysaccharide, exists in at least 97 different variants, so called capsular serotypes. The pneumococcal capsule is a major virulence determinant of pneumococci, affecting phagocytosis of the bacteria by host cells, and influence bacterial interactions with the innate immune response.

Pneumolysin (termed Ply herein) is a 53 kDa cholesterol dependent cytolysin released by Streptococcus pneumoniae upon lysis. It is one of the major virulence factors of this bacterium. It forms pores in all eukaryotic cells that have cholesterol in their membranes. The formation of pores by Ply frequently results in host cell death as membrane integrity is destroyed. Ply plays a central role in protecting the pneumococcus from complement attack and aiding its spread to other tissues/organs. Ply is able to activate the classical complement pathway, even in the absence of Ply specific antibody (2). A reference sequence from strain TIGR4 is presented in SEQ ID NO: 1.

LytA is the major autolysin of Streptococcus pneumoniae. Lysis is caused by cleaving the lactyl-amide bond between the stem peptides and the glycan strands of peptidoglycan, resulting in hydrolysis of the cell wall. The contribution of LytA to pneumococcal virulence is still unclear. It is possible that LytA-mediated lysis releases other virulence factors such as pneumolysin. LytA could also be released to lyse neighboring non-competent pneumococcal cells in a fratricidal manner. This would potentially facilitate genetic exchange between naturally competent pneumococcal populations that easily take up and incorporate DNA by homologous recombination. A third possibility is that LytA mediates lysis to release proteins involved in immune evasion or cell wall components that may interfere with the host immune response (3). A reference sequence from strain TIGR4 is presented in SEQ ID NO: 2.

PspC/CbpA (choline binding protein A) is a protein that binds the phosphocholine present in the teichoic acid and the lipoteichoic acid of the cell membrane and the cell wall. It is a major pneumococcal adhesin. It promotes pneumococcal adherence via a human-specific interaction with the ectodomain of the polymeric Ig receptor. It also prevents activation of C3b and complement-mediated opsonophagocytosis of pneumococci (4). The pspC locus is highly polymorphic and 11 major groups of this protein have been identified. Single PspC proteins are identified by sequential numbers separated from the group number by a dot (5) as follows: PspC1.1, PspC2.1, PspC2.2, PspC3.1, PspC3.4, PspC4.2/PspC10.1, PspC5.1, PspC6.1/PspC9.1, PspC7.1, PspC8.1, PspC11.1 and PspC11.4 (SEQ ID NOs: 3-14, respectively).

RrgB is the major subunit and stalk protein of the pneumococcal pilus. S. pneumoniae pilus 1 is encoded by a genetic islet (PI-1) present in 30 to 50% of the pneumococcal strains and is implicated in adhesion to epithelial cells, lung infection, and virulence. Pilus 1 is composed of the backbone subunit RrgB, the minor pilin subunits RrgA, and RrgC (6). A reference sequence from strain TIGR4 is presented in SEQ ID NO: 15.

RrgA is a minor pilin subunit of the pneumococcal pilus. RrgA is the tip protein of the pilus and has been shown to mediate adhesion to epithelial cells (6, 7). Recently, RrgA was shown to promote meningitis development in mice (8). A reference sequence is presented in SEQ ID NO: 16.

PhtD is a polyhistidine triad protein of S. pneumoniae. Polyhistidine triad (Pht) proteins are streptococcal surface proteins that contain multiple copies of a characteristic HxxHxH sequence, designed as histidine triads, which were predicted to bind divalent metal cations. Despite the increasing amount of biochemical, structural and physiological data, the functional role of PhtD and the other Pht proteins remains unclear. PhtD could be involved in zinc uptake. Alternatively, Pht proteins could play a role in protecting the pneumococcus from toxic effects of high Zn²⁺ concentrations by scavenging, storing or trapping Zn²⁺ ions (9). A reference sequence from strain TIGR4 is presented in SEQ ID NO: 17.

IgA refers to Immunoglobulin A1 protease of S. pneumoniae (10). Said IgA1-protease is a highly specific enzyme that cleaves amino acid sequences of certain proteins. The natural substrate of the IgA protease is immunoglobulin A1, hence its name. The enzyme is in fact capable of cleavage of proteins with the amino acid sequence N—X-Z-Pro-Pro/-Y-Pro-C, where the X in the sequence is preferably a Proline or Serine; the Y=Threonine, Serine or Alanine; and Z preferably is Arginine or Threonine. Thus, the IgA1 protease acts by cleaving the proline-rich hinge region of the heavy chain of IgA1. Release of the IgA1 protease by the pathogen allows adherence to mucous membranes by degrading host immunoglobulin A. A reference sequence from strain TIGR4 is presented in SEQ ID NO: 18.

The term protective immunity in the present context refers to immunization measures resulting in any degree of reduction in the likelihood of developing the condition for which the protective immunity is relevant, including a minor, substantial or major reduction in likelihood of developing the condition as well as total prevention. Preferably, the degree of likelihood reduction is at least a minor reduction.

The term sequence identity expressed in percentage is defined as the value determined by comparing two optimally aligned sequences over a comparison window, wherein a portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison and multiplying the result by 100 to yield the percentage of sequence identity. Unless indicated otherwise, the comparison window is the entire length of the sequence being referred to. In this context, optimal alignment is the alignment produced by the BLASTP algorithm as implemented online by the US National Center for Biotechnology Information (see The NCBI Handbook [Internet], Chapter 16), with the following input parameters: Word length=3, Matrix=BLOSUM62, Gap cost=11, Gap extension cost=1.

### BRIEF DESCRIPTION OF THE FIGURES

FIGS. 1A through 1D: Membrane-derived vesicles and microparticles produced by S. pneumoniae TIGR4 (T4). (FIG. 1A) Electron micrographs and atomic force micrographs (insets) of membrane vesicles and microparticles after isolation and density gradient purification from liquid culture (MV_{L}) and microparticles from plate-grown (MP) bacteria. (FIG. 1B) Size distribution of MV_{L} MPs from TIGR4 and MPs from serotype 3. (FIG. 1C) Electron micrograph of a membrane budding from S. pneumoniae T4R and (FIG. 1D) the same electron micrograph in larger magnification. Released particle is indicated by arrow.

FIGS. 2A through 2D: Identification of proteins present in pneumococcal MV_{L} and MP. (FIG. 1A) Preparations of MV_{L} and MP were separated by SDS-PAGE and proteins visualized by coomassie staining. In comparison, proteins present in a whole cell lysate (WC) and supernatant (SN) of a liquid bacterial culture were visualized. (FIG. 1B) Venn diagram displaying the number of proteins identified by mass spectrometry (repeated twice) exclusively found in MV_{L} and MP preparations and common to both. (FIG. 1C) Mass spectrometry identification of proteins from MV_{L} and MP. Numbers indicate percentages of proteins based on their subcellular localization, defined as cytosolic proteins, membrane-associated proteins (lipoproteins), proteins with one transmembrane domain (N-terminally anchored), transmembrane proteins, cell wall associated proteins and secreted proteins. (FIG. 1D) Immunoblot detection of pneumococcal proteins and virulence factors present in MV_{L} and MP isolated from S. pneumoniae T4 WT, pneumolysin-(T4Δply) and LytA-deficient (T4ΔlytA) strains.

FIG. 3: Pneumolysin associated with pneumococcal MV_{L} and MP is biologically active. Quantification of in vitro hemolytic activity of purified MV_{L} and MP isolated from S. pneumoniae T4 and T4Δply. Erythrocytes from buffy coat blood were incubated for 60 minutes with different concentrations (0.1, 0.8, 2, 6 and 10 μg protein) of MV_{L} and MP. As control treatments, erythrocytes were incubated with PBS (−) or 1% Triton X-100 (+). Data are represented as means+/−SEM of three independent experiments. *=P<0.05; **=P<0.01; ***=P<0.001.

FIGS. 4A through 4E: Microparticles (MP) are internalized by A549 epithelial cells.

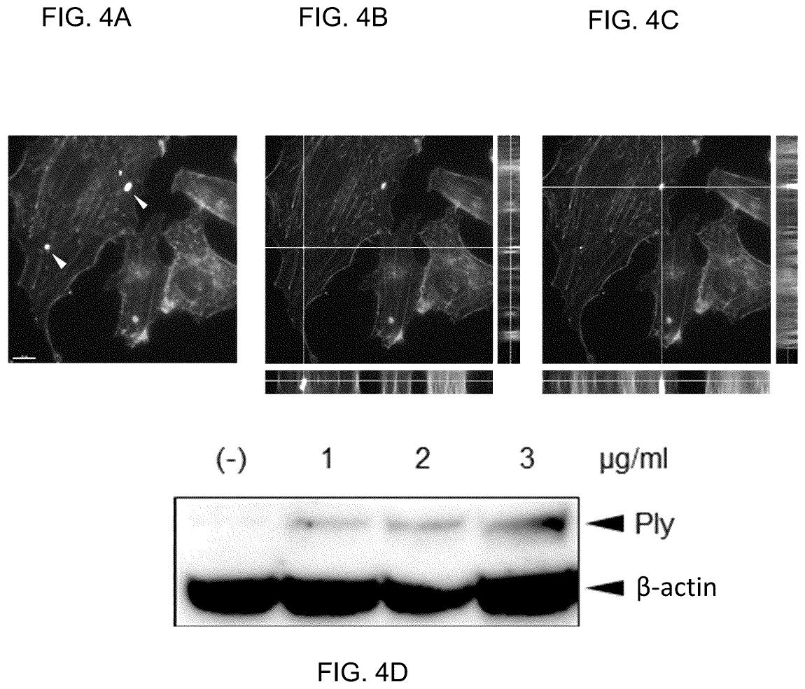

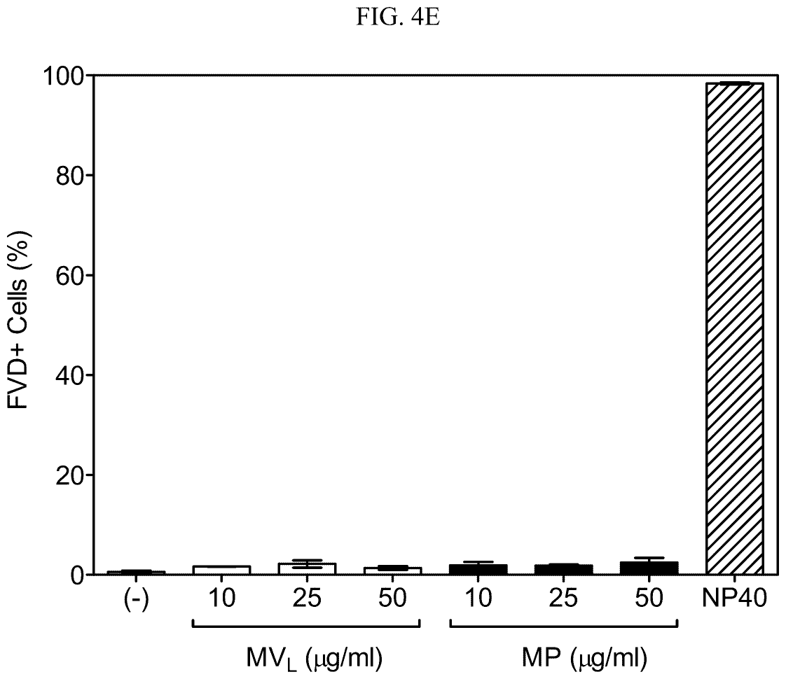

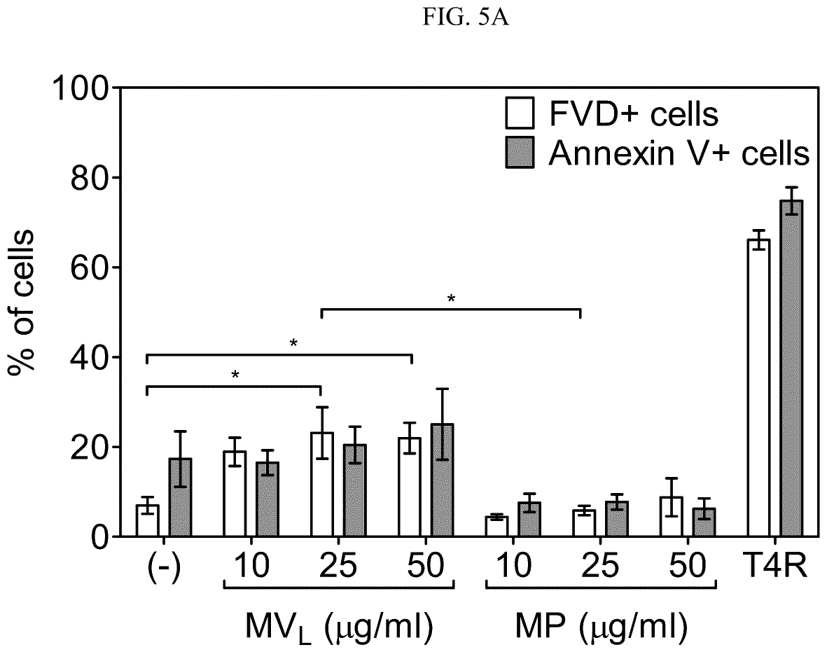

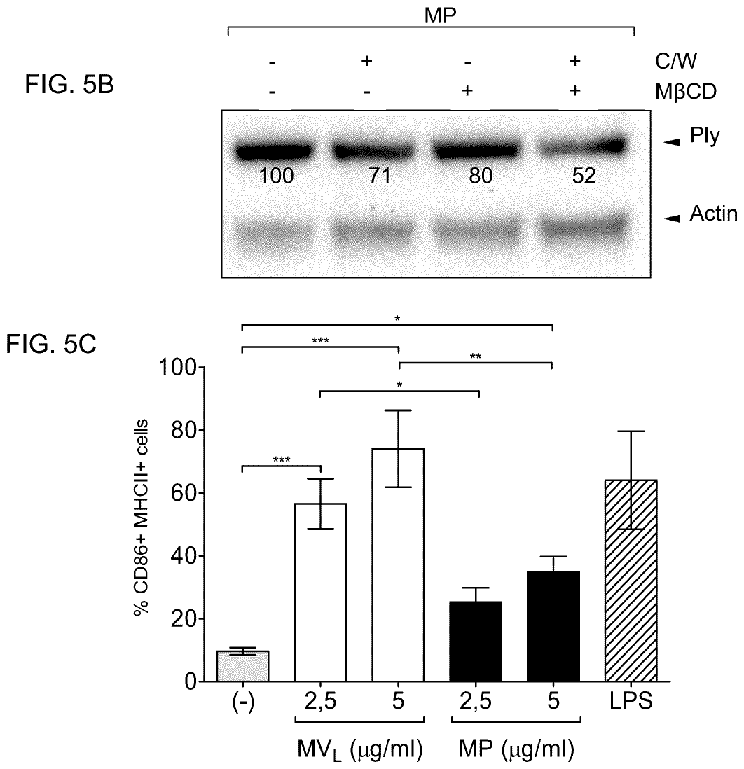

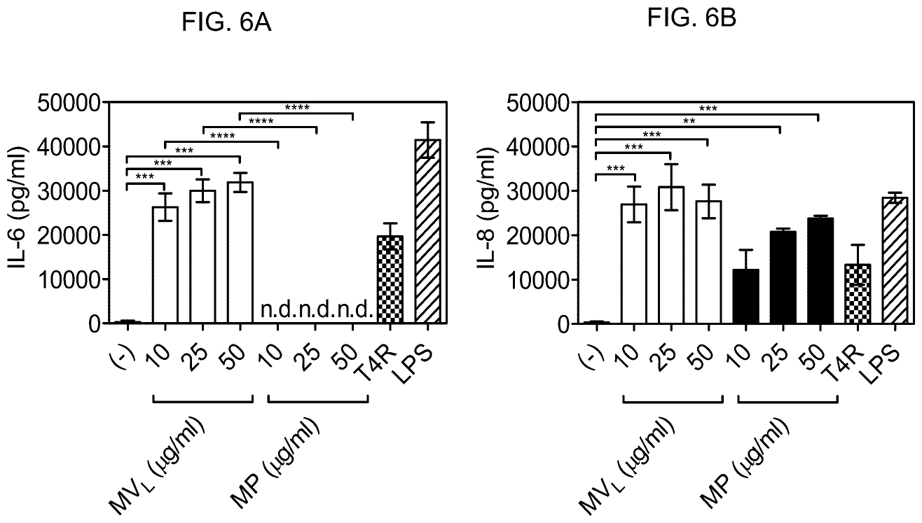

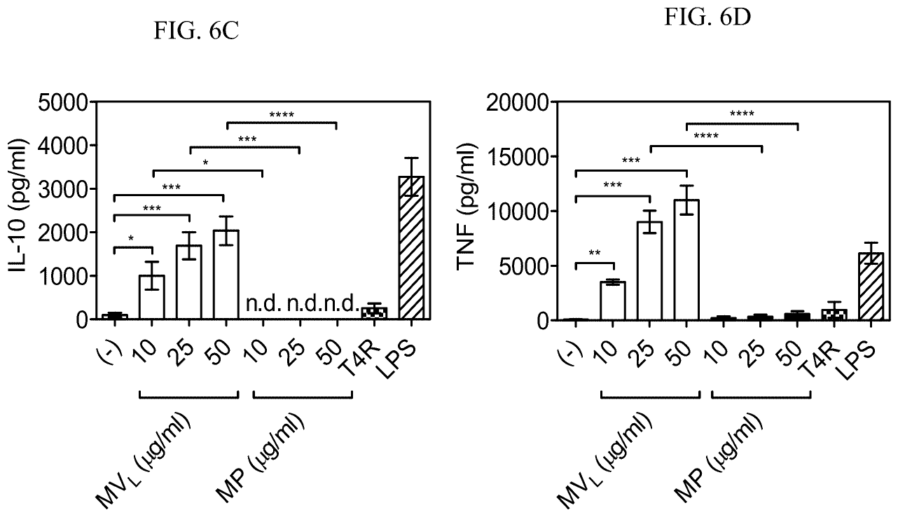

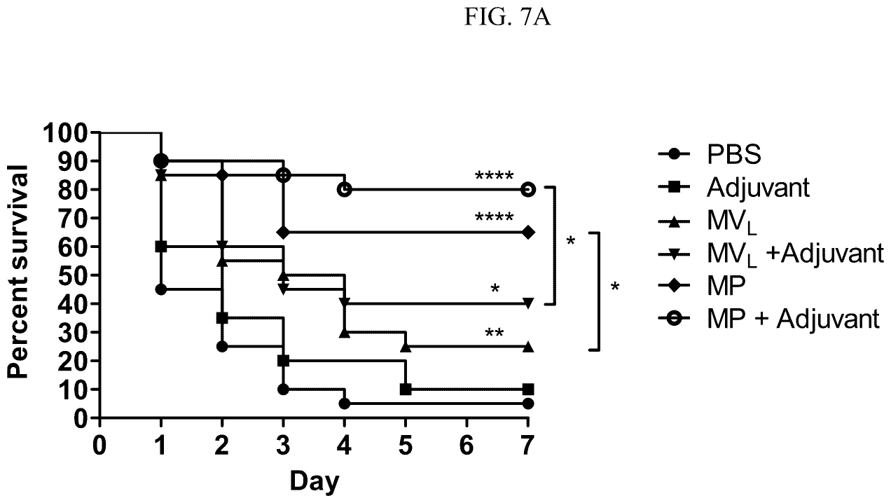

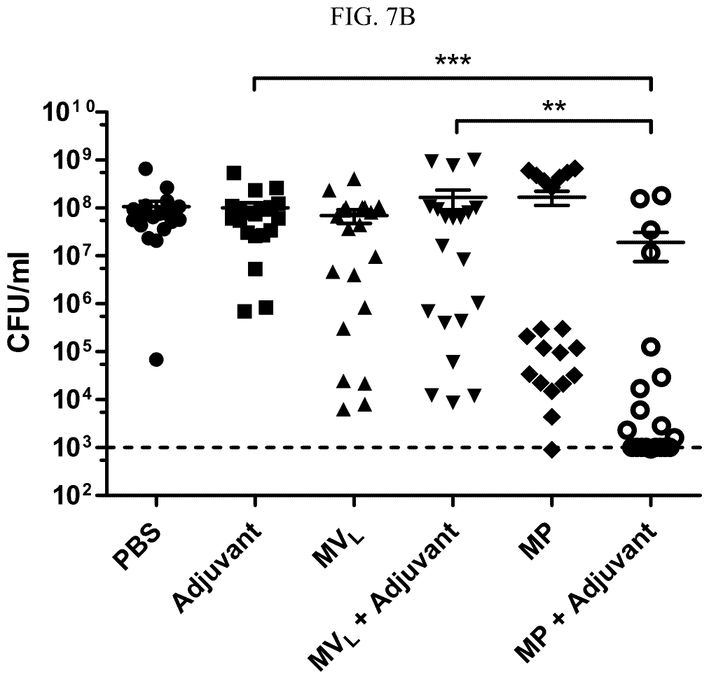

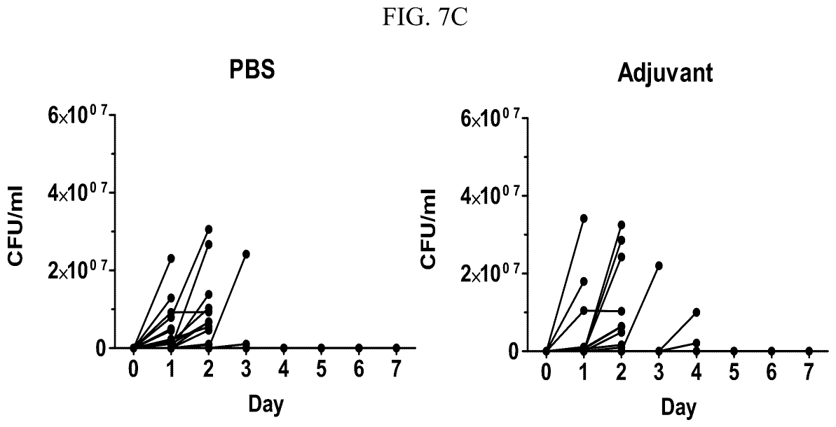

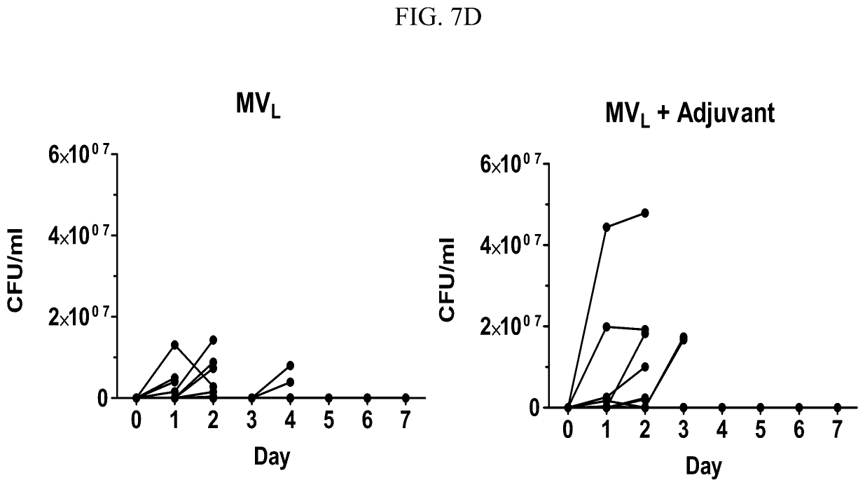

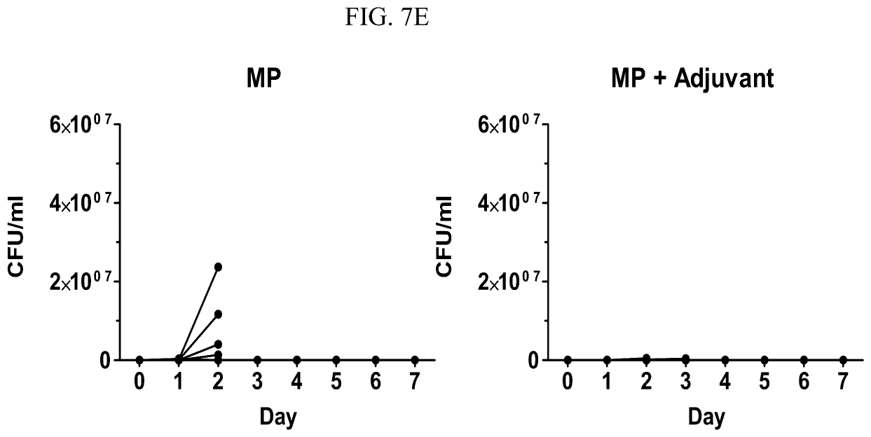

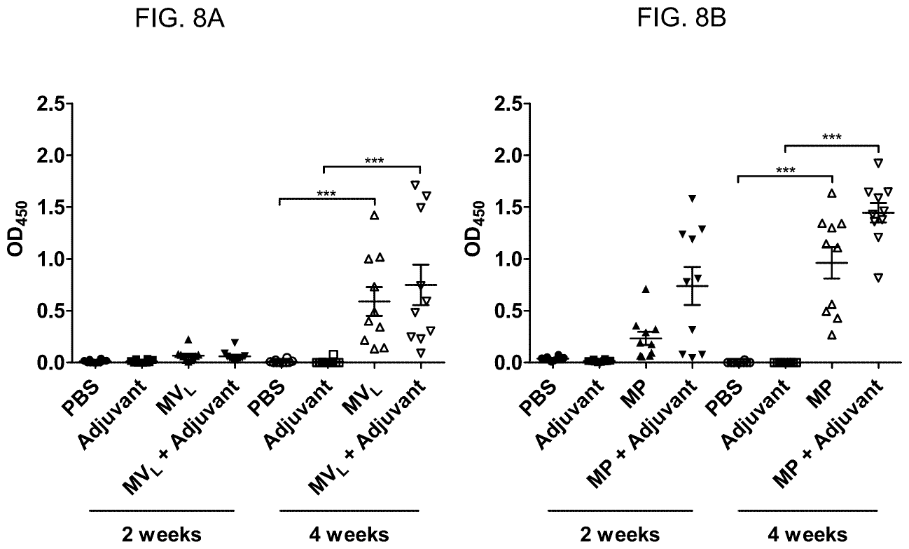

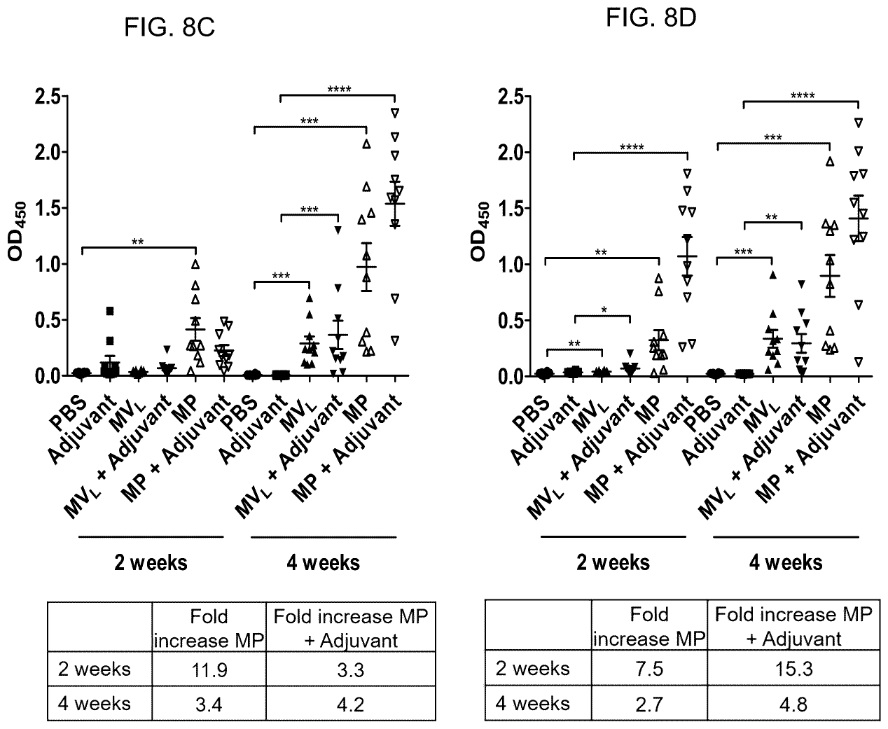

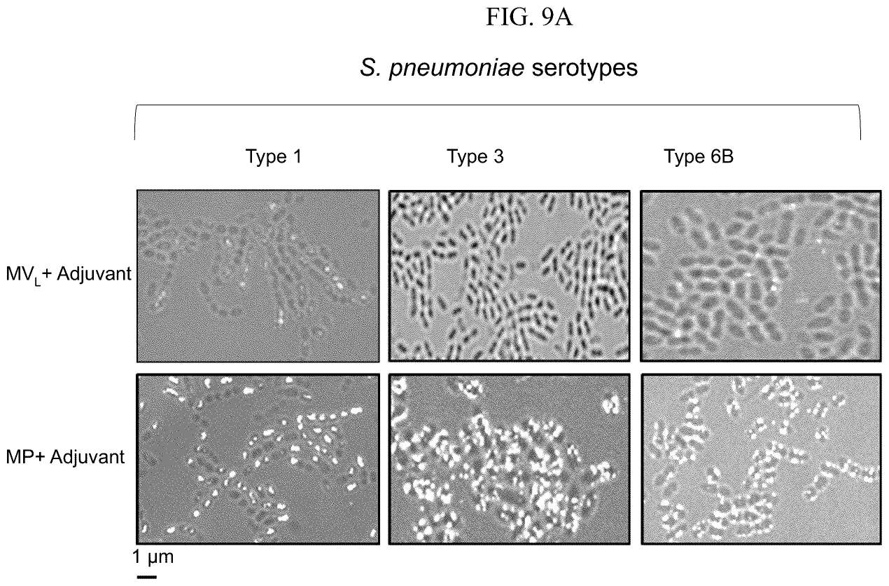

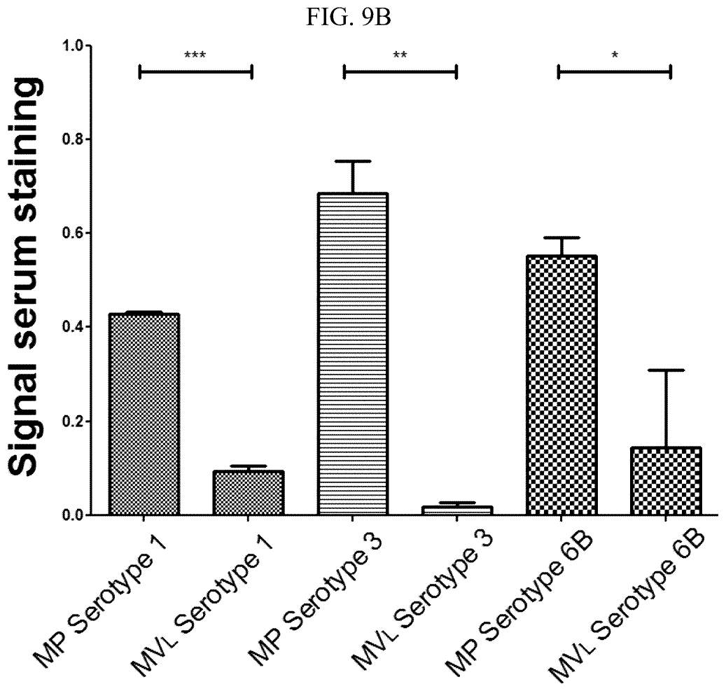

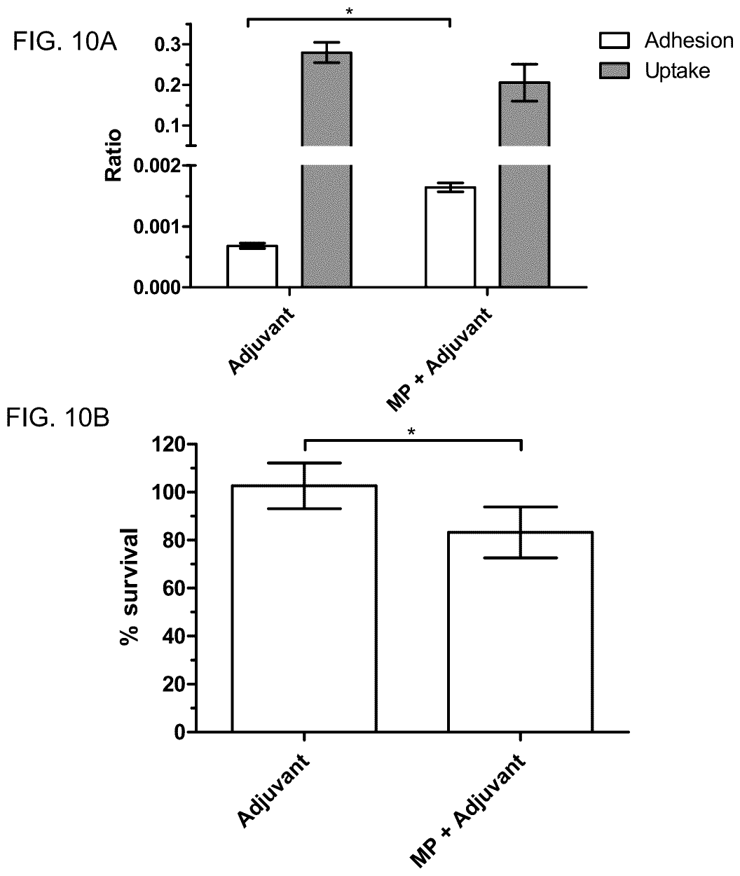

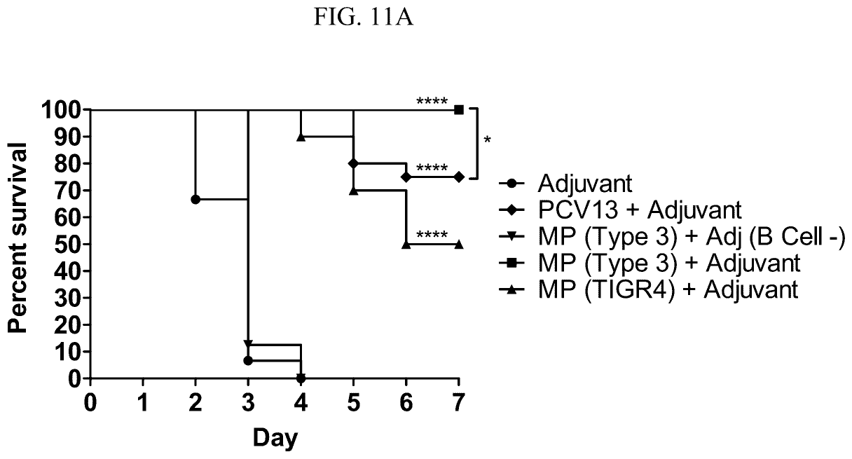

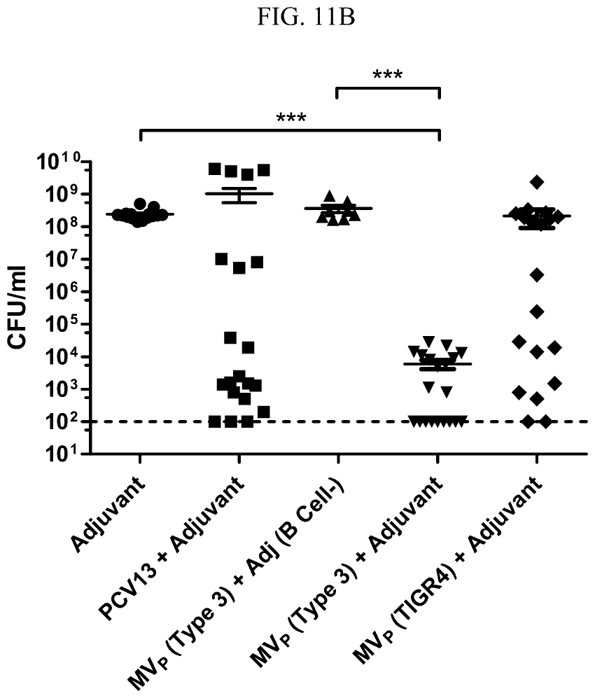

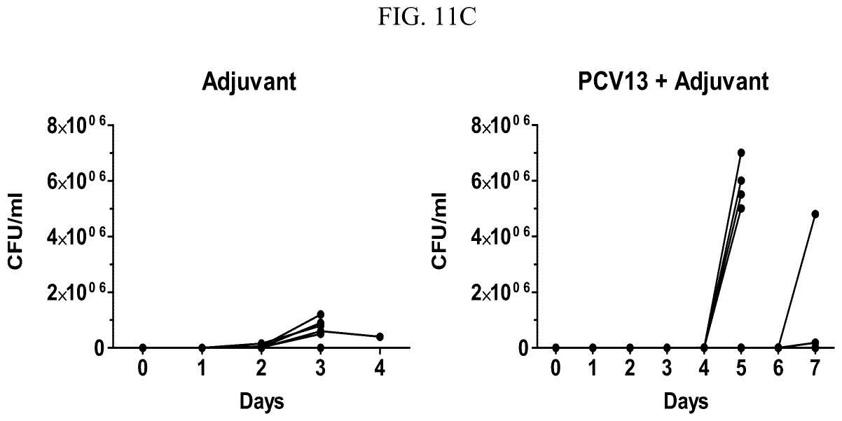

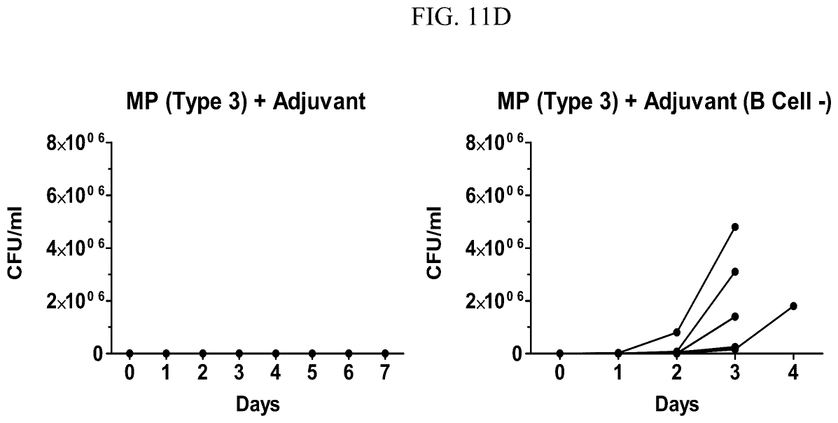

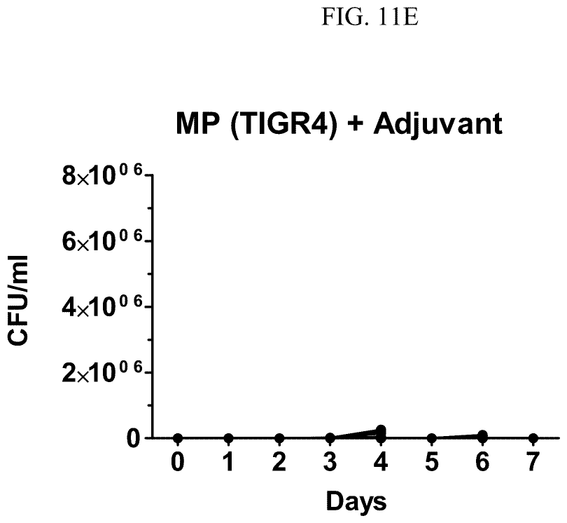

(FIGS. 4A, 4B and 4C) Immunofluorescence pictures of A549 cells treated with MP (10 μg/ml) for 24 hours. Cells were stained for F-actin, MP are detected with anti-pneumolysin and LytA antibodies. (FIG. 4A) Z-stack image (nr. stacks=21) of MP-treated cells. Internalized MP are indicated by arrows. (FIGS. 4B and 4C) Orthogonal views of the same picture, taken at stack nr. 11, for the two indicated MP, further showing their intracellular localization.

(FIG. 4D) Immunoblot detection of anti-Ply, and anti-β-actin as loading control, in A549 cell lysates after incubation with increasing concentrations (1, 2, 3 μg/ml) of MP. As control treatment, A549 cells were incubated with PBS (−). (FIG. 4E) Viability of A549 cells examined by flow cytometry of fixable viability dye (FVD) positive cells after 24 hours of incubation with MV_{L} or MP. As control treatment, A549 cells were incubated with PBS (−) or 0.02% NP40 in PBS. Data are represented as means+/−SEM of three independent experiments.

FIGS. 5A through 5C: MV_{L} and microparticles (MP) activate human monocyte-derived dendritic cells (DCs). (FIG. 5A) Percentages of FVD and Annexin V-positive cells analyzed by flow cytometry. Cells were incubated for 24 hours with different concentrations (10, 25, 50 μg/ml) of MV_{L}, MP, PBS (−) or the unencapsulated mutant of TIGR4 (T4R). Data are represented as means+/−SEM of three independent experiments. *=P<0.05. (FIG. 5B) Immunoblot detection of anti-Ply, and anti-β-actin as loading control, in cell lysates after incubation of DCs with MP (4 μg/ml) in presence or absence of the inhibitors Cytochalasin D and Wortmannin (C/W), to block active phagocytosis, or methyl-β-cyclodextrin (MBCD), to block lipid rafts and membrane fusion. Numbers represent the relative intensity of bands corresponding to Ply, adjusted to the loading control bands, in three independent experiments. (FIG. 5C) DC activation measured by flow cytometry of MHCII and CD86-positive cells after 24 hours incubation with different concentrations (2.5, 5 μg/ml) of MV_{L}, MP, PBS (−) or LPS (1 μg/ml). Data are represented as means+/−SEM of three independent experiments. *=P<0.05; ***=P<0.001.

FIGS. 6A through 6D: Cytokine release by human monocyte-derived dendritic cells upon incubation with MV_{L} or MP. (FIG. 6A) IL-6, (FIG. 6B) IL-8, (FIG. 6C) IL-10 and (FIG. 6D) TNF released by dendritic cells after 24 hours of incubation with different concentrations (10, 25, 50 μg/ml) of MV_{L} or MP. Control treatments include PBS (−), the unencapsulated mutant of TIGR4 (T4R) and LPS (1 μg/ml). Not detectable (n.d.) amounts of cytokines are indicated. Data are represented as means+/−SEM of three independent experiments. *=P<0.05; **=P<0.01; ***=P<0.001; ****=P<0.0001.

FIGS. 7A through 7E: Intranasal immunization of C57BL/6 mice with MV_{L} or MP of S. pneumoniae TIGR4 increase survival against intranasal infection with serotype 1.

(FIG. 7A) Percentage of mice survived after challenge. 20 mice per group. *=P<0.05; **=P<0.01; ****=P<0.0001. (FIG. 7B) CFUs in lungs of mice after sacrifice. Each dot represents one mouse. **=P<0.01; ***=P<0.001. (C-E) CFUs in blood of mice. Each line represents one mouse.

FIGS. 8A through 8D: Intranasal immunization of mice with MV or MPs stimulate the production of pneumoccal-specific IgG. Detection of MV-reactive (FIG. 8A), MP-reactive (FIG. 8B), T4-reactive (FIG. 8C) and T4R-reactive (FIG. 8D) mouse IgG in immunized mice sera using ELISA assay. Each dot represents one mouse serum. *=P<0.05; **=P<0.01; ***P<0.001; ****=P<0.0001.

FIGS. 9A and 9B: Staining of serotypes 1, 3 and 6B using antibodies raised against MP from a serotype 4 strain. (FIG. 9A) Immunofluorescence pictures of pneumococcal strains belonging to serotypes 1, 3 and 6B stained using sera, as primary antibodies, from mice immunized either with MV_{L}+adjuvant or MP+adjuvant. The white spots on the bacterial surface indicates the presence of specific antibodies against pneumococci in the sera of the immunized mice. (FIG. 9B) Quantification of the signal (white spots) detected on the bacteria; the signal ratio was calculated by dividing the area of the signal detected on the bacteria (white spots) by the total area detected occupied by the bacteria. The area covered by the bacteria and the area covered by the signal detected on the bacteria after staining with sera were selected, defined and measured using the functions Image>Adjust>Threshold and Analyze>Measure of ImageJ. *=P<0.05; **=P<0.01; ***=P<0.001.

FIGS. 10A and 10B: Opsonophagocytic activity of antibodies in mice sera. (FIG. 10A) RAW mouse macrophages were incubated with Streptococcus pneumoniae type 1, pre-incubated with mouse sera from the adjuvant or MP+adjuvant groups. Indicated are the ratios of adherence of bacteria to cells, or uptake of bacteria inside cells. Data are represented as means+/−SEM of three independent experiments. *=P<0.05. (FIG. 10B) RAW mouse macrophages have been incubated with Streptococcus pneumoniae type 1, pre-incubated with mouse sera from the adjuvant or MP+adjuvant groups. Indicated is the percentage of taken up bacteria surviving inside the cells after 1 hour. Data are represented as means+/−SEM of three independent experiments. *=P<0.05.

FIGS. 11A through 11E: Intranasal immunization of C57BL/6 mice with MP of S. pneumoniae increases survival against intranasal infection with serotype 3. In particular, immunization with MPs of serotype 3 confers 100% protection against serotype 3 infection in comparison to PCV13 immunization, which confers 75% protection. B-cell deficient mice immunized with MPs of serotype 3 are not protected against intranasal infection with serotype 3, strongly suggesting that protection provided by MPs is antibody-dependent.

(FIG. 11A) Percentage of mice that survived after pneumococcal challenge. 10 mice in the group of “B Cell-(deficient mice)”, 10 mice in the group “Adjuvant”, 20 mice per all other groups. *=P<0.05; ****=P<0.0001. (FIG. 11B) CFUs in lungs of mice after sacrifice. Each dot represents one mouse. ***=P<0.001. (FIGS. 11C-11E) CFUs in blood of mice. Each line represents one mouse.

### SUMMARY OF THE INVENTION

The present invention relates to the following items. The subject matter disclosed in the items below should be regarded disclosed in the same manner as if the subject matter were disclosed in patent claims.
1. An isolated Streptococcus pneumoniae membrane vesicle microparticle (MP), wherein said MP comprises:
   i. the protein Ply at the level of ≥0.070 μg/mg total protein in the MP;
   ii. the protein LytA at the level of ≥0.070 μg/mg total protein in the MP;
   iii. the protein PspC at the level of ≥0.130 μg/mg total protein in the MP; or
   iv. the protein RrgB at the level of ≥0.020 μg/mg total protein in the MP.
2. The microparticle according to item 1, further comprising a capsular polysaccharide of a capsular serotype of Streptococcus pneumoniae, preferably at a level of ≥0.001, more preferably ≥0.01, most preferably ≥0.1 μg/μg total protein in the MP.
3. The microparticle according to any of the preceding items, comprising the protein Ply at the level of ≥0.070 μg/mg total protein in the MP.
4. The microparticle according to any of the preceding items, comprising the protein Ply at the level of ≥0.15, preferably ≥0.2, more preferably ≥0.3, most preferably ≥0.35 μg/mg total protein in the MP.
5. The microparticle according to any of the preceding items, comprising the protein LytA at the level of ≥0.070 μg/mg total protein in the MP.
6. The microparticle according to any of the preceding items, comprising the protein LytA at the level of ≥0.08, preferably ≥0.09, yet more preferably ≥0.10, most preferably ≥0.20 μg/mg total protein in the MP.
7. The microparticle according to any of the preceding items, comprising the protein PspC at the level of ≥0.130 μg/mg total protein in the MP.
8. The microparticle according to any of the preceding items, comprising the protein PspC at the level of ≥0.15, preferably ≥0.18, more preferably ≥0.20, most preferably ≥0.3 μg/mg total protein in the MP.
9. The microparticle according to any of the preceding items, comprising the protein RrgB at the level of ≥0.020 μg/mg total protein in the MP.
10. The microparticle according to any of the preceding items, comprising the protein RgrB at the level of ≥0.022, preferably ≥0.025, most preferably ≥0.028 μg/mg total protein in the MP.
11. The microparticle according to any of the preceding items, further comprising the protein PhtD.
12. The microparticle according to any of the preceding items, comprising the protein PhtD at a level being at least 2-fold, more preferably at least 2.5-fold compared to Streptococcus pneumoniae membrane vesicles obtained in liquid culture in terms of μg/mg total protein in the particle or the vesicle, respectively.
13. The microparticle according to any of the preceding items, further comprising the protein RrgA.
14. The microparticle according to any of the preceding items, comprising the protein RrgA at the level of ≥0.02, preferably ≥0.05, more preferably ≥0.10, most preferably ≥0.2 μg/mg total protein in the MP.
15. The microparticle according to any of the preceding items, comprising the protein IgA.
16. The microparticle according to any of the preceding items, comprising the protein IgA at the level of >0.02, preferably ≥0.05, more preferably ≥0.10, most preferably ≥0.2 μg/mg total protein in the MP.
17. The microparticle according to any of the preceding items, wherein the protein Ply comprises a sequence having at least 80%, preferably 85%, more preferably 90%, yet more preferably 95%, most preferably 100% sequence identity to SEQ ID NO: 1.
18. The microparticle according to any of the preceding items, wherein the protein LytA comprises a sequence having at least 80%, preferably 85%, more preferably 90%, yet more preferably 95%, most preferably 100% sequence identity to SEQ ID NO: 2.
19. The microparticle according to any of the preceding items, wherein the protein PspC comprises a sequence having at least 40%, preferably 80%, more preferably 85%, yet more preferably 90%, still more preferably 95%, most preferably 100% sequence identity to SEQ ID NOs: 3.
20. The microparticle according to any of the preceding items, wherein the protein PspC comprises a sequence having at least 80%, preferably 85%, more preferably 90%, yet more preferably 95%, most preferably 100% sequence identity to any one of SEQ ID NOs: 3-14.
21. The microparticle according to any of the preceding items, wherein the protein RgrB comprises a sequence having at least 80%, preferably 85%, more preferably 90%, yet more preferably 95%, most preferably 100% sequence identity to SEQ ID NO: 15.
22. The microparticle according to any of the preceding items, wherein the protein PhtD comprises a sequence having at least 80%, preferably 85%, more preferably 90%, yet more preferably 95%, most preferably 100% sequence identity to SEQ ID NO: 16.
23. The microparticle according to any of the preceding items, comprising the protein RgrA, wherein the protein RgrA comprises a sequence having at least 80%, preferably 85%, more preferably 90%, yet more preferably 95%, most preferably 100% sequence identity to SEQ ID NO: 17.
24. The microparticle according to any of the preceding items, comprising the protein IgA, wherein the protein IgA comprises a sequence having at least 80%, preferably 85%, more preferably 90%, yet more preferably 95%, most preferably 100% sequence identity to SEQ ID NO: 18.
25. The microparticle according to any of the preceding items, wherein the MP is 5-300 nm in diameter, preferably 15-175 nm.
26. The microparticle according to any of the preceding items, wherein the MP is 10-125 nm in diameter.
27. The microparticle according to any of the preceding items, wherein the MP is derived from a Streptococcus pneumoniae strain selected from any serotype 1 strain, any serotype 3 strain, TIGR4, P1031 and A66, preferably TIGR4.
28. The microparticle according to any of the preceding items, obtainable by:
   a. Culturing host cells of a Streptococcus pneumoniae strain on blood agar plates;
   b. harvesting the cultured host cells;
   c. centrifuging the harvested host cells at 17,000 g for 30 minutes at +4C;
   d. subjecting the supernatant to filtration through a 0.22 μm filter;
   e. centrifuging the filtered supernatant at 120,000 g for 2 h at +4C;
   f. washing the pellets from e) twice in phosphate-buffered saline with sedimentations at 120,000 g for 2 h at +4C;
   g. resuspending the pellet in 1 ml phosphate-buffered saline;
   h. adjusting the resuspended pellet to 50% (w/v) Optiprep™ density gradient medium in a total volume of 2 ml and overlaying with one fraction of 30% (w/v) Optiprep™ (9 ml) followed by a fraction of 5% (w/v) Optiprep™ (3 ml);
   i. centrifuging the gradients at 250,000×g for 3 hours at 4° C.;
   j. collecting the 4 ml top fraction, containing the membrane microparticle; and
   k. washing the microparticle three times phosphate-buffered saline with sedimentations of 250,000×g for 2 hours at +4° C.
29. A composition comprising a microparticle according to any of items 1-28.
30. The composition according to item 29, wherein the composition is devoid of whole Streptococcus pneumoniae cells.
31. The composition according to any of items 29-30, further comprising capsular polysaccharides from Streptococcus pneumoniae.
32. The composition according to any of items 29-31, comprising MPs in an amount of 1 μg/ml, preferably 5 μg/ml, more preferably 10 μg/ml, most preferably 100 μg/ml.
33. The composition according to any of items 29-32, further comprising an adjuvant.
34. The composition according to any of items 29-33, further comprising an adjuvant comprising aluminium hydroxide.
35. The composition according to any of items 29-34, being an immunogenic composition.
36. The composition according to any of items 29-35, being an immunogenic composition capable of eliciting opsonophagocitic antibodies against Streptococcus pneumoniae when administered to a mammalian host.
37. The composition according to any of items 29-36, being an immunogenic composition is capable of eliciting serotype independent antibodies against Streptococcus pneumoniae when administered to a mammalian host.
38. The composition according to any of items 29-37, being an immunogenic composition is capable of eliciting antibodies against Streptococcus pneumoniae serotype 3 when administered to a mammalian host.
39. The composition according to any of items 29-38, further comprising capsular polysaccharides of a capsular serotype of Streptococcus pneumoniae.
40. The composition according to any of items 29-39, being a vaccine.
41. The composition according to any of items 29-40, for use in a method for inducing protective immunity against Streptococcus pneumoniae in a subject.
42. The composition according to item 41, for use according to item 41, wherein the immunity is protective against a condition selected from pneumococcal sinusitis, pneumococcal otitis, pneumococcal pneumonia and invasive pneumococcal disease including but not limited to pneumococcal sepsis and pneumococcal meningitis, preferably invasive pneumococcal disease.
43. A use of a microparticle according to any of items 1-28 in an immunogenic composition.
44. The use according to item 43, wherein the immunogenic composition is a vaccine.
45. A use of a microparticle according to any of items 1-28 in the manufacture of a vaccine.
46. A method for manufacturing a vaccine, comprising:
   a. Providing a microparticle according to any of items 1-28;
   b. Providing an adjuvant;
   c. Mixing the microparticle and the adjuvant in a suitable vehicle in order to produce a vaccine.
47. A method for producing an isolated Streptococcus pneumoniae microparticle according to any of items 1-28, comprising:
   a. providing a Streptococcus pneumoniae host cell;
   b. culturing said host cell under conditions allowing the production of a microparticle according to any of items 1-28 by the host cell; and
   c. isolating the microparticle thus produced.
48. The method according to item 47, wherein the host cell is of a strain selected from any serotype 1 strain, any serotype 3 strain, TIGR4, P1031 and A66, preferably TIGR4.
49. The method according to any of items 47-48, wherein the culturing is carried out in solid phase.
50. The method according to any of items 47-49, wherein the culturing is carried out in solid phase on blood agar.
51. The method according to any of items 47-50, wherein isolating the microparticle comprises density gradient centrifugation.

### DETAILED DESCRIPTION

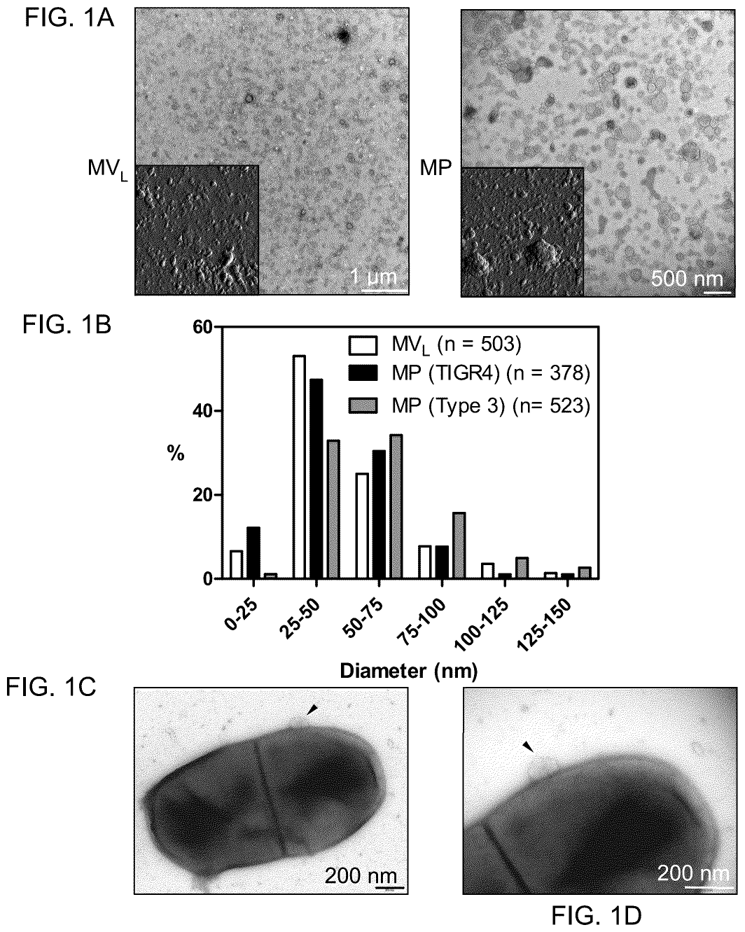

The inventors isolated membrane vesicles (MV_{L}) from S. pneumoniae of serotype 4 grown in liquid cultures as previously described in the literature (1). They also isolated a novel type of membrane particles termed microparticles (MP) using a method involving growth of the bacteria overnight on blood agar plates. FIG. 1 shows the appearance of the MV_{L} and MP using electron microscopy and atomic force microscopy as well as their size distribution. Microparticles from S. pneumoniae TIGR4 grown on plates (MP) were smaller in size on average than MV_{L} coming from liquid media (MV_{L}). However, MP from S. pneumoniae serotype 3 display a different size distribution, containing less of the smallest particles and more of the largest ones.

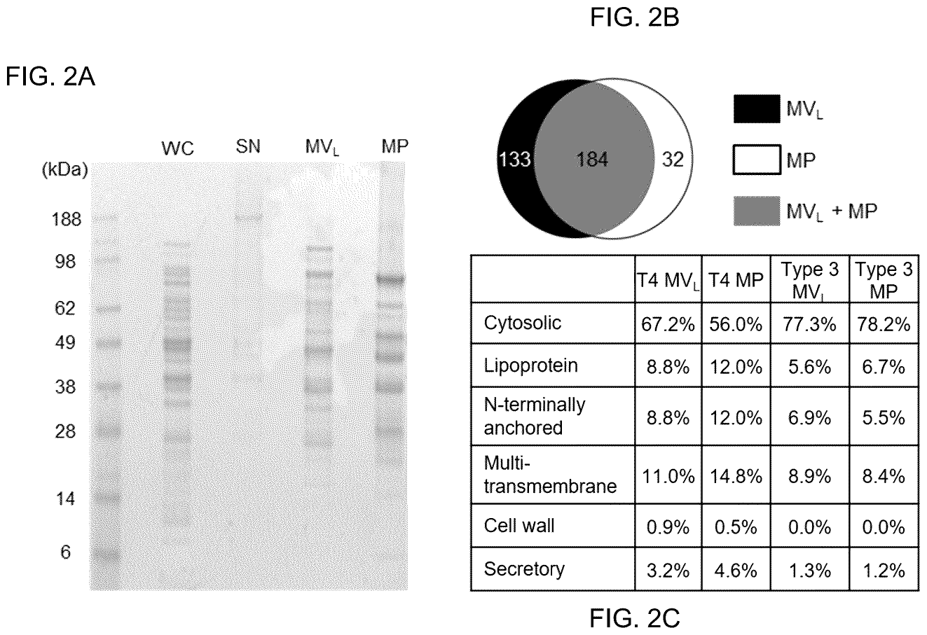

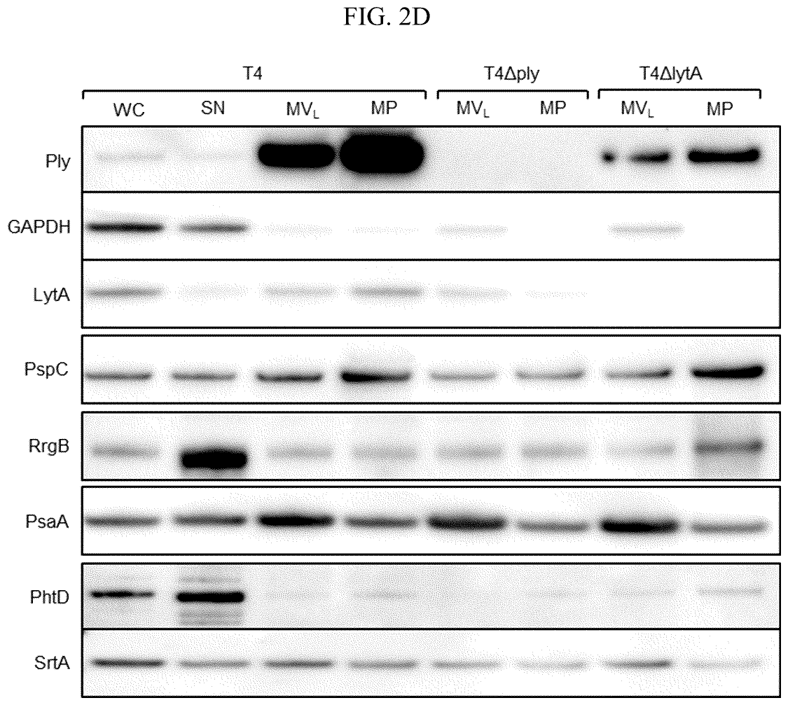

Biochemical comparison of MV_{L} and MP preparations from TIGR4 revealed different properties for the MP and MV_{L}. MP isolated from plates carried certain pneumococcal proteins to a higher extent than liquid grown MV_{L}. They were particularly enriched in pneumolysin, the major pore-forming toxin in S. pneumoniae, as shown in FIG. 2D.

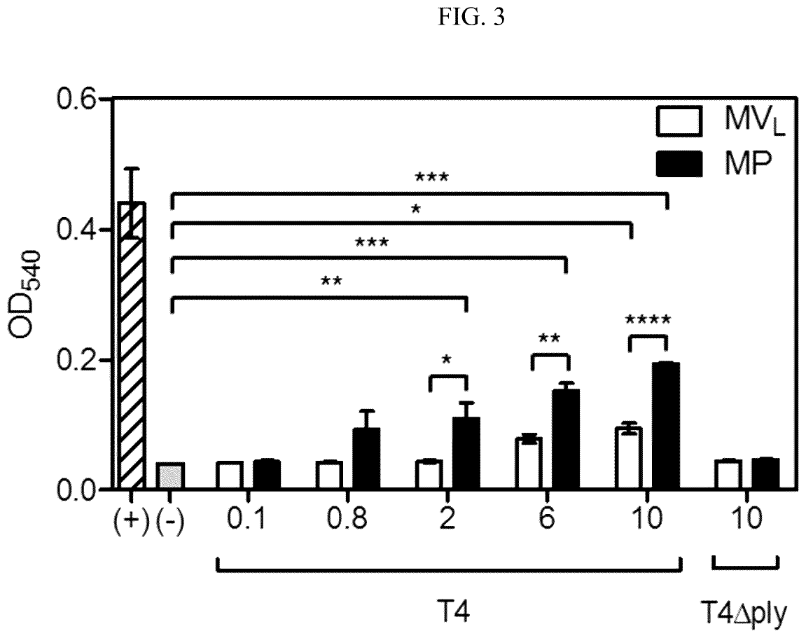

The novel MP were characterized using in vitro cell assays, and it was shown that consistent with the quantitated levels, the MP contain more biological pneumolysin activity than the MV_{L} (FIG. 3). It was further shown that the MP are taken up by epithelial cells (FIG. 4A-D) but are not cytotoxic (FIG. 4E). Using human monocyte-derived dendritic cells it was shown that both MP and MV_{L} are internalized into DCs, induce maturation of DCs (FIG. 5), and elicit differential cytokine responses (FIG. 6).

Next, the inventors investigated the potential of MP to protect mice using experimental models of pneumonia and invasive disease (FIGS. 7-11). The results showed that 80% of mice (wild type C57BL/6 mice) immunized with MP from serotype 4 (combined with aluminium hydroxide as adjuvant) survived after an intranasal pneumococcal infection caused by serotype 1 (heterologous challenge, cross protection). ELISA analysis showed that mice immunized with MP had a significantly higher MP-specific IgG response in comparison to control mice (treated only with the adjuvant) (FIG. 8). Immunization with serotype 3 and then challenge with serotype 3 led to a 100% protection (FIG. 11). Immunization with serotype 4 MP and then challenge with serotype 3 (a serotype that has been problematic in current vaccines) gave a protection of ca 50% (FIG. 11). Furthermore, immunofluorescence stainings showed that sera from mice immunized with MP from serotype 4 contained antibodies specific against multiple other serotypes, such as serotypes 1, 3 and 6B (FIG. 9).

In conclusion, it was found that MP protect mice against pneumococcal infection, and, most importantly, such protection is serotype-independent to a significant degree, in contrast to the protection conferred by currently available vaccines (polysaccharide or conjugated vaccines) that are, as mentioned in the background section, strictly serotype-dependent. The MP immunisation furthermore elicited protection against serotype 3 which is a challenge for current vaccines. Indeed, immunization with the currently clinically used vaccine PCV13 provided significantly less protection to mice than MP isolated from serotype 3 (FIG. 11).

Moreover, for protein based vaccines that are under development today, there is a problem in obtaining opsonophagocytic capability. Importantly, the present data show that using MP as a vaccine affects opsonophagocytosis (Example 3, FIG. 10). Last but not least, available pneumococcal vaccines (PCVs) are extremely important to fight pneumococcal diseases in developing countries. However, the costs to produce current vaccines are very high. An important benefit with the present approach is a drop in production costs since isolation of microparticles does not require high-cost preparations with conjugations, instead the method of the present invention is more simple based on bacterial growth combined with high speed-centrifugation.

Isolated Streptococcus pneumoniae Membrane Vesicles and Microparticles

As discussed in the Background section, a few types of Streptococcus pneumoniae membrane vesicles have been obtained in liquid culture (1). The solid phase culture method used by the inventors resulted in a novel type of particles termed microparticles having different and advantageous properties as discussed above and shown in the Examples. As shown in Table 1, the microparticles differ from the known membrane vesicles, MV_{L} in terms of relative protein expression.

TABLE 1
Quantification of proteins present in membrane vesicles, MV_{L}, from serotype
4 strain TIGR4 and microparticles, MP, from serotype 4 and 3. Percentage
sequence identities of protein homologues in reference strains for serotype
1 (P1031) and serotype 3 (A66) are shown. The amounts are indicated
in μg/μg total protein or relative amount in percentage.

| | | MP | | Homology in | Homology in |
|---|---|---|---|---|---|
| Protein | MV_{L} | (TIGR4) | MP (Type 3) | P1031* | A66** |
| Ply | 0.055 μg | 0.406 μg | 0.314 μg | 99% | 99% |
| LytA | 0.050 μg | 0.100 μg | 0.266 μg | 99% | 100% |
| PspC | 0.118 μg | 0.300 μg | Not determined* | 85% (PspC | 45% (PspC |
| (PspC3.4) | | | | 2.1) | 11.4) |
| RrgB | 0.016 μg | 0.028 μg | Absent | Absent | Absent |
| PhtD | 100% | 266% | 5793% | 95% | 87% |

*Because of the high genetic diversity of the pspC gene among pneumococcal serotypes it was not possible to use the anti TIGR4 PspC antibody (which was specific for the TIGR4 strain) to detect the serotype 3 PspC

Thus, in a first aspect of the present invention, there is provided isolated Streptococcus pneumoniae microparticle (MP) (a novel type of pneumococcal membrane vesicle particle), wherein said MP comprise either one, two, three or four of the following proteins at the designated levels:
i. the protein Ply at the level of >0.070 μg/mg total protein in the MP;
ii. the protein LytA at the level of ≥0.070 μg/mg total protein in the MP;
iii. the protein PspC at the level of >0.130 μg/mg total protein in the MP; and/or
iv. the protein RrgB at the level of >0.020 μg/mg total protein in the MP.

The microparticles may further comprise capsular polysaccharides of a capsular serotype of Streptococcus pneumoniae, preferably at a level of ≥0.001, more preferably ≥0.01, most preferably ≥0.1 μg/mg total protein in the MP.

The microparticle may comprise the protein Ply at the level of ≥0.070 μg/mg total protein in the MP. The microparticle may comprise the protein Ply at the level of ≥0.15, preferably ≥0.2, more preferably ≥0.3, most preferably ≥0.35 μg/mg total protein in the MP.

The microparticle may comprise the protein LytA at the level of ≥0.070 μg/mg total protein in the MP. The microparticle may comprise the protein LytA at the level of ≥0.08, preferably ≥0.09, yet more preferably ≥0.1 μg/mg, most preferably ≥0.2 μg/mg total protein in the MP.

The microparticle, MP, may comprise the protein PspC at the level of ≥0.130 μg/mg total protein in the MP. The microparticle may comprise comprising the protein PspC at the level of ≥0.15, preferably ≥0.18, more preferably ≥0.2, most preferably ≥0.3 μg/mg total protein in the MP.

The microparticle may comprise the protein RrgB at the level of ≥0.02 μg/mg total protein in the MP. The microparticle may comprise the protein RgrB at the level of ≥0.022, preferably ≥0.025, most preferably ≥0.028 μg/mg total protein in the MP.

The microparticle may further comprise the protein PhtD. The protein PhtD may be present at a level being at least 2-fold, more preferably at least 2.5-fold compared to Streptococcus pneumoniae membrane vesicles obtained in liquid culture in terms of μg/mg total protein in the particle or the vesicle, respectively.

The microparticle may further comprise the protein RrgA. The protein RrgA may be present at the level of ≥0.02, preferably ≥0.05, more preferably ≥0.10, most preferably ≥0.2 μg/mg total protein in the MP.

The microparticle may further comprise the Streptococcus pneumoniae protein IgA. The protein IgA may be present at the level of ≥0.02, preferably ≥0.05, more preferably ≥0.10, most preferably ≥0.2 μg/mg total protein in the MP.

The protein Ply may comprise a sequence having at least 70%, preferably 80%, more preferably 85%, yet more preferably 90%, still more preferably 95%, most preferably 100% sequence identity to SEQ ID NO: 1.

The protein LytA may comprise a sequence having at least 70%, preferably 80%, more preferably 85%, yet more preferably 90%, still more preferably 95%, most preferably 100% sequence identity to SEQ ID NO: 2.

The protein PspC may comprise a sequence having at least 40% or 70%, preferably 80%, more preferably 85%, yet more preferably 90%, still more preferably 95%, most preferably 100% sequence identity to SEQ ID NOs: 3.

The protein PspC may comprise a sequence having at least 70%, preferably 80%, more preferably 85%, yet more preferably 90%, still more preferably 95%, most preferably 100% sequence identity to any one of SEQ ID NOs: 3-14.

The protein RgrB may comprise a sequence having at least 70%, preferably 80%, more preferably 85%, yet more preferably 90%, still more preferably 95%, most preferably 100% sequence identity to SEQ ID NO: 15.

The protein PhtD may comprise a sequence having at least 70%, preferably 80%, more preferably 85%, yet more preferably 90%, still more preferably 95%, most preferably 100% sequence identity to SEQ ID NO: 16.

The protein RgrA may comprise a sequence having at least 70%, preferably 80%, more preferably 85%, yet more preferably 90%, still more preferably 95%, most preferably 100% sequence identity to SEQ ID NO: 17.

The protein IgA may comprise a sequence having at least 70%, preferably 80%, more preferably 85%, yet more preferably 90%, still more preferably 95%, most preferably 100% sequence identity to SEQ ID NO: 18.

Thus, the first aspect encompasses (but is not limited to) embodiments disclosed in Table 2 below.

TABLE 2
Minimum levels of designated proteins in various embodiments of the
first aspect. A blank cell indicates that the protein is optional i.e.
may be absent or present at any level. Units: μg/μg total protein,
except for PhtD, where the amount is in comparing to Streptococcus
pneumoniae membrane vesicles obtained in liquid culture in terms of
μg/μg total protein in the particle or the vesicle, respectively.

| Embodiment# | Ply | LytA | PspC | PhtD | RgrB |
|---|---|---|---|---|---|
| 1 | 0.070 | | | | |
| 2 | 0.070 | 0.070 | | | |
| 3 | 0.070 | | 0.130 | | |
| 4 | 0.070 | | | 2-FOLD | |
| 5 | 0.070 | 0.070 | 0.130 | | |
| 6 | 0.070 | 0.070 | | 2-FOLD | |
| 7 | 0.070 | 0.070 | 0.130 | 2-FOLD | |
| 8 | 0.070 | | 0.130 | 2-FOLD | |
| 9 | | 0.070 | | | |
| 10 | | | 0.130 | | |
| 11 | | | | 2-FOLD | |
| 12 | | 0.070 | 0.130 | | |
| 13 | | 0.070 | | 2-FOLD | |
| 14 | | 0.070 | 0.130 | 2-FOLD | |
| 15 | | | 0.130 | 2-FOLD | |
| 16 | 0.070 | | | | 0.020 |
| 17 | 0.070 | 0.070 | | | 0.020 |
| 18 | 0.070 | | 0.130 | | 0.020 |
| 19 | 0.070 | | | 2-FOLD | 0.020 |
| 20 | 0.070 | 0.070 | 0.130 | | 0.020 |
| 21 | 0.070 | 0.070 | | 2-FOLD | 0.020 |
| 22 | 0.070 | 0.070 | 0.130 | 2-FOLD | 0.020 |
| 23 | 0.070 | | 0.130 | 2-FOLD | 0.020 |
| 24 | | 0.070 | | | 0.020 |
| 25 | | | 0.130 | | 0.020 |
| 26 | | | | 2-FOLD | 0.020 |
| 27 | | 0.070 | 0.130 | | 0.020 |
| 28 | | 0.070 | | 2-FOLD | 0.020 |
| 29 | | 0.070 | 0.130 | 2-FOLD | 0.020 |
| 30 | | | 0.130 | 2-FOLD | 0.020 |
| 31 | 0.070 | | | | |
| 32 | 0.070 | 0.070 | | | |
| 33 | 0.070 | | 0.130 | | |
| 34 | 0.070 | | | 2-FOLD | |
| 35 | 0.070 | 0.070 | 0.130 | | |
| 36 | 0.070 | 0.070 | | 2-FOLD | |
| 37 | 0.070 | 0.070 | 0.130 | 2-FOLD | |
| 38 | 0.070 | | 0.130 | 2-FOLD | |
| 39 | | 0.070 | | | |
| 40 | | | 0.130 | | |
| 41 | | | | 2-FOLD | |
| 42 | | 0.070 | 0.130 | | |
| 43 | | 0.070 | | 2-FOLD | |
| 44 | | 0.070 | 0.130 | 2-FOLD | |
| 45 | | | 0.130 | 2-FOLD | |
| 46 | 0.070 | | | | 0.020 |
| 47 | 0.070 | 0.070 | | | 0.020 |
| 48 | 0.070 | | 0.130 | | 0.020 |
| 49 | 0.070 | | | 2-FOLD | 0.020 |
| 50 | 0.070 | 0.070 | 0.130 | | 0.020 |
| 51 | 0.070 | 0.070 | | 2-FOLD | 0.020 |
| 52 | 0.070 | 0.070 | 0.130 | 2-FOLD | 0.020 |
| 53 | 0.070 | | 0.130 | 2-FOLD | 0.020 |
| 54 | | 0.070 | | | 0.020 |
| 55 | | | 0.130 | | 0.020 |
| 56 | | | | 2-FOLD | 0.020 |
| 57 | | 0.070 | 0.130 | | 0.020 |
| 58 | | 0.070 | | 2-FOLD | 0.020 |
| 59 | | 0.070 | 0.130 | 2-FOLD | 0.020 |
| 60 | | | 0.130 | 2-FOLD | 0.020 |

The microparticles may be 5-300 nm in diameter, preferably 10-125 nm in diameter.

The microparticles may be derived from any Streptococcus pneumoniae strain, but is preferably selected from a group consisting of all serotype 3 strains, all serotype 1 strains, TIGR4, P1031 and A66, most preferably TIGR4.

The microparticles according to the first aspect may be obtainable by:
a. culturing host cells of a Streptococcus pneumoniae strain on blood agar plates or other plates;
b. harvesting the cultured host cells;
c. centrifuging the harvested host cells at 17,000 g for 30 minutes at +4° C.;
d. subjecting the supernatant to filtration through a 0.22 μm filter;
e. centrifuging the filtered supernatant at 120,000 g for 2 h at +4° C.;
f. washing the pellets from e) twice in phosphate-buffered saline with sedimentations at 120,000 g for 2 h at +4° C.;
g. resuspending the pellet in 1 ml phosphate-buffered saline;
h. adjusting the resuspended pellet to 50% (w/v) Optiprep™ density gradient medium in a total volume of 2 ml and overlaying with one fraction of 30% (w/v) Optiprep™ (9 ml) followed by a fraction of 5% (w/v) Optiprep™ (3 ml);
i. centrifuging the gradients at 250,000×g for 3 hours at 4° C.;
j. collecting the 4 ml top fraction, containing the membrane microparticles;
k. washing the microparticles three times phosphate-buffered saline with sedimentations of 250,000×g for 2 hours at +4° C.

### Compositions and Uses

In a second aspect of the present invention, there is provided a composition comprising a microparticle according to the first aspect. The composition may be devoid of whole Streptococcus pneumoniae cells. The composition may optionally comprise capsular polysaccharides from Streptococcus pneumoniae.

The composition according to the second aspect may comprise MP in an amount of 1 μg/ml, preferably 5 μg/ml, more preferably 10 μg/ml, most preferably 100 μg/ml (the concentrations refer to the total protein content of MP per ml).

The composition may further comprise an adjuvant. The adjuvant preferably comprises aluminium hydroxide.

The composition may be an immunogenic composition. Preferably, the immunogenic composition is capable of eliciting opsonophagocitic antibodies against Streptococcus pneumoniae when administered to a mammalian host. Preferably, the immunogenic composition is capable of eliciting serotype independent antibodies against Streptococcus pneumoniae when administered to a mammalian host. Preferably, the immunogenic composition is capable of eliciting antibodies against Streptococcus pneumoniae serotype 3 when administered to a mammalian host.

The composition may be formulated as a vaccine.

In a third aspect of the present invention, there is provided a composition according to the second aspect, for use in a method for inducing protective immunity against Streptococcus pneumoniae in a subject. The third aspect also encompasses a method for inducing protective immunity against Streptococcus pneumoniae in a subject in need thereof, comprising administering an effective amount of the composition according to the second aspect to the subject. The third aspect also encompasses the use of a composition according to the second aspect in the manufacture of a vaccine for immunization against Streptococcus pneumoniae.

The protective immunity may be an immunity reducing the likelihood of a condition selected from pneumococcal sinusitis, pneumococcal otitis, pneumococcal pneumonia and invasive pneumococcal disease including but not limited to pneumococcal sepsis and pneumococcal meningitis, preferably invasive pneumococcal disease. Preferably, the subject to be immunized is a young child (e.g. less than 7 years of age) or an elderly person (e.g. over 65 years of age), but also other age groups could be targeted.

For immunization, the composition may be administered to the subject in various manners known in the art, including but not limited to by way of injection (e.g. intramuscular, intracutaneous, subcutaneous, intravenous), buccal, oral and intranasal administration as well as inhalation.

In a fourth aspect there is provided a use of a microparticle according to the first aspect in an immunogenic composition. The immunogenic composition may be a vaccine.

In a fifth aspect, there is provided a use of a microparticle according to the first aspect in the manufacture of a vaccine.

In a sixth aspect, there is provided a method for manufacturing a vaccine, comprising:
a. Providing a microparticle according to the first aspect
b. Providing an adjuvant, such as aluminium hydroxide;
c. Mixing the microparticle and the adjuvant in a suitable vehicle in order to produce a vaccine.
   Methods for Producing Streptococcus pneumoniae Microparticles

In a sixth aspect of the present invention there is provided a method for producing an isolated Streptococcus pneumoniae microparticle according to the first aspect, comprising:
a. providing a Streptococcus pneumoniae bacterial cell;
b. culturing said bacterial cell under conditions allowing the production of a microparticle according to the first aspect by the bacterial cell; and
c. isolating the microparticle thus produced.

The bacterial cell may be from any pneumococcal strain, but is preferably TIGR4, a serotype 1 and/or a serotype 3 strain.

The culturing may be carried out using plates or liquid culture. Preferably, the culturing is carried out in solid phase, most preferably on blood agar or other plates. Isolating the microparticles may comprise a density gradient centrifugation step.

### General Aspects Relating to the Present Disclosure

The term “comprising” is to be interpreted as including, but not being limited to. All references are hereby incorporated by reference. The arrangement of the present disclosure into sections with headings and subheadings is merely to improve legibility and is not to be interpreted limiting in any way, in particular, the division does not in any way preclude or limit combining features under different headings and subheadings with each other.

### EXAMPLES

The following examples are not to be regarded as limiting. For further information on the experimental details, the skilled reader is directed to a separate section titled Materials and Methods.

### Example 1: Isolation and In Vitro Characterization of Novel Pneumococcal Microparticles

S. pneumoniae TIGR4 Produces Microparticles that Differ Depending on Bacterial Growth Conditions.

Membrane vesicles were isolated and purified from Streptococcus pneumoniae serotype 4 strain TIGR4 grown in liquid medium (MV_{L}). Microparticels (MP) were isolated from pneumococci grown on blood agar plates. Preparations were analyzed by transmission electron microscopy (TEM) and atomic force microscopy, and revealed spherical bodies surrounded by membranous structures (FIG. 1A). The distribution of the size varied between MV_{L}, MP from TIGR4 and MP from serotype 3 (FIG. 1B). In total 503 MV_{L}, 378 MP from TIGR4 and 523 MP from serotype 3 were analyzed. As a control we also studied an isogenic pneumolysin mutant strain (TIGR4Δply) and found a similar size distribution. To reveal shedding of MV_{L} or MP from the surface of bacteria by TEM we then used an isogenic unencapsulated mutant in TIGR4 (T4R) grown in liquid culture. In the absence of the thick bacterial capsule, MV_{L} were clearly visible, emanating from the plasma membrane of S. pneumoniae into the extracellular milieu (FIG. 1C-D).

Proteomic Analysis Revealed Differences in Protein Content Between MV_{L} from Liquid Culture or MP from Plates

To analyze the protein content of both pneumococcal preparations, purified MV_{L} and MP were subjected to SDS-PAGE analysis. Distinct protein patterns in MV_{L} and MPs as compared with proteins present in whole cell lysates or bacterial supernatants (FIG. 2A), were identified. Interestingly, the proteomic content in MV_{L} and MP fractions differed, suggesting a differential enrichment of proteins in the different types of particles. We further conducted proteomic analysis using tandem mass spectrometry. In total, in TIGR4, we identified 317 proteins in MV_{L} (Table 3), and 216 in MP (Table 4), of which 184 proteins were found in both preparations. 133 proteins were unique to MV_{L} and 32 to MP (FIG. 2B). In serotype 3 we identified 462 proteins in MV_{L} (Table 5) and 344 in MP (Table 6). All detected proteins were further classified according to their subcellular localization based on the combination of LocateP and GO annotation (FIG. 2C) (11). A majority of the proteins identified in MV_{L} and MP were cytosolic proteins, followed by multi-transmembrane proteins, lipoproteins, N-terminally anchored proteins, secretory proteins and cell wall-associated proteins. While MV_{L} appeared to be more enriched in cytosolic protein and cell wall-associated proteins than MP from TIGR4, the latter harbored a higher percentage of multi-transmembrane proteins, lipoproteins, N-terminally anchored proteins and secretory proteins.

We further analyzed the mass spectrometry data in order to determine the presence of 30 known pneumococcal virulence factors in the particles. Both types of particles contained approximately half of these virulence factors present in TIGR4. Just two were unique to MV_{L} CbpD (12) and BgaA (13), and one to MP, IgA (10).

Based on the proteomic results, we next performed immunoblot analysis on specific pneumococcal proteins and virulence factors (FIG. 2D). Among cytosolic proteins we investigated the presence of the cytotoxin pneumolysin (Ply), which is a pore-forming toxin and one of the most important virulence factors in pneumococcal pathogenesis (2), GAPDH as a cytosolic marker (14), and LytA, the major autolysin of Streptococcus pneumoniae (3). The most striking observation was the high enrichment of Ply especially in MP, which also contained more LytA than MV_{L}. Smaller differences were also observed in the enrichment of virulence factors belonging to other subcellular localizations. While MV_{L} displayed a higher amount of Pneumococcal surface antigen A (PsaA) (15) and Sortase A (SrtA) (16) than MP, the latter contained more Pneumococcal surface protein C (PspC) (17) and Polyhistidine triad protein D (PhtD) (9) (FIG. 2D). The major component of the pneumococcal pilus, RrgB (6), was equally found in both particle preparations.

These findings suggest the presence of a still uncharacterized mechanism for the selective secretion and enrichment of specific proteins and virulence factors of Streptococcus pneumoniae in these particle preparations.

### A Biologically Active Pneumolysin was Found in Membrane Microparticles

The enrichment of Ply in both MV_{L} and MP prompted us to determine whether particle-associated Ply was functional and able to lyse erythrocytes in a hemolytic assay. For both types of particles we observed a dose dependent hemolysis (FIG. 3), however, Ply from MP showed higher activity than from MV_{L}, which is consistent with the observation that MP contain more Ply. Notably, hemolysis seemed to be entirely caused by Ply, and particles prepared from a TIGR4 mutant lacking Ply (TIGR4Δply) exhibited no hemolytic activity.

### MP are Taken Up by A549 Epithelial Cells, but are not Cytotoxic.

MV in other Gram-positive bacteria have been described to function as vehicle to deliver vesicle-associated proteins to human cells (18) (19). To test whether pneumococcal MP can deliver pneumococcal proteins, we incubated MP from TIGR4 with A549 lung epithelial cells for 24 hours and visualized Ply and LytA by immunofluorescence staining (FIG. 4A-C). By analyzing orthogonal views of A549 cells treated with MP, we showed by immunofluorescence that MP can be taken up, with Ply and LytA detectable inside the cells (FIG. 4B-C).

To confirm our observation we further analyzed lysates of A549 cells treated with MP by immunoblotting with anti-Ply antibody (FIG. 4D). In order to minimize extracellular associated particles, treated cells were excessively washed before lysates were made. Consistent with our immunofluorescence results, western blotting revealed a dose dependent increase in Ply further suggesting that MP can be internalized.

Next we asked whether proteins associated with MV_{L} or MP, and in particular Ply, may contribute to cytotoxic effects on cultured human cells. To assess toxicity, A549 cells were treated with different concentrations of MV_{L} and MP for 24 hours, stained with fixable viability dye and analyzed by flow cytometry (FIG. 4E). The difference in cell death between samples incubated with MV_{L} or MPs and the negative control sample was not statistically significant, suggesting that Ply is not cytotoxic to the cells at concentrations present in MV_{L} or MP.

MV_{L} and MP are Internalized by Human Monocyte-Derived Dendritic Cells (DCs) and Induce their Maturation.

Since we found that MP can be internalized by epithelial cells, we then studied potential interactions of MV_{L} or MP with innate immune cells, focusing on DCs. First we assessed cytotoxicity by incubating DCs with MV_{L} or MP, stained with fixable viability dye and Annexin V, and then analyzed using flow cytometry (FIG. 5A). No significant increase in cytotoxicity was detected in samples incubated with MP, while addition of MV_{L} displayed cytotoxic effects at the two highest concentrations used. While both preparations were considerably less cytotoxic than live bacteria, with 70-80% cytotoxic cells after 24 hours of incubation, more studies are needed to assess the mechanism behind the increased cytotoxicity of MV_{L} from liquid cultures. Moreover, the percentage of apoptotic cells did not change after incubation with either of the particle preparations.

Then we investigated uptake of MP by DCs. First we incubated DCs with MP for 1 hour, lysed the cells and performed SDS-PAGE and immunoblotting on cell lysates to check for presence of Ply. Indeed, Ply was found in the cell lysates (FIG. 5B), suggesting that DCs internalize MP. Next, DCs were challenged for 30 minutes with inhibitors of actin polymerization and phosphatidylinositol 3-kinase (PI3K), cytochalasin and wortmannin, to block active phagocytosis, or with an inhibitor of lipid rafts, methyl-β-cyclodextrin, to block fusion of membranes. Subsequently, cells were incubated with MP for 1 hour, lysed, and the same protocol for immunoblotting was followed. Interestingly, blocking active phagocytosis or membrane fusion resulted in less Ply. However, when all inhibitors were used together, Ply still remained present in the lysates, suggesting either that the particles adhered to the cells or were internalized by another mechanism, not investigated in this study.

Taken together these data suggest that MP can function as a vehicle to deliver bacterial components to host cells. Since DCs are professional antigen presenting cells, we then assessed whether MV_{L} or MP could influence DC maturation. Cells were incubated for 24 hours with MV_{L} or MP, and then stained for MHCII and the co-stimulatory molecule CD86, and analyzed by flow cytometry (FIG. 5C). A dose-dependent increase in the presence of both markers on the surface of DCs was detected when cells were incubated with the particles. MV_{L} appeared to induce more maturation than MP, and similar levels of maturation were obtained with LPS.

### MV_{L} and MP Induce Pro-Inflammatory Cytokine Responses in DCs

Since OMVs from Gram-negative have been shown to trigger a potent innate immune response (20) (21), we studied whether also pneumococcal MV_{L} or MP could affect pro-inflammatory cytokine responses. DCs we incubated with MV_{L} or MP for 24 hours, and ELISA assays were used on supernatants to determine concentrations of IL-6 (FIG. 6A), IL-8 (FIG. 6B), IL-10 (FIG. 6C) and TNF (FIG. 6D). MP induced release of IL-8 and low levels of TNF, while no induction was seen for IL-6 and IL-10. In contrast, MV_{L} induced all four cytokines tested to levels higher than live bacteria. None of the particles induced IL-1β or IL-12 (data not shown).

TABLE 3
Proteins identified in MV_{L} from TIGR4. The accession
numbers refer to SwissProt on the date of filing.

| Accession | Coverage | # Peptides | #AAs | Score | Description | Localization |
|---|---|---|---|---|---|---|
| Q97RH0 | 76.86 | 47 | 350 | 2615.53 | Lipoprotein OS = Streptococcus | Lipid |
| | | | | | pneumoniae serotype 4 (strain ATCC | anchored |
| | | | | | BAA-334/TIGR4) GN = SP_0845 PE = 4 | |
| | | | | | SV = 1 − [Q97RH0_STRPN] | |
| Q2MGF6 | 82.45 | 59 | 490 | 1620.74 | Lysozyme OS = Streptococcus | Secretory |
| | | | | | pneumoniae serotype 4 (strain ATCC | (released) |
| | | | | | BAA-334/TIGR4) GN = lytC PE = 4 | (with CS) |
| | | | | | SV = 1 − [Q2MGF6_STRPN] | |
| Q97NK0 | 73.39 | 64 | 883 | 1364.43 | Aldehyde-alcohol dehydrogenase | Intracellular |
| | | | | | OS = Streptococcus pneumoniae | |
| | | | | | serotype 4 (strain ATCC BAA-334/ | |
| | | | | | TIGR4) GN = SP_2026 PE = 3 SV = 1 − | |
| | | | | | [Q97NK0_STRPN] | |
| P18791 | 72.99 | 48 | 659 | 1307.17 | Oligopeptide-binding protein AmiA | Lipid |
| | | | | | OS = Streptococcus pneumoniae | anchored |
| | | | | | serotype 4 (strain ATCC BAA-334/ | |
| | | | | | TIGR4) GN = amiA PE = 1 SV = 3 − | |
| | | | | | [AMIA_STRPN] | |
| Q9L7Q2 | 55.82 | 91 | 1906 | 1166.89 | Zinc metalloprotease ZmpB | Multi- |
| | | | | | OS = Streptococcus pneumoniae | trans- |
| | | | | | serotype 4 (strain ATCC BAA-334/ | membrane |
| | | | | | TIGR4) GN = zmpB PE = 3 SV = 2 − | |
| | | | | | [ZMPB_STRPN] | |
| Q97T80 | 58.3 | 87 | 1856 | 1012.57 | Zinc metalloprotease ZmpC | LP × TG |
| | | | | | OS = Streptococcus pneumoniae | Cell-wall |
| | | | | | serotype 4 (strain ATCC BAA-334/ | anchored |
| | | | | | TIGR4) GN = zmpC PE = 3 SV = 1 − | |
| | | | | | [ZMPC_STRPN] | |
| Q97NQ8 | 59.59 | 75 | 1225 | 936.07 | DNA-directed RNA polymerase subunit | Intracellular |
| | | | | | beta′ OS = Streptococcus pneumoniae | |
| | | | | | serotype 4 (strain ATCC BAA-334/ | |
| | | | | | TIGR4) GN = rpoC PE = 3 SV = 1 − | |
| | | | | | [RPOC_STRPN] | |
| P59213 | 74.47 | 48 | 423 | 838.2 | Maltose/maltodextrin-binding protein | Lipid |
| | | | | | OS = Streptococcus pneumoniae | anchored |
| | | | | | serotype 4 (strain ATCC BAA-334/ | |
| | | | | | TIGR4) GN = malX PE = 1 SV = 1 − | |
| | | | | | [MALX_STRPN] | |
| P64022 | 75.61 | 46 | 693 | 803.77 | Elongation factor G OS = Streptococcus | Intracellular |
| | | | | | pneumoniae serotype 4 (strain ATCC | |
| | | | | | BAA-334/TIGR4) GN = fusA PE = 3 | |
| | | | | | SV = 1 − [EFG_STRPN] | |
| P0C2J9 | 92.99 | 46 | 471 | 753.38 | Pneumolysin OS = Streptococcus | Intracellular |
| | | | | | pneumoniae serotype 4 (strain ATCC | |
| | | | | | BAA-334/TIGR4) GN = ply PE = 3 SV = 1 − | |
| | | | | | [TACY_STRPN] | |
| Q97N55 | 75 | 27 | 392 | 729.12 | Secreted 45 kd protein | Secretory |
| | | | | | OS = Streptococcus pneumoniae | (released) |
| | | | | | serotype 4 (strain ATCC BAA-334/ | (with CS) |
| | | | | | TIGR4) GN = usp45 PE = 4 SV = 1 − | |
| | | | | | [Q97N55_STRPN] | |
| Q97RY6 | 42.1 | 73 | 2140 | 715.85 | Serine protease, subtilase family | LP × TG |
| | | | | | OS = Streptococcus pneumoniae | Cell-wall |
| | | | | | serotype 4 (strain ATCC BAA-334/ | anchored |
| | | | | | TIGR4) GN = SP_0641 PE = 4 SV = 1 − | |
| | | | | | [Q97RY6_STRPN] | |
| P64030 | 68.84 | 32 | 398 | 684.61 | Elongation factor Tu OS = Streptococcus | Intracellular |
| | | | | | pneumoniae serotype 4 (strain ATCC | |
| | | | | | BAA-334/TIGR4) GN = tuf PE = 3 SV = 1 − | |
| | | | | | [EFTU_STRPN] | |
| Q97NQ7 | 66.42 | 61 | 1203 | 683.01 | DNA-directed RNA polymerase subunit | Intracellular |
| | | | | | beta OS = Streptococcus pneumoniae | |
| | | | | | serotype 4 (strain ATCC BAA-334/ | |
| | | | | | TIGR4) GN = rpoB PE = 3 SV = 1 − | |
| | | | | | [RPOB_STRPN] | |
| Q97PE6 | 77.38 | 35 | 442 | 645.41 | Sugar ABC transporter, sugar-binding | Lipid |
| | | | | | protein OS = Streptococcus pneumoniae | anchored |
| | | | | | serotype 4 (strain ATCC BAA-334/ | |
| | | | | | TIGR4) GN = SP_1683 PE = 4 SV = 1 − | |
| | | | | | [Q97PE6_STRPN] | |
| Q97PT1 | 74.59 | 45 | 551 | 641.52 | Uncharacterized protein | Intracellular |
| | | | | | OS = Streptococcus pneumoniae | |
| | | | | | serotype 4 (strain ATCC BAA-334/ | |
| | | | | | TIGR4) GN = SP_1518 PE = 4 SV = 1 − | |
| | | | | | [Q97PT1_STRPN] | |
| Q97RQ0 | 81.35 | 26 | 386 | 599.79 | Branched-chain amino acid ABC | Lipid |
| | | | | | transporter, amino acid-binding | anchored |
| | | | | | protein OS = Streptococcus pneumoniae | |
| | | | | | serotype 4 (strain ATCC BAA-334/ | |
| | | | | | TIGR4) GN = livJ PE = 4 SV = 1 − | |
| | | | | | [Q97RQ0_STRPN] | |
| Q97SV2 | 62.45 | 19 | 277 | 575.15 | 50 S ribosomal protein L2 OS = | Intracellular |
| | | | | | Streptococcus pneumoniae serotype 4 | |
| | | | | | (strain ATCC BAA-334/TIGR4) | |
| | | | | | GN = rplB PE = 3 SV = 1 − [RL2_STRPN] | |
| Q97R51 | 74.12 | 32 | 313 | 574.61 | Foldase protein PrsA OS = | Lipid |
| | | | | | Streptococcus pneumoniae serotype 4 | anchored |
| | | | | | (strain ATCC BAA-334/TIGR4) | |
| | | | | | GN = prsA PE = 3 SV = 1 − [PRSA_STRPN] | |
| Q97SV5 | 59.62 | 17 | 208 | 571.92 | 50 S ribosomal protein L3 OS = | Intracellular |
| | | | | | Streptococcus pneumoniae serotype 4 | |
| | | | | | (strain ATCC BAA-334/TIGR4) | |
| | | | | | GN = rpIC PE = 3 SV = 1 − [RL3_STRPN] | |
| Q04707 | 64.12 | 38 | 719 | 564.47 | Penicillin-binding protein 1 A OS = | N- |
| | | | | | Streptococcus pneumoniae serotype 4 | terminally |
| | | | | | (strain ATCC BAA-334/TIGR4) | anchored |
| | | | | | GN = ponA PE = 1 SV = 2 − [PBPA_STRPN] | (No CS) |
| Q97NL1 | 88.36 | 26 | 335 | 546.25 | Glyceraldehyde-3-phosphate | Intracellular |
| | | | | | dehydrogenase OS = Streptococcus | |
| | | | | | pneumoniae serotype 4 (strain ATCC | |
| | | | | | BAA-334/TIGR4) GN = gap PE = 3 SV = 1 − | |
| | | | | | [Q97NL1_STRPN] | |
| Q97PD6 | 64.87 | 46 | 837 | 501.57 | Protein translocase subunit SecA 1 | Intracellular |
| | | | | | OS = Streptococcus pneumoniae | |
| | | | | | serotype 4 (strain ATCC BAA-334/ | |
| | | | | | TIGR4) GN = secA1 PE = 3 SV = 1 − | |
| | | | | | [SECA1_STRPN] | |
| P35592 | 75.61 | 44 | 660 | 500.22 | Oligopeptide-binding protein AliA OS = | Lipid |
| | | | | | Streptococcus pneumoniae serotype 4 | anchored |
| | | | | | (strain ATCC BAA-334/TIGR4) | |
| | | | | | GN = aliA PE = 3 SV = 4 − [ALIA_STRPN] | |
| P0A4G2 | 72.49 | 21 | 309 | 482.72 | Manganese ABC transporter | Lipid |
| | | | | | substrate-binding lipoprotein OS = | anchored |
| | | | | | Streptococcus pneumoniae serotype 4 | |
| | | | | | (strain ATCC BAA-334/TIGR4) | |
| | | | | | GN = psaA PE = 1 SV = 1 − [MITSA_STRPN] | |
| Q54970 | 61.59 | 38 | 591 | 482.11 | Pyruvate oxidase OS = Streptococcus | Intracellular |
| | | | | | pneumoniae serotype 4 (strain ATCC | |
| | | | | | BAA-334/TIGR4) GN = spxB PE = 3 | |
| | | | | | SV = 2 − [POXB_STRPN] | |
| Q97R09 | 68.62 | 21 | 341 | 442.02 | Iron-compound ABC transporter, iron | Lipid |
| | | | | | compound-binding protein OS = | anchored |
| | | | | | Streptococcus pneumoniae serotype 4 | |
| | | | | | (strain ATCC BAA-334/TIGR4) | |
| | | | | | GN = SP_1032 PE = 1 SV = 1 − | |
| | | | | | [Q97R09_STRPN] | |
| Q97PU3 | 67.63 | 31 | 278 | 434.28 | Amino acid ABC transporter, amino | Lipid |
| | | | | | acid-binding protein OS = Streptococcus | anchored |
| | | | | | pneumoniae serotype 4 (strain ATCC | |
| | | | | | BAA-334/TIGR4) GN = aatB PE = 4 SV = | |
| | | | | | 1 − [Q97PU3_STRPN] | |
| P0A4C3 | 72.35 | 20 | 217 | 427.97 | 30 S ribosomal protein S3 | Intracellular |
| | | | | | OS = Streptococcus pneumoniae | |
| | | | | | serotype 4 (strain ATCC BAA-334/ | |
| | | | | | TIGR4) GN = rpsC PE = 3 SV = 1 − | |
| | | | | | [RS3_STRPN] | |
| Q97T39 | 36.42 | 28 | 744 | 407.46 | Pneumococcal surface protein A | N- |
| | | | | | OS = Streptococcus pneumoniae | terminally |
| | | | | | serotype 4 (strain ATCC BAA-334/ | anchored |
| | | | | | TIGR4) GN = pspA PE = 4 SV = 1 − | (with CS) |
| | | | | | [Q97T39_STRPN] | |
| I6L8V7 | 41.79 | 28 | 627 | 381.01 | Choline binding protein E OS = | Secretory |
| | | | | | Streptococcus pneumoniae serotype 4 | (released) |
| | | | | | (strain ATCC BAA-334/TIGR4) | (with CS) |
| | | | | | GN = cbpE PE = 4 SV = 1 − [16L8V7_STRPN] | |
| P0A475 | 54.74 | 11 | 137 | 354.71 | 50 S ribosomal protein L16 OS = | Intracellular |
| | | | | | Streptococcus pneumoniae serotype 4 | |
| | | | | | (strain ATCC BAA-334/TIGR4) | |
| | | | | | GN = rpIP PE = 1 SV = 1 − [RL16_STRPN] | |
| P66112 | 51.26 | 9 | 119 | 346.49 | 50 S ribosomal protein L20 OS = | Intracellular |
| | | | | | Streptococcus pneumoniae serotype 4 | |
| | | | | | (strain ATCC BAA-334/TIGR4) | |
| | | | | | GN = rpIT PE = 3 SV = 1 − [RL20_STRPN] | |
| Q97SV4 | 56.04 | 14 | 207 | 345.01 | 50 S ribosomal protein L4 OS = | Intracellular |
| | | | | | Streptococcus pneumoniae serotype 4 | |
| | | | | | (strain ATCC BAA-334/TIGR4) | |
| | | | | | GN = rpID PE = 3 SV = 1 − [RL4_STRPN] | |
| Q97T12 | 78.99 | 26 | 276 | 332.96 | ABC transporter, substrate-binding | Lipid |
| | | | | | protein OS = Streptococcus pneumoniae | anchored |
| | | | | | serotype 4 (strain ATCC BAA-334/ | |
| | | | | | TIGR4) GN = SP_0148 PE = 4 SV = 1 − | |
| | | | | | [Q97T12_STRPN] | |
| Q97N74 | 47.47 | 31 | 693 | 320.12 | Choline binding protein A OS = | N- |
| | | | | | Streptococcus pneumoniae serotype 4 | terminally |
| | | | | | (strain ATCC BAA-334/TIGR4) | anchored |
| | | | | | GN = cbpA PE = 1 SV = 1 − | (with CS) |
| | | | | | [Q97N74_STRPN] | |
| Q97QS2 | 58.06 | 22 | 434 | 315.73 | Enolase OS = Streptococcus | Intracellular |
| | | | | | pneumoniae serotype 4 (strain ATCC | |
| | | | | | BAA-334/TIGR4) GN = eno PE = 1 SV = 1 − | |
| | | | | | [ENO_STRPN] | |
| Q97S57 | 49.58 | 34 | 958 | 315.37 | Translation initiation factor IF-2 | Intracellular |
| | | | | | OS = Streptococcus pneumoniae | |
| | | | | | serotype 4 (strain ATCC BAA-334/ | |
| | | | | | TIGR4) GN = infB PE = 3 SV = 1 − | |
| | | | | | [IF2_STRPN] | |
| Q97T11 | 75 | 27 | 284 | 313.6 | Lipoprotein OS = Streptococcus | Lipid |
| | | | | | pneumoniae serotype 4 (strain ATCC | anchored |
| | | | | | BAA-334/TIGR4) GN = SP_0149 PE = 3 | |
| | | | | | SV = 1 − [Q97T11_STRPN] | |
| P66359 | 55.12 | 10 | 127 | 299.97 | 30 S ribosomal protein S11 OS = | Intracellular |
| | | | | | Streptococcus pneumoniae serotype 4 | |
| | | | | | (strain ATCC BAA-334/TIGR4) | |
| | | | | | GN = rpsK PE = 3 SV = 1 − [RS11_STRPN] | |
| Q97PT6 | 68.8 | 24 | 468 | 285.18 | ATP synthase subunit beta OS = | Intracellular |
| | | | | | Streptococcus pneumoniae serotype 4 | |
| | | | | | (strain ATCC BAA-334/TIGR4) | |
| | | | | | GN = atpD PE = 3 SV = 1 − [ATPB_STRPN] | |
| Q97SP2 | 60.24 | 18 | 332 | 279.63 | PTS system, mannose-specific IIAB | Intracellular |
| | | | | | components OS = Streptococcus | |
| | | | | | pneumoniae serotype 4 (strain ATCC | |
| | | | | | BAA-334/TIGR4) GN = manL PE = 4 | |
| | | | | | SV = 1 − [Q97SP2_STRPN] | |
| Q97RZ7 | 60.08 | 15 | 238 | 278.06 | Uncharacterized protein OS = | Lipid |
| | | | | | Streptococcus pneumoniae serotype 4 | anchored |
| | | | | | (strain ATCC BAA-334/TIGR4) | |
| | | | | | GN = SP_0629 PE = 1 SV = 1 − | |
| | | | | | [Q97RZ7_STRPN] | |
| Q97NL3 | 55.13 | 34 | 731 | 275.05 | Penicillin-binding protein 2A OS = | N- |
| | | | | | Streptococcus pneumoniae serotype 4 | terminally |
| | | | | | (strain ATCC BAA-334/TIGR4) | anchored |
| | | | | | GN = pbp2A PE = 4 SV = 1 − | (No CS) |
| | | | | | [Q97NL3_STRPN] | |
| Q97PE1 | 62.92 | 29 | 445 | 269.34 | ABC transporter, substrate-binding | Lipid |
| | | | | | protein OS = Streptococcus | anchored |
| | | | | | pneumoniae serotype 4 (strain ATCC | |
| | | | | | BAA-334/TIGR4) GN = SP_1690 PE = 4 | |
| | | | | | SV = 1 − [Q97PE1_STRPN] | |
| Q97SX2 | 81.48 | 19 | 189 | 268.65 | Uncharacterized protein OS = | Lipid |
| | | | | | Streptococcus pneumoniae serotype 4 | anchored |
| | | | | | (strain ATCC BAA-334/TIGR4) | |
| | | | | | GN = SP_0191 PE = 1 SV = 1 − | |
| | | | | | [Q97SX2_STRPN] | |
| Q97RN2 | 67.79 | 15 | 267 | 248.33 | Peptidyl-prolyl cis-trans isomerase, | Lipid |
| | | | | | cyclophilin-type OS = Streptococcus | anchored |
| | | | | | pneumoniae serotype 4 (strain ATCC | |
| | | | | | BAA-334/TIGR4) GN = SP_0771 PE = 3 | |
| | | | | | SV = 1 − [Q97RN2_STRPN] | |
| P65887 | 62.62 | 25 | 428 | 243.93 | Adenylosuccinate synthetase OS = | Intracellular |
| | | | | | Streptococcus pneumoniae serotype 4 | |
| | | | | | (strain ATCC BAA-334/TIGR4) | |
| | | | | | GN = purA PE = 1 SV = 1 − [PURA_STRPN] | |
| Q97QH2 | 33.7 | 23 | 721 | 242.51 | Amino acid ABC transporter, amino | Multi- |
| | | | | | acid-binding protein/permease | trans- |
| | | | | | protein OS = Streptococcus | membrane |
| | | | | | pneumoniae serotype 4 (strain ATCC | |
| | | | | | BAA-334/TIGR4) GN = SP_1241 PE = 1 | |
| | | | | | SV = 1 − [Q97QH2_STRPN] | |
| O69076 | 50.46 | 25 | 652 | 240.51 | ATP-dependent zinc metalloprotease | Multi- |
| | | | | | FtsH OS = Streptococcus pneumoniae | trans- |
| | | | | | serotype 4 (strain ATCC BAA-334/ | membrane |
| | | | | | TIGR4) GN = ftsH PE = 3 SV = 3 − | |
| | | | | | [FTSH_STRPN] | |
| Q97N37 | 62.6 | 22 | 393 | 236.55 | Serine protease OS = -Streptococcus | N- |
| | | | | | pneumoniae serotype 4 (strain ATCC | terminally |
| | | | | | BAA-334/TIGR4) GN = SP_2239 PE = 1 | anchored |
| | | | | | SV = 1 − [Q97N37_STRPN] | (No CS) |
| Q97T63 | 60.29 | 26 | 491 | 232.33 | ABC transporter, substrate-binding | Lipid |
| | | | | | protein OS = -Streptococcus | anchored |
| | | | | | pneumoniae serotype 4 (strain ATCC | |
| | | | | | BAA-334/TIGR4) GN = SP_0092 PE = 4 | |
| | | | | | SV = 1 − [Q97T63_STRPN] | |
| Q97NQ0 | 68.79 | 19 | 330 | 230.07 | Aspartate--ammonia ligase OS = | Intracellular |
| | | | | | Streptococcus pneumoniae serotype 4 | |
| | | | | | (strain ATCC BAA-334/TIGR4) | |
| | | | | | GN = asnA PE = 3 SV = 1 − [ASNA_STRPN] | |
| Q97SV1 | 86.67 | 12 | 180 | 229.35 | 50 S ribosomal protein L5 OS = | Intracellular |
| | | | | | Streptococcus pneumoniae serotype 4 | |
| | | | | | (strain ATCC BAA-334/TIGR4) | |
| | | | | | GN = rplE PE = 3 SV = 1 − [RL5_STRPN] | |
| Q97Q37 | 69.37 | 18 | 271 | 227.84 | Amino acid ABC transporter, amino | Lipid |
| | | | | | acid-binding protein OS = | anchored |
| | | | | | Streptococcus pneumoniae serotype 4 | |
| | | | | | (strain ATCC BAA-334/TIGR4) | |
| | | | | | GN = SP_1394 PE = 4 SV = 1 − | |
| | | | | | [Q97Q37_STRPN] | |
| P66907 | 42.63 | 9 | 380 | 223.83 | Queuine tRNA-ribosyltransferase OS = | Intracellular |
| | | | | | Streptococcus pneumoniae serotype 4 | |
| | | | | | (strain ATCC BAA-334/TIGR4) GN = tgt | |
| | | | | | PE = 3 SV = 1 − [TGT_STRPN] | |
| P59205 | 43.77 | 20 | 658 | 217.64 | Putative endo-beta-N- | Secretory |
| | | | | | acetylglucosaminidase OS = | (released) |
| | | | | | Streptococcus pneumoniae (strain | (with CS) |
| | | | | | ATCC BAA-334/TIGR4) GN = lytB PE = 1 | |
| | | | | | SV = 1 − [LYTB_STRPN] | |
| Q97SC6 | 49.35 | 27 | 774 | 216.35 | Formate acetyltransferase OS = | Intracellular |
| | | | | | Streptococcus pneumoniae serotype 4 | |
| | | | | | (strain ATCC BAA-334/TIGR4) GN = pfl | |
| | | | | | PE = 4 SV = 1 − [Q97SC6_STRPN] | |
| Q97NB5 | 42.19 | 22 | 621 | 215.44 | Choline binding protein PcpA OS = | Secretory |
| | | | | | Streptococcus pneumoniae serotype 4 | (released) |
| | | | | | (strain ATCC BAA-334/TIGR4) | (with CS) |
| | | | | | GN = pcpA PE = 1 SV = 1 − | |
| | | | | | [Q97NB5_STRPN] | |
| P0A2U8 | 49.58 | 19 | 355 | 215.01 | Oligopeptide transport ATP-binding | Intracellular |
| | | | | | protein AmiE OS = -- Streptococcus | |
| | | | | | pneumoniae serotype 4 (strain ATCC | |
| | | | | | BAA-334/TIGR4) GN = amiE PE = 3 | |
| | | | | | SV = 1 − [AMIE_STRPN] | |
| Q97PA9 | 39 | 21 | 659 | 212.01 | Serine/threonine-protein kinase StkP | Intracellular/ |
| | | | | | OS = -Streptococcus pneumoniae | TMH |
| | | | | | serotype 4 (strain ATCC BAA-334 | start |
| | | | | | TIGR4) GN = stkP PE = 1 SV = 1 − | AFTER 60 |
| | | | | | [STKP2_STRPN] | |
| Q97RF9 | 56.25 | 22 | 400 | 204.01 | Ribosomal protein S1 OS = | Intracellular |
| | | | | | Streptococcus pneumoniae serotype 4 | |
| | | | | | (strain ATCC BAA-334/TIGR4) | |
| | | | | | GN = rpsA PE = 4 SV = 1 − | |
| | | | | | [Q97RF9_STRPN] | |
| Q97SP4 | 43.89 | 12 | 303 | 202.88 | PTS system, mannose-specific IID | Multi- |
| | | | | | component OS = -Streptococcus | trans- |
| | | | | | pneumoniae serotype 4 (strain ATCC | membrane |
| | | | | | BAA-33/TIGR4) GN = SP_0282 PE = 4 | |
| | | | | | SV = 1 − [Q97SP4_STRPN] | |
| P0A4A7 | 58.39 | 11 | 137 | 202.86 | 30 S ribosomal protein S12 OS = | Intracellular |
| | | | | | Streptococcus pneumoniae serotype 4 | |
| | | | | | (strain ATCC BAA-334/TIGR4) | |
| | | | | | GN = rpsL PE = 3 SV = 1 − [RS12_STRPN] | |
| Q97Q31 | 55.82 | 13 | 292 | 202.43 | Phosphate-binding protein PstS 1 OS = | Lipid |
| | | | | | Streptococcus pneumoniae serotype 4 | anchored |
| | | | | | (strain ATCC BAA-334/TIGR4) | |
| | | | | | GN = pstS1 PE = 1 SV = 1 − [PSTS1_STRPN] | |
| Q97N56 | 69.11 | 20 | 259 | 201.44 | 30 S ribosomal protein S2 OS = | Intracellular |
| | | | | | Streptococcus pneumoniae serotype 4 | |
| | | | | | (strain ATCC BAA-334/TIGR4) | |
| | | | | | GN = rpsB PE = 3 SV = 1 − [RS2_STRPN] | |
| Q97RC6 | 48.66 | 14 | 335 | 201.21 | ATP-dependent 6- | Intracellular |
| | | | | | phosphofructokinase OS = | |
| | | | | | Streptococcus pneumoniae serotype 4 | |
| | | | | | (strain ATCC BAA-334/TIGR4) | |
| | | | | | GN = pfkA PE = 3 SV = 1 − [PFKA_STRPN] | |
| Q97QX5 | 68.02 | 15 | 344 | 200.45 | Uncharacterized protein OS = | Secretory |
| | | | | | Streptococcus pneumoniae serotype 4 | (released) |
| | | | | | (strain ATCC BAA-334/TIGR4) | (with CS) |
| | | | | | GN = SP_1069 PE = 1 SV = 1 − | |
| | | | | | [Q97QX5_STRPN] | |
| Q97NM6 | 60.64 | 17 | 404 | 197.5 | Aminotransferase, class I OS = | Intracellular |
| | | | | | Streptococcus pneumoniae serotype 4 | |
| | | | | | (strain ATCC BAA-334/TIGR4) | |
| | | | | | GN = SP_1994 PE = 4 SV = 1 − | |
| | | | | | [Q97NM6_STRPN] | |
| Q97N69 | 65.37 | 19 | 335 | 194.36 | Putative ABC transporter, substrate- | Lipid |
| | | | | | binding protein OS = -Streptococcus | anchored |
| | | | | | pneumoniae serotype 4 (strain ATCC | |
| | | | | | BAA-334/TIGR4) GN = SP_2197 PE = 4 | |
| | | | | | SV = 1 − [Q97N69_STRPN] | |
| Q97NS5 | 60.95 | 16 | 338 | 193.02 | Putative transcriptional regulator OS = | Secretory |
| | | | | | Streptococcus pneumoniae serotype 4 | (released) |
| | | | | | (strain ATCC BAA-334/TIGR4) | (with CS) |
| | | | | | GN = SP_1942 PE = 4 SV = 1 − | |
| | | | | | [Q97NS5_STRPN] | |
| P0A4M7 | 38.35 | 20 | 498 | 189.98 | Oligopeptide transport system | Multi- |
| | | | | | permease protein AmiC OS = = | trans- |
| | | | | | Streptococcus pneumoniae serotype 4 | membrane |
| | | | | | (strain ATCC BAA-334/TIGR4) | |
| | | | | | GN = amiC PE = 3 SV = 1 − [AMIC_STRPN] | |
| Q97QD2 | 56.41 | 20 | 523 | 188.15 | Signal recognition particle protein OS = | Intracellular |
| | | | | | Streptococcus pneumoniae serotype 4 | |
| | | | | | (strain ATCC BAA-334/TIGR4) GN = ffh | |
| | | | | | PE = 1 SV = 1 − [Q97QD2_STRPN] | |
| P14677 | 49.87 | 26 | 750 | 185.33 | Penicillin-binding protein 2 × OS = = | N- |
| | | | | | Streptococcus pneumoniae serotype 4 | terminally |
| | | | | | (strain ATCC BAA-334/TIGR4) | anchored |
| | | | | | GN = pbpX PE = 1 SV = 2 − [PBPX_STRPN] | (No CS) |
| Q97NQ3 | 51.88 | 14 | 345 | 183.26 | Uncharacterized protein OS = | N- |
| | | | | | Streptococcus pneumoniae serotype 4 | terminally |
| | | | | | (strain ATCC BAA-334/TIGR4) | anchored |
| | | | | | GN = SP_1967 PE = 4 SV = 1 − | (No CS) |
| | | | | | [Q97NQ3_STRPN] | |
| Q97RW9 | 59.34 | 16 | 332 | 182.68 | Putative pneumococcal surface | Lipid |
| | | | | | protein OS = -Streptococcus | anchored |
| | | | | | pneumoniae serotype 4 (strain ATCC | |
| | | | | | BAA-334/TIGR4) GN = SP_0667 PE = 1 | |
| | | | | | SV = 1 − [Q97RW9_STRPN] | |
| Q97RL9 | 66.17 | 16 | 399 | 180.86 | Uncharacterized protein OS = | N- |
| | | | | | Streptococcus pneumoniae serotype 4 | terminally |
| | | | | | (strain ATCC BAA-334/TIGR4) | anchored |
| | | | | | GN = SP_0785 PE = 4 SV = 1 − | (No CS) |
| | | | | | [Q97RL9_STRPN] | |
| P0A3M9 | 47.26 | 14 | 328 | 180.34 | L-lactate dehydrogenase OS = | Intracellular |
| | | | | | Streptococcus pneumoniae serotype 4 | |
| | | | | | (strain ATCC BAA-334/TIGR4) GN = Idh | |
| | | | | | PE = 3 SV = 2 − [LDH_STRPN] | |
| Q97PT4 | 39.72 | 17 | 501 | 180.17 | ATP synthase subunit alpha OS = | Multi- |
| | | | | | Streptococcus pneumoniae serotype 4 | trans- |
| | | | | | (strain ATCC BAA-334/TIGR4) | membrane |
| | | | | | GN = atpA PE = 3 SV = 1 − [ATPA_STRPN] | |
| Q97QH1 | 67.48 | 9 | 246 | 175.25 | Amino acid ABC transporter, ATP- | Intracellular |
| | | | | | binding protein OS = -Streptococcus | |
| | | | | | pneumoniae serotype 4 (strain ATCC | |
| | | | | | BAA-334/TIGR4) GN = SP_1242 PE = 3 | |
| | | | | | SV = 1 − [Q97QH1_STRPN] | |
| Q97NX9 | 72.9 | 19 | 321 | 174.95 | Iron-compound ABC transporter, iron- | Lipid |
| | | | | | compound-binding protein OS = | anchored |
| | | | | | Streptococcus pneumoniae serotype 4 | |
| | | | | | (strain ATCC BAA-334/TIGR4) | |
| | | | | | GN = SP_1872 PE = 4 SV = 1 − | |
| | | | | | [Q97NX9_STRPN] | |
| Q97QC6 | 66.09 | 14 | 115 | 173.94 | 50 S ribosomal protein L19 OS = | Intracellular |
| | | | | | Streptococcus pneumoniae serotype 4 | |
| | | | | | (strain ATCC BAA-334/TIGR4) | |
| | | | | | GN = rplS PE = 3 SV = 1 − [RL19_STRPN] | |
| Q97N99 | 63.14 | 18 | 274 | 172.57 | SPFH domain/Band 7 family OS = | Intracellular |
| | | | | | Streptococcus pneumoniae serotype 4 | |
| | | | | | (strain ATCC BAA-334/TIGR4) | |
| | | | | | GN = SP_2156 PE = 4 SV = 1 − | |
| | | | | | [Q97N99_STRPN] | |
| Q97PW1 | 50.97 | 17 | 463 | 169.74 | Peptidoglycan N-acetylglucosamine | N- |
| | | | | | deacetylase A OS = -Streptococcus | terminally |
| | | | | | pneumoniae serotype 4 (strain ATCC | anchored |
| | | | | | BAA-334/TIGR4) GN = pgdA PE = 4 | (No CS) |
| | | | | | SV = 1 − [Q97PW1_STRPN] | |
| Q97SE1 | 36.4 | 14 | 555 | 169.45 | Uncharacterized protein OS = | Intracellular |
| | | | | | Streptococcus pneumoniae serotype 4 | |
| | | | | | (strain ATCC BAA-334/TIGR4) | |
| | | | | | GN = SP_0443 PE = 1 SV = 1 − | |
| | | | | | [Q97SE1_STRPN] | |
| Q97PM5 | 59.04 | 16 | 376 | 168.92 | Sugar ABC transporter, ATP-binding | Intracellular |
| | | | | | protein OS = -Streptococcus | |
| | | | | | pneumoniae serotype 4 (strain ATCC | |
| | | | | | BAA-334/TIGR4) GN = msmK PE = 4 | |
| | | | | | SV = 1 − [Q97PM5_STRPN] | |
| Q97SN5 | 85.81 | 15 | 148 | 165.86 | 50 S ribosomal protein L13 OS = | Intracellular |
| | | | | | Streptococcus pneumoniae serotype 4 | |
| | | | | | (strain ATCC BAA-334/TIGR4) | |
| | | | | | GN = rplM PE = 3 SV = 1 − | |
| | | | | | [Q97SN5_STRPN] | |
| Q97PQ2 | 28.88 | 18 | 914 | 162.85 | Cation-transporting ATPase, E1-E2 | Multi- |
| | | | | | family OS = -Streptococcus pneumoniae | trans- |
| | | | | | serotype 4 (strain ATCC BAA-334/ | membrane |
| | | | | | TIGR4) GN = SP_1551 PE = 3 | |
| | | | | | SV = 1 − [Q97PQ2_STRPN] | |
| Q97NE4 | 32.16 | 21 | 821 | 161.61 | Penicillin-binding protein 1 B OS = | N- |
| | | | | | Streptococcus pneumoniae serotype 4 | terminally |
| | | | | | (strain ATCC BAA-334/TIGR4) | anchored |
| | | | | | GN = pbp1B PE = 4 SV = 1 − | (No CS) |
| | | | | | [Q97NE4_STRPN] | |
| Q97Q62 | 54.48 | 20 | 424 | 161.35 | Psr protein OS = -Streptococcus | Intracellular/ |
| | | | | | pneumoniae serotype 4 (strain ATCC | TMH |
| | | | | | BAA-334/TIGR4) GN = SP_1368 PE = 4 | start |
| | | | | | SV = 1 − [Q97Q62_STRPN] | AFTER 60 |
| P18766 | 50 | 13 | 308 | 160.11 | Oligopeptide transport ATP-binding | Intracellular |
| | | | | | protein AmiF OS = -Streptococcus | |
| | | | | | pneumoniae serotype 4 (strain ATCC | |
| | | | | | BAA-334/TIGR4) GN = amiF PE = 3 | |
| | | | | | SV = 2 − [AMIF_STRPN] | |
| I6L8U0 | 42.39 | 16 | 394 | 155.34 | UDP-N-acetylglucosamine 2- | Intracellular |
| | | | | | epimerase OS = -Streptococcus | |
| | | | | | pneumoniae serotype 4 (strain ATCC | |
| | | | | | BAA-334/TIGR4) GN = cps4L PE = 3 | |
| | | | | | SV = 1 − [I6L8U0_STRPN] | |
| P0CC08 | 55.21 | 15 | 288 | 154.74 | Acetyl-coenzyme A carboxylase | Intracellular |
| | | | | | carboxyl transferase subunit beta OS = | |
| | | | | | Streptococcus pneumoniae serotype 4 | |
| | | | | | (strain ATCC BAA-334/TIGR4) | |
| | | | | | GN = accD PE = 1 SV = 1 − [ACCD_STRPN] | |
| Q97RG9 | 51.27 | 21 | 511 | 154.04 | Sugar ABC transporter, ATP-binding | Intracellular |
| | | | | | protein OS = -Streptococcus | |
| | | | | | pneumoniae serotype 4 (strain ATCC | |
| | | | | | BAA-334/TIGR4) GN = SP_0846 PE = 3 | |
| | | | | | SV = 1 − [Q97RG9_STRPN] | |
| Q97SJ4 | 50.28 | 17 | 360 | 153.5 | Uncharacterized protein OS = | Intracellular |
| | | | | | Streptococcus pneumoniae serotype 4 | |
| | | | | | (strain ATCC BAA-334/TIGR4) | |
| | | | | | GN = SP_0355 PE = 4 | |
| | | | | | SV = 1 − [Q97SJ4_STRPN] | |
| P72524 | 33.82 | 22 | 822 | 148.92 | DNA gyrase subunit A OS = | Intracellular |
| | | | | | Streptococcus pneumoniae serotype 4 | |
| | | | | | (strain ATCC BAA-334/TIGR4) | |
| | | | | | GN = gyrA PE = 3 SV = 3 − [GYRA_STRPN] | |
| P66565 | 56.16 | 17 | 203 | 148.91 | 30 S ribosomal protein S4 OS = | Intracellular |
| | | | | | Streptococcus pneumoniae serotype 4 | |
| | | | | | (strain ATCC BAA-334/TIGR4) | |
| | | | | | GN = rpsD PE = 3 SV = 1 − [RS4_STRPN] | |
| P0A4G0 | 41.1 | 20 | 652 | 146.87 | Oligopeptide-binding protein AliB | Lipid |
| | | | | | OS = = -Streptococcus pneumoniae | anchored |
| | | | | | serotype 4 (strain ATCC BAA-334/ | |
| | | | | | TIGR4) GN = aliB PE = 3 SV = | |
| | | | | | 1 − [ALIB_STRPN] | |
| Q97SG0 | 57.78 | 18 | 424 | 144.79 | Serine--tRNA ligase OS = -Streptococcus | Intracellular |
| | | | | | pneumoniae serotype 4 (strain ATCC | |
| | | | | | BAA-334/TIGR4) GN = serS PE = 3 | |
| | | | | | SV = 1 − [SYS_STRPN] | |
| Q97PF9 | 51.79 | 20 | 419 | 144.46 | Cell division protein FtsZ OS = | Intracellular |
| | | | | | Streptococcus pneumoniae serotype 4 | |
| | | | | | (strain ATCC BAA-334/TIGR4) | |
| | | | | | GN = ftsZ PE = 3 SV = 1 − [Q97PF9_STRPN] | |
| Q97Q34 | 64.04 | 17 | 267 | 144.16 | Phosphate import ATP-binding protein | Intracellular |
| | | | | | PstB 2 OS = -Streptococcus pneumoniae | |
| | | | | | serotype 4 (strain ATCC BAA-334/ | |
| | | | | | TIGR4) GN = pstB2 PE = 3 SV = 1 − | |
| | | | | | [PSTB2_STRPN] | |
| I6L8N1 | 47.25 | 14 | 455 | 141.81 | Acetyl-CoA carboxylase, biotin | Intracellular |
| | | | | | carboxylase OS = -Streptococcus | |
| | | | | | pneumoniae serotype 4 (strain ATCC | |
| | | | | | BAA-334/TIGR4) GN = accC PE = 4 | |
| | | | | | SV = 1 − [I6L8N1_STRPN] | |
| Q97SR1 | 45.54 | 19 | 617 | 140.84 | Proline--tRNA ligase OS = = | Intracellular |
| | | | | | Streptococcus pneumoniae serotype 4 | |
| | | | | | (strain ATCC BAA-334/TIGR4) | |
| | | | | | GN = proS PE = 3 SV = 1 − [SYP_STRPN] | |
| Q97SI4 | 40.88 | 11 | 340 | 140.1 | Choline binding protein C OS = | N- |
| | | | | | Streptococcus pneumoniae serotype 4 | terminally |
| | | | | | (strain ATCC BAA-334/TIGR4) | anchored |
| | | | | | GN = cbpC PE = 4 SV = 1 − [Q97SI4_STRPN] | (with CS) |
| P0A4D7 | 40.84 | 17 | 524 | 138.63 | DEAD-box ATP-dependent RNA | Intracellular |
| | | | | | helicase CshA OS = -Streptococcus | |
| | | | | | pneumoniae serotype 4 (strain ATCC | |
| | | | | | BAA-334/TIGR4) GN = cshA PE = 3 | |
| | | | | | SV = 1 − [CSHA_STRPN] | |
| Q97RN3 | 44.44 | 17 | 513 | 136.66 | ABC transporter, ATP-binding protein | Intracellular |
| | | | | | OS = -Streptococcus pneumoniae | |
| | | | | | serotype 4 (strain ATCC BAA-334/ | |
| | | | | | TIGR4) GN = SP_0770 PE = 1 SV = | |
| | | | | | 1 − [Q97RN3_STRPN] | |
| Q97PR0 | 43.62 | 17 | 447 | 136.28 | Asparagine--tRNA ligase OS = | Intracellular |
| | | | | | Streptococcus pneumoniae serotype 4 | |
| | | | | | (strain ATCC BAA-334/TIGR4) | |
| | | | | | GN = asnS PE = 1 SV = 1 − [SYN_STRPN] | |
| P22976 | 40.73 | 18 | 658 | 132.04 | Probable transketolase OS = | Intracellular |
| | | | | | Streptococcus pneumoniae serotype 4 | |
| | | | | | (strain ATCC BAA-334/TIGR4) GN = tkt | |
| | | | | | PE = 3 SV = 2 − [TKT_STRPN] | |
| Q97PX1 | 34.2 | 16 | 459 | 132 | NADH oxidase OS = = -Streptococcus | Intracellular |
| | | | | | pneumoniae serotype 4 (strain ATCO | |
| | | | | | BAA-334/TIGR4) GN = nox PE = 4 | |
| | | | | | SV = 1 − [Q97PX1_STRPN] | |
| O05703 | 50.5 | 17 | 501 | 129.61 | Zinc-binding lipoprotein AdcA OS = | Lipid |
| | | | | | Streptococcus pneumoniae serotype 4 | anchored |
| | | | | | (strain ATCC BAA-334/TIGR4) | |
| | | | | | GN = adcA PE = 3 SV = 4 − [ADCA_STRPN] | |
| P66095 | 52.4 | 12 | 229 | 128.44 | 50 S ribosomal protein L1 OS = | Intracellular |
| | | | | | Streptococcus pneumoniae serotype 4 | |
| | | | | | (strain ATCC BAA-334/TIGR4) | |
| | | | | | GN = rplA PE = 3 SV = 1 − [RL1_STRPN] | |
| Q97RP4 | 55.65 | 12 | 230 | 127.75 | Cell division ABC transporter, ATP- | Intracellular |
| | | | | | binding protein FtsE OS = | |
| | | | | | Streptococcus pneumoniae serotype 4 | |
| | | | | | (strain ATCC BAA-334/TIGR4) | |
| | | | | | GN = ftsE PE = 3 SV = 1 − [Q97RP4_STRPN] | |
| Q97RP3 | 37.66 | 12 | 308 | 126.99 | Cell division protein FtsX OS = | Multi- |
| | | | | | Streptococcus pneumoniae serotype 4 | trans- |
| | | | | | (strain ATCC BAA-334/TIGR4) | membrane |
| | | | | | GN = ftsX PE = 3 SV = 1 − [Q97RP3_STRPN] | |
| Q97N72 | 38.4 | 23 | 810 | 122.4 | ATP-dependent Clp protease, ATP- | Intracellular |
| | | | | | binding subunit OS = -Streptococcus | |
| | | | | | pneumoniae serotype 4 (strain ATCC | |
| | | | | | BAA-334/TIGR4) GN = SP_2194 PE = 3 | |
| | | | | | SV = 1 − [Q97N72_STRPN] | |
| Q97Q67 | 43.9 | 14 | 344 | 121.11 | Conserved domain protein OS = | Multi- |
| | | | | | Streptococcus pneumoniae serotype 4 | trans- |
| | | | | | (strain ATCC BAA-334/TIGR4) | membrane |
| | | | | | GN = SP_1363 PE = 4 SV = 1 − | |
| | | | | | [Q97Q67_STRPN] | |
| P95830 | 50.79 | 15 | 378 | 120.38 | Chaperone protein DnaJ OS = | Intracellular |
| | | | | | Streptococcus pneumoniae serotype 4 | |
| | | | | | (strain ATCC BAA-334/TIGR4) | |
| | | | | | GN = dnaJ PE = 1 SV = 2 − [DNAJ_STRPN] | |
| Q97SJ6 | 53.04 | 8 | 230 | 119.51 | Capsular polysaccharide biosynthesis | Multi- |
| | | | | | protein CpsC OS = -Streptococcus | trans- |
| | | | | | pneumoniae serotype 4 (strain ATCC | membrane |
| | | | | | BAA-334/TIGR4) GN = cpsC PE = 3 | |
| | | | | | SV = 1 − [CPSC_STRPN] | |
| P63373 | 71.03 | 13 | 252 | 116.79 | Phosphate import ATP-binding protein | Intracellular |
| | | | | | PstB 1 OS = -Streptococcus pneumoniae | |
| | | | | | serotype 4 (strain ATCC BAA-334/ | |
| | | | | | TIGR4) GN = pstB1 PE = 3 | |
| | | | | | SV = 1 − [PSTB1_STRPN] | |
| I6L8Q3 | 32.35 | 12 | 340 | 114.44 | Choline binding protein F OS = | N- |
| | | | | | Streptococcus pneumoniae serotype 4 | terminally |
| | | | | | (strain ATCC BAA-334/TIGR4) | anchored |
| | | | | | GN = cbpF PE = 4 SV = 1 − [I6L8Q3_STRPN] | (with CS) |
| Q97RS8 | 62.96 | 16 | 378 | 110.09 | Lactate oxidase OS = -Streptococcus | Intracellular |
| | | | | | pneumoniae serotype 4 (strain ATCC | |
| | | | | | BAA-334/TIGR4) GN = IctO-2 PE = 4 | |
| | | | | | SV = 1 − [Q97RS8_STRPN] | |
| Q97N53 | 70.96 | 11 | 272 | 109.3 | Cell shape-determining protein MreC | N- |
| | | | | | OS = -Streptococcus pneumoniae | terminally |
| | | | | | serotype 4 (strain ATCC BAA-334/ | anchored |
| | | | | | TIGR4) GN = mreC PE = 3 SV = 1 − | (No CS) |
| | | | | | [Q97N53_STRPN] | |
| P0A3M5 | 41.32 | 18 | 680 | 109.25 | Penicillin-binding protein 2 B OS = | N- |
| | | | | | Streptococcus pneumoniae serotype 4 | terminally |
| | | | | | (strain ATCC BAA-334/TIGR4) | anchored |
| | | | | | GN = penA PE = 3 SV = 1 − [PBP2_STRPN] | (No CS) |
| P0A4S1 | 49.15 | 9 | 293 | 109 | Fructose-bisphosphate aldolase OS = | Intracellular |
| | | | | | Streptococcus pneumoniae serotype 4 | |
| | | | | | (strain ATCC BAA-334/TIGR4) GN = fba | |
| | | | | | PE = 3 SV = 1 − [ALF_STRPN] | |
| Q97RK0 | 29.57 | 13 | 575 | 107.28 | Septation ring formation regulator | N- |
| | | | | | EzrA OS = -Streptococcus pneumoniae | terminally |
| | | | | | serotype 4 (strain ATCC BAA-334/ | anchored |
| | | | | | TIGR4) GN = ezrA PE = 3 SV = 1 − | (No CS) |
| | | | | | [EZRA_STRPN] | |
| Q97SI9 | 60.55 | 7 | 109 | 107.21 | Cell cycle protein GpsB OS = | Intracellular |
| | | | | | Streptococcus pneumoniae serotype 4 | |
| | | | | | (strain ATCC BAA-334/TIGR4) | |
| | | | | | GN = gpsB PE = 3 SV = 1 − [GPSB_STRPN] | |
| P63413 | 36.36 | 12 | 396 | 106.75 | Acetate kinase OS = -Streptococcus | Intracellular |
| | | | | | pneumoniae serotype 4 (strain ATCC | |
| | | | | | BAA-334/TIGR4) GN = ackA PE = 3 | |
| | | | | | SV = 1 − [ACKA_STRPN] | |
| P0A2Z2 | 59.76 | 10 | 164 | 105.18 | ATP synthase subunit b OS = | Intracellular |
| | | | | | Streptococcus pneumoniae serotype 4 | |
| | | | | | (strain ATCC BAA-334/TIGR4) | |
| | | | | | GN = atpF PE = 3 SV = 1 − [ATPF_STRPN] | |
| Q97SQ4 | 39.74 | 8 | 156 | 103.23 | 30 S ribosomal protein S7 OS = | Intracellular |
| | | | | | Streptococcus pneumoniae serotype 4 | |
| | | | | | (strain ATCC BAA-334/TIGR4) | |
| | | | | | GN = rpsG PE = 3 SV = 1 − [RS7_STRPN] | |
| Q97QK5 | 48.6 | 19 | 607 | 101.3 | Elongation factor 4 OS = -Streptococcus | Intracellular |
| | | | | | pneumoniae serotype 4 (strain ATCC | |
| | | | | | BAA-334/TIGR4) GN = lepA PE = 3 | |
| | | | | | SV = 1 − [LEPA_STRPN] | |
| P65239 | 39.75 | 11 | 322 | 100.54 | Ribose-phosphate pyrophosphokinase | Intracellular |
| | | | | | 1 OS = -Streptococcus pneumoniae | |
| | | | | | serotype 4 (strain ATCC BAA-334/ | |
| | | | | | TIGR4) GN = prs1 PE = 3 SV = 1 − | |
| | | | | | [KPRS1_STRPN] | |
| P95829 | 36.41 | 14 | 607 | 100.51 | Chaperone protein Dnak OS = | Intracellular |
| | | | | | Streptococcus pneumoniae serotype 4 | |
| | | | | | (strain ATCC BAA-334/TIGR4) | |
| | | | | | GN = dnak PE = 3 SV = 2 − [DNAK_STRPN] | |
| I6L8U5 | 55.47 | 11 | 411 | 100.01 | 3-oxoacyl-[acyl-carrier-protein] | Intracellular |
| | | | | | synthase 2 OS = -Streptococcus | |
| | | | | | pneumoniae serotype 4 (strain ATCC | |
| | | | | | BAA-334/TIGR4) GN = fabF PE = 3 | |
| | | | | | SV = 1 − [16L8U5_STRPN] | |
| Q97PV1 | 21.8 | 6 | 289 | 93.98 | Putative glycerol uptake facilitator | Multi- |
| | | | | | protein OS = -Streptococcus | trans- |
| | | | | | pneumoniae serotype 4 (strain ATCC | membrane |
| | | | | | BAA-334/TIGR4) GN = SP_1491 PE = 3 | |
| | | | | | SV = 1 − [Q97PV1_STRPN] | |
| Q2MGH6 | 15.73 | 18 | 1767 | 93.68 | Endo-alpha-N-acetylgalactosaminidase | LP × TG |
| | | | | | OS = -Streptococcus pneumoniae | Cell-wall anchored |
| | | | | | serotype 4 (strain ATCC BAA-334/ | |
| | | | | | TIGR4) GN = SP_0368 PE = 1 | |
| | | | | | SV = 1 − [GH101_STRPN] | |
| Q97SQ9 | 36.05 | 13 | 602 | 92.56 | Glutamine--fructose-6-phosphate | Intracellular |
| | | | | | aminotransferase [isomerizing] OS = | |
| | | | | | Streptococcus pneumoniae serotype 4 | |
| | | | | | (strain ATCC BAA-334/TIGR4) | |
| | | | | | GN = glmS PE = 3 SV = 3 − [GLMS_STRPN] | |
| Q97P40 | 53.96 | 9 | 202 | 92.31 | Putative general stress protein 24 OS = | Intracellular |
| | | | | | Streptococcus pneumoniae serotype 4 | |
| | | | | | (strain ATCC BAA-334/TIGR4) | |
| | | | | | GN = SP_1804 PE = 4 SV = 1 − | |
| | | | | | [Q97P40_STRPN] | |
| Q97QE4 | 25.33 | 18 | 1058 | 90.95 | Carbamoyl-phosphate synthase large | Intracellular |
| | | | | | chain OS = -Streptococcus pneumoniae | |
| | | | | | serotype 4 (strain ATCC BAA-334/ | |
| | | | | | TIGR4) GN = carB PE = 3 SV = 1 − | |
| | | | | | [CARB_STRPN] | |
| I6L8Q9 | 40.04 | 11 | 457 | 90.76 | Cell division protein ftsA OS = | Intracellular |
| | | | | | Streptococcus pneumoniae serotype 4 | |
| | | | | | (strain ATCC BAA-334/TIGR4) | |
| | | | | | GN = ftsA PE = 3 SV = 1 − [I6L8Q9_STRPN] | |
| I6L8S9 | 45.52 | 10 | 413 | 90.26 | D-alanyl-D-alanine carboxypeptidase | N- |
| | | | | | OS = -Streptococcus pneumoniae | terminally |
| | | | | | serotype 4 (strain ATCC BAA-334/ | anchored |
| | | | | | TIGR4) GN = dacA PE = 3 SV = 1 − | (with CS) |
| | | | | | [I6L859_STRPN] | |
| Q97Q48 | 27.52 | 18 | 872 | 89.81 | Alanine--tRNA ligase OS = | Intracellular |
| | | | | | Streptococcus pneumoniae serotype 4 | |
| | | | | | (strain ATCC BAA-334/TIGR4) | |
| | | | | | GN = alaS PE = 3 SV = 1 − [SYA_STRPN] | |
| Q97QD5 | 64.52 | 12 | 186 | 89.81 | LemA protein OS = -Streptococcus | N- |
| | | | | | pneumoniae serotype 4 (strain ATCC | terminally |
| | | | | | BAA-334/TIGR4) GN = lemA PE = 1 | anchored |
| | | | | | SV = 1 − [Q97QD5_STRPN] | (No CS) |
| Q97NW2 | 48.69 | 13 | 419 | 88.69 | Sugar ABC transporter, sugar-binding | Lipid |
| | | | | | protein OS = -Streptococcus | anchored |
| | | | | | pneumoniae serotype 4 (strain ATCC | |
| | | | | | BAA-334/TIGR4) GN = rafE PE = 1 SV = 1 − | |
| | | | | | [Q97NW2_STRPN] | |
| I6L8N0 | 43.01 | 14 | 365 | 88.6 | UDP-N-acetylglucosamine-2- | Intracellular |
| | | | | | epimerase OS = -Streptococcus | |
| | | | | | pneumoniae serotype 4 (strain ATCC | |
| | | | | | BAA-334/TIGR4) GN = cps4l PE = 3 | |
| | | | | | SV = 1 − [I6L8N0_STRPN] | |
| Q97QW4 | 36.47 | 13 | 425 | 87.93 | Uncharacterized protein OS = | Intracellular |
| | | | | | Streptococcus pneumoniae serotype 4 | |
| | | | | | (strain ATCC BAA-334/TIGR4) | |
| | | | | | GN = SP_1083 PE = 4 SV = 1 − | |
| | | | | | [Q97QW4_STRPN] | |
| Q9FBB7 | 58.43 | 12 | 255 | 86.89 | Acetyl-coenzyme A carboxylase | Intracellular |
| | | | | | carboxyl transferase subunit alpha | |
| | | | | | OS = -Streptococcus pneumoniae | |
| | | | | | serotype 4 (strain ATCC BAA-334/ | |
| | | | | | TIGR4) GN = accA PE = 1 SV = 1 − | |
| | | | | | [ACCA_STRPN] | |
| P67266 | 42.42 | 4 | 99 | 86.55 | Nucleoid-associated protein SP _1102 | Intracellular |
| | | | | | OS = -Streptococcus pneumoniae | |
| | | | | | serotype 4 (strain ATCC BAA-334/ | |
| | | | | | TIGR4) GN = SP_1102 PE = 1 SV = 1 − | |
| | | | | | [Y1102_STRPN] | |
| Q97QM4 | 24.06 | 13 | 719 | 86.48 | Ribonucleoside-diphosphate | Intracellular |
| | | | | | reductase OS = -Streptococcus | |
| | | | | | pneumoniae serotype 4 (strain ATCC | |
| | | | | | BAA-334/TIGR4) GN = nrdE PE = 3 | |
| | | | | | SV = 1 − [Q97QM4_STRPN] | |
| Q97PF5 | 44.17 | 8 | 283 | 85.34 | Putative phosphosugar-binding | Intracellular |
| | | | | | transcriptional regulator OS = | |
| | | | | | Streptococcus pneumoniae serotype 4 | |
| | | | | | (strain ATCC BAA-334/TIGR4) | |
| | | | | | GN = SP_1674 PE = 4 SV = | |
| | | | | | 1 − [Q97PF5_STRPN] | |
| P0A3Y3 | 50.87 | 10 | 230 | 84.95 | 2,3-bisphosphoglycerate-dependent | Intracellular |
| | | | | | phosphoglycerate mutase OS = | |
| | | | | | Streptococcus pneumoniae serotype 4 | |
| | | | | | (strain ATCC BAA-334/TIGR4) | |
| | | | | | GN = gpmA PE = 3 SV = 1 − [GPMA_STRPN] | |
| Q97NE1 | 40.78 | 4 | 103 | 84.35 | Uncharacterized protein OS = | Intracellular |
| | | | | | Streptococcus pneumoniae serotype 4 | |
| | | | | | (strain ATCC BAA-334/TIGR4) | |
| | | | | | GN = SP_2102 PE = 4 SV = 1 − | |
| | | | | | [Q97NE1_STRPN] | |
| P67282 | 35.58 | 16 | 534 | 83.04 | Ribonuclease Y OS = -Streptococcus | N- |
| | | | | | pneumoniae serotype 4 (strain ATCC | terminally |
| | | | | | BAA-334/TIGR4) GN = rny PE = 3 SV = 1 − | anchored |
| | | | | | [RNY_STRPN] | (No CS) |
| Q97QX6 | 22.61 | 14 | 898 | 82.38 | Phosphoenolpyruvate carboxylase | Intracellular |
| | | | | | OS = -Streptococcus pneumoniae | |
| | | | | | serotype 4 (strain ATCC BAA-334/ | |
| | | | | | TIGR4) GN = ppc PE = 3 SV = 1 − | |
| | | | | | [CAPP_STRPN] | |
| Q97SD1 | 32.63 | 12 | 521 | 81.72 | Amino acid ABC transporter, amino | Multi- |
| | | | | | acid-binding protein/permease | trans- |
| | | | | | protein OS = -Streptococcus | membrane |
| | | | | | pneumoniae serotype 4 (strain ATCC | |
| | | | | | BAA-334/TIGR4) GN = SP_0453 PE = 4 | |
| | | | | | SV = 1 − [Q97SD1_STRPN] | |
| P66419 | 50.56 | 8 | 89 | 78.12 | 30 S ribosomal protein S14 OS = | Intracellular |
| | | | | | Streptococcus pneumoniae serotype 4 | |
| | | | | | (strain ATCC BAA-334/TIGR4) | |
| | | | | | GN = rpsN PE = 3 SV = 1 − [RS14_STRPN] | |
| P61182 | 61.4 | 8 | 114 | 77.58 | 50 S ribosomal protein L22 OS = | Intracellular |
| | | | | | Streptococcus pneumoniae serotype 4 | |
| | | | | | (strain ATCC BAA-334/TIGR4) | |
| | | | | | GN = rplV PE = 1 SV = 1 − [RL22_STRPN] | |
| P66278 | 33.33 | 4 | 66 | 77.34 | 50 S ribosomal protein L35 OS = | Intracellular |
| | | | | | Streptococcus pneumoniae serotype 4 | |
| | | | | | (strain ATCC BAA-334/TIGR4) | |
| | | | | | GN = rpml PE = 3 SV = 1 − [RL35_STRPN] | |
| Q97R14 | 34.57 | 10 | 324 | 76.27 | Uncharacterized protein OS = | Secretory |
| | | | | | Streptococcus pneumoniae serotype 4 | (released) |
| | | | | | (strain ATCC BAA-334/TIGR4) | (with CS) |
| | | | | | GN = SP_1027 PE = 4 SV = 1 − | |
| | | | | | [Q97R14_STRPN] | |
| Q97T52 | 22.4 | 12 | 616 | 70.86 | Putative capsular polysaccharide | Multi- |
| | | | | | biosynthesis protein OS = | trans- |
| | | | | | Streptococcus pneumoniae serotype 4 | membrane |
| | | | | | (strain ATCC BAA-334/TIGR4) | |
| | | | | | GN = SP_0103 PE = 4 SV = 1 − | |
| | | | | | [Q97T52_STRPN] | |
| Q97SW2 | 26.53 | 13 | 735 | 70.68 | Anaerobic ribonucleoside- | Intracellular |
| | | | | | triphosphate reductase OS = | |
| | | | | | Streptococcus pneumoniae serotype 4 | |
| | | | | | (strain ATCC BAA-334/TIGR4) | |
| | | | | | GN = nrdD PE = 4 SV = 1 − | |
| | | | | | [Q97SW2_STRPN] | |
| Q97SE4 | 32.3 | 12 | 514 | 70.62 | Peptide chain release factor 3 OS = | Intracellular |
| | | | | | Streptococcus pneumoniae serotype 4 | |
| | | | | | (strain ATCC BAA-334/TIGR4) | |
| | | | | | GN = prfC PE = 3 SV = 1 − [RF3_STRPN] | |
| Q97PC4 | 27.24 | 9 | 492 | 68.5 | ABC transporter, ATP-binding protein | Multi- |
| | | | | | OS = -Streptococcus pneumoniae | trans- |
| | | | | | serotype 4 (strain ATCC BAA-334/ | membrane |
| | | | | | TIGR4) GN = SP_1715 PE = 4 SV = 1 − | |
| | | | | | [Q97PC4_STRPN] | |
| I6L8V8 | 47.33 | 7 | 243 | 68.48 | 3-oxoacyl-[acyl-carrier protein] | Intracellular |
| | | | | | reductase OS = -Streptococcus | |
| | | | | | pneumoniae serotype 4 (strain ATCC | |
| | | | | | BAA-334/TIGR4) GN = fabG PE = 3 | |
| | | | | | SV = 1 − [I6L8V8_STRPN] | |
| Q97S28 | 21.17 | 11 | 737 | 68.26 | Polyribonucleotide | Intracellular |
| | | | | | nucleotidyltransferase OS = | |
| | | | | | Streptococcus pneumoniae serotype 4 | |
| | | | | | (strain ATCC BAA-334/TIGR4) | |
| | | | | | GN = pnp PE = 3 SV = 1 − [PNP_STRPN] | |
| Q97RC4 | 41.38 | 9 | 290 | 67.69 | Uncharacterized protein OS = | Lipid |
| | | | | | Streptococcus pneumoniae serotype 4 | anchored |
| | | | | | (strain ATCC BAA-334/TIGR4) | |
| | | | | | GN = SP_0899 PE = 4 SV = 1 − | |
| | | | | | [Q97RC4_STRPN] | |
| Q97S86 | 21.65 | 7 | 448 | 65.38 | Glutamine synthetase, type I OS = | Intracellular |
| | | | | | Streptococcus pneumoniae serotype 4 | |
| | | | | | (strain ATCC BAA-334/TIGR4) | |
| | | | | | GN = glnA PE = 3 SV = 1 − [Q97S86_STRPN] | |
| Q97NH4 | 41.48 | 9 | 352 | 65.13 | Alcohol dehydrogenase, zinc- | Intracellular |
| | | | | | containing OS = -Streptococcus | |
| | | | | | pneumoniae serotype 4 (strain ATCC | |
| | | | | | BAA-334/TIGR4) GN = SP_2055 PE = 3 | |
| | | | | | SV = 1 − [Q97NH4_STRPN] | |
| P0A495 | 65.79 | 3 | 38 | 64.65 | 50 S ribosomal protein L36 OS = | Intracellular |
| | | | | | Streptococcus pneumoniae serotype 4 | |
| | | | | | (strain ATCC BAA-334/TIGR4) | |
| | | | | | GN = rpmJ PE = 3 SV = 1 − [RL36_STRPN] | |
| Q97ND6 | 25.72 | 13 | 587 | 64.48 | Aspartate--tRNA ligase OS = | Intracellular |
| | | | | | Streptococcus pneumoniae serotype 4 | |
| | | | | | (strain ATCC BAA-334/TIGR4) | |
| | | | | | GN = aspS PE = 3 SV = 1 − [SYD_STRPN] | |
| P72525 | 18.1 | 10 | 823 | 63.42 | DNA topoisomerase 4 subunit A OS = | Intracellular |
| | | | | | Streptococcus pneumoniae serotype 4 | |
| | | | | | (strain ATCC BAA-334/TIGR4) | |
| | | | | | GN = parC PE = 1 SV = 3 − [PARC_STRPN] | |
| Q97PH2 | 23.11 | 12 | 740 | 63.08 | GTP pyrophosphokinase OS = | Intracellular |
| | | | | | Streptococcus pneumoniae serotype 4 | |
| | | | | | (strain ATCC BAA-334/TIGR4) | |
| | | | | | GN = relA PE = 3 SV = 1 − [Q97PH2_STRPN] | |
| Q97T46 | 32.63 | 4 | 95 | 63.06 | Putative bacteriocin OS = | Secretory |
| | | | | | Streptococcus pneumoniae serotype 4 | |
| | | | | | (strain ATCC BAA-334/TIGR4) | |
| | | | | | GN = SP_0109 PE = 4 SV = 1 − | |
| | | | | | [Q97T46_STRPN] | |
| I6L8V0 | 42.17 | 11 | 351 | 60.21 | Capsular polysaccharide biosynthesis | Intracellular |
| | | | | | protein Cps4J OS = -Streptococcus | |
| | | | | | pneumoniae serotype 4 (strain ATCC | |
| | | | | | BAA-334/TIGR4) GN = cap4J PE = 4 | |
| | | | | | SV = 1 − [I6L8V0_STRPN] | |
| Q97RQ6 | 23.84 | 6 | 281 | 60.06 | DegV domain-containing protein | Intracellular |
| | | | | | SP_0742 OS = -Streptococcus | |
| | | | | | pneumoniae serotype 4 (strain ATCC | |
| | | | | | BAA-334/TIGR4) GN = SP_0742 PE = 3 | |
| | | | | | SV = 1 − [Y742_STRPN] | |
| Q97SR2 | 35.08 | 9 | 419 | 60.03 | Putative zinc metalloprotease | Multi- |
| | | | | | SP_0263 OS = -Streptococcus | trans- |
| | | | | | pneumoniae serotype 4 (strain ATCC | membrane |
| | | | | | BAA-334/TIGR4) GN = SP_0263 PE = 3 | |
| | | | | | SV = 1 − [Y263_STRPN] | |
| Q97PB8 | 15.63 | 8 | 627 | 59.73 | PTS system IIABC components OS = | Multi- |
| | | | | | Streptococcus pneumoniae serotype 4 | trans- |
| | | | | | (strain ATCC BAA-334/TIGR4) | membrane |
| | | | | | GN = SP_1722 PE = 4 SV = 1 − | |
| | | | | | [Q97PB8_STRPN] | |
| Q97QE6 | 41.47 | 9 | 340 | 58.95 | Alcohol dehydrogenase, zinc- | Intracellular |
| | | | | | containing OS = -Streptococcus | |
| | | | | | pneumoniae serotype 4 (strain ATCC | |
| | | | | | BAA-334/TIGR4) GN = SP_1270 PE = 4 | |
| | | | | | SV = 1 − [Q97QE6_STRPN] | |
| Q97P07 | 34.56 | 10 | 408 | 58.93 | Putative capsular polysaccharide | Intracellular |
| | | | | | biosynthesis protein OS = | |
| | | | | | Streptococcus pneumoniae serotype 4 | |
| | | | | | (strain ATCC BAA-334/TIGR4) | |
| | | | | | GN = SP_1837 PE = 3 SV = 1 − | |
| | | | | | [Q97P07_STRPN] | |
| Q97T09 | 40.23 | 9 | 353 | 57.99 | Methionine import ATP-binding | Intracellular |
| | | | | | protein MetN OS = -Streptococcus | |
| | | | | | pneumoniae serotype 4 (strain ATCC | |
| | | | | | BAA-334/TIGR4) GN = metN PE = 3 | |
| | | | | | SV = 1 − [METN_STRPN] | |
| Q97NP5 | 18.83 | 7 | 308 | 57.43 | Membrane protein insertase YidC 1 | Multi- |
| | | | | | OS = -Streptococcus pneumoniae | trans- |
| | | | | | serotype 4 (strain ATCC BAA-334/ | membrane |
| | | | | | TIGR4) GN = yidC1 PE = 3 SV = 1 − | |
| | | | | | [YIDC1_STRPN] | |
| P0A451 | 41.49 | 12 | 388 | 57.14 | Protein RecA OS = -Streptococcus | Intracellular |
| | | | | | pneumoniae serotype 4 (strain ATCC | |
| | | | | | BAA-334/TIGR4) GN = recA PE = 2 | |
| | | | | | SV = 1 − [RECA_STRPN] | |
| Q97SU7 | 51.12 | 5 | 178 | 56.93 | 50 S ribosomal protein L6 OS = | Intracellular |
| | | | | | Streptococcus pneumoniae serotype 4 | |
| | | | | | (strain ATCC BAA-334/TIGR4) | |
| | | | | | GN = rplF PE = 3 SV = 1 − [RL6_STRPN] | |
| Q97NE2 | 18.49 | 9 | 687 | 56.54 | Cation-transporting ATPase, E1-E2 | Intracellular |
| | | | | | family OS = -Streptococcus pneumoniae | |
| | | | | | serotype 4 (strain ATCC BAA-334/ | |
| | | | | | TIGR4) GN = SP_2101 PE = 3 SV = 1 − | |
| | | | | | [Q97NE2_STRPN] | |
| Q97SN4 | 32.31 | 6 | 130 | 56.22 | 30 S ribosomal protein S9 OS = | Intracellular |
| | | | | | Streptococcus pneumoniae serotype 4 | |
| | | | | | (strain ATCC BAA-334/TIGR4) | |
| | | | | | GN = rpsl PE = 3 SV = 1 − [RS9_STRPN] | |
| P66392 | 51.24 | 7 | 121 | 56.15 | 30 S ribosomal protein S13 OS = | Intracellular |
| | | | | | Streptococcus pneumoniae serotype 4 | |
| | | | | | (strain ATCC BAA-334/TIGR4) | |
| | | | | | GN = rpsM PE = 3 SV = 1 − [RS13_STRPN] | |
| Q97S10 | 23.51 | 11 | 553 | 55.98 | Ribonuclease J OS = -Streptococcus | Intracellular |
| | | | | | pneumoniae serotype 4 (strain ATCC | |
| | | | | | BAA-334/TIGR4) GN = rnj PE = 3 SV = 1 − | |
| | | | | | [Q97S10_STRPN] | |
| Q97PG0 | 20.99 | 5 | 262 | 55.94 | Cell division protein DivIVA OS = | Intracellular |
| | | | | | Streptococcus pneumoniae serotype 4 | |
| | | | | | (strain ATCC BAA-334/TIGR4) | |
| | | | | | GN = divlVA PE = 4 SV = 1 − | |
| | | | | | [Q97PG0_STRPN] | |
| P35597 | 17.61 | 9 | 778 | 55.84 | Probable cation-transporting ATPase | Multi- |
| | | | | | exp7 OS = -Streptococcus pneumoniae | trans- |
| | | | | | serotype 4 (strain ATCC BAA-334/ | membrane |
| | | | | | TIGR4) GN = exp7 PE = 3 SV = 2 − | |
| | | | | | [EXP7_STRPN] | |
| Q97SJ2 | 43.52 | 12 | 409 | 55.75 | Capsular polysaccharide biosynthesis | Intracellular |
| | | | | | protein Cps4K OS = -Streptococcus | |
| | | | | | pneumoniae serotype 4 (strain ATCC | |
| | | | | | BAA-334/TIGR4) GN = cps4K PE = 4 | |
| | | | | | SV = 1 − [Q97SJ2_STRPN] | |
| Q97PI4 | 29.98 | 12 | 647 | 55.52 | Threonine--tRNA ligase OS = | Intracellular |
| | | | | | Streptococcus pneumoniae serotype 4 | |
| | | | | | (strain ATCC BAA-334/TIGR4) | |
| | | | | | GN = thrS PE = 1 SV = 1 − [SYT_STRPN] | |
| Q97QW8 | 25.81 | 8 | 434 | 55.41 | GTPase Obg OS = -Streptococcus | Intracellular |
| | | | | | pneumoniae serotype 4 (strain ATCC | |
| | | | | | BAA-334/TIGR4) GN = obg PE = 3 SV = 1 − | |
| | | | | | [OBG_STRPN] | |
| P67293 | 43.9 | 3 | 82 | 55.21 | UPF0154 protein SP_1882 OS = | N- |
| | | | | | Streptococcus pneumoniae serotype 4 | terminally |
| | | | | | (strain ATCC BAA-334/TIGR4) | anchored |
| | | | | | GN = SP_1882 PE = 3 SV = 1 − | (No CS) |
| | | | | | [Y1882_STRPN] | |
| P0A4L9 | 20.52 | 10 | 648 | 54.97 | DNA gyrase subunit B OS = | Intracellular |
| | | | | | Streptococcus pneumoniae serotype 4 | |
| | | | | | (strain ATCC BAA-334/TIGR4) | |
| | | | | | GN = gyrB PE = 3 SV = 1 − [GYRB_STRPN] | |
| Q97SJ8 | 33.2 | 11 | 494 | 54.78 | UPF0371 protein SP_0341 OS = | Intracellular |
| | | | | | Streptococcus pneumoniae serotype 4 | |
| | | | | | (strain ATCC BAA-334/TIGR4) | |
| | | | | | GN = SP_0341 PE = 3 SV = 1 − | |
| | | | | | [Y341_STRPN] | |
| Q54869 | 24.16 | 10 | 563 | 54.43 | Arginine--tRNA ligase OS = | Intracellular |
| | | | | | Streptococcus pneumoniae serotype 4 | |
| | | | | | (strain ATCC BAA-334/TIGR4) | |
| | | | | | GN = argS PE = 1 SV = 2 − [SYR_STRPN] | |
| P35595 | 14.88 | 7 | 726 | 53.64 | PTS system glucose-specific EIICBA | Multi- |
| | | | | | component OS = -Streptococcus | trans- |
| | | | | | pneumoniae serotype 4 (strain ATCC | membrane |
| | | | | | BAA-334/TIGR4) GN = exp5 PE = 3 | |
| | | | | | SV = 2 − [PTG3C_STRPN] | |
| Q97SI7 | 22.41 | 7 | 464 | 53.36 | Mid-cell-anchored protein Z OS = | Intracellular |
| | | | | | Streptococcus pneumoniae serotype 4 | |
| | | | | | (strain ATCC BAA-334/TIGR4) | |
| | | | | | GN = mapZ PE = 1 SV = 1 − | |
| | | | | | [Q97SI7_STRPN] | |
| P0A4B5 | 55.91 | 7 | 93 | 52.72 | 30 S ribosomal protein S19 OS = | Intracellular |
| | | | | | Streptococcus pneumoniae serotype 4 | |
| | | | | | (strain ATCC BAA-334/TIGR4) | |
| | | | | | GN = rpsS PE = 3 SV = 1 − [RS19_STRPN] | |
| Q97SU3 | 23.29 | 4 | 146 | 52.09 | 50 S ribosomal protein L15 OS = | Intracellular |
| | | | | | Streptococcus pneumoniae serotype 4 | |
| | | | | | (strain ATCC BAA-334/TIGR4) | |
| | | | | | GN = rplO PE = 3 SV = 1 − [RL15_STRPN] | |
| Q97RV5 | 27.57 | 11 | 613 | 51.74 | Elongation factor Tu family protein | Intracellular |
| | | | | | OS = -Streptococcus pneumoniae | |
| | | | | | serotype 4 (strain ATCC BAA-334/ | |
| | | | | | TIGR4) GN = SP_0681 PE = 4 SV = 1 − | |
| | | | | | [Q97RV5_STRPN] | |
| Q97NW9 | 8.4 | 5 | 655 | 50.97 | Trehalose PTS system, IIABC | Multi- |
| | | | | | components OS = -Streptococcus | trans- |
| | | | | | pneumoniae serotype 4 (strain ATCC | membrane |
| | | | | | BAA-334/TIGR4) GN = SP_1884 PE = 4 | |
| | | | | | SV = 1 − [Q97NW9_STRPN] | |
| Q97QE7 | 41.6 | 9 | 262 | 49.97 | Choline kinase OS = -Streptococcus | Intracellular |
| | | | | | pneumoniae serotype 4 (strain ATCC | |
| | | | | | BAA-334/TIGR4) GN = pck PE = 4 SV = 1 − | |
| | | | | | [Q97QE7_STRPN] | |
| P67595 | 31.96 | 8 | 341 | 48.09 | Tryptophan--tRNA ligase OS = | Intracellular |
| | | | | | Streptococcus pneumoniae serotype 4 | |
| | | | | | (strain ATCC BAA-334/TIGR4) | |
| | | | | | GN = trpS PE = 3 SV = 1 − [SYW_STRPN] | |
| P66581 | 55.49 | 6 | 164 | 47.77 | 30 S ribosomal protein S5 OS = | Intracellular |
| | | | | | Streptococcus pneumoniae serotype 4 | |
| | | | | | (strain ATCC BAA-334/TIGR4) | |
| | | | | | GN = rpsE PE = 3 SV = 1 − [RS5_STRPN] | |
| Q97R57 | 16.33 | 10 | 784 | 47.19 | Ribonuclease R OS = -Streptococcus | Intracellular |
| | | | | | pneumoniae serotype 4 (strain ATCC | |
| | | | | | BAA-334/TIGR4) GN = rnr PE = 3 SV = 1 − | |
| | | | | | [Q97R57_STRPN] | |
| Q97RP0 | 21.03 | 7 | 447 | 46.86 | DEAD-box ATP-dependent RNA | Intracellular |
| | | | | | helicase CshB OS = -Streptococcus | |
| | | | | | pneumoniae serotype 4 (strain ATCC | |
| | | | | | BAA-334/TIGR4) GN = cshB PE = 3 | |
| | | | | | SV = 1 − [Q97RP0_STRPN] | |
| P0A3R1 | 22.65 | 9 | 649 | 46.48 | DNA mismatch repair protein HexB | Intracellular |
| | | | | | OS = -Streptococcus pneumoniae | |
| | | | | | serotype 4 (strain ATCC BAA-334/ | |
| | | | | | TIGR4) GN = hexB PE = 3 SV = 1 − | |
| | | | | | [HEXB_STRPN] | |
| P0A3R3 | 18.48 | 11 | 844 | 45.87 | DNA mismatch repair protein HexA | Intracellular |
| | | | | | OS = -Streptococcus pneumoniae | |
| | | | | | serotype 4 (strain ATCC BAA-334/ | |
| | | | | | TIGR4) GN = hexA PE = 3 SV = 1 − | |
| | | | | | [HEXA_STRPN] | |
| Q97SF6 | 27.97 | 7 | 261 | 45.73 | Enoyl-CoA hydratase/isomerase family | Intracellular |
| | | | | | protein OS = -Streptococcus | |
| | | | | | pneumoniae serotype 4 (strain ATCC | |
| | | | | | BAA-334/TIGR4) GN = SP_0415 PE = 4 | |
| | | | | | SV = 1 − [Q97SF6_STRPN] | |
| P66708 | 45.98 | 9 | 311 | 45.6 | DNA-directed RNA polymerase subunit alpha | Intracellular |
| | | | | | OS = -Streptococcus pneumoniae | |
| | | | | | serotype 4 (strain ATCC BAA-334/ | |
| | | | | | TIGR4) GN = rpoA PE = 3 SV = 1 − | |
| | | | | | [RPOA_STRPN] | |
| I6L858 | 28.69 | 9 | 481 | 45.33 | Capsular polysaccharide biosynthesis | Multi- |
| | | | | | protein Cps4A OS = -Streptococcus | trans- |
| | | | | | pneumoniae serotype 4 (strain ATCC | membrane |
| | | | | | BAA-334/TIGR4) GN = cps4A PE = 4 | |
| | | | | | SV = 1 − [I6L8S8_STRPN] | |
| Q97N43 | 19.51 | 7 | 492 | 45.08 | Inosine-5′-monophosphate | Intracellular |
| | | | | | dehydrogenase OS = -Streptococcus | |
| | | | | | pneumoniae serotype 4 (strain ATCC | |
| | | | | | BAA-334/TIGR4) GN = guaB PE = 3 | |
| | | | | | SV = 1 − [Q97N43_STRPN] | |
| I6L8W8 | 16.87 | 6 | 332 | 44.57 | Choline binding protein J OS = | N- |
| | | | | | Streptococcus pneumoniae serotype 4 | terminally |
| | | | | | (strain ATCC BAA-334/TIGR4) | anchored |
| | | | | | GN = cbpJ PE = 4 SV = 1 − [I6L8W8_STRPN] | (with CS) |
| Q2MGG2 | 27.62 | 7 | 467 | 44.4 | Serine protease, subtilase family OS = | Secretory |
| | | | | | Streptococcus pneumoniae serotype 4 | (released) |
| | | | | | (strain ATCC BAA-334/TIGR4) | (with CS) |
| | | | | | GN = SP_1954 PE = 4 SV = 1 − | |
| | | | | | [Q2MGG2_STRPN] | |
| Q97SP0 | 42.22 | 7 | 270 | 44.36 | Cof family protein OS = -Streptococcus | Intracellular |
| | | | | | pneumoniae serotype 4 (strain ATCC | |
| | | | | | BAA-334/TIGR4) GN = SP_0286 PE = 4 | |
| | | | | | SV = 1 − [Q97SP0_STRPN] | |
| Q97PG9 | 38.75 | 7 | 240 | 44.16 | Manganese ABC transporter, ATP- | Intracellular |
| | | | | | binding protein OS = -Streptococcus | |
| | | | | | pneumoniae serotype 4 (strain ATCC | |
| | | | | | BAA-334/TIGR4) GN = psaB PE = 3 | |
| | | | | | SV = 1 − [Q97PG9_STRPN] | |
| Q97T72 | 31.67 | 6 | 221 | 44.14 | Potassium uptake protein, Trk family | N- |
| | | | | | OS = -Streptococcus pneumoniae | terminally |
| | | | | | serotype 4 (strain ATCC BAA-334/ | anchored |
| | | | | | TIGR4) GN = SP_0079 PE = 4 SV = 1 − | (No CS) |
| | | | | | [Q97T72_STRPN] | |
| Q97RS9 | 21.98 | 7 | 496 | 44.07 | Lysine--tRNA ligase OS = -Streptococcus | Intracellular |
| | | | | | pneumoniae serotype 4 (strain ATCC | |
| | | | | | BAA-334/TIGR4) GN = lysS PE = 1 | |
| | | | | | SV = 2 − [SYK_STRPN] | |
| Q97PK5 | 27.22 | 2 | 158 | 43.99 | Uncharacterized protein OS = | N- |
| | | | | | Streptococcus pneumoniae serotype 4 | terminally |
| | | | | | (strain ATCC BAA-334/TIGR4) | anchored |
| | | | | | GN = SP_1604 PE = 4 SV = 1 − | (No CS) |
| | | | | | [Q97PK5_STRPN] | |
| Q97RV8 | 38.89 | 6 | 126 | 43.74 | Uncharacterized protein OS = | N- |
| | | | | | Streptococcus pneumoniae serotype 4 | terminally |
| | | | | | (strain ATCC BAA-334/TIGR4) | anchored |
| | | | | | GN = SP_0678 PE = 4 SV = 1 − | (No CS) |
| | | | | | [Q97RV8_STRPN] | |
| P06653 | 26.1 | 6 | 318 | 43.37 | Autolysin OS = -Streptococcus | Intracellular |
| | | | | | pneumoniae serotype 4 (strain ATCC | |
| | | | | | BAA-334/TIGR4) GN = lytA PE = 1 | |
| | | | | | SV = 2 − [ALYS_STRPN] | |
| P63791 | 27.8 | 8 | 410 | 42.54 | ATP-dependent Clp protease ATP- | Intracellular |
| | | | | | binding subunit ClpX OS = | |
| | | | | | Streptococcus pneumoniae serotype 4 | |
| | | | | | (strain ATCC BAA-334/TIGR4) | |
| | | | | | GN = clpX PE = 3 SV = 1 − [CLPX_STRPN] | |
| Q97Q36 | 37.33 | 7 | 217 | 42.45 | Phosphate-specific transport system | Intracellular |
| | | | | | accessory protein PhoU OS = | |
| | | | | | Streptococcus pneumoniae serotype 4 | |
| | | | | | (strain ATCC BAA-334/TIGR4) | |
| | | | | | GN = SP_1395 PE = 3 SV = 1 − | |
| | | | | | [Q97Q36_STRPN] | |
| Q97R16 | 32.06 | 10 | 418 | 42.38 | Serine hydroxymethyltransferase OS = | Intracellular |
| | | | | | Streptococcus pneumoniae serotype 4 | |
| | | | | | (strain ATCC BAA-334/TIGR4) | |
| | | | | | GN = glyA PE = 3 SV = 1 − [GLYA_STRPN] | |
| Q97NM1 | 38.99 | 9 | 336 | 42.33 | Catabolite control protein A OS = | Intracellular |
| | | | | | Streptococcus pneumoniae serotype 4 | |
| | | | | | (strain ATCC BAA-334/TIGR4) | |
| | | | | | GN = ccpA PE = 4 SV = 1 − | |
| | | | | | [Q97NM1_STRPN] | |
| Q97PQ8 | 42.66 | 6 | 143 | 40.36 | Uncharacterized protein OS = | Intracellular |
| | | | | | Streptococcus pneumoniae serotype 4 | |
| | | | | | (strain ATCC BAA-334/TIGR4) | |
| | | | | | GN = SP_1545 PE = 4 SV = 1 − | |
| | | | | | [Q97PQ8_STRPN] | |
| Q97SU6 | 59.32 | 7 | 118 | 40.13 | 50 S ribosomal protein L18 OS = | Intracellular |
| | | | | | Streptococcus pneumoniae serotype 4 | |
| | | | | | (strain ATCC BAA-334/TIGR4) | |
| | | | | | GN = rplR PE = 3 SV = 1 − [RL18_STRPN] | |
| Q97RC9 | 30.77 | 3 | 156 | 40.01 | Arginine repressor OS = -Streptococcus | Intracellular |
| | | | | | pneumoniae serotype 4 (strain ATCC | |
| | | | | | BAA-334/TIGR4) GN = argR PE = 3 | |
| | | | | | SV = 1 − [Q97RC9_STRPN] | |
| Q97RC5 | 25.15 | 8 | 501 | 39.74 | Pyruvate kinase OS = -Streptococcus | Intracellular |
| | | | | | pneumoniae serotype 4 (strain ATCC | |
| | | | | | BAA-334/TIGR4) GN = pyk PE = 3 SV = 1 − | |
| | | | | | [Q97RC5_STRPN] | |
| Q97PM1 | 45.04 | 7 | 262 | 38.81 | GTP-sensing transcriptional pleiotropic | Intracellular |
| | | | | | repressor CodY OS = -Streptococcus | |
| | | | | | pneumoniae serotype 4 (strain ATCC | |
| | | | | | BAA-334/TIGR4) GN = codY PE = 1 | |
| | | | | | SV = 1 − [CODY_STRPN] | |
| I6L8N6 | 28.91 | 5 | 211 | 38.64 | Capsular polysaccharide biosynthesis | N- |
| | | | | | protein Cps4E OS = -Streptococcus | terminally |
| | | | | | pneumoniae serotype 4 (strain ATCC | anchored |
| | | | | | BAA-334/TIGR4) GN = cps4E PE = 4 | (No CS) |
| | | | | | SV = 1 − [I6L8N6_STRPN] | |
| Q97R36 | 38.38 | 7 | 185 | 38.61 | Thioredoxin family protein OS = | Lipid |
| | | | | | Streptococcus pneumoniae serotype 4 | anchored |
| | | | | | (strain ATCC BAA-334/TIGR4) | |
| | | | | | GN = SP_1000 PE = 1 SV = 1 − | |
| | | | | | [Q97R36_STRPN] | |
| Q97S93 | 17.76 | 7 | 535 | 37.74 | CTP synthase OS = -Streptococcus | Intracellular |
| | | | | | pneumoniae serotype 4 (strain ATCC | |
| | | | | | BAA-334/TIGR4) GN = pyrG PE = 3 | |
| | | | | | SV = 1 − [PYRG_STRPN] | |
| Q97QW1 | 20.71 | 9 | 763 | 37.7 | DNA helicase OS = -Streptococcus | Intracellular |
| | | | | | pneumoniae serotype 4 (strain ATCC | |
| | | | | | BAA-334/TIGR4) GN = pcrA PE = 4 | |
| | | | | | SV = 1 − [Q97QW1_STRPN] | |
| P63544 | 51.76 | 6 | 170 | 37.59 | Adenine phosphoribosyltransferase | Intracellular |
| | | | | | OS = -Streptococcus pneumoniae | |
| | | | | | serotype 4 (strain ATCC BAA-334/ | |
| | | | | | TIGR4) GN = apt PE = 1 | |
| | | | | | SV = 1 − [APT_STRPN] | |
| P65241 | 18.18 | 5 | 319 | 37.57 | Ribose-phosphate pyrophosphokinase | Intracellular |
| | | | | | 2 OS = -Streptococcus pneumoniae | |
| | | | | | serotype 4 (strain ATCC BAA-334/ | |
| | | | | | TIGR4) GN = prs2 PE = 3 | |
| | | | | | SV = 1 − [KPRS2_STRPN] | |
| Q97RE5 | 17.08 | 6 | 650 | 37.17 | PTS system, fructose specific IIABC | Multi- |
| | | | | | components OS = -Streptococcus | trans- |
| | | | | | pneumoniae serotype 4 (strain ATCC | membrane |
| | | | | | BAA-334/TIGR4) GN = SP_0877 PE = 4 | |
| | | | | | SV = 1 − [Q97RE5_STRPN] | |
| P35596 | 21.38 | 9 | 608 | 37.12 | Alpha-glycerophosphate oxidase OS = | N- |
| | | | | | Streptococcus pneumoniae serotype 4 | terminally |
| | | | | | (strain ATCC BAA-334/TIGR4) | anchored |
| | | | | | GN = glpO PE = 3 SV = 2 − [GLPO_STRPN] | (No CS) |
| Q97R12 | 19.52 | 7 | 543 | 37.11 | Uncharacterized RNA | Intracellular |
| | | | | | methyltransferase SP_1029 OS = | |
| | | | | | Streptococcus pneumoniae serotype 4 | |
| | | | | | (strain ATCC BAA-334/TIGR4) | |
| | | | | | GN = SP_1029 PE = 3 SV = 1 − | |
| | | | | | [Y1029_STRPN] | |
| P64166 | 12.91 | 7 | 767 | 36.81 | DNA translocase Ftsk OS = | Multi- |
| | | | | | Streptococcus pneumoniae serotype 4 | trans- |
| | | | | | (strain ATCC BAA-334/TIGR4) | membrane |
| | | | | | GN = ftsK PE = 3 SV = 1 − [FTSK_STRPN] | |
| P35594 | 17.15 | 10 | 752 | 36.7 | ATP-dependent Clp protease ATP- | Intracellular |
| | | | | | binding subunit ClpE OS = | |
| | | | | | Streptococcus pneumoniae serotype 4 | |
| | | | | | (strain ATCC BAA-334/TIGR4) | |
| | | | | | GN = clpE PE = 3 SV = 2 − [CLPE_STRPN] | |
| P64072 | 53.33 | 8 | 195 | 36.63 | Probable GTP-binding protein EngB | Intracellular |
| | | | | | OS = -Streptococcus pneumoniae | |
| | | | | | serotype 4 (strain ATCC BAA-334/ | |
| | | | | | TIGR4) GN = engB PE = 3 SV = 1 − | |
| | | | | | [ENGB_STRPN] | |
| Q97TC4 | 31.11 | 5 | 180 | 36.56 | Hypoxanthine-guanine | Intracellular |
| | | | | | phosphoribosyltransferase OS = | |
| | | | | | Streptococcus pneumoniae serotype 4 | |
| | | | | | (strain ATCC BAA-334/TIGR4) GN = hpt | |
| | | | | | PE = 3 SV = 1 − [HPRT_STRPN] | |
| Q97PU2 | 37.8 | 4 | 209 | 36.45 | Amino acid ABC transporter, ATP- | Intracellular |
| | | | | | binding protein OS = -Streptococcus | |
| | | | | | pneumoniae serotype 4 (strain ATCC | |
| | | | | | BAA-334/TIGR4) GN = SP_1501 PE = 3 | |
| | | | | | SV = 1 − [Q97PU2_STRPN] | |
| P0A3S3 | 38.69 | 6 | 274 | 36.43 | DNA-entry nuclease OS = | N- |
| | | | | | Streptococcus pneumoniae serotype 4 | terminally |
| | | | | | (strain ATCC BAA-334/TIGR4) | anchored |
| | | | | | GN = endA PE = 1 SV = 1 − [NUCE_STRPN] | (No CS) |
| I6L8N9 | 25.49 | 8 | 506 | 36.2 | Choline transporter OS = -Streptococcus | Multi- |
| | | | | | pneumoniae serotype 4 (strain ATCC | trans- |
| | | | | | BAA-334/TIGR4) GN = proWX PE = 3 | membrane |
| | | | | | SV = 1 − [16L8N9_STRPN] | |
| Q97PQ5 | 25.99 | 8 | 531 | 36.19 | Uncharacterized protein OS = | Multi- |
| | | | | | Streptococcus pneumoniae serotype 4 | trans- |
| | | | | | (strain ATCC BAA-334/TIGR4) | membrane |
| | | | | | GN = SP_1548 PE = 4 SV = 1 − | |
| | | | | | [Q97PQ5_STRPN] | |
| Q97NI7 | 28.66 | 6 | 328 | 36.13 | Putative jag protein OS = -Streptococcus | Intracellular |
| | | | | | pneumoniae serotype 4 (strain ATCC | |
| | | | | | BAA-334/TIGR4) GN = SP_2040 PE = 4 | |
| | | | | | SV = 1 − [Q97NI7_STRPN] | |
| P63742 | 19.32 | 7 | 502 | 36.12 | Glycerol kinase OS = -Streptococcus | Intracellular |
| | | | | | pneumoniae serotype 4 (strain ATCC | |
| | | | | | BAA-334/TIGR4) GN = glpK PE = 3 | |
| | | | | | SV = 1 − [GLPK_STRPN] | |
| Q97NT7 | 32.91 | 4 | 158 | 36 | Uncharacterized protein OS = | Multi- |
| | | | | | Streptococcus pneumoniae serotype 4 | trans- |
| | | | | | (strain ATCC BAA-334/TIGR4) | membrane |
| | | | | | GN = SP_1926 PE = 4 SV = 1 − | |
| | | | | | [Q97NT7_STRPN] | |
| P64297 | 23.85 | 7 | 520 | 35.92 | GMP synthase [glutamine-hydrolyzing] | Intracellular |
| | | | | | OS = -Streptococcus pneumoniae | |
| | | | | | serotype 4 (strain ATCC BAA-334/ | |
| | | | | | TIGR4) GN = guaA PE = 3 SV = 1 − | |
| | | | | | [GUAA_STRPN] | |
| Q97R84 | 28.38 | 7 | 444 | 35.81 | Methylenetetrahydrofolate--tRNA- | Intracellular |
| | | | | | (uracil-5-)-methyltransferase TrmFO | |
| | | | | | OS = -Streptococcus pneumoniae | |
| | | | | | serotype 4 (strain ATCC BAA-334/ | |
| | | | | | TIGR4) GN = trmFO PE = 3 SV = 2 − | |
| | | | | | [TRMFO_STRPN] | |
| Q97PK2 | 19.17 | 5 | 339 | 35.39 | UDP-glucose 4-epimerase OS = | Intracellular |
| | | | | | Streptococcus pneumoniae serotype 4 | |
| | | | | | (strain ATCC BAA-334/TIGR4) | |
| | | | | | GN = galE-1 PE = 3 SV = 1 − | |
| | | | | | [Q97PK2_STRPN] | |
| Q97RP6 | 41.53 | 6 | 236 | 35.16 | Branched-chain amino acid ABC | Intracellular |
| | | | | | transporter, ATP-binding protein OS = | |
| | | | | | Streptococcus pneumoniae serotype 4 | |
| | | | | | (strain ATCC BAA-334/TIGR4) GN = livF | |
| | | | | | PE = 3 SV = 1 − [Q97RP6_STRPN] | |
| Q97NM3 | 30.74 | 10 | 462 | 35.12 | Cof family protein OS = -Streptococcus | Intracellular |
| | | | | | pneumoniae serotype 4 (strain ATCC | |
| | | | | | BAA-334/TIGR4) GN = SP_1997 PE = 4 | |
| | | | | | SV = 1 − [Q97NM3_STRPN] | |
| Q97S34 | 10.86 | 7 | 801 | 34.77 | Phenylalanine--tRNA ligase beta | Intracellular |
| | | | | | subunit OS = -Streptococcus | |
| | | | | | pneumoniae serotype 4 (strain ATCC | |
| | | | | | BAA-334/TIGR4) GN = pheT PE = 3 | |
| | | | | | SV = 1 − [SYFB_STRPN] | |
| Q97NQ4 | 31.38 | 9 | 427 | 34.05 | UDP-N-acetylglucosamine 1- | Intracellular |
| | | | | | carboxyvinyltransferase 1 OS = | |
| | | | | | Streptococcus pneumoniae serotype 4 | |
| | | | | | (strain ATCC BAA-334/TIGR4) | |
| | | | | | GN = murA1 PE = 3 SV = 1 − | |
| | | | | | [MURA1_STRPN] | |
| Q97PY0 | 33.2 | 6 | 247 | 34.04 | Amino acid ABC transporter, ATP- | Intracellular |
| | | | | | binding protein OS = -Streptococcus | |
| | | | | | pneumoniae serotype 4 (strain ATCC | |
| | | | | | BAA-334/TIGR4) GN = SP_1460 PE = 3 | |
| | | | | | SV = 1 − [Q97PY0_STRPN] | |
| Q97RQ3 | 44.5 | 6 | 209 | 33.91 | Uracil phosphoribosyltransferase OS = | Intracellular |
| | | | | | Streptococcus pneumoniae serotype 4 | |
| | | | | | (strain ATCC BAA-334/TIGR4) | |
| | | | | | GN = upp PE = 3 SV = 1 − [UPP_STRPN] | |
| Q97QP0 | 15.87 | 7 | 567 | 33.86 | Dihydrolipoyl dehydrogenase OS = | Intracellular |
| | | | | | Streptococcus pneumoniae serotype 4 | |
| | | | | | (strain ATCC BAA-334/TIGR4) | |
| | | | | | GN = SP_1161 PE = 4 SV = 1 − | |
| | | | | | [Q97QP0_STRPN] | |
| P64062 | 24.77 | 8 | 436 | 33.49 | GTPase Der OS = -Streptococcus | Intracellular |
| | | | | | pneumoniae serotype 4 (strain ATCC | |
| | | | | | BAA-334/TIGR4) GN = der PE = 3 | |
| | | | | | SV = 1 − [DER_STRPN] | |
| Q97N48 | 22.83 | 6 | 276 | 33.26 | Uncharacterized protein OS = | Intracellular/ |
| | | | | | Streptococcus pneumoniae serotype 4 | TMH |
| | | | | | (strain ATCC BAA-334/TIGR4) | start |
| | | | | | GN = SP_2223 PE = 4 SV = 1 − | AFTER 60 |
| | | | | | [Q97N48_STRPN] | |
| Q97SJ5 | 22.98 | 7 | 409 | 32.72 | Capsular polysaccharide biosynthesis | Multi- |
| | | | | | protein Cps4F OS = -Streptococcus | trans- |
| | | | | | pneumoniae serotype 4 (strain ATCC | membrane |
| | | | | | BAA-334/TIGR4) GN = cps4F PE = 4 | |
| | | | | | SV = 1 − [Q97SJ5_STRPN] | |
| P65832 | 36.73 | 6 | 275 | 32.69 | Pur operon repressor OS = | Intracellular |
| | | | | | Streptococcus pneumoniae serotype 4 | |
| | | | | | (strain ATCC BAA-334/TIGR4) | |
| | | | | | GN = purR PE = 3 SV = 1 − [PURR_STRPN] | |
| P63384 | 12.09 | 9 | 943 | 31.63 | UvrABC system protein A OS = | Intracellular |
| | | | | | Streptococcus pneumoniae serotype 4 | |
| | | | | | (strain ATCC BAA-334/TIGR4) | |
| | | | | | GN = uvrA PE = 3 SV = 1 − [UVRA_STRPN] | |
| I6L8W3 | 33.88 | 8 | 242 | 31.53 | Choline transporter OS = -Streptococcus | Intracellular |
| | | | | | pneumoniae serotype 4 (strain ATCC | |
| | | | | | BAA-334/TIGR4) GN = proV PE = 3 | |
| | | | | | SV = 1 − [I6L8W3_STRPN] | |
| Q9AHD2 | 40.97 | 8 | 227 | 31.44 | Tyrosine-protein kinase CpsD OS = | Intracellular |
| | | | | | Streptococcus pneumoniae serotype 4 | |
| | | | | | (strain ATCC BAA-334/TIGR4) | |
| | | | | | GN = cpsD PE = 3 SV = 1 − [CPSD_STRPN] | |
| O08397 | 23.84 | 8 | 453 | 31.17 | Chromosomal replication initiator | Intracellular |
| | | | | | protein DnaA OS = -Streptococcus | |
| | | | | | pneumoniae serotype 4 (strain ATCC | |
| | | | | | BAA-334/TIGR4) GN = dnaA PE = 3 | |
| | | | | | SV = 2 − [DNAA_STRPN] | |
| Q97PT5 | 23.63 | 7 | 292 | 30.59 | ATP synthase gamma chain OS = | Intracellular |
| | | | | | Streptococcus pneumoniae serotype 4 | |
| | | | | | (strain ATCC BAA-334/TIGR4) | |
| | | | | | GN = atpG PE = 3 SV = 1 − [ATPG_STRPN] | |
| Q97PJ1 | 22.09 | 5 | 249 | 30.29 | Acyltransferase family protein OS = | Multi- |
| | | | | | Streptococcus pneumoniae serotype 4 | trans- |
| | | | | | (strain ATCC BAA-334/TIGR4) | membrane |
| | | | | | GN = SP_1624 PE = 4 SV = 1 − | |
| | | | | | [Q97PJ1_STRPN] | |
| Q97NF6 | 14.36 | 6 | 564 | 30.07 | ABC transporter, ATP- | Multi- |
| | | | | | binding/permease protein OS = | trans- |
| | | | | | Streptococcus pneumoniae serotype 4 | membrane |
| | | | | | (strain ATCC BAA-334/TIGR4) | |
| | | | | | GN = SP_2075 PE = 3 SV = 1 − | |
| | | | | | [Q97NF6_STRPN] | |
| Q97PW8 | 18.36 | 6 | 414 | 30.06 | Putative oxidoreductase OS = | Intracellular |
| | | | | | Streptococcus pneumoniae serotype 4 | |
| | | | | | (strain ATCC BAA-334/TIGR4) | |
| | | | | | GN = SP_1472 PE = 4 SV = 1 − | |
| | | | | | [Q97PW8_STRPN] | |
| Q97QT0 | 15.61 | 5 | 474 | 29.86 | Glyceraldehyde-3-phosphate | Intracellular |
| | | | | | dehydrogenase, NADP-dependent | |
| | | | | | OS = Streptococcus pneumoniae serotype 4 | |
| | | | | | (strain ATCC BAA-334/TIGR4) | |
| | | | | | GN = gapN PE = 3 SV = 1 − | |
| | | | | | [Q97QT0_STRPN] | |
| Q97PW6 | 12.24 | 6 | 678 | 29.5 | Glycine--tRNA ligase beta subunit | Intracellular |
| | | | | | OS = Streptococcus pneumoniae serotype 4 | |
| | | | | | (strain ATCC BAA-334/TIGR4) | |
| | | | | | GN = glyS PE = 3 SV = 1 − [SYGB_STRPN] | |
| Q97T93 | 25.93 | 8 | 432 | 29.32 | Adenylosuccinate lyase OS = | Intracellular |
| | | | | | Streptococcus pneumoniae serotype 4 | |
| | | | | | (strain ATCC BAA-334/TIGR4) | |
| | | | | | GN = purB PE = 3 SV = 1 − | |
| | | | | | [Q97T93_STRPN] | |
| Q97RG3 | 5.68 | 2 | 229 | 29.15 | Putative membrane protein OS = | Multi- |
| | | | | | Streptococcus pneumoniae serotype 4 | trans- |
| | | | | | (strain ATCC BAA-334/TIGR4) | membrane |
| | | | | | GN = SP_0858 PE = 4 SV = 1 − | |
| | | | | | [Q97RG3_STRPN] | |
| Q97TA6 | 12.42 | 3 | 330 | 28.21 | Phosphate acyltransferase OS = | Intracellular |
| | | | | | Streptococcus pneumoniae serotype 4 | |
| | | | | | (strain ATCC BAA-334/TIGR4) | |
| | | | | | GN = plsX PE = 3 SV = 1 − [PLSX_STRPN] | |
| Q97TA8 | 19.02 | 6 | 389 | 27.91 | Aromatic amino acid aminotransferase | Intracellular |
| | | | | | OS = -Streptococcus pneumoniae | |
| | | | | | serotype 4 (strain ATCC BAA-334/ | |
| | | | | | TIGR4) GN = araT PE = 3 SV = 1 − | |
| | | | | | [Q97TA8_STRPN] | |
| P0A3B7 | 23.99 | 5 | 346 | 27.48 | Elongation factor Ts OS = | Intracellular |
| | | | | | Streptococcus pneumoniae serotype 4 | |
| | | | | | (strain ATCC BAA-334/TIGR4) GN = tsf | |
| | | | | | PE = 1 SV = 2 − [EFTS_STRPN] | |
| I6L8Q7 | 20.99 | 4 | 324 | 27.27 | Enoyl-(Acyl-carrier-protein) reductase | Intracellular |
| | | | | | OS = -Streptococcus pneumoniae | |
| | | | | | serotype 4 (strain ATCC BAA-334/ | |
| | | | | | TIGR4) GN = fabK PE = 4 SV = 1 − | |
| | | | | | [I6L8Q7_STRPN] | |
| Q97SG5 | 9.38 | 5 | 778 | 26.77 | Endonuclease MutS2 OS = | Intracellular |
| | | | | | Streptococcus pneumoniae serotype 4 | |
| | | | | | (strain ATCC BAA-334/TIGR4) | |
| | | | | | GN = mutS2 PE = 3 SV = 1 − | |
| | | | | | [MUTS2_STRPN] | |
| Q97S79 | 15.4 | 5 | 487 | 26.69 | Type I restriction-modification system, | Intracellular |
| | | | | | M subunit OS = -Streptococcus | |
| | | | | | pneumoniae serotype 4 (strain ATCC | |
| | | | | | BAA-334/TIGR4) GN = hsdM PE = 4 | |
| | | | | | SV = 1 − [Q97S79_STRPN] | |
| Q97SF8 | 12.78 | 4 | 454 | 26.54 | Aspartokinase OS = -Streptococcus | Intracellular |
| | | | | | pneumoniae serotype 4 (strain ATCC | |
| | | | | | BAA-334/TIGR4) GN = SP_0413 PE = 3 | |
| | | | | | SV = 1 − [Q97SF8_STRPN] | |
| Q97S89 | 19.1 | 5 | 398 | 26.46 | Phosphoglycerate kinase OS = | Intracellular |
| | | | | | Streptococcus pneumoniae serotype 4 | |
| | | | | | (strain ATCC BAA-334/TIGR4) GN = pgk | |
| | | | | | PE = 3 SV = 1 − [PGK_STRPN] | |
| Q97QH0 | 22.14 | 6 | 429 | 26.44 | Signal recognition particle receptor | Intracellular |
| | | | | | FtsY OS = -Streptococcus pneumoniae | |
| | | | | | serotype 4 (strain ATCC BAA-334/ | |
| | | | | | TIGR4) GN = ftsY PE = 1 SV = 1 − | |
| | | | | | [Q97QH0_STRPN] | |
| P0CB59 | 5.83 | 3 | 326 | 26.31 | Phospho-N-acetylmuramoyl- | Multi- |
| | | | | | pentapeptide-transferase OS = | trans- |
| | | | | | Streptococcus pneumoniae serotype 4 | membrane |
| | | | | | (strain ATCC BAA-334/TIGR4) | |
| | | | | | GN = mraY PE = 3 SV = 1 − [MRAY_STRPN] | |
| Q97P92 | 23.64 | 5 | 368 | 26.11 | GTP-binding protein OS = | Intracellular |
| | | | | | Streptococcus pneumoniae serotype 4 | |
| | | | | | (strain ATCC BAA-334/TIGR4) | |
| | | | | | GN = SP_1749 PE = 4 SV = 1 − | |
| | | | | | [Q97P92_STRPN] | |
| P63733 | 20.06 | 6 | 359 | 25.98 | Carbamoyl-phosphate synthase small | Intracellular |
| | | | | | chain OS = -Streptococcus pneumoniae | |
| | | | | | serotype 4 (strain ATCC BAA-334/ | |
| | | | | | TIGR4) GN = carA PE = 3 SV = 1 − | |
| | | | | | [CARA_STRPN] | |
| Q97SP1 | 28.61 | 6 | 339 | 25.89 | Alcohol dehydrogenase, zinc- | Intracellular |
| | | | | | containing OS = -Streptococcus | |
| | | | | | pneumoniae serotype 4 (strain ATCC | |
| | | | | | BAA-334/TIGR4) GN = SP_0285 PE = 3 | |
| | | | | | SV = 1 − [Q97SP1_STRPN] | |
| Q97PP2 | 19.92 | 4 | 256 | 25.75 | Uncharacterized protein OS = | Multi- |
| | | | | | Streptococcus pneumoniae serotype 4 | trans- |
| | | | | | (strain ATCC BAA-334/TIGR4) | membrane |
| | | | | | GN = SP_1561 PE = 1 SV = 1 − | |
| | | | | | [Q97PP2_STRPN] | |
| P66339 | 48.04 | 4 | 102 | 25.74 | 30 S ribosomal protein S10 OS = | Intracellular |
| | | | | | Streptococcus pneumoniae serotype 4 | |
| | | | | | (strain ATCC BAA-334/TIGR4) | |
| | | | | | GN = rpsJ PE = 3 SV = 1 − [RS10_STRPN] | |
| P0A335 | 13.52 | 6 | 540 | 25.62 | 60 kDa chaperonin OS = -Streptococcus | Intracellular |
| | | | | | pneumoniae serotype 4 (strain ATCC | |
| | | | | | BAA-334/TIGR4) GN = groL PE = 1 | |
| | | | | | SV = 1 − [CH60_STRPN] | |
| O07344 | 15.69 | 3 | 204 | 25.56 | Signal peptidase I OS = -Streptococcus | N- |
| | | | | | pneumoniae serotype 4 (strain ATCC | terminally |
| | | | | | BAA-334/TIGR4) GN = lepB PE = 3 | anchored |
| | | | | | SV = 2 − [LEP_STRPN] | (No CS) |
| Q97PP5 | 41.41 | 3 | 128 | 25.55 | Uncharacterized protein OS = | Intracellular |
| | | | | | Streptococcus pneumoniae serotype 4 | |
| | | | | | (strain ATCC BAA-334/TIGR4) | |
| | | | | | GN = SP_1558 PE = 1 SV = 1 − | |
| | | | | | [Q97PP5_STRPN] | |
| Q97RP7 | 15.75 | 2 | 254 | 25.33 | Branched-chain amino acid ABC | Intracellular |
| | | | | | transporter, ATP-binding protein OS = | |
| | | | | | Streptococcus pneumoniae serotype 4 | |
| | | | | | (strain ATCC BAA-334/TIGR4) | |
| | | | | | GN = livG PE = 3 SV = 1 − [Q97RP7_STRPN] | |
| Q97Q12 | 13.37 | 5 | 486 | 24.99 | Nicotinate phosphoribosyltransferase | Intracellular |
| | | | | | OS = -Streptococcus pneumoniae | |
| | | | | | serotype 4 (strain ATCC BAA-334/ | |
| | | | | | TIGR4) GN = SP_1421 PE = 3 SV = 1 − | |
| | | | | | [Q97Q12_STRPN] | |
| P67506 | 26.07 | 5 | 211 | 24.79 | tRNA (guanine-N(7)-)- | Intracellular |
| | | | | | methyltransferase OS = -Streptococcus | |
| | | | | | pneumoniae serotype 4 (strain ATCC | |
| | | | | | BAA-334/TIGR4) GN = trmB PE = 1 | |
| | | | | | SV = 1 − [TRMB_STRPN] | |
| P66155 | 17.74 | 2 | 62 | 24.21 | 50 S ribosomal protein L28 OS = | Intracellular |
| | | | | | Streptococcus pneumoniae serotype 4 | |
| | | | | | (strain ATCC BAA-334/TIGR4) | |
| | | | | | GN = rpmB PE = 3 SV = 1 − [RL28_STRPN] | |
| P66200 | 71.25 | 3 | 80 | 23.95 | 50 S ribosomal protein L31 type B OS = | Intracellular |
| | | | | | Streptococcus pneumoniae serotype 4 | |
| | | | | | (strain ATCC BAA-334/TIGR4) | |
| | | | | | GN = rpmE2 PE = 3 SV = 1 − | |
| | | | | | [RL31B_STRPN] | |
| Q97NH2 | 17.69 | 5 | 605 | 23.83 | Uncharacterized protein OS = | Multi- |
| | | | | | Streptococcus pneumoniae serotype 4 | trans- |
| | | | | | (strain ATCC BAA-334/TIGR4) | membrane |
| | | | | | GN = SP_2057 PE = 4 SV = 1 − | |
| | | | | | [Q97NH2_STRPN] | |
| Q97PP4 | 12.89 | 4 | 450 | 23.67 | Phosphoglucosamine mutase OS = | Intracellular |
| | | | | | Streptococcus pneumoniae serotype 4 | |
| | | | | | (strain ATCC BAA-334/TIGR4) | |
| | | | | | GN = glmM PE = 3 SV = 1 − | |
| | | | | | [GLMM_STRPN] | |
| Q97R44 | 50.43 | 4 | 115 | 23.65 | Uncharacterized protein OS = | Intracellular/ |
| | | | | | Streptococcus pneumoniae serotype 4 | TMH |
| | | | | | (strain ATCC BAA-334/TIGR4) | start |
| | | | | | GN = SP_0990 PE = 4 SV = 1 − | AFTER 60 |
| | | | | | [Q97R44_STRPN] | |
| Q97QB4 | 15.18 | 4 | 448 | 23.57 | Glutamate dehydrogenase OS = | Intracellular |
| | | | | | Streptococcus pneumoniae serotype 4 | |
| | | | | | (strain ATCC BAA-334/TIGR4) | |
| | | | | | GN = gdhA PE = 3 SV = 1 − | |
| | | | | | [Q97QB4_STRPN] | |
| Q97SW6 | 23.68 | 2 | 152 | 23.55 | Uncharacterized protein OS = | Lipid |
| | | | | | Streptococcus pneumoniae serotype 4 | anchored |
| | | | | | (strain ATCC BAA-334/TIGR4) | |
| | | | | | GN = SP_0198 PE = 1 SV = 1 − | |
| | | | | | [Q97SW6_STRPN] | |
| Q97PW5 | 16.07 | 4 | 305 | 23.51 | Glycine--tRNA ligase alpha subunit | Intracellular |
| | | | | | OS = -Streptococcus pneumoniae | |
| | | | | | serotype 4 (strain ATCC BAA-334/ | |
| | | | | | TIGR4) GN = glyQ PE = 3 SV = 1 − | |
| | | | | | [SYGA_STRPN] | |
| Q97Q69 | 19.63 | 5 | 428 | 23.47 | Homoserine dehydrogenase OS = | Intracellular |
| | | | | | Streptococcus pneumoniae serotype 4 | |
| | | | | | (strain ATCC BAA-334/TIGR4) | |
| | | | | | GN = hom PE = 3 SV = 1 − | |
| | | | | | [Q97Q69_STRPN] | |
| Q97QT2 | 12.27 | 6 | 652 | 23.37 | DNA ligase OS = -Streptococcus | Intracellular |
| | | | | | pneumoniae serotype 4 (strain ATCC | |
| | | | | | BAA-334/TIGR4) GN = ligA PE = 3 SV = 1 − | |
| | | | | | [DNLJ_STRPN] | |
| P0A4B3 | 33.72 | 3 | 86 | 23.26 | 30 S ribosomal protein S17 OS = | Intracellular |
| | | | | | Streptococcus pneumoniae serotype 4 | |
| | | | | | (strain ATCC BAA-334/TIGR4) | |
| | | | | | GN = rpsQ PE = 3 SV = 1 − [RS17_STRPN] | |
| Q97NG0 | 14.92 | 4 | 449 | 23.07 | Glucose-6-phosphate isomerase OS = | Intracellular |
| | | | | | Streptococcus pneumoniae serotype 4 | |
| | | | | | (strain ATCC BAA-334/TIGR4) GN = pgi | |
| | | | | | PE = 3 SV = 1 − [G6PI_STRPN] | |
| Q97T58 | 14.41 | 3 | 354 | 22.73 | Conserved domain protein OS = | Multi- |
| | | | | | Streptococcus pneumoniae serotype 4 | trans- |
| | | | | | (strain ATCC BAA-334/TIGR4) | membrane |
| | | | | | GN = SP_0097 PE = 4 SV = 1 − | |
| | | | | | [Q97T58_STRPN] | |
| Q97P32 | 16.46 | 4 | 407 | 22.04 | Tryptophan synthase beta chain OS = | Intracellular |
| | | | | | Streptococcus pneumoniae serotype 4 | |
| | | | | | (strain ATCC BAA-334/TIGR4) | |
| | | | | | GN = trpB PE = 3 SV = 1 − [TRPB_STRPN] | |
| Q97QX3 | 28.57 | 5 | 252 | 21.94 | ABC transporter, ATP-binding protein | Intracellular |
| | | | | | OS = -Streptococcus pneumoniae | |
| | | | | | serotype 4 (strain ATCC BAA-334/ | |
| | | | | | TIGR4) GN = SP_1071 PE = 3 SV = 1 − | |
| | | | | | [Q97QX3_STRPN] | |
| Q97ST6 | 32.03 | 2 | 128 | 21.84 | 50 S ribosomal protein L17 OS = | Intracellular |
| | | | | | Streptococcus pneumoniae serotype 4 | |
| | | | | | (strain ATCC BAA-334/TIGR4) | |
| | | | | | GN = rplQ PE = 3 SV = 1 − [RL17_STRPN] | |
| Q97RB6 | 8.74 | 5 | 709 | 21.38 | Putative transcriptional regulator OS = | Intracellular |
| | | | | | Streptococcus pneumoniae serotype 4 | |
| | | | | | (strain ATCC BAA-334/TIGR4) | |
| | | | | | GN = SP_0908 PE = 4 SV = 1 − | |
| | | | | | [Q97RB6_STRPN] | |
| Q97NB9 | 17.91 | 4 | 335 | 21.02 | Uncharacterized protein OS = | Multi- |
| | | | | | Streptococcus pneumoniae serotype 4 | trans- |
| | | | | | (strain ATCC BAA-334/TIGR4) | membrane |
| | | | | | GN = SP_2132 PE = 4 SV = 1 − | |
| | | | | | [Q97NB9_STRPN] | |
| P66524 | 37.93 | 3 | 58 | 20.52 | 30 S ribosomal protein S21 OS = | Intracellular |
| | | | | | Streptococcus pneumoniae serotype 4 | |
| | | | | | (strain ATCC BAA-334/TIGR4) | |
| | | | | | GN = rpsU PE = 3 SV = 1 − [RS21_STRPN] | |
| Q97SI6 | 14.98 | 5 | 474 | 20.22 | 6-phosphogluconate dehydrogenase, | Intracellular |
| | | | | | decarboxylating OS = -Streptococcus | |
| | | | | | pneumoniae serotype 4 (strain ATCC | |
| | | | | | BAA-334/TIGR4) GN = gnd PE = 3 SV = 1 − | |
| | | | | | [Q97SI6_STRPN] | |
| Q97R47 | 30.45 | 4 | 266 | 20.09 | Uncharacterized protein OS = | N- |
| | | | | | Streptococcus pneumoniae serotype 4 | terminally |
| | | | | | (strain ATCC BAA-334/TIGR4) | anchored |
| | | | | | GN = SP_0987 PE = 1 SV = 1 − | (No CS) |
| | | | | | [Q97R47_STRPN] | |

TABLE 4
Proteins identified in MP from TIGR4. The accession numbers refer to SwissProt on the date of filing.

| Accession # | Coverage | # Peptides | #AAs | Score | Description | Localization |
|---|---|---|---|---|---|---|
| Q97RH0 | 76.86 | 46 | 350 | 2085.84 | Lipoprotein OS = -Streptococcus | Lipid |
| | | | | | pneumoniae serotype 4 (strain ATCC | anchored |
| | | | | | BAA-334/TIGR4) GN = SP_0845 PE = 4 | |
| | | | | | SV = 1 - [Q97RH0_STRPN] | |
| Q97PE6 | 90.27 | 56 | 442 | 1745.9 | Sugar ABC transporter, sugar-binding | Lipid |
| | | | | | protein OS = -Streptococcus | anchored |
| | | | | | pneumoniae serotype 4 (strain ATCC | |
| | | | | | BAA-334/TIGR4) GN = SP_1683 PE = 4 | |
| | | | | | SV = 1 - [Q97PE6_STRPN] | |
| Q97T39 | 61.29 | 50 | 744 | 1569.47 | Pneumococcal surface protein A OS = | N- |
| | | | | | Streptococcus pneumoniae serotype 4 | terminally |
| | | | | | (strain ATCC BAA-334/TIGR4) | anchored |
| | | | | | GN = pspA PE = 4 SV = 1 - | (with CS) |
| | | | | | [Q97T39_STRPN] | |
| P59213 | 82.74 | 62 | 423 | 1500.08 | Maltose/maltodextrin-binding protein | Lipid |
| | | | | | OS = -Streptococcus pneumoniae | anchored |
| | | | | | serotype 4 (strain ATCC BAA-334/ | |
| | | | | | TIGR4) GN = malX PE = 1 SV = 1 - | |
| | | | | | [MALX_STRPN] | |
| Q97N74 | 53.25 | 47 | 693 | 1294.91 | Choline binding protein A OS = | N- |
| | | | | | Streptococcus pneumoniae serotype 4 | terminally |
| | | | | | (strain ATCC BAA-334/TIGR4) | anchored |
| | | | | | GN = cbpA PE = 1 SV = 1 - | (with CS) |
| | | | | | [Q97N74_STRPN] | |
| Q97R51 | 87.86 | 45 | 313 | 1201.93 | Foldase protein PrsA OS = | Lipid |
| | | | | | Streptococcus pneumoniae serotype 4 | anchored |
| | | | | | (strain ATCC BAA-334/TIGR4) | |
| | | | | | GN = prsA PE = 3 SV = 1 - [PRSA_STRPN] | |
| Q54970 | 70.05 | 54 | 591 | 1000.77 | Pyruvate oxidase OS = -Streptococcus | Intracellular |
| | | | | | pneumoniae serotype 4 (strain ATCC | |
| | | | | | BAA-334/TIGR4) GN = spxB PE = 3 SV = 2 - | |
| | | | | | [POXB_STRPN] | |
| Q2MGF6 | 75.51 | 50 | 490 | 957.09 | Lysozyme OS = -Streptococcus | Secretory |
| | | | | | pneumoniae serotype 4 (strain ATCC | (released) |
| | | | | | BAA-334/TIGR4) GN = lytC PE = 4 SV = 1 - | (with CS) |
| | | | | | [Q2MGF6_STRPN] | |
| Q97PT1 | 77.13 | 52 | 551 | 947.11 | Uncharacterized protein OS = | Intracellular/ |
| | | | | | Streptococcus pneumoniae serotype 4 | TMH start |
| | | | | | (strain ATCC BAA-334/TIGR4) | AFTER 60 |
| | | | | | GN = SP_1518 PE = 4 SV = 1 - | |
| | | | | | [Q97PT1_STRPN] | |
| Q97QS2 | 75.35 | 31 | 434 | 783.92 | Enolase OS = -Streptococcus | Intracellular |
| | | | | | pneumoniae serotype 4 (strain ATCC | |
| | | | | | BAA-334/TIGR4) GN = eno PE = 1 SV = 1 - | |
| | | | | | [ENO_STRPN] | |
| P64022 | 76.77 | 48 | 693 | 749.91 | Elongation factor G OS = -Streptococcus | Intracellular |
| | | | | | pneumoniae serotype 4 (strain ATCC | |
| | | | | | BAA-334/TIGR4) GN = fusA PE = 3 SV = 1 - | |
| | | | | | [EFG_STRPN] | |
| Q04707 | 72.6 | 47 | 719 | 715.79 | Penicillin-binding protein 1A OS = | N- |
| | | | | | Streptococcus pneumoniae serotype 4 | terminally |
| | | | | | (strain ATCC BAA-334/TIGR4) | anchored |
| | | | | | GN = ponA PE = 1 SV = 2 - [PBPA_STRPN] | (No CS) |
| P0C2J9 | 87.47 | 49 | 471 | 703.79 | Pneumolysin OS = -Streptococcus | Intracellular |
| | | | | | pneumoniae serotype 4 (strain ATCC | |
| | | | | | BAA-334/TIGR4) GN = ply PE = 3 SV = 1 - | |
| | | | | | [TACY_STRPN] | |
| Q97PE1 | 73.93 | 35 | 445 | 655.97 | ABC transporter, substrate-binding | Lipid |
| | | | | | protein OS = -Streptococcus | anchored |
| | | | | | pneumoniae serotype 4 (strain ATCC | |
| | | | | | BAA-334/TIGR4) GN = SP_1690 PE = 4 | |
| | | | | | SV = 1 - [Q97PE1_STRPN] | |
| Q97NQ8 | 61.8 | 68 | 1225 | 650.84 | DNA-directed RNA polymerase subunit | Intracellular |
| | | | | | beta′ OS = -Streptococcus pneumoniae | |
| | | | | | serotype 4 (strain ATCC BAA-334/ | |
| | | | | | TIGR4) GN = rpoC PE = 3 SV = 1 - | |
| | | | | | [RPOC_STRPN] | |
| Q97NL1 | 79.1 | 29 | 335 | 611.32 | Glyceraldehyde-3-phosphate | Intracellular |
| | | | | | dehydrogenase OS = -Streptococcus | |
| | | | | | pneumoniae serotype 4 (strain ATCC | |
| | | | | | BAA-334/TIGR4) GN = gap PE = 3 SV = 1 - | |
| | | | | | [Q97NL1_STRPN] | |
| Q97T12 | 82.97 | 33 | 276 | 589.52 | ABC transporter, substrate-binding | Lipid |
| | | | | | protein OS = -Streptococcus | anchored |
| | | | | | pneumoniae serotype 4 (strain ATCC | |
| | | | | | BAA-334/TIGR4) GN = SP_0148 PE = 4 | |
| | | | | | SV = 1 - [Q97T12_STRPN] | |
| Q97T63 | 83.3 | 36 | 491 | 576.45 | ABC transporter, substrate-binding | Lipid |
| | | | | | protein OS = -Streptococcus | anchored |
| | | | | | pneumoniae serotype 4 (strain ATCC | |
| | | | | | BAA-334/TIGR4) GN = SP_0092 PE = 4 | |
| | | | | | SV = 1 - [Q97T63_STRPN] | |
| I6L8V7 | 44.02 | 33 | 627 | 457.19 | Choline binding protein E OS = | Secretory |
| | | | | | Streptococcus pneumoniae serotype 4 | (released) |
| | | | | | (strain ATCC BAA-334/TIGR4) | (with CS) |
| | | | | | GN = cbpE PE = 4 SV = 1 - [I6L8V7_STRPN] | |
| Q97NQ7 | 55.61 | 50 | 1203 | 449.03 | DNA-directed RNA polymerase subunit | Intracellular |
| | | | | | beta OS = -Streptococcus pneumoniae | |
| | | | | | serotype 4 (strain ATCC BAA-334/ | |
| | | | | | TIGR4) GN = rpoB PE = 3 SV = 1 - | |
| | | | | | [RPOB_STRPN] | |
| Q97SV2 | 64.62 | 18 | 277 | 395.51 | 50S ribosomal protein L2 OS = | Intracellular |
| | | | | | Streptococcus pneumoniae serotype 4 | |
| | | | | | (strain ATCC BAA-334/TIGR4) GN = rplB | |
| | | | | | PE = 3 SV = 1 - [RL2_STRPN] | |
| P59205 | 60.33 | 29 | 658 | 388.39 | Putative endo-beta-N- | Secretory |
| | | | | | acetylglucosaminidase OS = | (released) |
| | | | | | Streptococcus pneumoniae serotype 4 | (with CS) |
| | | | | | (strain ATCC BAA-334/TIGR4) GN = lytB | |
| | | | | | PE = 1 SV = 1 - [LYTB_STRPN] | |
| O69076 | 55.98 | 31 | 652 | 385.64 | ATP-dependent zinc metalloprotease | Multi- |
| | | | | | FtsH OS = -Streptococcus pneumoniae | transmembrane |
| | | | | | serotype 4 (strain ATCC BAA-334/ | |
| | | | | | TIGR4) GN = ftsH PE = 3 SV = 3 - | |
| | | | | | [FTSH_STRPN] | |
| Q97SP2 | 67.47 | 25 | 332 | 369.57 | PTS system, mannose-specific IIAB | Intracellular |
| | | | | | components OS = -Streptococcus | |
| | | | | | pneumoniae serotype 4 (strain ATCC | |
| | | | | | BAA-334/TIGR4) GN = manL PE = 4 SV = 1 - | |
| | | | | | [Q97SP2_STRPN] | |
| Q97T11 | 66.55 | 23 | 284 | 361.41 | Lipoprotein OS = -Streptococcus | Lipid |
| | | | | | pneumoniae serotype 4 (strain ATCC | anchored |
| | | | | | BAA-334/TIGR4) GN = SP_0149 PE = 3 | |
| | | | | | SV = 1 - [Q97T11_STRPN] | |
| I6L8Q3 | 52.65 | 17 | 340 | 354.19 | Choline binding protein F OS = | N- |
| | | | | | Streptococcus pneumoniae serotype 4 | terminally |
| | | | | | (strain ATCC BAA-334/TIGR4) | anchored |
| | | | | | GN = cbpF PE = 4 SV = 1 - [I6L8Q3_STRPN] | (with CS) |
| Q97NX9 | 80.69 | 25 | 321 | 336.18 | Iron-compound ABC transporter, iron- | Lipid anchored |
| | | | | | compound-binding protein OS = | |
| | | | | | Streptococcus pneumoniae serotype 4 | |
| | | | | | (strain ATCC BAA-334/TIGR4) | |
| | | | | | GN = SP_1872 PE = 4 SV = 1 - | |
| | | | | | [Q97NX9_STRPN] | |
| P64030 | 57.29 | 25 | 398 | 336.06 | Elongation factor Tu OS = -Streptococcus | Intracellular |
| | | | | | pneumoniae serotype 4 (strain ATCC | |
| | | | | | BAA-334/TIGR4) GN = tuf PE = 3 SV = 1 - | |
| | | | | | [EFTU_STRPN] | |
| Q97NW2 | 64.44 | 25 | 419 | 331.55 | Sugar ABC transporter, sugar-binding | Lipid |
| | | | | | protein OS = -Streptococcus | anchored |
| | | | | | pneumoniae serotype 4 (strain ATCC | |
| | | | | | BAA-334/TIGR4) GN = rafE PE = 1 SV = 1 - | |
| | | | | | [Q97NW2_STRPN] | |
| Q97QH2 | 42.86 | 29 | 721 | 330.61 | Amino acid ABC transporter, amino | Multi- |
| | | | | | acid-binding protein/permease protein | transmembrane |
| | | | | | OS = -Streptococcus pneumoniae | |
| | | | | | serotype 4 (strain ATCC BAA-334/ | |
| | | | | | TIGR4) GN = SP_1241 PE = 1 SV = 1 - | |
| | | | | | [Q97QH2_STRPN] | |
| Q97RS8 | 78.31 | 29 | 378 | 328.02 | Lactate oxidase OS = -Streptococcus | Intracellular |
| | | | | | pneumoniae serotype 4 (strain ATCC | |
| | | | | | BAA-334/TIGR4) GN = lctO-2 PE = 4 | |
| | | | | | SV = 1 - [Q97RS8_STRPN] | |
| Q97PW1 | 63.71 | 25 | 463 | 316.66 | Peptidoglycan N-acetylglucosamine | N- |
| | | | | | deacetylase A OS = -Streptococcus | terminally |
| | | | | | pneumoniae serotype 4 (strain ATCC | anchored |
| | | | | | BAA-334/TIGR4) GN = pgdA PE = 4 SV = 1 - | (No CS) |
| | | | | | [Q97PW1_STRPN] | |
| Q97PT6 | 75.85 | 26 | 468 | 315.76 | ATP synthase subunit beta OS = | Intracellular |
| | | | | | Streptococcus pneumoniae serotype 4 | |
| | | | | | (strain ATCC BAA-334/TIGR4) | |
| | | | | | GN = atpD PE = 3 SV = 1 - [ATPB_STRPN] | |
| Q97NQ3 | 58.55 | 22 | 345 | 310 | Uncharacterized protein OS = | N- |
| | | | | | Streptococcus pneumoniae serotype 4 | terminally |
| | | | | | (strain ATCC BAA-334/TIGR4) | anchored |
| | | | | | GN = SP_1967 PE = 4 SV = 1 - | (No CS) |
| | | | | | [Q97NQ3_STRPN] | |
| Q97R09 | 58.94 | 17 | 341 | 309.62 | Iron-compound ABC transporter, iron | Lipid |
| | | | | | compound-binding protein OS = | anchored |
| | | | | | Streptococcus pneumoniae serotype 4 | |
| | | | | | (strain ATCC BAA-334/TIGR4) | |
| | | | | | GN = SP_1032 PE = 1 SV = 1 - | |
| | | | | | [Q97R09_STRPN] | |
| P18791 | 69.8 | 28 | 659 | 298.67 | Oligopeptide-binding protein AmiA | Lipid |
| | | | | | OS = -Streptococcus pneumoniae | anchored |
| | | | | | serotype 4 (strain ATCC BAA-334/ | |
| | | | | | TIGR4) GN = amiA PE = 1 SV = 3 - | |
| | | | | | [AMIA_STRPN] | |
| Q97RZ7 | 63.03 | 15 | 238 | 283.45 | Uncharacterized protein OS = | Lipid |
| | | | | | Streptococcus pneumoniae serotype 4 | anchored |
| | | | | | (strain ATCC BAA-334/TIGR4) | |
| | | | | | GN = SP_0629 PE = 1 SV = 1- | |
| | | | | | [Q97RZ7_STRPN] | |
| P0A4G2 | 65.05 | 18 | 309 | 281.58 | Manganese ABC transporter substrate- | Lipid |
| | | | | | binding lipoprotein OS = -Streptococcus | anchored |
| | | | | | pneumoniae serotype 4 (strain ATCC | |
| | | | | | BAA-334/TIGR4) GN = psaA PE = 1 SV = 1 - | |
| | | | | | [MTSA_STRPN] | |
| P66112 | 60.5 | 10 | 119 | 280.56 | 50S ribosomal protein L20 OS = | Intracellular |
| | | | | | Streptococcus pneumoniae serotype 4 | |
| | | | | | (strain ATCC BAA-334/TIGR4) GN = rplT | |
| | | | | | PE = 3 SV = 1 - [RL20_STRPN] | |
| Q97PM5 | 63.3 | 23 | 376 | 278.8 | Sugar ABC transporter, ATP-binding | Intracellular |
| | | | | | protein OS = -Streptococcus | |
| | | | | | pneumoniae serotype 4 (strain ATCC | |
| | | | | | BAA-334/TIGR4) GN = msmK PE = 4 | |
| | | | | | SV = 1 - [Q97PM5_STRPN] | |
| P0A4M7 | 41.57 | 23 | 498 | 271.04 | Oligopeptide transport system | Multi- |
| | | | | | permease protein AmiC OS = | transmembrane |
| | | | | | Streptococcus pneumoniae serotype 4 | |
| | | | | | (strain ATCC BAA-334/TIGR4) | |
| | | | | | GN = amiC PE = 3 SV = 1 - [AMIC_STRPN] | |
| Q97SP4 | 48.18 | 12 | 303 | 262.56 | PTS system, mannose-specific IID | Multi- |
| | | | | | component OS = -Streptococcus | transmembrane |
| | | | | | pneumoniae serotype 4 (strain ATCC | |
| | | | | | BAA-334/TIGR4) GN = SP_0282 PE = 4 | |
| | | | | | SV = 1 - [Q97SP4_STRPN] | |
| Q97NL3 | 60.74 | 34 | 731 | 258.29 | Penicillin-binding protein 2A OS = | N- |
| | | | | | Streptococcus pneumoniae serotype 4 | terminally |
| | | | | | (strain ATCC BAA-334/TIGR4) | anchored |
| | | | | | GN = pbp2A PE = 4 SV = 1 - | (No CS) |
| | | | | | [Q97NL3_STRPN] | |
| P14677 | 61.33 | 32 | 750 | 257.72 | Penicillin-binding protein 2x OS = | N- |
| | | | | | Streptococcus pneumoniae serotype 4 | terminally |
| | | | | | (strain ATCC BAA-334/TIGR4) | anchored |
| | | | | | GN = pbpX PE = 1 SV = 2 - [PBPX_STRPN] | (No CS) |
| Q97PT4 | 38.92 | 21 | 501 | 249.37 | ATP synthase subunit alpha OS = | Multi- |
| | | | | | Streptococcus pneumoniae serotype 4 | transmembrane |
| | | | | | (strain ATCC BAA-334/TIGR4) | |
| | | | | | GN = atpA PE = 3 SV = 1 - [ATPA_STRPN] | |
| Q97NB5 | 52.66 | 30 | 621 | 236.54 | Choline binding protein PcpA OS = | Secretory |
| | | | | | Streptococcus pneumoniae serotype 4 | (released) |
| | | | | | (strain ATCC BAA-334/TIGR4) | (with CS) |
| | | | | | GN = pcpA PE = 1 SV = 1 - | |
| | | | | | [Q97NB5_STRPN] | |
| Q97PQ2 | 33.26 | 21 | 914 | 236.23 | Cation-transporting ATPase, E1-E2 | Multi- |
| | | | | | family OS = -Streptococcus pneumoniae | transmembrane |
| | | | | | serotype 4 (strain ATCC BAA-334/ | |
| | | | | | TIGR4) GN = SP_1551 PE = 3 SV = 1 - | |
| | | | | | [Q97PQ2_STRPN] | |
| P0A475 | 50.36 | 8 | 137 | 229.6 | 50S ribosomal protein L16 OS = | Intracellular |
| | | | | | Streptococcus pneumoniae serotype 4 | |
| | | | | | (strain ATCC BAA-334/TIGR4) GN = rplP | |
| | | | | | PE = 1 SV = 1 - [RL16_STRPN] | |
| Q97PU3 | 64.03 | 23 | 278 | 227.7 | Amino acid ABC transporter, amino | Lipid |
| | | | | | acid-binding protein OS = -Streptococcus | anchored |
| | | | | | pneumoniae serotype 4 (strain ATCC | |
| | | | | | BAA-334/TIGR4) GN = aatB PE = 4 SV = 1 - | |
| | | | | | [Q97PU3_STRPN] | |
| Q97RC5 | 50.3 | 20 | 501 | 223.13 | Pyruvate kinase OS = -Streptococcus | Intracellular |
| | | | | | pneumoniae serotype 4 (strain ATCC | |
| | | | | | BAA-334/TIGR4) GN = pyk PE = 3 SV = 1 - | |
| | | | | | [Q97RC5_STRPN] | |
| Q97NS5 | 65.68 | 19 | 338 | 221.3 | Putative transcriptional regulator OS = | Secretory |
| | | | | | Streptococcus pneumoniae serotype 4 | (released) |
| | | | | | (strain ATCC BAA-334/TIGR4) | (with CS) |
| | | | | | GN = SP_1942 PE = 4 SV = 1 - | |
| | | | | | [Q97NS5_STRPN] | |
| Q97RP4 | 75.22 | 14 | 230 | 213.93 | Cell division ABC transporter, ATP- | Intracellular |
| | | | | | binding protein FtsE OS = -Streptococcus | |
| | | | | | pneumoniae serotype 4 (strain ATCC | |
| | | | | | BAA-334/TIGR4) GN = ftsE PE = 3 SV = 1 - | |
| | | | | | [Q97RP4_STRPN] | |
| Q97RN2 | 66.67 | 13 | 267 | 207.19 | Peptidyl-prolyl cis-trans isomerase, | Lipid |
| | | | | | cyclophilin-type OS = -Streptococcus | anchored |
| | | | | | pneumoniae serotype 4 (strain ATCC | |
| | | | | | BAA-334/TIGR4) GN = SP_0771 PE = 3 | |
| | | | | | SV = 1 - [Q97RN2_STRPN] | |
| P66565 | 61.58 | 21 | 203 | 205.82 | 30S ribosomal protein S4 OS = | Intracellular |
| | | | | | Streptococcus pneumoniae serotype 4 | |
| | | | | | (strain ATCC BAA-334/TIGR4) | |
| | | | | | GN = rpsD PE = 3 SV = 1 - [RS4_STRPN] | |
| Q97QH1 | 61.79 | 10 | 246 | 202.96 | Amino acid ABC transporter, ATP- | Intracellular |
| | | | | | binding protein OS = -Streptococcus | |
| | | | | | pneumoniae serotype 4 (strain ATCC | |
| | | | | | BAA-334/TIGR4) GN = SP_1242 PE = 3 | |
| | | | | | SV = 1 - [Q97QH1_STRPN] | |
| I6L8N9 | 40.91 | 18 | 506 | 201.9 | Choline transporter OS = -Streptococcus | Multi- |
| | | | | | pneumoniae serotype 4 (strain ATCC | transmembrane |
| | | | | | BAA-334/TIGR4) GN = proWX PE = 3 | |
| | | | | | SV = 1 - [I6L8N9_STRPN] | |
| Q97N69 | 55.22 | 15 | 335 | 201.71 | Putative ABC transporter, substrate- | Lipid |
| | | | | | binding protein OS = -Streptococcus | anchored |
| | | | | | pneumoniae serotype 4 (strain ATCC | |
| | | | | | BAA-334/TIGR4) GN = SP_2197 PE = 4 | |
| | | | | | SV = 1 - [Q97N69_STRPN] | |
| P65887 | 53.97 | 23 | 428 | 199.01 | Adenylosuccinate synthetase OS = | Intracellular |
| | | | | | Streptococcus pneumoniae serotype 4 | |
| | | | | | (strain ATCC BAA-334/TIGR4) | |
| | | | | | GN = purA PE = 1 SV = 1 - [PURA_STRPN] | |
| P18766 | 60.71 | 13 | 308 | 198.48 | Oligopeptide transport ATP-binding | Intracellular |
| | | | | | protein AmiF OS = -Streptococcus | |
| | | | | | pneumoniae serotype 4 (strain ATCC | |
| | | | | | BAA-334/TIGR4) GN = amiF PE = 3 SV = 2 - | |
| | | | | | [AMIF_STRPN] | |
| Q97QB4 | 61.83 | 23 | 448 | 197.42 | Glutamate dehydrogenase OS = | Intracellular |
| | | | | | Streptococcus pneumoniae serotype 4 | |
| | | | | | (strain ATCC BAA-334/TIGR4) | |
| | | | | | GN = gdhA PE = 3 SV = 1 - | |
| | | | | | [Q97QB4_STRPN] | |
| Q97SP5 | 55.18 | 19 | 444 | 196.27 | Aminopeptidase C OS = -Streptococcus | Intracellular |
| | | | | | pneumoniae serotype 4 (strain ATCC | |
| | | | | | BAA-334/TIGR4) GN = pepC PE = 4 SV = 1 - | |
| | | | | | [Q97SP5_STRPN] | |
| Q97RP3 | 40.58 | 17 | 308 | 193.01 | Cell division protein FtsX OS = | Multi- |
| | | | | | Streptococcus pneumoniae serotype 4 | transmembrane |
| | | | | | (strain ATCC BAA-334/TIGR4) GN = ftsX | |
| | | | | | PE = 3 SV = 1 - [Q97RP3_STRPN] | |
| Q97T46 | 36.84 | 5 | 95 | 189.78 | Putative bacteriocin OS = -Streptococcus | Secretory |
| | | | | | pneumoniae serotype 4 (strain ATCC | (released) |
| | | | | | BAA-334/TIGR4) GN = SP_0109 PE = 4 | (no CS) |
| | | | | | SV = 1 - [Q97T46_STRPN] | |
| Q97PD6 | 51.85 | 31 | 837 | 188.69 | Protein translocase subunit SecA 1 OS = | Intracellular |
| | | | | | Streptococcus pneumoniae serotype 4 | |
| | | | | | (strain ATCC BAA-334/TIGR4) | |
| | | | | | GN = secA1 PE = 3 SV = 1 - [SECA1_STRPN] | |
| P0A4A7 | 60.58 | 13 | 137 | 187.87 | 30S ribosomal protein S12 OS = | Intracellular |
| | | | | | Streptococcus pneumoniae serotype 4 | |
| | | | | | (strain ATCC BAA-334/TIGR4) | |
| | | | | | GN = rpsL PE = 3 SV = 1 - [RS12_STRPN] | |
| P0A2U8 | 52.96 | 20 | 355 | 185.88 | Oligopeptide transport ATP-binding | Intracellular |
| | | | | | protein AmiE OS = -Streptococcus | |
| | | | | | pneumoniae serotype 4 (strain ATCC | |
| | | | | | BAA-334/TIGR4) GN = amiE PE = 3 SV = 1 - | |
| | | | | | [AMIE_STRPN] | |
| Q97RQ0 | 70.21 | 18 | 386 | 182.7 | Branched-chain amino acid ABC | Lipid |
| | | | | | transporter, amino acid-binding | anchored |
| | | | | | protein OS = -Streptococcus | |
| | | | | | pneumoniae serotype 4 (strain ATCC | |
| | | | | | BAA-334/TIGR4) GN = livJ PE = 4 SV = 1 - | |
| | | | | | [Q97RQ0_STRPN] | |
| P0A3M9 | 67.68 | 16 | 328 | 176.61 | L-lactate dehydrogenase OS = | Intracellular |
| | | | | | Streptococcus pneumoniae serotype 4 | |
| | | | | | (strain ATCC BAA-334/TIGR4) GN = ldh | |
| | | | | | PE = 3 SV = 2 - [LDH_STRPN] | |
| Q97RG9 | 49.51 | 20 | 511 | 174.4 | Sugar ABC transporter, ATP-binding | Intracellular |
| | | | | | protein OS = -Streptococcus | |
| | | | | | pneumoniae serotype 4 (strain ATCC | |
| | | | | | BAA-334/TIGR4) GN = SP_0846 PE = 3 | |
| | | | | | SV = 1 - [Q97RG9_STRPN] | |
| P0A3M5 | 43.38 | 22 | 680 | 166.84 | Penicillin-binding protein 2B OS = | N- |
| | | | | | Streptococcus pneumoniae serotype 4 | terminally |
| | | | | | (strain ATCC BAA-334/TIGR4) | anchored |
| | | | | | GN = penA PE = 3 SV = 1 - [PBP2_STRPN] | (No CS) |
| Q97N53 | 63.24 | 12 | 272 | 163.14 | Cell shape-determining protein MreC | N- |
| | | | | | OS = -Streptococcus pneumoniae | terminally |
| | | | | | serotype 4 (strain ATCC BAA-334/ | anchored |
| | | | | | TIGR4) GN = mreC PE = 3 SV = 1 - | (No CS) |
| | | | | | [Q97N53_STRPN] | |
| P95829 | 49.26 | 20 | 607 | 161.66 | Chaperone protein Dnak OS = | Intracellular |
| | | | | | Streptococcus pneumoniae serotype 4 | |
| | | | | | (strain ATCC BAA-334/TIGR4) | |
| | | | | | GN = dnaK PE = 3 SV = 2 - [DNAK_STRPN] | |
| Q97S19 | 67.89 | 7 | 109 | 156.35 | Cell cycle protein GpsB OS = | Intracellular |
| | | | | | Streptococcus pneumoniae serotype 4 | |
| | | | | | (strain ATCC BAA-334/TIGR4) | |
| | | | | | GN = gpsB PE = 3 SV = 1 - [GPSB_STRPN] | |
| Q97NE4 | 36.3 | 26 | 821 | 153.09 | Penicillin-binding protein 1B OS = | N- |
| | | | | | Streptococcus pneumoniae serotype 4 | terminally |
| | | | | | (strain ATCC BAA-334/TIGR4) | anchored |
| | | | | | GN = pbp1B PE = 4 SV = 1 - | (No CS) |
| | | | | | [Q97NE4_STRPN] | |
| I6L8W3 | 71.49 | 15 | 242 | 152.01 | Choline transporter OS = -Streptococcus | Intracellular |
| | | | | | pneumoniae serotype 4 (strain ATCC | |
| | | | | | BAA-334/TIGR4) GN = proV PE = 3 SV = 1 - | |
| | | | | | [I6L8W3_STRPN] | |
| P0A335 | 52.04 | 21 | 540 | 151.44 | 60 kDa chaperonin OS = -Streptococcus | Intracellular |
| | | | | | pneumoniae serotype 4 (strain ATCC | |
| | | | | | BAA-334/TIGR4) GN = groL PE = 1 SV = 1 - | |
| | | | | | [CH60_STRPN] | |
| Q97NG0 | 44.77 | 17 | 449 | 145.04 | Glucose-6-phosphate isomerase OS = | Intracellular |
| | | | | | Streptococcus pneumoniae serotype 4 | |
| | | | | | (strain ATCC BAA-334/TIGR4) GN = pgi | |
| | | | | | PE = 3 SV = 1 - [G6PI_STRPN] | |
| Q97Q31 | 57.53 | 12 | 292 | 143.85 | Phosphate-binding protein PstS 1 OS = | Lipid |
| | | | | | Streptococcus pneumoniae serotype 4 | anchored |
| | | | | | (strain ATCC BAA-334/TIGR4) | |
| | | | | | GN = pstS1 PE = 1 SV = 1 - [PSTS1_STRPN] | |
| Q97RW9 | 51.81 | 16 | 332 | 139.95 | Putative pneumococcal surface protein | Lipid |
| | | | | | OS = -Streptococcus pneumoniae | anchored |
| | | | | | serotype 4 (strain ATCC BAA-334/ | |
| | | | | | TIGR4) GN = SP_0667 PE = 1 SV = 1 - | |
| | | | | | [Q97RW9_STRPN] | |
| P22976 | 44.98 | 18 | 658 | 139.63 | Probable transketolase OS = | Intracellular |
| | | | | | Streptococcus pneumoniae serotype 4 | |
| | | | | | (strain ATCC BAA-334/TIGR4) GN = tkt | |
| | | | | | PE = 3 SV = 2 - [TKT_STRPN] | |
| Q97SQ9 | 42.52 | 17 | 602 | 138.65 | Glutamine--fructose-6-phosphate | Intracellular |
| | | | | | aminotransferase [isomerizing] OS = | |
| | | | | | Streptococcus pneumoniae serotype 4 | |
| | | | | | (strain ATCC BAA-334/TIGR4) | |
| | | | | | GN = glmS PE = 3 SV = 3 - [GLMS_STRPN] | |
| Q97PA9 | 33.84 | 18 | 659 | 132.79 | Serine/threonine-protein kinase StkP | Intracellular/ |
| | | | | | OS = -Streptococcus pneumoniae | TMH start |
| | | | | | serotype 4 (strain ATCC BAA-334/ | AFTER 60 |
| | | | | | TIGR4) GN = stkP PE = 1 SV = 1 - | |
| | | | | | [STKP2_STRPN] | |
| Q97Q34 | 70.41 | 18 | 267 | 131.91 | Phosphate import ATP-binding protein | Intracellular |
| | | | | | PstB 2 OS = -Streptococcus pneumoniae | |
| | | | | | serotype 4 (strain ATCC BAA-334/ | |
| | | | | | TIGR4) GN = pstB2 PE = 3 SV = 1 - | |
| | | | | | [PSTB2_STRPN] | |
| Q97QX5 | 55.81 | 14 | 344 | 130.66 | Uncharacterized protein OS = | Secretory( |
| | | | | | Streptococcus pneumoniae serotype 4 | released) |
| | | | | | (strain ATCC BAA-334/TIGR4) | (with CS) |
| | | | | | GN = SP_1069 PE = 1 SV = 1 - | |
| | | | | | [Q97QX5_STRPN] | |
| I6L8V0 | 55.84 | 18 | 351 | 130.62 | Capsular polysaccharide biosynthesis | Intracellular |
| | | | | | protein Cps4J OS = -Streptococcus | |
| | | | | | pneumoniae serotype 4 (strain ATCC | |
| | | | | | BAA-334/TIGR4) GN = cap4J PE = 4 SV = 1 - | |
| | | | | | [I6L8V0_STRPN] | |
| Q97SD1 | 43.38 | 17 | 521 | 119.96 | Amino acid ABC transporter, amino | Multi- |
| | | | | | acid-binding protein/permease protein | transmembrane |
| | | | | | OS = -Streptococcus pneumoniae | |
| | | | | | serotype 4 (strain ATCC BAA-334/ | |
| | | | | | TIGR4) GN = SP_0453 PE = 4 SV = 1 - | |
| | | | | | [Q97SD1_STRPN] | |
| Q97SR1 | 50.73 | 21 | 617 | 119.02 | Proline--tRNA ligase OS = -Streptococcus | Intracellular |
| | | | | | pneumoniae serotype 4 (strain ATCC | |
| | | | | | BAA-334/TIGR4) GN = proS PE = 3 SV = 1 - | |
| | | | | | [SYP_STRPN] | |
| P0A4D7 | 36.07 | 14 | 524 | 117.93 | DEAD-box ATP-dependent RNA | Intracellular |
| | | | | | helicase CshA OS = -Streptococcus | |
| | | | | | pneumoniae serotype 4 (strain ATCC | |
| | | | | | BAA-334/TIGR4) GN = cshA PE = 3 SV = 1 - | |
| | | | | | [CSHA_STRPN] | |
| Q97P40 | 68.81 | 12 | 202 | 117.92 | Putative general stress protein 24 OS = | Intracellular |
| | | | | | Streptococcus pneumoniae serotype 4 | |
| | | | | | (strain ATCC BAA-334/TIGR4) | |
| | | | | | GN = SP_1804 PE = 4 SV = 1 - | |
| | | | | | [Q97P40_STRPN] | |
| Q97NK0 | 41 | 21 | 883 | 117.4 | Aldehyde-alcohol dehydrogenase OS = | Intracellular |
| | | | | | Streptococcus pneumoniae serotype 4 | |
| | | | | | (strain ATCC BAA-334/TIGR4) | |
| | | | | | GN = SP_2026 PE = 3 SV = 1 - | |
| | | | | | [Q97NK0_STRPN] | |
| Q97N99 | 51.82 | 14 | 274 | 115.56 | SPFH domain/Band 7 family OS = | Intracellular |
| | | | | | Streptococcus pneumoniae serotype 4 | |
| | | | | | (strain ATCC BAA-334/TIGR4) | |
| | | | | | GN = SP_2156 PE = 4 SV = 1 - | |
| | | | | | [Q97N99_STRPN] | |
| Q9L7Q2 | 17.63 | 23 | 190 6 | 114.8 | Zinc metalloprotease ZmpB OS = | Multi- |
| | | | | | Streptococcus pneumoniae serotype 4 | transmembrane |
| | | | | | (strain ATCC BAA-334/TIGR4) | |
| | | | | | GN = zmpB PE = 3 SV = 2 - [ZMPB_STRPN] | |
| P67293 | 43.9 | 4 | 82 | 114.56 | UPF0154 protein SP_1882 OS = | N- |
| | | | | | Streptococcus pneumoniae serotype 4 | terminally |
| | | | | | (strain ATCC BAA-334/TIGR4) | anchored |
| | | | | | GN = SP_1882 PE = 3 SV = 1 - | (No CS) |
| | | | | | [Y1882_STRPN] | |
| Q97N37 | 61.83 | 14 | 393 | 114.16 | Serine protease OS = -Streptococcus | N- |
| | | | | | pneumoniae serotype 4 (strain ATCC | terminally |
| | | | | | BAA-334/TIGR4) GN = SP_2239 PE = 1 | anchored |
| | | | | | SV = 1 - [Q97N37_STRPN] | (No CS) |
| P66359 | 55.12 | 8 | 127 | 112.68 | 30S ribosomal protein S11 OS = | Intracellular |
| | | | | | Streptococcus pneumoniae serotype 4 | |
| | | | | | (strain ATCC BAA-334/TIGR4) | |
| | | | | | GN = rpsK PE = 3 SV = 1 - [RS11_STRPN] | |
| Q97PX1 | 30.72 | 12 | 459 | 110.91 | NADH oxidase OS = -Streptococcus | Intracellular |
| | | | | | pneumoniae serotype 4 (strain ATCC | |
| | | | | | BAA-334/TIGR4) GN = nox PE = 4 SV = 1 - | |
| | | | | | [Q97PX1_STRPN] | |
| P35592 | 43.64 | 21 | 660 | 109.44 | Oligopeptide-binding protein AliA OS = | Lipid |
| | | | | | Streptococcus pneumoniae serotype 4 | anchored |
| | | | | | (strain ATCC BAA-334/TIGR4) GN = aliA | |
| | | | | | PE = 3 SV = 4 - [ALIA_STRPN] | |
| Q97PC4 | 28.66 | 11 | 492 | 108.41 | ABC transporter, ATP-binding protein | Multi- |
| | | | | | OS = -Streptococcus pneumoniae | transmembrane |
| | | | | | serotype 4 (strain ATCC BAA-334/ | |
| | | | | | TIGR4) GN = SP_1715 PE = 4 SV = 1 - | |
| | | | | | [Q97PC4_STRPN] | |
| Q97P68 | 69.23 | 8 | 104 | 104.87 | Thioredoxin OS = -Streptococcus | Intracellular |
| | | | | | pneumoniae serotype 4 (strain ATCC | |
| | | | | | BAA-334/TIGR4) GN = trx PE = 3 SV = 1 - | |
| | | | | | [Q97P68_STRPN] | |
| Q97PL8 | 41.55 | 12 | 438 | 101 | Oxidoreductase, pyridine nucleotide- | Intracellular |
| | | | | | disulfide, class I OS = -Streptococcus | |
| | | | | | pneumoniae serotype 4 (strain ATCC | |
| | | | | | BAA-334/TIGR4) GN = SP_1588 PE = 3 | |
| | | | | | SV = 1 - [Q97PL8_STRPN] | |
| Q975X2 | 68.78 | 17 | 189 | 97.17 | Uncharacterized protein OS = | Lipid |
| | | | | | Streptococcus pneumoniae serotype 4 | anchored |
| | | | | | (strain ATCC BAA-334/TIGR4) | |
| | | | | | GN = SP_0191 PE = 1 SV = 1 - | |
| | | | | | [Q97SX2_STRPN] | |
| P63413 | 43.94 | 14 | 396 | 96.43 | Acetate kinase OS = -Streptococcus | Intracellular |
| | | | | | pneumoniae serotype 4 (strain ATCC | |
| | | | | | BAA-334/TIGR4) GN = ackA PE = 3 SV = 1 - | |
| | | | | | [ACKA_STRPN] | |
| Q97SJ6 | 52.61 | 11 | 230 | 96.34 | Capsular polysaccharide biosynthesis | Multi- |
| | | | | | protein CpsC OS = -Streptococcus | transmembrane |
| | | | | | pneumoniae serotype 4 (strain ATCC | |
| | | | | | BAA-334/TIGR4) GN = cpsC PE = 3 SV = 1 - | |
| | | | | | [CPSC_STRPN] | |
| Q97Q67 | 38.08 | 11 | 344 | 95.87 | Conserved domain protein OS = | Multi- |
| | | | | | Streptococcus pneumoniae serotype 4 | transmembrane |
| | | | | | (strain ATCC BAA-334/TIGR4) | |
| | | | | | GN = SP_1363 PE = 4 SV = 1 - | |
| | | | | | [Q97Q67_STRPN] | |
| P0A4S1 | 49.15 | 10 | 293 | 93.48 | Fructose-bisphosphate aldolase OS = | Intracellular |
| | | | | | Streptococcus pneumoniae serotype 4 | |
| | | | | | (strain ATCC BAA-334/TIGR4) GN = fba | |
| | | | | | PE = 3 SV = 1 - [ALF_STRPN] | |
| Q97SR2 | 37.71 | 11 | 419 | 93.31 | Putative zinc metalloprotease SP_0263 | Multi- |
| | | | | | OS = -Streptococcus pneumoniae | transmembrane |
| | | | | | serotype 4 (strain ATCC BAA-334/ | |
| | | | | | TIGR4) GN = SP_0263 PE = 3 SV = 1 - | |
| | | | | | [Y263_STRPN] | |
| Q97NP5 | 25.65 | 9 | 308 | 92.53 | Membrane protein insertase YidC 1 | Multi- |
| | | | | | OS = -Streptococcus pneumoniae | transmembrane |
| | | | | | serotype 4 (strain ATCC BAA-334/ | |
| | | | | | TIGR4) GN = yidC1 PE = 3 SV = 1 - | |
| | | | | | [YIDC1_STRPN] | |
| Q97SJ4 | 44.44 | 15 | 360 | 91.77 | Uncharacterized protein OS = | Intracellular |
| | | | | | Streptococcus pneumoniae serotype 4 | |
| | | | | | (strain ATCC BAA-334/TIGR4) | |
| | | | | | GN = SP_0355 PE = 4 SV = 1 - | |
| | | | | | [Q97SJ4_STRPN] | |
| Q97RK0 | 19.83 | 10 | 575 | 91.07 | Septation ring formation regulator | N- |
| | | | | | EzrA OS = = -Streptococcus pneumoniae | terminally |
| | | | | | serotype 4 (strain ATCC BAA-334/ | anchored |
| | | | | | TIGR4) GN = ezrA PE = 3 SV = 1 - | (No CS) |
| | | | | | [EZRA_STRPN] | |
| P63373 | 71.43 | 11 | 252 | 91.01 | Phosphate import ATP-binding protein | Intracellular |
| | | | | | PstB 1 OS = -Streptococcus pneumoniae | |
| | | | | | serotype 4 (strain ATCC BAA-334/ | |
| | | | | | TIGR4) GN = pstB1 PE = 3 SV = 1 - | |
| | | | | | [PSTB1_STRPN] | |
| Q97S89 | 51.01 | 13 | 398 | 89.47 | Phosphoglycerate kinase OS = | Intracellular |
| | | | | | Streptococcus pneumoniae serotype 4 | |
| | | | | | (strain ATCC BAA-334/TIGR4) GN = pgk | |
| | | | | | PE = 3 SV = 1 - [PGK_STRPN] | |
| Q97PT5 | 47.26 | 12 | 292 | 85.91 | ATP synthase gamma chain OS = | Intracellular |
| | | | | | Streptococcus pneumoniae serotype 4 | |
| | | | | | (strain ATCC BAA-334/TIGR4) | |
| | | | | | GN = atpG PE = 3 SV = 1 - [ATPG_STRPN] | |
| Q97514 | 32.65 | 9 | 340 | 85.66 | Choline binding protein C OS = | N- |
| | | | | | Streptococcus pneumoniae serotype 4 | terminally |
| | | | | | (strain ATCC BAA-334/TIGR4) | anchored |
| | | | | | GN = cbpC PE = 4 SV = 1 - [Q97SI4_STRPN] | (with CS) |
| I6L858 | 39.71 | 14 | 481 | 85.62 | Capsular polysaccharide biosynthesis | Multi- |
| | | | | | protein Cps4A OS = -Streptococcus | transmembrane |
| | | | | | pneumoniae serotype 4 (strain ATCC | |
| | | | | | BAA-334/TIGR4) GN = cps4A PE = 4 | |
| | | | | | SV = 1 - [I6L8S8_STRPN] | |
| Q97PY0 | 65.59 | 13 | 247 | 85.07 | Amino acid ABC transporter, ATP- | Intracellular |
| | | | | | binding protein OS = -Streptococcus | |
| | | | | | pneumoniae serotype 4 (strain ATCC | |
| | | | | | BAA-334/TIGR4) GN = SP_1460 PE = 3 | |
| | | | | | SV = 1 - [Q97PYO_STRPN] | |
| Q97SQ4 | 46.79 | 9 | 156 | 83.39 | 30S ribosomal protein S7 OS = | Intracellular |
| | | | | | Streptococcus pneumoniae serotype 4 | |
| | | | | | (strain ATCC BAA-334/TIGR4) | |
| | | | | | GN = rpsG PE = 3 SV = 1 - [RS7_STRPN] | |
| P0A3Y3 | 60 | 10 | 230 | 82.54 | 2,3-bisphosphoglycerate-dependent | Intracellular |
| | | | | | phosphoglycerate mutase OS = | |
| | | | | | Streptococcus pneumoniae serotype 4 | |
| | | | | | (strain ATCC BAA-334/TIGR4) | |
| | | | | | GN = gpmA PE = 3 SV = 1 - [GPMA_STRPN] | |
| P0A4C3 | 59.91 | 11 | 217 | 82.05 | 30S ribosomal protein S3 OS = | Intracellular |
| | | | | | Streptococcus pneumoniae serotype 4 | |
| | | | | | (strain ATCC BAA-334/TIGR4) | |
| | | | | | GN = rpsC PE = 3 SV = 1 - [RS3_STRPN] | |
| P65607 | 38.46 | 11 | 338 | 79.74 | Ornithine carbamoyltransferase, | Intracellular |
| | | | | | catabolic OS = -Streptococcus | |
| | | | | | pneumoniae serotype 4 (strain ATCC | |
| | | | | | BAA-334/TIGR4) GN = arcB PE = 3 SV = 1 - | |
| | | | | | [OTCC_STRPN] | |
| O05703 | 39.92 | 12 | 501 | 79.68 | Zinc-binding lipoprotein AdcA OS = | Lipid |
| | | | | | Streptococcus pneumoniae serotype 4 | anchored |
| | | | | | (strain ATCC BAA-334/TIGR4) | |
| | | | | | GN = adcA PE = 3 SV = 4 - [ADCA_STRPN] | |
| I6L8U0 | 27.16 | 11 | 394 | 79.39 | UDP-N-acetylglucosamine 2-epimerase | Intracellular |
| | | | | | OS = -Streptococcus pneumoniae | |
| | | | | | serotype 4 (strain ATCC BAA-334/ | |
| | | | | | TIGR4) GN = cps4L PE = 3 SV = 1 - | |
| | | | | | [I6L8U0_STRPN] | |
| Q97SI7 | 28.23 | 8 | 464 | 78.19 | Mid-cell-anchored protein Z OS = | Intracellular |
| | | | | | Streptococcus pneumoniae serotype 4 | |
| | | | | | (strain ATCC BAA-334/TIGR4) | |
| | | | | | GN = mapZ PE = 1 SV = 1 - | |
| | | | | | [Q97SI7_STRPN] | |
| Q97N56 | 58.3 | 15 | 259 | 78.0 | 30S ribosomal protein S2 OS = | Intracellular |
| | | | | | Streptococcus pneumoniae serotype 4 | |
| | | | | | (strain ATCC BAA-334/TIGR4) | |
| | | | | | GN = rpsB PE = 3 SV = 1 - [RS2_STRPN] | |
| Q97SI6 | 35.44 | 13 | 474 | 77.15 | 6-phosphogluconate dehydrogenase, | Intracellular |
| | | | | | decarboxylating OS = -Streptococcus | |
| | | | | | pneumoniae serotype 4 (strain ATCC | |
| | | | | | BAA-334/TIGR4) GN = gnd PE = 3 SV = 1 - | |
| | | | | | [Q97SI6_STRPN] | |
| Q97PG0 | 27.1 | 7 | 262 | 77.07 | Cell division protein DivIVA OS = | Intracellular |
| | | | | | Streptococcus pneumoniae serotype 4 | |
| | | | | | (strain ATCC BAA-334/TIGR4) | |
| | | | | | GN = divIVA PE = 4 SV = 1 - | |
| | | | | | [Q97PG0_STRPN] | |
| Q97RF9 | 32 | 11 | 400 | 76.46 | Ribosomal protein S1 OS = | Intracellular |
| | | | | | Streptococcus pneumoniae serotype 4 | |
| | | | | | (strain ATCC BAA-334/TIGR4) | |
| | | | | | GN = rpsA PE = 4 SV = 1 - [Q97RF9_STRPN] | |
| Q97T80 | 15.68 | 17 | 1856 | 76.28 | Zinc metalloprotease ZmpC OS = | LPXTG |
| | | | | | Streptococcus pneumoniae serotype 4 | Cell-wall |
| | | | | | (strain ATCC BAA-334/TIGR4) | anchored |
| | | | | | GN = zmpC PE = 3 SV = 1 - [ZMPC_STRPN] | |
| P0A2Z2 | 62.8 | 9 | 164 | 75.56 | ATP synthase subunit b OS = | Intracellular |
| | | | | | Streptococcus pneumoniae serotype 4 | |
| | | | | | (strain ATCC BAA-334/TIGR4) | |
| | | | | | GN = atpF PE = 3 SV = 1 - [ATPF_STRPN] | |
| I6L859 | 47.7 | 10 | 413 | 73.22 | D-alanyl-D-alanine carboxypeptidase | N- |
| | | | | | OS = -Streptococcus pneumoniae | terminally |
| | | | | | serotype 4 (strain ATCC BAA-334/ | anchored |
| | | | | | TIGR4) GN = dacA PE = 3 SV = 1 - | (with CS) |
| | | | | | [I6L859_STRPN] | |
| Q97PU4 | 80 | 7 | 115 | 71.8 | Bacterocin transport accessory protein | Intracellular |
| | | | | | OS = -Streptococcus pneumoniae | |
| | | | | | serotype 4 (strain ATCC BAA-334/ | |
| | | | | | TIGR4) GN = bta PE = 4 SV = 1 - | |
| | | | | | [Q97PU4_STRPN] | |
| Q97SU6 | 70.34 | 7 | 118 | 71.64 | 50S ribosomal protein L18 OS = | Intracellular |
| | | | | | Streptococcus pneumoniae serotype 4 | |
| | | | | | (strain ATCC BAA-334/TIGR4) GN = rplR | |
| | | | | | PE = 3 SV = 1 - [RL18_STRPN] | |
| P63742 | 35.46 | 11 | 502 | 71.21 | Glycerol kinase OS = -Streptococcus | Intracellular |
| | | | | | pneumoniae serotype 4 (strain ATCC | |
| | | | | | BAA-334/TIGR4) GN = glpK PE = 3 SV = 1 - | |
| | | | | | [GLPK_STRPN] | |
| Q97SN4 | 32.31 | 6 | 130 | 68.89 | 30S ribosomal protein S9 OS = | Intracellular |
| | | | | | Streptococcus pneumoniae serotype 4 | |
| | | | | | (strain ATCC BAA-334/TIGR4) GN = rpsI | |
| | | | | | PE = 3 SV = 1 - [RS9_STRPN] | |
| P66907 | 31.84 | 6 | 380 | 68.13 | Queuine tRNA-ribosyltransferase OS = | Intracellular |
| | | | | | Streptococcus pneumoniae serotype 4 | |
| | | | | | (strain ATCC BAA-334/TIGR4) GN = tgt | |
| | | | | | PE = 3 SV = 1 - [TGT_STRPN] | |
| Q97SF8 | 32.16 | 12 | 454 | 65.06 | Aspartokinase OS = -Streptococcus | Intracellular |
| | | | | | pneumoniae serotype 4 (strain ATCC | |
| | | | | | BAA-334/TIGR4) GN = SP_0413 PE = 3 | |
| | | | | | SV = 1 - [Q97SF8_STRPN] | |
| P0A471 | 82.79 | 9 | 122 | 62.07 | 50S ribosomal protein L7/L12 OS = | Intracellular |
| | | | | | Streptococcus pneumoniae serotype 4 | |
| | | | | | (strain ATCC BAA-334/TIGR4) GN = rplL | |
| | | | | | PE = 1 SV = 2 - [RL7_STRPN] | |
| I6L8W8 | 21.08 | 8 | 332 | 61.96 | Choline binding protein J OS = | N- |
| | | | | | Streptococcus pneumoniae serotype 4 | terminally |
| | | | | | (strain ATCC BAA-334/TIGR4) | anchored |
| | | | | | GN = cbpJ PE = 4 SV = 1 - [I6L8W8_STRPN] | (with CS) |
| P0A4J6 | 40.8 | 5 | 201 | 61.71 | Superoxide dismutase [Mn] OS = | Intracellular |
| | | | | | Streptococcus pneumoniae serotype 4 | |
| | | | | | (strain ATCC BAA-334/TIGR4) | |
| | | | | | GN = sodA PE = 1 SV = 2 - [SODM_STRPN] | |
| Q97T52 | 30.03 | 14 | 616 | 60.5 | Putative capsular polysaccharide | Multi- |
| | | | | | biosynthesis protein OS = -Streptococcus | transmembrane |
| | | | | | pneumoniae serotype 4 (strain ATCC | |
| | | | | | BAA-334/TIGR4) GN = SP_0103 PE = 4 | |
| | | | | | SV = 1 - [Q97T52_STRPN] | |
| Q97RE5 | 17.08 | 6 | 650 | 60.23 | PTS system, fructose specific IIABC | Multi- |
| | | | | | components OS = -Streptococcus | transmembrane |
| | | | | | pneumoniae serotype 4 (strain ATCC | |
| | | | | | BAA-334/TIGR4) GN = SP_0877 PE = 4 | |
| | | | | | SV = 1 - [Q97RE5_STRPN] | |
| Q97SV4 | 54.11 | 9 | 207 | 60.13 | 50S ribosomal protein L4 OS = | Intracellular |
| | | | | | Streptococcus pneumoniae serotype 4 | |
| | | | | | (strain ATCC BAA-334/TIGR4) | |
| | | | | | GN = rplD PE = 3 SV = 1 - [RL4_STRPN] | |
| Q97RK7 | 19.46 | 12 | 848 | 59.83 | Aminopeptidase N OS = -Streptococcus | Intracellular |
| | | | | | pneumoniae serotype 4 (strain ATCC | |
| | | | | | BAA-334/TIGR4) GN = pepN PE = 4 SV = 1 - | |
| | | | | | [Q97RK7_STRPN] | |
| Q97Q62 | 34.67 | 11 | 424 | 59.21 | Psr protein OS = -Streptococcus | Intracellular/ |
| | | | | | pneumoniae serotype 4 (strain ATCC | TMH start |
| | | | | | BAA-334/TIGR4) GN = SP_1368 PE = 4 | AFTER 60 |
| | | | | | SV = 1 - [Q97Q62_STRPN] | |
| O07344 | 26.96 | 5 | 204 | 58.64 | Signal peptidase I OS = -Streptococcus | N- |
| | | | | | pneumoniae serotype 4 (strain ATCC | terminally |
| | | | | | BAA-334/TIGR4) GN = lepB PE = 3 SV = 2 - | anchored |
| | | | | | [LEP_STRPN] | (No CS) |
| P0A3S3 | 34.67 | 6 | 274 | 57.63 | DNA-entry nuclease OS = -Streptococcus | N- |
| | | | | | pneumoniae serotype 4 (strain ATCC | terminally |
| | | | | | BAA-334/TIGR4) GN = endA PE = 1 SV = 1 - | anchored |
| | | | | | [NUCE_STRPN] | (No CS) |
| Q97PU5 | 29.55 | 10 | 572 | 57.38 | Phosphoglucomutase OS = | Intracellular |
| | | | | | Streptococcus pneumoniae serotype 4 | |
| | | | | | (strain ATCC BAA-334/TIGR4) | |
| | | | | | GN = pgm PE = 3 SV = 1 - [Q97PU5_STRPN] | |
| Q97RC6 | 31.34 | 8 | 335 | 57.3 | ATP-dependent 6- | Intracellular |
| | | | | | phosphofructokinase OS = | |
| | | | | | Streptococcus pneumoniae serotype 4 | |
| | | | | | (strain ATCC BAA-334/TIGR4) | |
| | | | | | GN = pfkA PE = 3 SV = 1 - [PFKA_STRPN] | |
| Q97SV5 | 37.5 | 6 | 208 | 56.96 | 50S ribosomal protein L3 OS = | Intracellular |
| | | | | | Streptococcus pneumoniae serotype 4 | |
| | | | | | (strain ATCC BAA-334/TIGR4) GN = rplC | |
| | | | | | PE = 3 SV = 1 - [RL3_STRPN] | |
| Q97RL9 | 25.56 | 6 | 399 | 56.21 | Uncharacterized protein OS = | N- |
| | | | | | Streptococcus pneumoniae serotype 4 | terminally |
| | | | | | (strain ATCC BAA-334/TIGR4) | anchored |
| | | | | | GN = SP_0785 PE = 4 SV = 1 - | (No CS) |
| | | | | | [Q97RL9_STRPN] | |
| P66278 | 18.18 | 2 | 66 | 55.84 | 50S ribosomal protein L35 OS = | Intracellular |
| | | | | | Streptococcus pneumoniae serotype 4 | |
| | | | | | (strain ATCC BAA-334/TIGR4) | |
| | | | | | GN = rpmI PE = 3 SV = 1 - [RL35_STRPN] | |
| I6L8N0 | 40.82 | 12 | 365 | 55.32 | UDP-N-acetylglucosamine-2-epimerase | Intracellular |
| | | | | | OS = -Streptococcus pneumoniae | |
| | | | | | serotype 4 (strain ATCC BAA-334/ | |
| | | | | | TIGR4) GN = cps4I PE = 3 SV = 1 - | |
| | | | | | [I6L8N0_STRPN] | |
| Q97PQ8 | 57.34 | 6 | 143 | 54.74 | Uncharacterized protein OS = | Intracellular |
| | | | | | Streptococcus pneumoniae serotype 4 | |
| | | | | | (strain ATCC BAA-334/TIGR4) | |
| | | | | | GN = SP_1545 PE = 4 SV = 1 - | |
| | | | | | [Q97PQ8_STRPN] | |
| Q97TA6 | 33.64 | 10 | 330 | 52.71 | Phosphate acyltransferase OS = | Intracellular |
| | | | | | Streptococcus pneumoniae serotype 4 | |
| | | | | | (strain ATCC BAA-334/TIGR4) GN = plsX | |
| | | | | | PE = 3 SV = 1 - [PLSX_STRPN] | |
| Q97PK5 | 27.22 | 2 | 158 | 52.32 | Uncharacterized protein OS = | N- |
| | | | | | Streptococcus pneumoniae serotype 4 | terminally |
| | | | | | (strain ATCC BAA-334/TIGR4) | anchored |
| | | | | | GN = SP_1604 PE = 4 SV = 1 - | (No CS) |
| | | | | | [Q97PK5_STRPN] | |
| Q97SN5 | 67.57 | 8 | 148 | 51.12 | 50S ribosomal protein L13 OS = | Intracellular |
| | | | | | Streptococcus pneumoniae serotype 4 | |
| | | | | | (strain ATCC BAA-334/TIGR4) | |
| | | | | | GN = rplM PE = 3 SV = 1 - | |
| | | | | | [Q97SN5_STRPN] | |
| Q97RV8 | 60.32 | 8 | 126 | 49.28 | Uncharacterized protein OS = | N- |
| | | | | | Streptococcus pneumoniae serotype 4 | terminally |
| | | | | | (strain ATCC BAA-334/TIGR4) | anchored |
| | | | | | GN = SP_0678 PE = 4 SV = 1 - | (No CS) |
| | | | | | [Q97RV8_STRPN] | |
| Q97R14 | 30.25 | 7 | 324 | 48.6 | Uncharacterized protein OS = | Secretory |
| | | | | | Streptococcus pneumoniae serotype 4 | (released) |
| | | | | | (strain ATCC BAA-334/TIGR4) | (with CS) |
| | | | | | GN = SP_1027 PE = 4 SV = 1 - | |
| | | | | | [Q97R14_STRPN] | |
| P35597 | 15.04 | 7 | 778 | 48.34 | Probable cation-transporting ATPase | Multi- |
| | | | | | exp7 OS = -Streptococcus pneumoniae | transmembrane |
| | | | | | serotype 4 (strain ATCC BAA-334/ | |
| | | | | | TIGR4) GN = exp7 PE = 3 SV = 2 - | |
| | | | | | [EXP7_STRPN] | |
| P65239 | 41.61 | 9 | 322 | 48.08 | Ribose-phosphate pyrophosphokinase | Intracellular |
| | | | | | 1 OS = -Streptococcus pneumoniae | |
| | | | | | serotype 4 (strain ATCC BAA-334/ | |
| | | | | | TIGR4) GN = prs1 PE = 3 SV = 1 - | |
| | | | | | [KPRS1_STRPN] | |
| Q97Q37 | 46.13 | 7 | 271 | 47.67 | Amino acid ABC transporter, amino | Lipid |
| | | | | | acid-binding protein OS = -Streptococcus | anchored |
| | | | | | pneumoniae serotype 4 (strain ATCC | |
| | | | | | BAA-334/TIGR4) GN = SP_1394 PE = 4 | |
| | | | | | SV = 1 - [Q97Q37_STRPN] | |
| Q97R36 | 52.43 | 8 | 185 | 47.65 | Thioredoxin family protein OS = | Lipid |
| | | | | | Streptococcus pneumoniae serotype 4 | anchored |
| | | | | | (strain ATCC BAA-334/TIGR4) | |
| | | | | | GN = SP_1000 PE = 1 SV = 1 - | |
| | | | | | [Q97R36_STRPN] | |
| Q97N48 | 26.81 | 5 | 276 | 45.87 | Uncharacterized protein OS = | Intracellular/ |
| | | | | | Streptococcus pneumoniae serotype 4 | TMH start |
| | | | | | (strain ATCC BAA-334/TIGR4) | AFTER 60 |
| | | | | | GN = SP_2223 PE = 4 SV = 1 - | |
| | | | | | [Q97N48_STRPN] | |
| I6L8R1 | 22.79 | 4 | 215 | 45.06 | ABC transporter, ATP-binding protein | Intracellular |
| | | | | | Vexp2 OS = -Streptococcus pneumoniae | |
| | | | | | serotype 4 (strain ATCC BAA-334/ | |
| | | | | | TIGR4) GN = vex2 PE = 3 SV = 1 - | |
| | | | | | [I6L8R1_STRPN] | |
| Q97SC6 | 20.93 | 10 | 774 | 44.93 | Formate acetyltransferase OS = | Intracellular |
| | | | | | Streptococcus pneumoniae serotype 4 | |
| | | | | | (strain ATCC BAA-334/TIGR4) GN = pfl | |
| | | | | | PE = 4 SV = 1 - [Q97SC6_STRPN] | |
| Q97QD5 | 51.08 | 8 | 186 | 44.75 | LemA protein OS = -Streptococcus | N- |
| | | | | | pneumoniae serotype 4 (strain ATCC | terminally |
| | | | | | BAA-334/TIGR4) GN = lemA PE = 1 SV = 1 - | anchored |
| | | | | | [Q97QD5_STRPN] | (No CS) |
| Q97PF9 | 37.71 | 9 | 419 | 44.71 | Cell division protein FtsZ OS = | Intracellular |
| | | | | | Streptococcus pneumoniae serotype 4 | |
| | | | | | (strain ATCC BAA-334/TIGR4) GN = ftsZ | |
| | | | | | PE = 3 SV = 1 - [Q97PF9_STRPN] | |
| P0A3B7 | 36.71 | 7 | 346 | 44.56 | Elongation factor Ts OS = -Streptococcus | Intracellular |
| | | | | | pneumoniae serotype 4 (strain ATCC | |
| | | | | | BAA-334/TIGR4) GN = tsf PE = 1 SV = 2 - | |
| | | | | | [EFTS_STRPN] | |
| P72524 | 20.56 | 10 | 822 | 44.46 | DNA gyrase subunit A OS = | Intracellular |
| | | | | | Streptococcus pneumoniae serotype 4 | |
| | | | | | (strain ATCC BAA-334/TIGR4) | |
| | | | | | GN = gyrA PE = 3 SV = 3 - [GYRA_STRPN] | |
| P67282 | 26.22 | 11 | 534 | 43.28 | Ribonuclease Y OS = -Streptococcus | N- |
| | | | | | pneumoniae serotype 4 (strain ATCC | terminally |
| | | | | | BAA-334/TIGR4) GN = rny PE = 3 SV = 1 - | anchored |
| | | | | | [RNY_STRPN] | (No CS) |
| P66419 | 38.2 | 6 | 89 | 42.98 | 30S ribosomal protein S14 OS = | Intracellular |
| | | | | | Streptococcus pneumoniae serotype 4 | |
| | | | | | (strain ATCC BAA-334/TIGR4) | |
| | | | | | GN = rpsN PE = 3 SV = 1 - [RS14_STRPN] | |
| Q97SV1 | 48.89 | 6 | 180 | 42.64 | 50S ribosomal protein L5 OS = | Intracellular |
| | | | | | Streptococcus pneumoniae serotype 4 | |
| | | | | | (strain ATCC BAA-334/TIGR4) GN = rplE | |
| | | | | | PE = 3 SV = 1 - [RL5_STRPN] | |
| Q97NW9 | 7.48 | 5 | 655 | 42.08 | Trehalose PTS system, IIABC | Multi- |
| | | | | | components OS = -Streptococcus | transmembrane |
| | | | | | pneumoniae serotype 4 (strain ATCC | |
| | | | | | BAA-334/TIGR4) GN = SP_1884 PE = 4 | |
| | | | | | SV = 1 - [Q97NW9_STRPN] | |
| Q97Q18 | 91.06 | 6 | 123 | 40.86 | Uncharacterized protein OS = | Intracellular |
| | | | | | Streptococcus pneumoniae serotype 4 | |
| | | | | | (strain ATCC BAA-334/TIGR4) | |
| | | | | | GN = SP_1218 PE = 4 SV = 1 - | |
| | | | | | [Q97Q18_STRPN] | |
| Q97T04 | 11.92 | 2 | 193 | 39.59 | Uncharacterized protein OS = | Multi- |
| | | | | | Streptococcus pneumoniae serotype 4 | transmembrane |
| | | | | | (strain ATCC BAA-334/TIGR4) | |
| | | | | | GN = SP_0157 PE = 4 SV = 1 - | |
| | | | | | [Q97T04_STRPN] | |
| Q97PB8 | 9.09 | 4 | 627 | 39.49 | PTS system IIABC components OS = | Multi- |
| | | | | | Streptococcus pneumoniae serotype 4 | transmembrane |
| | | | | | (strain ATCC BAA-334/TIGR4) | |
| | | | | | GN = SP_1722 PE = 4 SV = 1 - | |
| | | | | | [Q97PB8_STRPN] | |
| Q97P19 | 57.3 | 5 | 89 | 39.48 | 30S ribosomal protein S15 OS = | Intracellular |
| | | | | | Streptococcus pneumoniae serotype 4 | |
| | | | | | (strain ATCC BAA-334/TIGR4) | |
| | | | | | GN = rpsO PE = 3 SV = 1 - [RS15_STRPN] | |
| P61182 | 40.35 | 4 | 114 | 39.35 | 50S ribosomal protein L22 OS = | Intracellular |
| | | | | | Streptococcus pneumoniae serotype 4 | |
| | | | | | (strain ATCC BAA-334/TIGR4) GN = rplV | |
| | | | | | PE = 1 SV = 1 - [RL22_STRPN] | |
| Q97SU7 | 51.69 | 6 | 178 | 38.14 | 50S ribosomal protein L6 OS = | Intracellular |
| | | | | | Streptococcus pneumoniae serotype 4 | |
| | | | | | (strain ATCC BAA-334/TIGR4) GN = rplF | |
| | | | | | PE = 3 SV = 1 - [RL6_STRPN] | |
| Q97NB9 | 39.1 | 8 | 335 | 38.04 | Uncharacterized protein OS = | Multi- |
| | | | | | Streptococcus pneumoniae serotype 4 | transmembrane |
| | | | | | (strain ATCC BAA-334/TIGR4) | |
| | | | | | GN = SP_2132 PE = 4 SV = 1 - | |
| | | | | | [Q97NB9_STRPN] | |
| Q97QP7 | 5.34 | 8 | 2004 | 37.84 | Immunoglobulin A1 protease OS = | Multi- |
| | | | | | Streptococcus pneumoniae serotype 4 | transmembrane |
| | | | | | (strain ATCC BAA-334/TIGR4) GN = iga | |
| | | | | | PE = 3 SV = 1 - [IGA1A_STRPN] | |
| Q97N55 | 23.72 | 6 | 392 | 37.16 | Secreted 45 kd protein OS = | Secretory |
| | | | | | Streptococcus pneumoniae serotype 4 | (released) |
| | | | | | (strain ATCC BAA-334/TIGR4) | (with CS) |
| | | | | | GN = usp45 PE = 4 SV = 1 - | |
| | | | | | [Q97N55_STRPN] | |
| P66942 | 18.25 | 4 | 252 | 36.63 | Triosephosphate isomerase OS = | Intracellular |
| | | | | | Streptococcus pneumoniae serotype 4 | |
| | | | | | (strain ATCC BAA-334/TIGR4) GN = tpiA | |
| | | | | | PE = 3 SV = 1 - [TPIS_STRPN] | |
| Q97QC6 | 66.09 | 7 | 115 | 35.81 | 50S ribosomal protein L19 OS = | Intracellular |
| | | | | | Streptococcus pneumoniae serotype 4 | |
| | | | | | (strain ATCC BAA-334/TIGR4) GN = rplS | |
| | | | | | PE = 3 SV = 1 - [RL19_STRPN] | |
| I6L8S7 | 14.6 | 5 | 459 | 35.54 | Transmembrane protein Vexp3 OS = | Multi- |
| | | | | | Streptococcus pneumoniae serotype 4 | transmembrane |
| | | | | | (strain ATCC BAA-334/TIGR4) | |
| | | | | | GN = vex3 PE = 4 SV = 1 - [I6L8S7_STRPN] | |
| P35595 | 12.4 | 6 | 726 | 35.44 | PTS system glucose-specific EIICBA | Multi- |
| | | | | | component OS = -Streptococcus | transmembrane |
| | | | | | pneumoniae serotype 4 (strain ATCC | |
| | | | | | BAA-334/TIGR4) GN = exp5 PE = 3 SV = 2 - | |
| | | | | | [PTG3C_STRPN] | |
| Q97PV1 | 14.88 | 3 | 289 | 34.89 | Putative glycerol uptake facilitator | Multi- |
| | | | | | protein OS = -Streptococcus | transmembrane |
| | | | | | pneumoniae serotype 4 (strain ATCC | |
| | | | | | BAA-334/TIGR4) GN = SP_1491 PE = 3 | |
| | | | | | SV = 1 - [Q97PV1_STRPN] | |
| Q97NM6 | 21.04 | 6 | 404 | 33.79 | Aminotransferase, class I OS = | Intracellular |
| | | | | | Streptococcus pneumoniae serotype 4 | |
| | | | | | (strain ATCC BAA-334/TIGR4) | |
| | | | | | GN = SP_1994 PE = 4 SV = 1 - | |
| | | | | | [Q97NM6_STRPN] | |
| Q97SD2 | 45.93 | 5 | 246 | 33.44 | Amino acid ABC transporter, ATP- | Intracellular |
| | | | | | binding protein OS = -Streptococcus | |
| | | | | | pneumoniae serotype 4 (strain ATCC | |
| | | | | | BAA-334/TIGR4) GN = SP_0452 PE = 3 | |
| | | | | | SV = 1 - [Q97SD2_STRPN] | |
| Q97NN3 | 36.15 | 6 | 213 | 33.1 | ABC transporter, ATP-binding protein | Intracellular |
| | | | | | OS = -Streptococcus pneumoniae | |
| | | | | | serotype 4 (strain ATCC BAA-334/ | |
| | | | | | TIGR4) GN = SP_1987 PE = 1 SV = 1 - | |
| | | | | | [Q97NN3_STRPN] | |
| Q97RJ0 | 23.77 | 5 | 244 | 33.09 | Amino acid ABC transporter, ATP- | Intracellular |
| | | | | | binding protein OS = -Streptococcus | |
| | | | | | pneumoniae serotype 4 (strain ATCC | |
| | | | | | BAA-334/TIGR4) GN = SP_0824 PE = 3 | |
| | | | | | SV = 1 - [Q97RJ0_STRPN] | |
| Q97NJ7 | 27.27 | 3 | 99 | 32.76 | Preprotein translocase, YajC subunit | N- |
| | | | | | OS = -Streptococcus pneumoniae | terminally |
| | | | | | serotype 4 (strain ATCC BAA-334/ | anchored |
| | | | | | TIGR4) GN = yajC-2 PE = 4 SV = 1 - | (No CS) |
| | | | | | [Q97NJ7_STRPN] | |
| P66095 | 34.5 | 8 | 229 | 32.4 | 50S ribosomal protein L1 OS = | Intracellular |
| | | | | | Streptococcus pneumoniae serotype 4 | |
| | | | | | (strain ATCC BAA-334/TIGR4) | |
| | | | | | GN = rplA PE = 3 SV = 1 - [RL1_STRPN] | |
| Q97NT7 | 38.61 | 6 | 158 | 32.3 | Uncharacterized protein OS = | Multi- |
| | | | | | Streptococcus pneumoniae serotype 4 | transmembrane |
| | | | | | (strain ATCC BAA-334/TIGR4) | |
| | | | | | GN = SP_1926 PE = 4 SV = 1 - | |
| | | | | | [Q97NT7_STRPN] | |
| P63667 | 29.5 | 4 | 139 | 31.7 | ATP synthase epsilon chain OS = | Intracellular |
| | | | | | Streptococcus pneumoniae serotype 4 | |
| | | | | | (strain ATCC BAA-334/TIGR4) | |
| | | | | | GN = atpC PE = 3 SV = 1 - [ATPE_STRPN] | |
| Q97T58 | 18.08 | 4 | 354 | 31.6 | Conserved domain protein | Multi- |
| | | | | | OS = Streptococcus pneumoniae | transmembrane |
| | | | | | serotype 4 (strain ATCC BAA-334/ | |
| | | | | | TIGR4) GN = SP_0097 PE = 4 SV = 1 - | |
| | | | | | [Q97T58_STRPN] | |
| Q97R06 | 36.74 | 6 | 264 | 31.17 | Iron-compound ABC transporter, ATP- | Intracellular |
| | | | | | binding protein OS = -Streptococcus | |
| | | | | | pneumoniae serotype 4 (strain ATCC | |
| | | | | | BAA-334/TIGR4) GN = SP_1035 PE = 3 | |
| | | | | | SV = 1 - [Q97R06_STRPN] | |
| Q97PQ5 | 20.34 | 6 | 531 | 30.81 | Uncharacterized protein OS = | Multi- |
| | | | | | Streptococcus pneumoniae serotype 4 | transmembrane |
| | | | | | (strain ATCC BAA-334/TIGR4) | |
| | | | | | GN = SP_1548 PE = 4 SV = 1 - | |
| | | | | | [Q97PQ5_STRPN] | |
| Q97R44 | 33.91 | 2 | 115 | 30.62 | Uncharacterized protein OS = | Intracellular/ |
| | | | | | Streptococcus pneumoniae serotype 4 | TMH start |
| | | | | | (strain ATCC BAA-334/TIGR4) | AFTER 60 |
| | | | | | GN = SP_0990 PE = 4 SV = 1 - | |
| | | | | | [Q97R44_STRPN] | |
| Q97SW3 | 47.73 | 3 | 88 | 29.98 | Uncharacterized protein OS = | Intracellular |
| | | | | | Streptococcus pneumoniae serotype 4 | |
| | | | | | (strain ATCC BAA-334/TIGR4) | |
| | | | | | GN = SP_0201 PE = 4 SV = 1 - | |
| | | | | | [Q97SW3_STRPN] | |
| I6L8N6 | 27.01 | 4 | 211 | 29.5 | Capsular polysaccharide biosynthesis | N- |
| | | | | | protein Cps4E OS = -Streptococcus | terminally |
| | | | | | pneumoniae serotype 4 (strain ATCC | anchored |
| | | | | | BAA-334/TIGR4) GN = cps4E PE = 4 SV = 1 - | (No CS) |
| | | | | | [I6L8N6_STRPN] | |
| Q97SU3 | 34.25 | 5 | 146 | 28.53 | 50S ribosomal protein L15 OS = | Intracellular |
| | | | | | Streptococcus pneumoniae serotype 4 | |
| | | | | | (strain ATCC BAA-334/TIGR4) | |
| | | | | | GN = rplO PE = 3 SV = 1 - [RL15_STRPN] | |
| Q97QS6 | 25.86 | 6 | 379 | 28.15 | Glycogen biosynthesis protein GlgD | Intracellular |
| | | | | | OS = -Streptococcus pneumoniae | |
| | | | | | serotype 4 (strain ATCC BAA-334/ | |
| | | | | | TIGR4) GN = glgD PE = 4 SV = 1 - | |
| | | | | | [Q97QS6_STRPN] | |
| Q97P39 | 52.24 | 2 | 67 | 27.23 | UPF0337 protein SP_1805 OS = | Intracellular |
| | | | | | Streptococcus pneumoniae serotype 4 | |
| | | | | | (strain ATCC BAA-334/TIGR4) | |
| | | | | | GN = SP_1805 PE = 3 SV = 1 - | |
| | | | | | [Y1805_STRPN] | |
| P06653 | 20.44 | 6 | 318 | 25.81 | Autolysin OS = -Streptococcus | Intracellular |
| | | | | | pneumoniae serotype 4 (strain ATCC | |
| | | | | | BAA-334/TIGR4) GN = lytA PE = 1 SV = 2 - | |
| | | | | | [ALYS_STRPN] | |
| Q97PX9 | 12.78 | 5 | 266 | 25.41 | Amino acid ABC transporter, permease | Multi- |
| | | | | | protein OS = -Streptococcus | transmembrane |
| | | | | | pneumoniae serotype 4 (strain ATCC | |
| | | | | | BAA-334/TIGR4) GN = SP_1461 PE = 3 | |
| | | | | | SV = 1 - [Q97PX9_STRPN] | |
| Q97QW4 | 17.65 | 5 | 425 | 24.84 | Uncharacterized protein OS = | Intracellular |
| | | | | | Streptococcus pneumoniae serotype 4 | |
| | | | | | (strain ATCC BAA-334/TIGR4) | |
| | | | | | GN = SP_1083 PE = 4 SV = 1 - | |
| | | | | | [Q97QW4_STRPN] | |
| Q97S93 | 13.27 | 7 | 535 | 23.79 | CTP synthase OS = -Streptococcus | Intracellular |
| | | | | | pneumoniae serotype 4 (strain ATCC | |
| | | | | | BAA-334/TIGR4) GN = pyrG PE = 3 SV = 1 - | |
| | | | | | [PYRG_STRPN] | |
| Q97SX1 | 26.14 | 2 | 88 | 22 | UPF0297 protein SP_0192 OS = | Intracellular |
| | | | | | Streptococcus pneumoniae serotype 4 | |
| | | | | | (strain ATCC BAA-334/TIGR4) | |
| | | | | | GN = SP_0192 PE = 1 SV = 1 - | |
| | | | | | [Y192_STRPN] | |
| Q97RH7 | 50 | 4 | 78 | 21.34 | 30S ribosomal protein S20 OS = | Intracellular |
| | | | | | Streptococcus pneumoniae serotype 4 | |
| | | | | | (strain ATCC BAA-334/TIGR4) | |
| | | | | | GN = rpsT PE = 3 SV = 1 - [RS20_STRPN] | |
| P66050 | 45.78 | 5 | 166 | 20.98 | 50S ribosomal protein L10 OS = | Intracellular |
| | | | | | Streptococcus pneumoniae serotype 4 | |
| | | | | | (strain ATCC BAA-334/TIGR4) GN = rplJ | |
| | | | | | PE = 3 SV = 2 - [RL10_STRPN] | |
| Q97RW6 | 32.14 | 3 | 56 | 20.85 | Uncharacterized protein OS = | Secretory |
| | | | | | Streptococcus pneumoniae serotype 4 | (released) |
| | | | | | (strain ATCC BAA-334/TIGR4) | (with CS) |
| | | | | | GN = SP_0670 PE = 4 SV = 1 - | |
| | | | | | [Q97RW6_STRPN] | |
| Q9FBB7 | 29.41 | 4 | 255 | 20.65 | Acetyl-coenzyme A carboxylase | Intracellular |
| | | | | | carboxyl transferase subunit alpha OS = | |
| | | | | | Streptococcus pneumoniae serotype 4 | |
| | | | | | (strain ATCC BAA-334/TIGR4) | |
| | | | | | GN = accA PE = 1 SV = 1 - [ACCA_STRPN] | |
| I6L8V8 | 30.45 | 4 | 243 | 20.58 | 3-oxoacyl-[acyl-carrier protein] | Intracellular |
| | | | | | reductase OS = -Streptococcus | |
| | | | | | pneumoniae serotype 4 (strain ATCC | |
| | | | | | BAA-334/TIGR4) GN = fabG PE = 3 SV = 1 - | |
| | | | | | [I6L8V8_STRPN] | |
| P66581 | 41.46 | 4 | 164 | 20.46 | 30S ribosomal protein S5 OS = | Intracellular |
| | | | | | Streptococcus pneumoniae serotype 4 | |
| | | | | | (strain ATCC BAA-334/TIGR4) | |
| | | | | | GN = rpsE PE = 3 SV = 1 - [RS5_STRPN] | |
| Q97T45 | 11.82 | 5 | 694 | 20.39 | Uncharacterized protein OS = | Multi- |
| | | | | | Streptococcus pneumoniae serotype 4 | transmembrane |
| | | | | | (strain ATCC BAA-334/TIGR4) | |
| | | | | | GN = SP_0110 PE = 4 SV = 1 - | |
| | | | | | [Q97T45_STRPN] | |
| Q97P18 | 18.31 | 4 | 355 | 20.06 | ABC transporter, substrate-binding | Lipid |
| | | | | | protein OS = -Streptococcus | anchored |
| | | | | | pneumoniae serotype 4 (strain ATCC | |
| | | | | | BAA-334/TIGR4) GN = SP_1826 PE = 4 | |
| | | | | | SV = 1 - [Q97P18_STRPN] | |

TABLE 5
Proteins identified in MV_{L} from serotype 3. The accession numbers refer to SwissProt on the date of filing.

| Accession # | Coverage | # Peptides | #AAs | Score | Description | Localization |
|---|---|---|---|---|---|---|
| B2IQV2 | 91.09 | 50 | 404 | 5251.94 | Elongation factor Tu OS = | Intracellular |
| | | | | | Streptococcus pneumoniae (strain | |
| | | | | | CGSP14) GN = tuf PE = 3 SV = 1 - | |
| | | | | | [B2IQV2_STRPS] | |
| B2IPX8 | 95.62 | 40 | 434 | 2291.84 | Enolase OS = -Streptococcus | Intracellular |
| | | | | | pneumoniae (strain CGSP14) | |
| | | | | | GN = eno PE = 3 SV = 1 - [ENO_STRPS] | |
| B2INW2 | 70.86 | 42 | 374 | 2221.7 | Lipoprotein OS = -Streptococcus | Lipid |
| | | | | | pneumoniae (strain CGSP14) | anchored |
| | | | | | GN = SPCG_0787 PE = 4 SV = 1 - | |
| | | | | | [B2INW2_STRPS] | |
| B2ISJ9 | 85.28 | 67 | 693 | 1851.66 | Elongation factor G OS = | Intracellular |
| | | | | | Streptococcus pneumoniae (strain | |
| | | | | | CGSP14) GN = fusA PE = 3 SV = 1 - | |
| | | | | | [EFG_STRPS] | |
| B2IMI7 | 73.82 | 33 | 359 | 1675.28 | Glyceraldehyde-3-phosphate | Intracellular |
| | | | | | dehydrogenase OS = -Streptococcus | |
| | | | | | pneumoniae (strain CGSP14) | |
| | | | | | GN = gapA PE = 3 SV = 1 - | |
| | | | | | [B2IMI7_STRPS] | |
| B2IM39 | 82.94 | 101 | 1225 | 1348.46 | DNA-directed RNA polymerase | Intracellular |
| | | | | | subunit beta′ OS = -Streptococcus | |
| | | | | | pneumoniae (strain CGSP14) | |
| | | | | | GN = rpoC PE = 3 SV = 1 - | |
| | | | | | [RPOC_STRPS] | |
| B2IMJ9 | 86.85 | 68 | 890 | 1321.16 | Aldehyde-alcohol dehydrogenase | Intracellular |
| | | | | | OS = -Streptococcus pneumoniae (strain | |
| | | | | | CGSP14) GN = adhE PE = 3 SV = 1 - | |
| | | | | | [B2IMJ9_STRPS] | |
| B2IPD4 | 73.16 | 38 | 313 | 1171.98 | Foldase protein PrsA OS = | Lipid |
| | | | | | Streptococcus pneumoniae (strain | anchored |
| | | | | | CGSP14) GN = prsA PE = 3 SV = 1 - | |
| | | | | | [PRSA_STRPS] | |
| B2INY1 | 91 | 53 | 400 | 1141.84 | 30S ribosomal protein S1 OS = | Intracellular |
| | | | | | Streptococcus pneumoniae (strain | |
| | | | | | CGSP14) GN = rpsA PE = 4 SV = 1 - | |
| | | | | | [B2INY1_STRPS] | |
| B2IP53 | 87.46 | 27 | 335 | 830.14 | ATP-dependent 6- | Intracellular |
| | | | | | phosphofructokinase OS = | |
| | | | | | Streptococcus pneumoniae (strain | |
| | | | | | CGSP14) GN = pfkA PE = 3 SV = 1 - | |
| | | | | | [PFKA_STRPS] | |
| B2IQX0 | 90.6 | 38 | 468 | 772.66 | ATP synthase subunit beta OS = | Intracellular |
| | | | | | Streptococcus pneumoniae (strain | |
| | | | | | CGSP14) GN = atpD PE = 3 SV = 1 - | |
| | | | | | [ATPB_STRPS] | |
| B2IRV7 | 72.76 | 62 | 837 | 760.12 | Protein translocase subunit SecA | Intracellular |
| | | | | | OS = -Streptococcus pneumoniae (strain | |
| | | | | | CGSP14) GN = secA PE = 3 SV = 1 - | |
| | | | | | [B2IRV7_STRPS] | |
| B2IN25 | 78.22 | 51 | 450 | 751.01 | Maltose/maltodextrin ABC | Lipid |
| | | | | | transporter, maltose/maltodextrin- | anchored |
| | | | | | binding protein OS = -Streptococcus | |
| | | | | | pneumoniae (strain CGSP14) | |
| | | | | | GN = SPCG_2072 PE = 4 SV = 1 - | |
| | | | | | [B2IN25_STRPS] | |
| B2INB2 | 72.76 | 41 | 591 | 749.62 | Pyruvate oxidase OS = -Streptococcus | Intracellular |
| | | | | | pneumoniae (strain CGSP14) | |
| | | | | | GN = spxB PE = 3 SV = 1 - | |
| | | | | | [B2INB2_STRPS] | |
| B2ISZ0 | 69.04 | 47 | 659 | 746.61 | Oligopeptide ABC transporter, | Lipid |
| | | | | | oligopeptide-binding protein AmiA | anchored |
| | | | | | OS = -Streptococcus pneumoniae (strain | |
| | | | | | CGSP14) GN = amiA PE = 4 SV = 1 - | |
| | | | | | [B2ISZ0_STRPS] | |
| B2ISK8 | 56.44 | 22 | 303 | 734.11 | PTS system, mannose-specific IID | Multi- |
| | | | | | component OS = -Streptococcus | transmembrane |
| | | | | | pneumoniae (strain CGSP14) | |
| | | | | | GN = manN PE = 4 SV = 1 - | |
| | | | | | [B2ISK8_STRPS] | |
| B2INN2 | 85.33 | 30 | 259 | 682.23 | 30S ribosomal protein S2 OS = | Intracellular |
| | | | | | Streptococcus pneumoniae (strain | |
| | | | | | CGSP14) GN = rpsB PE = 3 SV = 2 - | |
| | | | | | [RS2_STRPS] | |
| B2IP54 | 76.05 | 39 | 501 | 676.55 | Pyruvate kinase OS = -Streptococcus | Intracellular |
| | | | | | pneumoniae (strain CGSP14) | |
| | | | | | GN = SPCG_0873 PE = 3 SV = 1 - | |
| | | | | | [B2IP54_STRPS] | |
| B2IR47 | 69.48 | 50 | 652 | 673.91 | Oligopeptide ABC transporter, | Lipid |
| | | | | | oligopeptide-binding protein AliB | anchored |
| | | | | | OS = -Streptococcus pneumoniae (strain | |
| | | | | | CGSP14) GN = aliB PE = 4 SV = 1 - | |
| | | | | | [B2IR47_STRPS] | |
| B2IR00 | 66.26 | 44 | 652 | 636.95 | ATP-dependent zinc | Multi- |
| | | | | | metalloprotease FtsH OS = | transmembrane |
| | | | | | Streptococcus pneumoniae (strain | |
| | | | | | CGSP14) GN = ftsH PE = 3 SV = 1 - | |
| | | | | | [B2IR00_STRPS] | |
| B2IM40 | 77.67 | 50 | 824 | 633.52 | DNA-directed RNA polymerase | Intracellular |
| | | | | | subunit beta OS = -Streptococcus | |
| | | | | | pneumoniae (strain CGSP14) | |
| | | | | | GN = rpoB PE = 3 SV = 1 - | |
| | | | | | [B2IM40_STRPS] | |
| B2IS43 | 70.76 | 23 | 277 | 575.93 | 50S ribosomal protein L2 OS = | Intracellular |
| | | | | | Streptococcus pneumoniae (strain | |
| | | | | | CGSP14) GN = rplB PE = 3 SV = 1 - | |
| | | | | | [RL2_STRPS] | |
| B2IS46 | 79.26 | 27 | 217 | 569.56 | 30S ribosomal protein S3 OS = | Intracellular |
| | | | | | Streptococcus pneumoniae (strain | |
| | | | | | CGSP14) GN = rpsC PE = 3 SV = 1 - | |
| | | | | | [RS3_STRPS] | |
| B2IME4 | 64.95 | 51 | 930 | 553.42 | Translation initiation factor IF-2 OS = | Intracellular |
| | | | | | Streptococcus pneumoniae (strain | |
| | | | | | CGSP14) GN = infB PE = 3 SV = 1 - | |
| | | | | | [IF2_STRPS] | |
| B2IQY1 | 73.14 | 43 | 551 | 550.86 | Uncharacterized protein OS = | Intracellular/ |
| | | | | | Streptococcus pneumoniae (strain | TMH start |
| | | | | | CGSP14) GN = SPCG_1503 PE = 4 SV = 1 - | AFTER 60 |
| | | | | | [B2IQY1_STRPS] | |
| B2INT1 | 58.9 | 34 | 584 | 531.45 | Septation ring formation regulator | N- |
| | | | | | EzrA OS = -Streptococcus pneumoniae | terminally |
| | | | | | (strain CGSP14) GN = ezrA PE = 3 SV = 1 - | anchored |
| | | | | | [B2INT1_STRPS] | (No CS) |
| B2IPY8 | 67.93 | 32 | 474 | 511.28 | Glyceraldehyde-3-phosphate | Intracellular |
| | | | | | dehydrogenase, NADP-dependent | |
| | | | | | OS = -Streptococcus pneumoniae (strain | |
| | | | | | CGSP14) GN = gapN PE = 3 SV = 1 - | |
| | | | | | [B2IPY8_STRPS] | |
| B2IRQ6 | 79.23 | 20 | 313 | 461.56 | Manganese ABC transporter, | Lipid |
| | | | | | manganese-binding adhesion | anchored |
| | | | | | liprotein OS = -Streptococcus | |
| | | | | | pneumoniae (strain CGSP14) | |
| | | | | | GN = SPCG_1623 PE = 3 SV = 1 - | |
| | | | | | [B2IRQ6_STRPS] | |
| B2IPU8 | 63.14 | 36 | 719 | 457.32 | Ribonucleoside-diphosphate | Intracellular |
| | | | | | reductase OS = -Streptococcus | |
| | | | | | pneumoniae (strain CGSP14) | |
| | | | | | GN = nrdE PE = 3 SV = 1 - | |
| | | | | | [B2IPU8_STRPS] | |
| B2IM49 | 72.42 | 27 | 330 | 449.87 | Aspartate--ammonia ligase OS = | Intracellular |
| | | | | | Streptococcus pneumoniae (strain | |
| | | | | | CGSP14) GN = asnA PE = 3 SV = 1 - | |
| | | | | | [ASNA_STRPS] | |
| B2IQX2 | 68.86 | 31 | 501 | 447.54 | ATP synthase subunit alpha OS = | Intracellular |
| | | | | | Streptococcus pneumoniae (strain | |
| | | | | | CGSP14) GN = atpA PE = 3 SV = 1 - | |
| | | | | | [ATPA_STRPS] | |
| B2IMK3 | 70.36 | 32 | 658 | 435.4 | Transketolase OS = -Streptococcus | Intracellular |
| | | | | | pneumoniae (strain CGSP14) | |
| | | | | | GN = tktA PE = 3 SV = 1 - | |
| | | | | | [B2IMK3_STRPS] | |
| B2IRH9 | 64.45 | 36 | 616 | 434.01 | Capsular polysaccharide | Multi- |
| | | | | | biosynthesis protein, putative OS = | transmembrane |
| | | | | | Streptococcus pneumoniae (strain | |
| | | | | | CGSP14) GN = SPCG_0098 PE = 4 SV = 1 - | |
| | | | | | [B2IRH9_STRPS] | |
| B2IN95 | 82.06 | 37 | 496 | 432.16 | Lysine--tRNA ligase OS = | Intracellular |
| | | | | | Streptococcus pneumoniae (strain | |
| | | | | | CGSP14) GN = lysS PE = 3 SV = 1 - | |
| | | | | | [SYK_STRPS] | |
| B2IS90 | 71.32 | 40 | 537 | 413.57 | Ribonuclease Y OS = -Streptococcus | Intracellular |
| | | | | | pneumoniae (strain CGSP14) GN = rny | |
| | | | | | PE = 3 SV = 1 - [B2IS90_STRPS] | |
| B2IRT9 | 66.06 | 32 | 442 | 412.96 | Sugar ABC transporter, sugar- | Lipid |
| | | | | | binding protein OS = -Streptococcus | anchored |
| | | | | | pneumoniae (strain CGSP14) | |
| | | | | | GN = SPCG_1656 PE = 4 SV = 1 - | |
| | | | | | [B2IRT9_STRPS] | |
| B2IN10 | 73.37 | 15 | 338 | 401 | Glycerol-3-phosphate | Intracellular |
| | | | | | dehydrogenase [NAD(P)+] OS = | |
| | | | | | Streptococcus pneumoniae (strain | |
| | | | | | CGSP14) GN = gpsA PE = 3 SV = 1 - | |
| | | | | | [GPDA_STRPS] | |
| B2IP07 | 38.46 | 23 | 650 | 398.03 | PTS system, fructose specific IIABC | Multi- |
| | | | | | components OS = -Streptococcus | transmembrane |
| | | | | | pneumoniae (strain CGSP14) | |
| | | | | | GN = fruA PE = 4 SV = 1 - | |
| | | | | | [B2IP07_STRPS] | |
| B2IPB5 | 52.94 | 10 | 119 | 388.03 | 50S ribosomal protein L20 OS = | Intracellular |
| | | | | | Streptococcus pneumoniae (strain | |
| | | | | | CGSP14) GN = rplT PE = 3 SV = 1 - | |
| | | | | | [RL20_STRPS] | |
| B2IS19 | 66.29 | 35 | 617 | 385.71 | Proline--tRNA ligase OS = | Intracellular |
| | | | | | Streptococcus pneumoniae (strain | |
| | | | | | CGSP14) GN = proS PE = 3 SV = 1 - | |
| | | | | | [SYP_STRPS] | |
| B2INH5 | 83.95 | 31 | 299 | 370.13 | SPFH domain/Band 7 family OS = | N- |
| | | | | | Streptococcus pneumoniae (strain | terminally |
| | | | | | CGSP14) GN = SPCG_2124 PE = 4 SV = 1 - | anchored |
| | | | | | [B2INH5_STRPS] | (No CS) |
| B2IQ40 | 64.14 | 46 | 898 | 367.23 | Phosphoenolpyruvate carboxylase | Intracellular |
| | | | | | OS = -Streptococcus pneumoniae (strain | |
| | | | | | CGSP14) GN = ppc PE = 3 SV = 1 - | |
| | | | | | [B2IQ40_STRPS] | |
| B2IML8 | 76.35 | 25 | 406 | 358.33 | Acetate kinase OS = -Streptococcus | Intracellular |
| | | | | | pneumoniae (strain CGSP14) | |
| | | | | | GN = ackA PE = 3 SV = 1 - | |
| | | | | | [B2IML8_STRPS] | |
| B2IRS1 | 53.94 | 28 | 419 | 341.4 | Cell division protein FtsZ OS = | Intracellular |
| | | | | | Streptococcus pneumoniae (strain | |
| | | | | | CGSP14) GN = ftsZ PE = 3 SV = 1 - | |
| | | | | | [B2IRS1_STRPS] | |
| B2IMN0 | 96.59 | 22 | 352 | 339.61 | Alcohol dehydrogenase, zinc- | Intracellular |
| | | | | | containing OS = -Streptococcus | |
| | | | | | pneumoniae (strain CGSP14) | |
| | | | | | GN = adh PE = 3 SV = 1 - | |
| | | | | | [B2IMNO_STRPS] | |
| B2ISJ7 | 51.09 | 13 | 137 | 337.74 | 30S ribosomal protein S12 OS = | Intracellular |
| | | | | | Streptococcus pneumoniae (strain | |
| | | | | | CGSP14) GN = rpsL PE = 3 SV = 1 - | |
| | | | | | [RS12_STRPS] | |
| B2IS41 | 63.29 | 16 | 207 | 337.71 | 50S ribosomal protein L4 OS = | Intracellular |
| | | | | | Streptococcus pneumoniae (strain | |
| | | | | | CGSP14) GN = rplD PE = 3 SV = 1 - | |
| | | | | | [RL4_STRPS] | |
| B2ILW7 | 80.68 | 18 | 414 | 332.02 | 3-oxoacyl-[acyl-carrier-protein] | Intracellular |
| | | | | | synthase 2 OS = -Streptococcus | |
| | | | | | pneumoniae (strain CGSP14) | |
| | | | | | GN = fabF PE = 3 SV = 1 - | |
| | | | | | [B2ILW7_STRPS] | |
| B2IRM9 | 70.29 | 22 | 276 | 330.56 | ABC transporter, substrate-binding | Lipid |
| | | | | | protein OS = -Streptococcus | anchored |
| | | | | | pneumoniae (strain CGSP14) | |
| | | | | | GN = SPCG_0152 PE = 4 SV = 1 - | |
| | | | | | [B2IRM9_STRPS] | |
| B2ISY9 | 47.99 | 21 | 498 | 319.21 | Oligopeptide ABC transporter, | Multi- |
| | | | | | permease protein Amic OS = | transmembrane |
| | | | | | Streptococcus pneumoniae (strain | |
| | | | | | CGSP14) GN = amiC PE = 3 SV = 1 - | |
| | | | | | [B2ISY9_STRPS] | |
| B2IQT6 | 62.75 | 22 | 459 | 313.52 | NADH oxidase OS = -Streptococcus | Intracellular |
| | | | | | pneumoniae (strain CGSP14) | |
| | | | | | GN = nox PE = 4 SV = 1 - | |
| | | | | | [B2IQT6_STRPS] | |
| B21PP2 | 47.02 | 32 | 721 | 312 | Amino acid ABC transporter, amino | Multi- |
| | | | | | acid-binding protein/permease | transmembrane |
| | | | | | protein OS = -Streptococcus | |
| | | | | | pneumoniae (strain CGSP14) | |
| | | | | | GN = SPCG_1062 PE = 3 SV = 1 - | |
| | | | | | [B2IPP2_STRPS] | |
| B2IS47 | 63.5 | 13 | 137 | 300.82 | 50S ribosomal protein L16 OS = | Intracellular |
| | | | | | Streptococcus pneumoniae (strain | |
| | | | | | CGSP14) GN = rplP PE = 3 SV = 1 - | |
| | | | | | [RL16_STRPS] | |
| B2IRC1 | 66.67 | 19 | 339 | 299.62 | UDP-glucose 4-epimerase OS = | Intracellular |
| | | | | | Streptococcus pneumoniae (strain | |
| | | | | | CGSP14) GN = galE PE = 4 SV = 1 - | |
| | | | | | [B2IRC1_STRPS] | |
| B2IQW3 | 71.22 | 25 | 278 | 296.15 | Amino acid ABC transporter, amino | Lipid |
| | | | | | acid-binding protein OS = | anchored |
| | | | | | Streptococcus pneumoniae (strain | |
| | | | | | CGSP14) GN = SPCG_1485 PE = 4 SV = 1 - | |
| | | | | | [B2IQW3_STRPS] | |
| B2IRR1 | 67.83 | 19 | 230 | 295.06 | 2,3-bisphosphoglycerate- | Intracellular |
| | | | | | dependent phosphoglycerate | |
| | | | | | mutase OS = -Streptococcus | |
| | | | | | pneumoniae (strain CGSP14) | |
| | | | | | GN = gpmA PE = 3 SV = 1 - | |
| | | | | | [GPMA_STRPS] | |
| B2IS34 | 61.87 | 39 | 737 | 290.83 | Anaerobic ribonucleoside | Intracellular |
| | | | | | triphosphate reductase OS = | |
| | | | | | Streptococcus pneumoniae (strain | |
| | | | | | CGSP14) GN = SPCG_0212 PE = 4 SV = 1 - | |
| | | | | | [B2IS34_STRPS] | |
| B2IS52 | 87.78 | 18 | 180 | 288.42 | 50S ribosomal protein L5 OS = | Intracellular |
| | | | | | Streptococcus pneumoniae (strain | |
| | | | | | CGSP14) GN = rplE PE = 3 SV = 1 - | |
| | | | | | [RL5_STRPS] | |
| B2IQU1 | 65.63 | 33 | 678 | 288.36 | Glycine--tRNA ligase beta subunit | Intracellular |
| | | | | | OS = -Streptococcus pneumoniae (strain | |
| | | | | | CGSP14) GN = glyS PE = 3 SV = 1 - | |
| | | | | | [SYGB_STRPS] | |
| B2ILY5 | 60.36 | 21 | 555 | 284.08 | Uncharacterized protein OS = | Intracellular |
| | | | | | Streptococcus pneumoniae (strain | |
| | | | | | CGSP14) GN = SPCG_0438 PE = 4 SV = 1 - | |
| | | | | | [B2ILY5_STRPS] | |
| B2IS40 | 59.62 | 14 | 208 | 283.69 | 50S ribosomal protein L3 OS = | Intracellular |
| | | | | | Streptococcus pneumoniae (strain | |
| | | | | | CGSP14) GN = rplC PE = 3 SV = 1 - | |
| | | | | | [RL3_STRPS] | |
| B 21M73 | 32.44 | 18 | 521 | 281.46 | Amino acid ABC transporter, amino | Multi- |
| | | | | | acid-binding protein/permease | transmembrane |
| | | | | | protein OS = -Streptococcus | |
| | | | | | pneumoniae (strain CGSP14) | |
| | | | | | GN = gInH PE = 3 SV = 1 - | |
| | | | | | [B2IM73_STRPS] | |
| B2IR19 | 52.65 | 17 | 302 | 275.76 | Amino acid ABC transporter, | Lipid |
| | | | | | periplasmic amino acid-binding | anchored |
| | | | | | protein, putative OS = -Streptococcus | |
| | | | | | pneumoniae (strain CGSP14) | |
| | | | | | GN = SPCG_0108 PE = 4 SV = 1 - | |
| | | | | | [B2IR19_STRPS] | |
| B2IRN0 | 57.75 | 22 | 284 | 275.2 | Lipoprotein OS = -Streptococcus | Lipid |
| | | | | | pneumoniae (strain CGSP14) | anchored |
| | | | | | GN = SPCG_0153 PE = 3 SV = 1 - | |
| | | | | | [B2IRNO_STRPS] | |
| B2IM81 | 70.93 | 36 | 774 | 270.81 | Formate acetyltransferase OS = | Intracellular |
| | | | | | Streptococcus pneumoniae (strain | |
| | | | | | CGSP14) GN = pfl PE = 4 SV = 1 - | |
| | | | | | [B2IM81_STRPS] | |
| B2IRN7 | 65.61 | 30 | 660 | 269.02 | Threonine--tRNA ligase OS = | Intracellular |
| | | | | | Streptococcus pneumoniae (strain | |
| | | | | | CGSP14) GN = thrS PE = 3 SV = 1 - | |
| | | | | | [B2IRN7_STRPS] | |
| B2IMN6 | 63.36 | 22 | 494 | 268.42 | Threonine synthase OS = | Intracellular |
| | | | | | Streptococcus pneumoniae (strain | |
| | | | | | CGSP14) GN = thrC PE = 4 SV = 1 - | |
| | | | | | [B2IMN6_STRPS] | |
| B2IR98 | 69.68 | 20 | 376 | 261.66 | Sugar ABC transporter, ATP-binding | Intracellular |
| | | | | | protein OS = -Streptococcus | |
| | | | | | pneumoniae (strain CGSP14) | |
| | | | | | GN = SPCG_1565 PE = 3 SV = 1 - | |
| | | | | | [B2IR98_STRPS] | |
| B2IMG8 | 58.42 | 23 | 404 | 253.09 | Aspartate aminotransferase OS = | Intracellular |
| | | | | | Streptococcus pneumoniae (strain | |
| | | | | | CGSP14) GN = aspC PE = 4 SV = 1 - | |
| | | | | | [B2IMG8_STRPS] | |
| B2ILW6 | 65.43 | 13 | 243 | 252.82 | 3-ketoacyl-(Acyl-carrier-protein) | Intracellular |
| | | | | | reductase OS = -Streptococcus | |
| | | | | | pneumoniae (strain CGSP14) | |
| | | | | | GN = fabG PE = 4 SV = 1 - | |
| | | | | | [B2ILW6_STRPS] | |
| B2INF2 | 70.18 | 25 | 513 | 248.48 | ABC transporter, ATP-binding | Intracellular |
| | | | | | protein OS = -Streptococcus | |
| | | | | | pneumoniae (strain CGSP14) | |
| | | | | | GN = SPCG_0719 PE = 4 SV = 1 - | |
| | | | | | [B2INF2_STRPS] | |
| B2IRS2 | 78.12 | 20 | 457 | 247.37 | Cell division protein FtsA OS = | Multi- |
| | | | | | Streptococcus pneumoniae (strain | transmembrane |
| | | | | | CGSP14) GN = ftsZ PE = 3 SV = 1 - | |
| | | | | | [B2IRS2_STRPS] | |
| B2IM41 | 70.82 | 22 | 377 | 246.54 | Uncharacterized protein OS = | Intracellular |
| | | | | | Streptococcus pneumoniae (strain | |
| | | | | | CGSP14) GN = SPCG_1926 PE = 3 SV = 1 - | |
| | | | | | [B2IM41_STRPS] | |
| B2IQ88 | 66.09 | 13 | 115 | 240.71 | 50S ribosomal protein L19 OS = | Intracellular |
| | | | | | Streptococcus pneumoniae (strain | |
| | | | | | CGSP14) GN = rplS PE = 3 SV = 1 - | |
| | | | | | [RL19_STRPS] | |
| B2IQ10 | 56.1 | 22 | 344 | 240.32 | Uncharacterized protein OS = | Multi- |
| | | | | | Streptococcus pneumoniae (strain | transmembrane |
| | | | | | CGSP14) GN = SPCG_1352 PE = 4 SV = 1 - | |
| | | | | | [B2IQ10_STRPS] | |
| B2IND2 | 63.73 | 18 | 386 | 236.96 | Branched-chain amino acid ABC | Lipid |
| | | | | | transporter, amino acid-binding | anchored |
| | | | | | protein OS = -Streptococcus | |
| | | | | | pneumoniae (strain CGSP14) | |
| | | | | | GN = SPCG_0699 PE = 4 SV = 1 - | |
| | | | | | [B2IND2_STRPS] | |
| B2INP5 | 71.34 | 24 | 492 | 236.94 | Inosine-5′-monophosphate | Intracellular |
| | | | | | dehydrogenase OS = -Streptococcus | |
| | | | | | pneumoniae (strain CGSP14) | |
| | | | | | GN = imdH PE = 3 SV = 1 - | |
| | | | | | [B2INP5_STRPS] | |
| B2ISL2 | 67.26 | 18 | 339 | 235.53 | Alcohol dehydrogenase OS = | Intracellular |
| | | | | | Streptococcus pneumoniae (strain | |
| | | | | | CGSP14) GN = adhP PE = 3 SV = 1 - | |
| | | | | | [B2ISL2_STRPS] | |
| B2ISM4 | 59.23 | 9 | 130 | 228.51 | 30S ribosomal protein S9 OS = | Intracellular |
| | | | | | Streptococcus pneumoniae (strain | |
| | | | | | CGSP14) GN = rpsI PE = 3 SV = 1 - | |
| | | | | | [RS9_STRPS] | |
| B2IR71 | 40.26 | 23 | 914 | 227.01 | Cation-transporting ATPase, E1-E2 | Multi- |
| | | | | | family OS = -Streptococcus pneumoniae | transmembrane |
| | | | | | (strain CGSP14) GN = pacL PE = 3 | |
| | | | | | SV = 1 - [B2IR71_STRPS] | |
| B2IPP1 | 85.77 | 17 | 246 | 225.41 | Amino acid ABC transporter, ATP- | Intracellular |
| | | | | | binding protein OS = -Streptococcus | |
| | | | | | pneumoniae (strain CGSP14) | |
| | | | | | GN = SPCG_1061 PE = 4 SV = 1 - | |
| | | | | | [B2IPP1_STRPS] | |
| B2ISY7 | 58.31 | 18 | 355 | 221.22 | Oligopeptide ABC transporter, ATP- | Intracellular |
| | | | | | binding protein AmiE OS = | |
| | | | | | Streptococcus pneumoniae (strain | |
| | | | | | CGSP14) GN = amiE PE = 3 SV = 1 - | |
| | | | | | [B2ISY7_STRPS] | |
| B2IQL1 | 56.83 | 16 | 271 | 219.73 | Amino acid ABC transporter, amino | Lipid |
| | | | | | acid-binding protein OS = | anchored |
| | | | | | Streptococcus pneumoniae (strain | |
| | | | | | CGSP14) GN = SPCG_1383 PE = 4 SV = 1 - | |
| | | | | | [B2IQL1_STRPS] | |
| B2ISG3 | 77.23 | 19 | 202 | 219.45 | General stress protein 24, putative | Intracellular |
| | | | | | OS = -Streptococcus pneumoniae (strain | |
| | | | | | CGSP14) GN = SPCG_1781 PE = 4 SV = 1 - | |
| | | | | | [B2ISG3_STRPS] | |
| B2IMA2 | 70.1 | 20 | 398 | 216.35 | Phosphoglycerate kinase OS = | Intracellular |
| | | | | | Streptococcus pneumoniae (strain | |
| | | | | | CGSP14) GN = pgk PE = 3 SV = 1 - | |
| | | | | | [PGK_STRPS] | |
| B2IN71 | 49.03 | 25 | 620 | 212.01 | Elongation factor Tu family protein | Intracellular |
| | | | | | OS = -Streptococcus pneumoniae (strain | |
| | | | | | CGSP14) GN = SPCG_0638 PE = 4 SV = 1 - | |
| | | | | | [B2IN71_STRPS] | |
| B2IPR1 | 75.6 | 16 | 332 | 210.14 | L-lactate dehydrogenase OS = | Intracellular |
| | | | | | Streptococcus pneumoniae (strain | |
| | | | | | CGSP14) GN = Idh PE = 3 SV = 1 - | |
| | | | | | [B2IPR1_STRPS] | |
| B2IQK0 | 51.83 | 30 | 872 | 209.16 | Alanine--tRNA ligase OS = | Intracellular |
| | | | | | Streptococcus pneumoniae (strain | |
| | | | | | CGSP14) GN = alaS PE = 3 SV = 1 - | |
| | | | | | [SYA_STRPS] | |
| B2IS44 | 61.29 | 12 | 93 | 208.67 | 30S ribosomal protein S19 OS = | Intracellular |
| | | | | | Streptococcus pneumoniae (strain | |
| | | | | | CGSP14) GN = rpsS PE = 3 SV = 1 - | |
| | | | | | [RS19_STRPS] | |
| B2IMN9 | 48.47 | 17 | 425 | 208.24 | Uncharacterized protein OS = | Intracellular |
| | | | | | Streptococcus pneumoniae (strain | |
| | | | | | CGSP14) GN = SPCG_2034 PE = 4 SV = 1 - | |
| | | | | | [B2IMN9_STRPS] | |
| B2IR17 | 72.27 | 16 | 339 | 208.13 | Ribose-phosphate | Intracellular |
| | | | | | pyrophosphokinase OS = | |
| | | | | | Streptococcus pneumoniae (strain | |
| | | | | | CGSP14) GN = prsA PE = 3 SV = 1 - | |
| | | | | | [B2IR17_STRPS] | |
| B2IQR1 | 62.69 | 25 | 520 | 208 | GMP synthase [glutamine- | Intracellular |
| | | | | | hydrolyzing] OS = -Streptococcus | |
| | | | | | pneumoniae (strain CGSP14) | |
| | | | | | GN = guaA PE = 3 SV = 1 - | |
| | | | | | [GUAA_STRPS] | |
| B2INL3 | 50.25 | 23 | 810 | 202.66 | ATP-dependent Clp protease, ATP- | Intracellular |
| | | | | | binding subunit OS = -Streptococcus | |
| | | | | | pneumoniae (strain CGSP14) | |
| | | | | | GN = SPCG_2162 PE = 3 SV = 1 - | |
| | | | | | [B2INL3_STRPS] | |
| B2IN11 | 58.53 | 17 | 299 | 197.31 | UTP--glucose-1-phosphate | Secretory |
| | | | | | uridylyltransferase OS = | (released) |
| | | | | | Streptococcus pneumoniae (strain | (with CS) |
| | | | | | CGSP14) GN = galU PE = 3 SV = 1 - | |
| | | | | | [B2IN11_STRPS] | |
| B2IQU5 | 44.38 | 17 | 480 | 197.17 | Peptidoglycan N-acetylglucosamine | N- |
| | | | | | deacetylase A OS = -Streptococcus | terminally |
| | | | | | pneumoniae (strain CGSP14) | anchored |
| | | | | | GN = pgdA PE = 4 SV = 1 - | (with CS) |
| | | | | | [B2IQU5_STRPS] | |
| B2INE4 | 57.49 | 19 | 447 | 196.45 | DEAD-box ATP-dependent RNA | Intracellular |
| | | | | | helicase CshB OS = -Streptococcus | |
| | | | | | pneumoniae (strain CGSP14) | |
| | | | | | GN = rheB PE = 3 SV = 1 - | |
| | | | | | [B2INE4_STRPS] | |
| B2IMU6 | 65.97 | 15 | 238 | 196.17 | Uncharacterized protein OS = | Lipid |
| | | | | | Streptococcus pneumoniae (strain | anchored |
| | | | | | CGSP14) GN = SPCG_0590 PE = 4 SV = 1 - | |
| | | | | | [B2IMU6_STRPS] | |
| B2INT7 | 54.92 | 27 | 752 | 195.47 | ATP-dependent Clp protease, ATP- | Intracellular |
| | | | | | binding subunit ClpE OS = | |
| | | | | | Streptococcus pneumoniae (strain | |
| | | | | | CGSP14) GN = clpE PE = 3 SV = 1 - | |
| | | | | | [B2INT7_STRPS] | |
| B2IM28 | 44.59 | 17 | 388 | 194.98 | Protein RecA OS = -Streptococcus | Intracellular |
| | | | | | pneumoniae (strain CGSP14) | |
| | | | | | GN = recA PE = 3 SV = 1 - [RECA_STRPS] | |
| B2IQL4 | 79.78 | 18 | 267 | 192.86 | Phosphate import ATP-binding | Intracellular |
| | | | | | protein PstB OS = -Streptococcus | |
| | | | | | pneumoniae (strain CGSP14) | |
| | | | | | GN = pstB PE = 3 SV = 1 - | |
| | | | | | [B2IQL4_STRPS] | |
| B2ISL0 | 43.67 | 13 | 332 | 190.43 | PTS system, mannose-specific IIAB | Intracellular |
| | | | | | components OS = -Streptococcus | |
| | | | | | pneumoniae (strain CGSP14) | |
| | | | | | GN = manL PE = 4 SV = 1 - | |
| | | | | | [B2ISLO_STRPS] | |
| B2INW3 | 54.6 | 19 | 511 | 188.38 | Sugar ABC transporter, ATP-binding | Intracellular |
| | | | | | protein OS = -Streptococcus | |
| | | | | | pneumoniae (strain CGSP14) | |
| | | | | | GN = SPCG_0788 PE = 4 SV = 1 - | |
| | | | | | [B2INW3_STRPS] | |
| B2INT0 | 40.9 | 22 | 648 | 185.21 | DNA gyrase subunit B OS = | Intracellular |
| | | | | | Streptococcus pneumoniae (strain | |
| | | | | | CGSP14) GN = gyrB PE = 3 SV = 1 - | |
| | | | | | [B2INTO_STRPS] | |
| B2IP17 | 57.18 | 13 | 341 | 184.52 | Iron-compound ABC transporter, | Lipid |
| | | | | | iron compound-binding protein OS = | anchored |
| | | | | | Streptococcus pneumoniae (strain | |
| | | | | | CGSP14) GN = SPCG_1009 PE = 4 SV = 1 - | |
| | | | | | [B2IPI7_STRPS] | |
| B2IPT0 | 48.09 | 22 | 628 | 183.98 | Elongation factor 4 OS = | Intracellular |
| | | | | | Streptococcus pneumoniae (strain | |
| | | | | | CGSP14) GN = lepA PE = 3 SV = 1 - | |
| | | | | | [B2IPTO_STRPS] | |
| B2IN23 | 39.89 | 17 | 752 | 181.97 | Alpha-1,4 glucan phosphorylase | Intracellular |
| | | | | | OS = -Streptococcus pneumoniae (strain | |
| | | | | | CGSP14) GN = maIP PE = 3 SV = 1 - | |
| | | | | | [B2IN23_STRPS] | |
| B2IQZ2 | 64.71 | 20 | 374 | 180.03 | Ribosome-binding ATPase YchF OS = | Intracellular |
| | | | | | Streptococcus pneumoniae (strain | |
| | | | | | CGSP14) GN = ychF PE = 3 SV = 1 - | |
| | | | | | [B2IQZ2_STRPS] | |
| B2IMU8 | 66.81 | 17 | 229 | 179.85 | 50S ribosomal protein L1 OS = | Intracellular |
| | | | | | Streptococcus pneumoniae (strain | |
| | | | | | CGSP14) GN = rplA PE = 3 SV = 1 - | |
| | | | | | [RL1_STRPS] | |
| B2ISY6 | 51.06 | 14 | 331 | 178.98 | Oligopeptide ABC transporter, ATP- | Intracellular |
| | | | | | binding protein AmiF OS = | |
| | | | | | Streptococcus pneumoniae (strain | |
| | | | | | CGSP14) GN = amiF PE = 3 SV = 1 - | |
| | | | | | [B2ISY6_STRPS] | |
| B2IR87 | 54.15 | 18 | 410 | 170.5 | ATP-dependent Clp protease ATP- | Intracellular |
| | | | | | binding subunit ClpX OS = | |
| | | | | | Streptococcus pneumoniae (strain | |
| | | | | | CGSP14) GN = clpX PE = 3 SV = 1 - | |
| | | | | | [CLPX_STRPS] | |
| B2IR07 | 58.6 | 24 | 442 | 169.98 | Adenylosuccinate synthetase OS = | Intracellular |
| | | | | | Streptococcus pneumoniae (strain | |
| | | | | | CGSP14) GN = purA PE = 3 SV = 1 - | |
| | | | | | [B2IR07_STRPS] | |
| B2ILV9 | 61.23 | 20 | 454 | 169.93 | Aspartokinase OS = -Streptococcus | Intracellular |
| | | | | | pneumoniae (strain CGSP14) | |
| | | | | | GN = lysC PE = 3 SV = 1 - | |
| | | | | | [B2ILV9_STRPS] | |
| B2IMZ6 | 42.12 | 25 | 857 | 169.84 | DNA mismatch repair protein MutS | Intracellular |
| | | | | | OS = -Streptococcus pneumoniae (strain | |
| | | | | | CGSP14) GN = hexA PE = 3 SV = 1 - | |
| | | | | | [B2IMZ6_STRPS] | |
| B2IS57 | 84.76 | 17 | 164 | 169.34 | 30S ribosomal protein S5 OS = | Intracellular |
| | | | | | Streptococcus pneumoniae (strain | |
| | | | | | CGSP14) GN = rpsE PE = 3 SV = 1 - | |
| | | | | | [B2IS57_STRPS] | |
| B2ILR1 | 29.62 | 20 | 719 | 166.63 | Penicillin-binding protein 1A OS = | N- |
| | | | | | Streptococcus pneumoniae (strain | terminally |
| | | | | | CGSP14) GN = pbp1A PE = 4 SV = 1 - | anchored |
| | | | | | [B2ILR1_STRPS] | (with CS) |
| B2IQ78 | 53.73 | 22 | 523 | 165.23 | Signal recognition particle protein | Intracellular |
| | | | | | OS = -Streptococcus pneumoniae (strain | |
| | | | | | CGSP14) GN = ffh PE = 3 SV = 1 - | |
| | | | | | [B2IQ78_STRPS] | |
| B2ISM3 | 78.38 | 17 | 148 | 163.82 | 50S ribosomal protein L13 OS = | Intracellular |
| | | | | | Streptococcus pneumoniae (strain | |
| | | | | | CGSP14) GN = rplM PE = 3 SV = 1 - | |
| | | | | | [B2ISM3_STRPS] | |
| B2INJ2 | 70.02 | 21 | 427 | 163.61 | DltD protein OS = -Streptococcus | N- |
| | | | | | pneumoniae (strain CGSP14) | terminally |
| | | | | | GN = dltD PE = 4 SV = 1 - | anchored |
| | | | | | [B2INJ2_STRPS] | (with CS) |
| B2ISJ1 | 48.84 | 19 | 602 | 159.21 | Glutamine--fructose-6-phosphate | Intracellular |
| | | | | | aminotransferase [isomerizing] OS = | |
| | | | | | Streptococcus pneumoniae (strain | |
| | | | | | CGSP14) GN = glmS PE = 3 SV = 1 - | |
| | | | | | [B2ISJ1_STRPS] | |
| B2IPM7 | 52.69 | 18 | 577 | 159.04 | Phosphoenolpyruvate-protein | Intracellular |
| | | | | | phosphotransferase OS = | |
| | | | | | Streptococcus pneumoniae (strain | |
| | | | | | CGSP14) GN = SPCG_1121 PE = 3 SV = 1 | |
| | | | | | - [B2IPM7_STRPS] | |
| B2IQ29 | 61.01 | 17 | 436 | 157.98 | GTPase Obg OS = -Streptococcus | Intracellular |
| | | | | | pneumoniae (strain CGSP14) | |
| | | | | | GN = obg PE = 3 SV = 1 - [OBG_STRPS] | |
| B2IS66 | 74.8 | 9 | 127 | 157.55 | 30S ribosomal protein S11 OS = | Intracellular |
| | | | | | Streptococcus pneumoniae (strain | |
| | | | | | CGSP14) GN = rpsK PE = 3 SV = 1 - | |
| | | | | | [RS11_STRPS] | |
| B2IQ67 | 33.36 | 24 | 1058 | 157.35 | Carbamoyl-phosphate synthase | Intracellular |
| | | | | | large chain OS = -Streptococcus | |
| | | | | | pneumoniae (strain CGSP14) | |
| | | | | | GN = carB PE = 3 SV = 1 - [CARB_STRPS] | |
| B2IQ25 | 62.29 | 16 | 419 | 154.75 | UDP-N-acetylglucosamine 1- | Intracellular |
| | | | | | carboxyvinyltransferase OS = | |
| | | | | | Streptococcus pneumoniae (strain | |
| | | | | | CGSP14) GN = murZ PE = 3 SV = 1 - | |
| | | | | | [B2IQ25_STRPS] | |
| B2IQX4 | 82.93 | 14 | 164 | 154.18 | ATP synthase subunit b OS = | N- |
| | | | | | Streptococcus pneumoniae (strain | terminally |
| | | | | | CGSP14) GN = atpF PE = 3 SV = 1 - | anchored |
| | | | | | [ATPF_STRPS] | (with CS) |
| B2IM72 | 69.51 | 10 | 246 | 154.11 | Amino acid ABC transporter, ATP- | Intracellular |
| | | | | | binding protein OS = -Streptococcus | |
| | | | | | pneumoniae (strain CGSP14) | |
| | | | | | GN = glnQ PE = 4 SV = 1 - | |
| | | | | | [B2IM72_STRPS] | |
| B2INR2 | 35.79 | 19 | 679 | 153.3 | Methionine--tRNA ligase OS = | Intracellular |
| | | | | | Streptococcus pneumoniae (strain | |
| | | | | | CGSP14) GN = metG PE = 3 SV = 1 - | |
| | | | | | [B2INR2_STRPS] | |
| B2IS42 | 81.63 | 6 | 98 | 152.99 | 50S ribosomal protein L23 OS = | Intracellular |
| | | | | | Streptococcus pneumoniae (strain | |
| | | | | | CGSP14) GN = rplW PE = 3 SV = 1 - | |
| | | | | | [RL23_STRPS] | |
| B2IPI0 | 44.02 | 18 | 418 | 152.53 | Serine hydroxymethyltransferase | Intracellular |
| | | | | | OS = -Streptococcus pneumoniae (strain | |
| | | | | | CGSP14) GN = glyA PE = 3 SV = 1 - | |
| | | | | | [GLYA_STRPS] | |
| B2IRG4 | 73.89 | 21 | 203 | 151.28 | 30S ribosomal protein S4 OS = | Intracellular |
| | | | | | Streptococcus pneumoniae (strain | |
| | | | | | CGSP14) GN = rpsD PE = 3 SV = 1 - | |
| | | | | | [RS4_STRPS] | |
| B2ILV7 | 59.29 | 21 | 452 | 150.33 | Serine--tRNA ligase OS = | Intracellular |
| | | | | | Streptococcus pneumoniae (strain | |
| | | | | | CGSP14) GN = serS PE = 3 SV = 1 - | |
| | | | | | [B2ILV7_STRPS] | |
| B2INC8 | 60.65 | 13 | 216 | 146.88 | Uracil phosphoribosyltransferase | Intracellular |
| | | | | | OS = -Streptococcus pneumoniae (strain | |
| | | | | | CGSP14) GN = upp PE = 3 SV = 1 - | |
| | | | | | [B2INC8_STRPS] | |
| B2IQL3 | 70.63 | 16 | 252 | 145.61 | Phosphate import ATP-binding | Intracellular |
| | | | | | protein PstB OS = -Streptococcus | |
| | | | | | pneumoniae (strain CGSP14) | |
| | | | | | GN = pstB PE = 3 SV = 1 - | |
| | | | | | [B2IQL3_STRPS] | |
| B2IMZ5 | 31.91 | 19 | 564 | 144.21 | ABC transporter, ATP- | Multi- |
| | | | | | binding/permease protein OS = | transmembrane |
| | | | | | Streptococcus pneumoniae (strain | |
| | | | | | CGSP14) GN = SPCG_2042 PE = 4 SV = 1 - | |
| | | | | | [B2IMZ5_STRPS] | |
| B2IS39 | 59.8 | 8 | 102 | 143.18 | 30S ribosomal protein S10 | Intracellular |
| | | | | | OS = Streptococcus pneumoniae | |
| | | | | | (strain CGSP14) GN = rpsJ PE = 3 SV = 1 - | |
| | | | | | [RS10_STRPS] | |
| B2IP78 | 53.87 | 13 | 375 | 141.59 | Carboxynorspermidine | Intracellular |
| | | | | | decarboxylase OS = -Streptococcus | |
| | | | | | pneumoniae (strain CGSP14) | |
| | | | | | GN = nspC PE = 4 SV = 1 - | |
| | | | | | [B2IP78_STRPS] | |
| B2IM60 | 54.81 | 13 | 416 | 139.41 | Diaminopimelate decarboxylase | Intracellular |
| | | | | | OS = -Streptococcus pneumoniae (strain | |
| | | | | | CGSP14) GN = lysA PE = 3 SV = 1 - | |
| | | | | | [B2IM60_STRPS] | |
| B2IMH3 | 65.03 | 14 | 346 | 139.16 | Catabolite control protein A OS = | Intracellular |
| | | | | | Streptococcus pneumoniae (strain | |
| | | | | | CGSP14) GN = ccpA PE = 4 SV = 1 - | |
| | | | | | [B2IMH3_STRPS] | |
| B2IS68 | 57.03 | 8 | 128 | 139.07 | 50S ribosomal protein L17 OS = | Intracellular |
| | | | | | Streptococcus pneumoniae (strain | |
| | | | | | CGSP14) GN = rplQ PE = 3 SV = 1 - | |
| | | | | | [RL17_STRPS] | |
| B2INE0 | 34.95 | 13 | 329 | 136.77 | Cell division protein FtsX OS = | Multi- |
| | | | | | Streptococcus pneumoniae (strain | transmembrane |
| | | | | | CGSP14) GN = ftsX PE = 3 SV = 1 - | |
| | | | | | [B2INEO_STRPS] | |
| B2ILV2 | 41.65 | 20 | 778 | 134.89 | Endonuclease MutS2 OS = | Intracellular |
| | | | | | Streptococcus pneumoniae (strain | |
| | | | | | CGSP14) GN = mutS2 PE = 3 SV = 1 - | |
| | | | | | [MUTS2_STRPS] | |
| B2IQU4 | 63.93 | 15 | 280 | 133.96 | Oxidoreductase, aldo/keto | Intracellular |
| | | | | | reductase family OS = -Streptococcus | |
| | | | | | pneumoniae (strain CGSP14) | |
| | | | | | GN = SPCG_1466 PE = 4 SV = 1 - | |
| | | | | | [B2IQU4_STRPS] | |
| B2IS55 | 71.35 | 14 | 178 | 129.78 | 50S ribosomal protein L6 OS = | Intracellular |
| | | | | | Streptococcus pneumoniae (strain | |
| | | | | | CGSP14) GN = rplF PE = 3 SV = 1 - | |
| | | | | | [RL6_STRPS] | |
| B2ISJ8 | 67.95 | 15 | 156 | 129.16 | 30S ribosomal protein S7 OS = | Intracellular |
| | | | | | Streptococcus pneumoniae (strain | |
| | | | | | CGSP14) GN = rpsG PE = 3 SV = 1 - | |
| | | | | | [RS7_STRPS] | |
| B2IRR6 | 24.81 | 8 | 266 | 128.96 | Cell division protein DivIVA OS = | Intracellular |
| | | | | | Streptococcus pneumoniae (strain | |
| | | | | | CGSP14) GN = SPCG_1633 PE = 4 SV = 1 - | |
| | | | | | [B2IRR6_STRPS] | |
| B2IS18 | 43.68 | 14 | 419 | 128.49 | Zinc metalloprotease OS = | Multi- |
| | | | | | Streptococcus pneumoniae (strain | transmembrane |
| | | | | | CGSP14) GN = eep PE = 3 SV = 1 - | |
| | | | | | [B2IS18_STRPS] | |
| B2INF3 | 47.94 | 9 | 267 | 128.19 | Peptidyl-prolyl cis-trans isomerase | Lipid |
| | | | | | OS = -Streptococcus pneumoniae (strain | anchored |
| | | | | | CGSP14) GN = ppiA PE = 3 SV = 1 - | |
| | | | | | [B2INF3_STRPS] | |
| B2IR96 | 96.59 | 13 | 176 | 127.15 | Adenine phosphoribosyltransferase | Intracellular |
| | | | | | OS = -Streptococcus pneumoniae (strain | |
| | | | | | CGSP14) GN = apt PE = 3 SV = 1 - | |
| | | | | | [B2IR96_STRPS] | |
| B2IQX1 | 63.36 | 12 | 292 | 124.75 | ATP synthase gamma chain OS = | Intracellular |
| | | | | | Streptococcus pneumoniae (strain | |
| | | | | | CGSP14) GN = atpG PE = 3 SV = 1 - | |
| | | | | | [ATPG_STRPS] | |
| B2IQL7 | 40.41 | 12 | 292 | 124.49 | Phosphate ABC transporter, | Lipid |
| | | | | | phosphate-binding protein, | anchored |
| | | | | | putative OS = -Streptococcus | |
| | | | | | pneumoniae (strain CGSP14) | |
| | | | | | GN = SPCG_1389 PE = 4 SV = 1 - | |
| | | | | | [B2IQL7_STRPS] | |
| B2IS49 | 69.77 | 10 | 86 | 124.47 | 30S ribosomal protein S17 OS = | Intracellular |
| | | | | | Streptococcus pneumoniae (strain | |
| | | | | | CGSP14) GN = rpsQ PE = 3 SV = 1 - | |
| | | | | | [RS17_STRPS] | |
| B2IQV3 | 35.99 | 8 | 289 | 123.64 | Glycerol uptake facilitator protein, | Multi- |
| | | | | | putative OS = -Streptococcus | transmembrane |
| | | | | | pneumoniae (strain CGSP14) | |
| | | | | | GN = glpF PE = 3 SV = 1 - | |
| | | | | | [B2IQV3_STRPS] | |
| B21M98 | 46.54 | 14 | 535 | 123.63 | CTP synthase OS = Streptococcus | Intracellular |
| | | | | | pneumoniae (strain CGSP14) | |
| | | | | | GN = pyrG PE = 3 SV = 1 - | |
| | | | | | [B2IM98_STRPS] | |
| B2IQ39 | 47.38 | 9 | 344 | 123.25 | Uncharacterized protein OS = | Secretory |
| | | | | | Streptococcus pneumoniae (strain | (released) |
| | | | | | CGSP14) GN = SPCG_1211 PE = 4 SV = 1 - | (with CS) |
| | | | | | [B2IQ39_STRPS] | |
| B2IP00 | 65.99 | 14 | 397 | 123.01 | Serine protease OS = -Streptococcus | N- |
| | | | | | pneumoniae (strain CGSP14) | terminally |
| | | | | | GN = sphtra PE = 4 SV = 1 - | anchored |
| | | | | | [B2IP00_STRPS] | (No CS) |
| B2IN41 | 35.2 | 16 | 429 | 122.13 | Histidine--tRNA ligase OS = | Intracellular |
| | | | | | Streptococcus pneumoniae (strain | |
| | | | | | CGSP14) GN = hisS PE = 3 SV = 1 - | |
| | | | | | [SYH_STRPS] | |
| B2IRW4 | 40.6 | 15 | 436 | 120.41 | GTPase Der OS = -Streptococcus | Intracellular |
| | | | | | pneumoniae (strain CGSP14) GN = der | |
| | | | | | PE = 3 SV = 1 - [DER_STRPS] | |
| B2IR68 | 35.97 | 13 | 531 | 119.86 | Uncharacterized protein OS = | Multi- |
| | | | | | Streptococcus pneumoniae (strain | transmembrane |
| | | | | | CGSP14) GN = SPCG_1535 PE = 4 SV = 1 - | |
| | | | | | [B2IR68_STRPS] | |
| B2IPR2 | 27.94 | 18 | 841 | 118.97 | DNA gyrase subunit A OS = | Intracellular |
| | | | | | Streptococcus pneumoniae (strain | |
| | | | | | CGSP14) GN = gyrA PE = 3 SV = 1 - | |
| | | | | | [B2IPR2_STRPS] | |
| B2IRA1 | 44.08 | 17 | 524 | 118.46 | DEAD-box ATP-dependent RNA | Intracellular |
| | | | | | helicase CshA OS = -Streptococcus | |
| | | | | | pneumoniae (strain CGSP14) | |
| | | | | | GN = cshA PE = 3 SV = 1 - | |
| | | | | | [B2IRA1_STRPS] | |
| B2IM46 | 55.94 | 11 | 345 | 117.68 | Uncharacterized protein OS = | N- |
| | | | | | Streptococcus pneumoniae (strain | terminally |
| | | | | | CGSP14) GN = SPCG_1931 PE = 4 SV = 1 - | anchored |
| | | | | | [B2IM46_STRPS] | (with CS) |
| B2ILR7 | 48.44 | 16 | 481 | 117.48 | 6-phosphogluconate | Intracellular |
| | | | | | dehydrogenase, decarboxylating | |
| | | | | | OS = -Streptococcus pneumoniae (strain | |
| | | | | | CGSP14) GN = gnd PE = 3 SV = 1 - | |
| | | | | | [B2ILR7_STRPS] | |
| B2IR57 | 62.02 | 19 | 466 | 116.97 | Cof family protein/peptidyl-prolyl | Intracellular |
| | | | | | cis-trans isomerase, cyclophilin | |
| | | | | | type OS = -Streptococcus pneumoniae | |
| | | | | | (strain CGSP14) GN = SPCG_1524 | |
| | | | | | PE = 4 SV = 1 - [B2IR57_STRPS] | |
| B2IND9 | 76.09 | 12 | 230 | 114.4 | Cell division ABC transporter, ATP- | Intracellular |
| | | | | | binding protein FtsE OS = | |
| | | | | | Streptococcus pneumoniae (strain | |
| | | | | | CGSP14) GN = ftsE PE = 4 SV = 1 - | |
| | | | | | [B2IND9_STRPS] | |
| B2IPU7 | 45 | 13 | 320 | 113.45 | Ribonucleoside-diphosphate | Intracellular |
| | | | | | reductase subunit beta OS = | |
| | | | | | Streptococcus pneumoniae (strain | |
| | | | | | CGSP14) GN = nrdF PE = 3 SV = 1 - | |
| | | | | | [B2IPU7_STRPS] | |
| B2IMS9 | 43.58 | 20 | 553 | 112.79 | Ribonuclease J OS = -Streptococcus | Intracellular |
| | | | | | pneumoniae (strain CGSP14) GN = rnj | |
| | | | | | PE = 3 SV = 1 - [B2IMS9_STRPS] | |
| B2ISQ9 | 52.02 | 15 | 494 | 111.9 | UPF0371 protein SPCG_0344 OS = | Intracellular |
| | | | | | Streptococcus pneumoniae (strain | |
| | | | | | CGSP14) GN = SPCG_0344 PE = 3 SV = 1 - | |
| | | | | | [Y344_STRPS] | |
| B2IPF4 | 53.38 | 14 | 311 | 110.86 | Adhesion lipoprotein OS = | Lipid |
| | | | | | Streptococcus pneumoniae (strain | anchored |
| | | | | | CGSP14) GN = Imb PE = 3 SV = 1 - | |
| | | | | | [B2IPF4_STRPS] | |
| B2IM45 | 50.82 | 12 | 427 | 110.79 | UDP-N-acetylglucosamine 1- | Intracellular |
| | | | | | carboxyvinyltransferase OS = | |
| | | | | | Streptococcus pneumoniae (strain | |
| | | | | | CGSP14) GN = murA PE = 3 SV = 1 - | |
| | | | | | [B2IM45_STRPS] | |
| B2INN5 | 58.46 | 10 | 272 | 109.88 | Cell shape-determining protein | N- |
| | | | | | MreC OS = -Streptococcus pneumoniae | terminally |
| | | | | | (strain CGSP14) GN = mreC PE = 3 | anchored |
| | | | | | SV = 1 - [B2INN5_STRPS] | (with CS) |
| B2IM15 | 46.84 | 10 | 158 | 109.73 | Uncharacterized protein OS = | Multi- |
| | | | | | Streptococcus pneumoniae (strain | transmembrane |
| | | | | | CGSP14) GN = SPCG_1901 PE = 4 SV = 1 - | |
| | | | | | [B2IM15_STRPS] | |
| B2IRP9 | 25.68 | 14 | 740 | 109.17 | GTP pyrophosphokinase OS = | Intracellular |
| | | | | | Streptococcus pneumoniae (strain | |
| | | | | | CGSP14) GN = reIA PE = 3 SV = 1 - | |
| | | | | | [B2IRP9_STRPS] | |
| B2INE1 | 34.71 | 11 | 726 | 108.73 | PTS system, IIABC components OS = | Multi- |
| | | | | | Streptococcus pneumoniae (strain | transmembrane |
| | | | | | CGSP14) GN = ptsG PE = 4 SV = 1 - | |
| | | | | | [B2INE1_STRPS] | |
| B2IST7 | 48.44 | 12 | 417 | 108.26 | Capsular polysaccharide | Intracellular |
| | | | | | biosynthesis protein, putative OS = | |
| | | | | | Streptococcus pneumoniae (strain | |
| | | | | | CGSP14) GN = SPCG_1812 PE = 3 SV = 1 - | |
| | | | | | [B2IST7_STRPS] | |
| B2IP76 | 71.33 | 14 | 286 | 106.74 | Polyamine aminopropyltransferase | Intracellular |
| | | | | | OS = -Streptococcus pneumoniae (strain | |
| | | | | | CGSP14) GN = speE PE = 3 SV = 1 - | |
| | | | | | [B2IP76_STRPS] | |
| B2IS23 | 34.49 | 22 | 954 | 105.34 | UvrABC system protein A OS = | Intracellular |
| | | | | | Streptococcus pneumoniae (strain | |
| | | | | | CGSP14) GN = uvrA PE = 3 SV = 1 - | |
| | | | | | [B2IS23_STRPS] | |
| B2IS53 | 52.81 | 7 | 89 | 105.15 | 30S ribosomal protein S14 OS = | Intracellular |
| | | | | | Streptococcus pneumoniae (strain | |
| | | | | | CGSP14) GN = rpsN PE = 3 SV = 1 - | |
| | | | | | [RS14_STRPS] | |
| B2IQN9 | 49.79 | 20 | 486 | 105.03 | Nicotinate | Intracellular |
| | | | | | phosphoribosyltransferase OS = | |
| | | | | | Streptococcus pneumoniae (strain | |
| | | | | | CGSP14) GN = SPCG_1408 PE = 3 SV = 1 - | |
| | | | | | [B2IQN9_STRPS] | |
| B2ISZ6 | 49.88 | 13 | 419 | 104.34 | Sugar ABC transporter, sugar- | Lipid |
| | | | | | binding protein OS = -Streptococcus | anchored |
| | | | | | pneumoniae (strain CGSP14) | |
| | | | | | GN = SPCG_1871 PE = 4 SV = 1 - | |
| | | | | | [B2ISZ6_STRPS] | |
| B2IMA9 | 47.02 | 17 | 487 | 101.63 | Type I restriction-modification | Intracellular |
| | | | | | system, M subunit OS = | |
| | | | | | Streptococcus pneumoniae (strain | |
| | | | | | CGSP14) GN = hsdM PE = 4 SV = 1 - | |
| | | | | | [B2IMA9_STRPS] | |
| B2IQH2 | 72.19 | 13 | 187 | 100.73 | 50S ribosomal protein L10 OS = | Intracellular |
| | | | | | Streptococcus pneumoniae (strain | |
| | | | | | CGSP14) GN = rplJ PE = 3 SV = 1 - | |
| | | | | | [B2IQH2_STRPS] | |
| B2IR61 | 39.82 | 14 | 447 | 100.57 | Asparagine--tRNA ligase OS = | Intracellular |
| | | | | | Streptococcus pneumoniae (strain | |
| | | | | | CGSP14) GN = asnS PE = 3 SV = 1 - | |
| | | | | | [B2IR61_STRPS] | |
| B2IM30 | 50 | 15 | 338 | 100.22 | Transcriptional regulator, putative | Secretory |
| | | | | | OS = -Streptococcus pneumoniae (strain | (released) |
| | | | | | CGSP14) GN = lytR PE = 4 SV = 1 - | (with CS) |
| | | | | | [B2IM30_STRPS] | |
| B2ISV9 | 32.29 | 10 | 511 | 99.16 | Choline transporter OS = | Multi- |
| | | | | | Streptococcus pneumoniae (strain | transmembrane |
| | | | | | CGSP14) GN = SPCG_1834 PE = 3 SV = 1 - | |
| | | | | | [B2ISV9_STRPS] | |
| B2IS51 | 78.22 | 12 | 101 | 98.15 | 50S ribosomal protein L24 OS = | Intracellular |
| | | | | | Streptococcus pneumoniae (strain | |
| | | | | | CGSP14) GN = rplX PE = 3 SV = 1 - | |
| | | | | | [RL24_STRPS] | |
| B2IRX0 | 76.67 | 11 | 240 | 97.84 | ABC transporter, ATP-binding | Intracellular |
| | | | | | protein OS = -Streptococcus | |
| | | | | | pneumoniae (strain CGSP14) | |
| | | | | | GN = SPCG_1687 PE = 4 SV = 1 - | |
| | | | | | [B2IRX0_STRPS] | |
| B2ISX8 | 46.73 | 11 | 336 | 97.53 | Non-canonical purine NTP | Intracellular |
| | | | | | pyrophosphatase OS = -Streptococcus | |
| | | | | | pneumoniae (strain CGSP14) | |
| | | | | | GN = SPCG_1853 PE = 3 SV = 1 - | |
| | | | | | [B2ISX8_STRPS] | |
| B2IN24 | 45.35 | 14 | 505 | 97.46 | 4-alpha-glucanotransferase OS = | Intracellular |
| | | | | | Streptococcus pneumoniae (strain | |
| | | | | | CGSP14) GN = maIM PE = 3 SV = 1 - | |
| | | | | | [B2IN24_STRPS] | |
| B2IM61 | 54.55 | 12 | 275 | 97.4 | Purine operon repressor OS = | Intracellular |
| | | | | | Streptococcus pneumoniae (strain | |
| | | | | | CGSP14) GN = purR PE = 4 SV = 1 - | |
| | | | | | [B2IM61_STRPS] | |
| B2ILX0 | 55.38 | 16 | 455 | 97.1 | Acetyl-CoA carboxylase OS = | Intracellular |
| | | | | | Streptococcus pneumoniae (strain | |
| | | | | | CGSP14) GN = accC PE = 4 SV = 1 - | |
| | | | | | [B2ILXO_STRPS] | |
| B2IQ19 | 35.91 | 15 | 763 | 96.38 | DNA helicase OS = -Streptococcus | Intracellular |
| | | | | | pneumoniae (strain CGSP14) | |
| | | | | | GN = pcrA PE = 3 SV = 1 - | |
| | | | | | [B2IQ19_STRPS] | |
| B2IPZ6 | 69.23 | 10 | 91 | 95.95 | DNA-binding protein HU OS = | Intracellular |
| | | | | | Streptococcus pneumoniae (strain | |
| | | | | | CGSP14) GN = hlpA PE = 3 SV = 1 - | |
| | | | | | [B2IPZ6_STRPS] | |
| B2INE5 | 31.57 | 12 | 396 | 95.45 | S-adenosylmethionine synthase | Intracellular |
| | | | | | OS = -Streptococcus pneumoniae (strain | |
| | | | | | CGSP14) GN = metK PE = 3 SV = 1 - | |
| | | | | | [METK_STRPS] | |
| B2IS82 | 28.53 | 15 | 659 | 94.22 | Serine/threonine protein kinase | Intracellular/ |
| | | | | | OS = -Streptococcus pneumoniae (strain | TMH start |
| | | | | | CGSP14) GN = pkn2 PE = 3 SV = 1 - | AFTER 60 |
| | | | | | [B2IS82_STRPS] | |
| B2IS65 | 58.68 | 10 | 121 | 93.19 | 30S ribosomal protein S13 OS = | Intracellular |
| | | | | | Streptococcus pneumoniae (strain | |
| | | | | | CGSP14) GN = rpsM PE = 3 SV = 1 - | |
| | | | | | [RS13_STRPS] | |
| B21MI4 | 35.02 | 18 | 731 | 92.99 | Penicillin-binding protein 2A OS = | N- |
| | | | | | Streptococcus pneumoniae (strain | terminally |
| | | | | | CGSP14) GN = pbp2A PE = 4 SV = 1 - | anchored |
| | | | | | [B2IMI4_STRPS] | (with CS) |
| B2IRK4 | 37.05 | 17 | 637 | 92.87 | tRNA uridine 5- | Intracellular |
| | | | | | carboxymethylaminomethyl | |
| | | | | | modification enzyme MnmG OS = | |
| | | | | | Streptococcus pneumoniae (strain | |
| | | | | | CGSP14) GN = mnmG PE = 3 SV = 1 - | |
| | | | | | [B2IRK4_STRPS] | |
| B2ISW0 | 69.01 | 11 | 242 | 92.71 | Choline transporter OS = | Intracellular |
| | | | | | Streptococcus pneumoniae (strain | |
| | | | | | CGSP14) GN = SPCG_1835 PE = 4 SV = 1 - | |
| | | | | | [B2ISWO_STRPS] | |
| B2INP6 | 39.59 | 10 | 341 | 92.59 | Tryptophan--tRNA ligase OS = | Intracellular |
| | | | | | Streptococcus pneumoniae (strain | |
| | | | | | CGSP14) GN = trpS PE = 3 SV = 1 - | |
| | | | | | [B2INP6_STRPS] | |
| B2INC5 | 39.5 | 11 | 281 | 92.15 | Uncharacterized protein OS = | Intracellular |
| | | | | | Streptococcus pneumoniae (strain | |
| | | | | | CGSP14) GN = SPCG_0692 PE = 4 SV = 1 - | |
| | | | | | [B2INC5_STRPS] | |
| B2IP66 | 36.64 | 17 | 715 | 91.51 | Transcriptional regulator, putative | Intracellular |
| | | | | | OS = -Streptococcus pneumoniae (strain | |
| | | | | | CGSP14) GN = SPCG_0885 PE = 4 SV = 1 - | |
| | | | | | [B2IP66_STRPS] | |
| B2ISY1 | 46.58 | 14 | 541 | 91.27 | Dextran glucosidase DexS, putative | Intracellular |
| | | | | | OS = -Streptococcus pneumoniae (strain | |
| | | | | | CGSP14) GN = dexS PE = 4 SV = 1 - | |
| | | | | | [B2ISY1_STRPS] | |
| B2ISK7 | 38.51 | 11 | 444 | 91.26 | Aminopeptidase C OS = -Streptococcus | Intracellular |
| | | | | | pneumoniae (strain CGSP14) | |
| | | | | | GN = pepC PE = 4 SV = 1 - | |
| | | | | | [B2ISK7_STRPS] | |
| B2INL6 | 30.75 | 8 | 335 | 91.2 | ABC transporter, substrate-binding | Lipid |
| | | | | | protein, putative OS = -Streptococcus | anchored |
| | | | | | pneumoniae (strain CGSP14) | |
| | | | | | GN = SPCG_2165 PE = 4 SV = 1 - | |
| | | | | | [B2INL6_STRPS] | |
| B2IQY9 | 25.83 | 11 | 453 | 90.37 | Chromosomal replication initiator | Intracellular |
| | | | | | protein DnaA OS = -Streptococcus | |
| | | | | | pneumoniae (strain CGSP14) | |
| | | | | | GN = dnaA PE = 3 SV = 1 - | |
| | | | | | [DNAA_STRPS] | |
| B2IRS8 | 22.34 | 13 | 685 | 90.12 | Penicillin-binding protein 2B OS = | N- |
| | | | | | Streptococcus pneumoniae (strain | terminally |
| | | | | | CGSP14) GN = pbp2B PE = 4 SV = 1 - | anchored |
| | | | | | [B2IRS8_STRPS] | (with CS) |
| B2IS56 | 53.44 | 8 | 131 | 88.46 | 50S ribosomal protein L18 OS = | Intracellular |
| | | | | | Streptococcus pneumoniae (strain | |
| | | | | | CGSP14) GN = rplR PE = 3 SV = 1 - | |
| | | | | | [B2IS56_STRPS] | |
| B2IQ12 | 40.13 | 9 | 319 | 88.41 | Ribose-phosphate | Intracellular |
| | | | | | pyrophosphokinase OS = | |
| | | | | | Streptococcus pneumoniae (strain | |
| | | | | | CGSP14) GN = prs PE = 3 SV = 1 - | |
| | | | | | [B2IQ12_STRPS] | |
| B2IS67 | 52.73 | 9 | 311 | 88.17 | DNA-directed RNA polymerase | Intracellular |
| | | | | | subunit alpha OS = -Streptococcus | |
| | | | | | pneumoniae (strain CGSP14) | |
| | | | | | GN = rpoA PE = 3 SV = 1 - | |
| | | | | | [B2IS67_STRPS] | |
| B2IS59 | 64.38 | 9 | 146 | 88.05 | 50S ribosomal protein L15 OS = | Intracellular |
| | | | | | Streptococcus pneumoniae (strain | |
| | | | | | CGSP14) GN = rplO PE = 3 SV = 1 - | |
| | | | | | [RL15_STRPS] | |
| B2IQ61 | 41.52 | 9 | 289 | 87.7 | Choline kinase OS = -Streptococcus | Intracellular |
| | | | | | pneumoniae (strain CGSP14) GN = pck | |
| | | | | | PE = 4 SV = 1 - [B2IQ61_STRPS] | |
| B2ISY2 | 24.4 | 10 | 705 | 87.27 | Trehalose PTS system, IIABC | Multi- |
| | | | | | components OS = -Streptococcus | transmembrane |
| | | | | | pneumoniae strain CGSP14) GN = treP | |
| | | | | | PE = 4 SV = 1 - [B2ISY2_STRPS] | |
| B2IMQ3 | 25.97 | 12 | 801 | 86.8 | Phenylalanine--tRNA ligase beta | Intracellular |
| | | | | | subunit OS = -Streptococcus | |
| | | | | | pneumoniae (strain CGSP14) | |
| | | | | | GN = pheT PE = 3 SV = 1 - | |
| | | | | | [B2IMQ3_STRPS] | |
| B2IQ02 | 60.82 | 9 | 97 | 86.49 | 50S ribosomal protein L27 OS = | Intracellular |
| | | | | | Streptococcus pneumoniae (strain | |
| | | | | | CGSP14) GN = rpmA PE = 3 SV = 1 - | |
| | | | | | [RL27_STRPS] | |
| B2IN58 | 40.3 | 11 | 335 | 86.4 | Uncharacterized protein OS = | Multi- |
| | | | | | Streptococcus pneumoniae (strain | transmembrane |
| | | | | | CGSP14) GN = SPCG_2101 PE = 4 SV = 1 - | |
| | | | | | [B2IN58_STRPS] | |
| B2IQW4 | 62.2 | 10 | 209 | 84.4 | Amino acid ABC transporter, ATP- | Intracellular |
| | | | | | binding protein OS = -Streptococcus | |
| | | | | | pneumoniae (strain CGSP14) | |
| | | | | | GN = SPCG_1486 PE = 4 SV = 1 - | |
| | | | | | [B2IQW4_STRPS] | |
| B2IP74 | 35.23 | 13 | 491 | 84.24 | Lysine decarboxylase OS = | Intracellular |
| | | | | | Streptococcus pneumoniae (strain | |
| | | | | | CGSP14) GN = cad PE = 4 SV = 1 - | |
| | | | | | [B2IP74_STRPS] | |
| B2IS70 | 42.49 | 11 | 433 | 83.11 | UPF0210 protein SPCG_0246 OS = | Intracellular |
| | | | | | Streptococcus pneumoniae (strain | |
| | | | | | CGSP14) GN = SPCG_0246 PE = 3 SV = 1 - | |
| | | | | | [B2IS70_STRPS] | |
| B2IQ66 | 64.29 | 13 | 280 | 83.05 | LicD2 protein OS = -Streptococcus | Intracellular |
| | | | | | pneumoniae (strain CGSP14) | |
| | | | | | GN = licD2 PE = 4 SV = 1 - | |
| | | | | | [B2IQ66_STRPS] | |
| B2IRK5 | 34.17 | 12 | 559 | 81.76 | Ribonuclease J OS = -Streptococcus | Intracellular |
| | | | | | pneumoniae (strain CGSP14) GN = rnj | |
| | | | | | PE = 3 SV = 1 - [B2IRK5_STRPS] | |
| B2IR84 | 68.24 | 12 | 296 | 81.42 | Nucleotide-binding protein | Intracellular |
| | | | | | SPCG_1551 OS = -Streptococcus | |
| | | | | | pneumoniae (strain CGSP14) | |
| | | | | | GN = SPCG_1551 PE = 3 SV = 1 - | |
| | | | | | [Y1551_STRPS] | |
| B2IMB5 | 34.93 | 15 | 607 | 81.24 | Chaperone protein Dnak OS = | Intracellular |
| | | | | | Streptococcus pneumoniae (strain | |
| | | | | | CGSP14) GN = dnaK PE = 3 SV = 1 - | |
| | | | | | [DNAK_STRPS] | |
| B2IQ62 | 58.09 | 12 | 346 | 80.31 | Alcohol dehydrogenase, zinc- | Intracellular |
| | | | | | containing OS = -Streptococcus | |
| | | | | | pneumoniae (strain CGSP14) | |
| | | | | | GN = SPCG_1234 PE = 4 SV = 1 - | |
| | | | | | [B2IQ62_STRPS] | |
| B2IMB9 | 37.86 | 11 | 243 | 79.94 | ABC transporter, ATP-binding | Intracellular |
| | | | | | protein OS = -Streptococcus | |
| | | | | | pneumoniae (strain CGSP14) | |
| | | | | | GN = SPCG_0494 PE = 4 SV = 1 - | |
| | | | | | [B2IMB9_STRPS] | |
| B2IRD4 | 56.18 | 5 | 89 | 79.7 | 30S ribosomal protein S15 OS = | Intracellular |
| | | | | | Streptococcus pneumoniae (strain | |
| | | | | | CGSP14) GN = rpsO PE = 3 SV = 1 - | |
| | | | | | [RS15_STRPS] | |
| B2IN68 | 50.79 | 8 | 126 | 79.14 | Uncharacterized protein OS = | N- |
| | | | | | Streptococcus pneumoniae (strain | terminally |
| | | | | | CGSP14) GN = SPCG_0635 PE = 4 SV = 1 - | anchored |
| | | | | | [B2IN68_STRPS] | (with CS) |
| BEIQN0 | 44.09 | 3 | 127 | 78.99 | Uncharacterized protein OS = | N- |
| | | | | | Streptococcus pneumoniae (strain | terminally |
| | | | | | CGSP14) GN = SPCG_1399 PE = 4 SV = 1 | anchored |
| | | | | | [B2IQNO_STRPS] | (with CS) |
| B2ILY0 | 36.58 | 14 | 514 | 78.96 | Peptide chain release factor 3 OS = | Intracellular |
| | | | | | Streptococcus pneumoniae (strain | |
| | | | | | CGSP14) GN = prfC PE = 3 SV = 1 - | |
| | | | | | [RF3_STRPS] | |
| B2IS54 | 57.58 | 8 | 132 | 78.42 | 30S ribosomal protein S8 OS = | Intracellular |
| | | | | | Streptococcus pneumoniae (strain | |
| | | | | | CGSP14) GN = rpsH PE = 3 SV = 1 - | |
| | | | | | [RS8_STRPS] | |
| B2IMB6 | 35.19 | 13 | 378 | 78.27 | Chaperone protein DnaJ OS- | Intracellular |
| | | | | | Streptococcus pneumoniae (strain | |
| | | | | | CGSP14) GN = dnaJ PE = 3 SV = 1 - | |
| | | | | | [B2IMB6_STRPS] | |
| B2IQS5 | 42.11 | 12 | 247 | 78.16 | Amino acid ABC transporter, ATP- | Intracellular |
| | | | | | binding protein OS = -Streptococcus | |
| | | | | | pneumoniae (strain CGSP14) | |
| | | | | | GN = SPCG_1447 PE = 4 SV = 1 - | |
| | | | | | [B2IQS5_STRPS] | |
| B2IRH0 | 33.01 | 12 | 512 | 76.84 | ABC transporter, substrate-binding | Lipid |
| | | | | | protein OS = -Streptococcus | anchored |
| | | | | | pneumoniae (strain CGSP14) | |
| | | | | | GN = SPCG_0089 PE = 4 SV = 1 - | |
| | | | | | [B2IRHO_STRPS] | |
| B21PW9 | 50.88 | 11 | 283 | 76.72 | Ribosome biogenesis GTPase A OS = | Intracellular |
| | | | | | Streptococcus pneumoniae (strain | |
| | | | | | CGSP14) GN = SPCG_1141 PE = 3 SV = 1 - | |
| | | | | | [B2IPW9_STRPS] | |
| B2IN18 | 48.09 | 11 | 418 | 76.44 | Tyrosine--tRNA ligase OS = | Intracellular |
| | | | | | Streptococcus pneumoniae (strain | |
| | | | | | CGSP14) GN = tyrS PE = 3 SV = 1 - | |
| | | | | | [SYY_STRPS] | |
| B2IQV9 | 33.39 | 13 | 572 | 75.14 | Phosphoglucomutase OS = | Intracellular |
| | | | | | Streptococcus pneumoniae (strain | |
| | | | | | CGSP14) GN = pgm PE = 3 SV = 1 - | |
| | | | | | [B2IQV9_STRPS] | |
| B2IP77 | 38.9 | 9 | 419 | 74.94 | Uncharacterized protein OS = | Intracellular |
| | | | | | Streptococcus pneumoniae (strain | |
| | | | | | CGSP14) GN = SPCG_0896 PE = 4 SV = 1 - | |
| | | | | | [B2IP77_STRPS] | |
| B2IQ63 | 53.19 | 8 | 235 | 74.22 | 2-C-methyl-D-erythritol 4- | Intracellular |
| | | | | | phosphate cytidylyltransferase OS = | |
| | | | | | Streptococcus pneumoniae (strain | |
| | | | | | CGSP14) GN = ispD PE = 3 SV = 1 - | |
| | | | | | [ISPD_STRPS] | |
| B2IRQ4 | 65.34 | 11 | 251 | 74.2 | Manganese ABC transporter, ATP- | Intracellular |
| | | | | | binding protein OS = -Streptococcus | |
| | | | | | pneumoniae (strain CGSP14) | |
| | | | | | GN = SPCG_1621 PE = 4 SV = 1 - | |
| | | | | | [B2IRQ4_STRPS] | |
| B2IQX3 | 62.36 | 8 | 178 | 73.32 | ATP synthase subunit delta OS = | Intracellular |
| | | | | | Streptococcus pneumoniae (strain | |
| | | | | | CGSP14) GN = atpH PE = 3 SV = 1 - | |
| | | | | | [ATPD_STRPS] | |
| B2INX4 | 27.48 | 12 | 826 | 73.21 | DNA topoisomerase 4 subunit A | Intracellular |
| | | | | | OS = -Streptococcus pneumoniae (strain | |
| | | | | | CGSP14) GN = parC PE = 3 SV = 1 - | |
| | | | | | [B2INX4_STRPS] | |
| B2IN76 | 40.67 | 12 | 450 | 72.88 | UDP-N-acetylmuramoylalanine--D- | Intracellular |
| | | | | | glutamate ligase OS = -Streptococcus | |
| | | | | | pneumoniae (strain CGSP14) | |
| | | | | | GN = murD PE = 3 SV = 1 - | |
| | | | | | [MURD_STRPS] | |
| B2IPC3 | 41.47 | 10 | 299 | 72.75 | GTPase Era OS = -Streptococcus | Intracellular |
| | | | | | pneumoniae (strain CGSP14) GN = era | |
| | | | | | PE = 3 SV = 1 - [ERA_STRPS] | |
| B2IRX7 | 40.79 | 13 | 353 | 72.1 | Methionine import ATP-binding | Intracellular |
| | | | | | protein MetN OS = -Streptococcus | |
| | | | | | pneumoniae (strain CGSP14) | |
| | | | | | GN = metN PE = 3 SV = 1 - | |
| | | | | | [B2IRX7_STRPS] | |
| B2INU1 | 50.65 | 9 | 306 | 70.6 | Bifunctional protein FoID OS = | Lipid |
| | | | | | Streptococcus pneumoniae (strain | anchored |
| | | | | | CGSP14) GN = foID PE = 3 SV = 1 - | |
| | | | | | [B2INU1_STRPS] | |
| B2ILW4 | 36.11 | 6 | 324 | 69.62 | Enoyl-(Acyl-carrier-protein) | Intracellular |
| | | | | | reductase OS = -Streptococcus | |
| | | | | | pneumoniae (strain CGSP14) | |
| | | | | | GN = fabK PE = 4 SV = 1 - | |
| | | | | | [B2ILW4_STRPS] | |
| B2IS45 | 40.35 | 3 | 114 | 69.56 | 50S ribosomal protein L22 OS = | Intracellular |
| | | | | | Streptococcus pneumoniae (strain | |
| | | | | | CGSP14) GN = rplV PE = 3 SV = 1 - | |
| | | | | | [RL22_STRPS] | |
| B2IPZ1 | 30.21 | 13 | 652 | 67.59 | DNA ligase OS = -Streptococcus | Intracellular |
| | | | | | pneumoniae (strain CGSP14) | |
| | | | | | GN = ligA PE = 3 SV = 1 - [DNLJ_STRPS] | |
| B2IMZ3 | 34.66 | 12 | 603 | 66.96 | ABC transporter, ATP- | Multi- |
| | | | | | binding/permease protein OS = | transmembrane |
| | | | | | Streptococcus pneumoniae (strain | |
| | | | | | CGSP14) GN = SPCG_2040 PE = 4 SV = 1 - | |
| | | | | | [B2IMZ3_STRPS] | |
| B2IS63 | 54.72 | 6 | 212 | 66.76 | Adenylate kinase OS = -Streptococcus | Intracellular |
| | | | | | pneumoniae (strain CGSP14) GN = adk | |
| | | | | | PE = 3 SV = 1 - [KAD_STRPS] | |
| B2INV5 | 53.01 | 6 | 83 | 65.46 | 30S ribosomal protein S20 OS = | Intracellular |
| | | | | | Streptococcus pneumoniae (strain | |
| | | | | | CGSP14) GN = rpsT PE = 3 SV = 1 - | |
| | | | | | [B2INV5_STRPS] | |
| B2IRK2 | 38.07 | 10 | 373 | 65.45 | tRNA-specific 2-thiouridylase | Intracellular |
| | | | | | MnmA OS = -Streptococcus | |
| | | | | | pneumoniae (strain CGSP14) | |
| | | | | | GN = mnmA PE = 3 SV = 2 - | |
| | | | | | [MNMA_STRPS] | |
| B2IRB7 | 27.22 | 2 | 158 | 65.21 | Uncharacterized protein OS = | N- |
| | | | | | Streptococcus pneumoniae (strain | terminally |
| | | | | | CGSP14) GN = SPCG_1584 PE = 4 SV = 1 - | anchored |
| | | | | | [B2IRB7_STRPS] | (with CS) |
| B2INE6 | 56.27 | 10 | 311 | 64.68 | Dihydroorotate dehydrogenase OS = | Intracellular |
| | | | | | Streptococcus pneumoniae (strain | |
| | | | | | CGSP14) GN = pyrD PE = 3 SV = 1 - | |
| | | | | | [B2INE6_STRPS] | |
| B2IMB0 | 27.41 | 15 | 777 | 64.13 | Type I restriction-modification | Intracellular |
| | | | | | system, R subunit OS = -Streptococcus | |
| | | | | | pneumoniae (strain CGSP14) | |
| | | | | | GN = hsdR PE = 4 SV = 1 - | |
| | | | | | [B2IMBO_STRPS] | |
| B2ISQ4 | 20.27 | 11 | 750 | 63.95 | Penicillin-binding protein 2X OS = | N- |
| | | | | | Streptococcus pneumoniae (strain | terminally |
| | | | | | CGSP14) GN = pbp2X PE = 4 SV = 1 - | anchored |
| | | | | | [B2ISQ4_STRPS] | (with CS) |
| B2IPQ3 | 27.88 | 12 | 556 | 63.73 | Formate--tetrahydrofolate ligase | Intracellular |
| | | | | | OS = -Streptococcus pneumoniae (strain | |
| | | | | | CGSP14) GN = fhs PE = 3 SV = 1 - | |
| | | | | | [FTHS_STRPS] | |
| B2IMU7 | 41.84 | 5 | 141 | 63.38 | 50S ribosomal protein L11 OS = | Intracellular |
| | | | | | Streptococcus pneumoniae (strain | |
| | | | | | CGSP14) GN = rplK PE = 3 SV = 1 - | |
| | | | | | [RL11_STRPS] | |
| B2ISY0 | 38 | 4 | 100 | 62.82 | UPF0154 protein SPCG_1855 OS = | N- |
| | | | | | Streptococcus pneumoniae (strain | terminally |
| | | | | | CGSP14) GN = SPCG_1855 PE = 3 SV = 1 - | anchored |
| | | | | | [B2ISYO_STRPS] | (with CS) |
| B2IQP4 | 29.49 | 9 | 434 | 62.33 | Peptidase, U32 family OS = | Intracellular |
| | | | | | Streptococcus pneumoniae (strain | |
| | | | | | CGSP14) GN = SPCG_1416 PE = 4 SV = 1 - | |
| | | | | | [B2IQP4_STRPS] | |
| B2IR79 | 57.14 | 11 | 259 | 62.31 | Uncharacterized protein OS = | N- |
| | | | | | Streptococcus pneumoniae (strain | terminally |
| | | | | | CGSP14) GN = SPCG_1546 PE = 4 SV = 1 - | anchored |
| | | | | | [B2IR79_STRPS] | (with CS) |
| B2ILW2 | 21.6 | 8 | 324 | 62.19 | 3-oxoacyl-[acyl-carrier-protein] | Intracellular |
| | | | | | synthase 3 OS = -Streptococcus | |
| | | | | | pneumoniae (strain CGSP14) | |
| | | | | | GN = fabH PE = 3 SV = 1 - | |
| | | | | | [FABH_STRPS] | |
| B2IPZ9 | 43.28 | 9 | 305 | 61.07 | Riboflavin biosynthesis protein OS = | Intracellular |
| | | | | | Streptococcus pneumoniae (strain | |
| | | | | | CGSP14) GN = mreA PE = 3 SV = 1 - | |
| | | | | | [B2IPZ9_STRPS] | |
| B2IN32 | 29.98 | 13 | 587 | 60.72 | Aspartate--tRNA ligase OS = | Intracellular |
| | | | | | Streptococcus pneumoniae (strain | |
| | | | | | CGSP14) GN = aspS PE = 3 SV = 1 - | |
| | | | | | [SYD_STRPS] | |
| B2IRR9 | 42.46 | 7 | 179 | 60.72 | Cell division protein SepF OS = | Intracellular |
| | | | | | Streptococcus pneumoniae (strain | |
| | | | | | CGSP14) GN = sepF PE = 3 SV = 1 - | |
| | | | | | [SEPF_STRPS] | |
| B2IN19 | 28.12 | 13 | 690 | 60.07 | Cation-transporting ATPase, El-E2 | Intracellular |
| | | | | | family OS = -Streptococcus pneumoniae | |
| | | | | | (strain CGSP14) GN = ctpC PE = 3 SV = 1 - | |
| | | | | | [B2IN19_STRPS] | |
| B2IQY8 | 36.71 | 9 | 444 | 59.95 | UDP-N-acetylmuramate--L-alanine | Intracellular |
| | | | | | ligase OS = -Streptococcus pneumoniae | |
| | | | | | (strain CGSP14) GN = murC PE = 3 | |
| | | | | | SV = 1 - [MURC_STRPS] | |
| B2IS26 | 33.86 | 8 | 189 | 59.81 | Uncharacterized protein OS = | Lipid |
| | | | | | Streptococcus pneumoniae (strain | anchored |
| | | | | | CGSP14) GN = SPCG_0204 PE = 4 SV = 1 | |
| | | | | | [B2IS26_STRPS] | |
| B2INY7 | 45.25 | 8 | 263 | 59.27 | ABC transporter, ATP-binding | Intracellular |
| | | | | | protein OS = -Streptococcus | |
| | | | | | pneumoniae (strain CGSP14) | |
| | | | | | GN = SPCG_0812 PE = 4 SV = 1 - | |
| | | | | | [B2INY7_STRPS] | |
| B2ILR6 | 14.66 | 6 | 464 | 58.78 | Mid-cell-anchored protein Z OS = | N- |
| | | | | | Streptococcus pneumoniae (strain | terminally |
| | | | | | CGSP14) GN = mapZ PE = 3 SV = 1 - | anchored |
| | | | | | [B2ILR6_STRPS] | (with CS) |
| B2ILX8 | 36.68 | 10 | 488 | 58.59 | Glutamyl-tRNA(Gln) | Intracellular |
| | | | | | amidotransferase subunit A OS = | |
| | | | | | Streptococcus pneumoniae (strain | |
| | | | | | CGSP14) GN = gatA PE = 3 SV = 1 - | |
| | | | | | [GATA_STRPS] | |
| B2IMN3 | 35.26 | 7 | 380 | 58.5 | Queuine tRNA-ribosyltransferase | Intracellular |
| | | | | | OS = -Streptococcus pneumoniae (strain | |
| | | | | | CGSP14) GN = tgt PE = 3 SV = 1 - | |
| | | | | | [TGT_STRPS] | |
| B2IN17 | 21.09 | 12 | 825 | 58.4 | Penicillin-binding protein 1B OS = | Intracellular/ |
| | | | | | Streptococcus pneumoniae (strain | TMH start |
| | | | | | CGSP14) GN = pbp1B PE = 4 SV = 1 - | AFTER 60 |
| | | | | | [B2IN17_STRPS] | |
| B2IR27 | 45.45 | 8 | 330 | 57.61 | Phosphate acyltransferase OS = | Intracellular |
| | | | | | Streptococcus pneumoniae (strain | |
| | | | | | CGSP14) GN = plsX PE = 3 SV = 1 - | |
| | | | | | [PLSX_STRPS] | |
| B2INF7 | 73.21 | 6 | 112 | 56.77 | ATP cone domain-containing | Intracellular |
| | | | | | protein OS = -Streptococcus | |
| | | | | | pneumoniae (strain CGSP14) | |
| | | | | | GN = SPCG_0728 PE = 4 SV = 1 - | |
| | | | | | [B2INF7_STRPS] | |
| B2IMZ8 | 20.78 | 9 | 563 | 56.13 | Arginine--tRNA ligase OS = | Intracellular |
| | | | | | Streptococcus pneumoniae (strain | |
| | | | | | CGSP14) GN = argS PE = 3 SV = 1 - | |
| | | | | | [SYR_STRPS] | |
| B2ILU5 | 35.6 | 10 | 427 | 55.96 | Trigger factor OS = -Streptococcus | Intracellular |
| | | | | | pneumoniae (strain CGSP14) GN = tig | |
| | | | | | PE = 3 SV = 1 - [TIG_STRPS] | |
| B2INK8 | 56.55 | 9 | 336 | 55.86 | tRNA-dihydrouridine synthase OS = | Intracellular |
| | | | | | Streptococcus pneumoniae (strain | |
| | | | | | CGSP14) GN = SPCG_2157 PE = 3 SV = 1 | |
| | | | | | [B2INK8_STRPS] | |
| B2IM90 | 37.14 | 11 | 560 | 55.51 | ABC transporter, ATP-binding | Intracellular |
| | | | | | protein OS = -Streptococcus | |
| | | | | | pneumoniae (strain CGSP14) | |
| | | | | | GN = SPCG_0465 PE = 4 SV = 1 - | |
| | | | | | [B2IM90_STRPS] | |
| B2IQA4 | 37.72 | 12 | 448 | 54.94 | Glutamate dehydrogenase OS = | Intracellular |
| | | | | | Streptococcus pneumoniae (strain | |
| | | | | | CGSP14) GN = gdhA PE = 3 SV = 1 - | |
| | | | | | [B2IQA4_STRPS] | |
| B2IPP0 | 30.1 | 8 | 495 | 54.8 | Glucose-6-phosphate 1- | Intracellular |
| | | | | | dehydrogenase OS = -Streptococcus | |
| | | | | | pneumoniae (strain CGSP14) GN = zwf | |
| | | | | | PE = 3 SV = 1 - [B2IPPO_STRPS] | |
| B2IPZ5 | 24.33 | 13 | 633 | 54.29 | ABC transporter, ATP-binding | Intracellular |
| | | | | | protein OS = -Streptococcus | |
| | | | | | pneumoniae (strain CGSP14) | |
| | | | | | GN = SPCG_1167 PE = 4 SV = 1 - | |
| | | | | | [B2IPZ5_STRPS] | |
| B2IS10 | 23.73 | 8 | 649 | 54.27 | DNA mismatch repair protein MutL | Intracellular |
| | | | | | OS = -Streptococcus pneumoniae (strain | |
| | | | | | CGSP14) GN = mutL PE = 3 SV = 1 - | |
| | | | | | [MUTL_STRPS] | |
| B2IN96 | 47.62 | 10 | 378 | 54.14 | Lactate oxidase OS = -Streptococcus | Intracellular |
| | | | | | pneumoniae (strain CGSP14) | |
| | | | | | GN = IctO PE = 4 SV = 1 - | |
| | | | | | [B2IN96_STRPS] | |
| B2IND6 | 51.69 | 6 | 236 | 53.89 | Branched-chain amino acid ABC | Intracellular |
| | | | | | transporter, ATP-binding protein | |
| | | | | | OS = -Streptococcus pneumoniae (strain | |
| | | | | | CGSP14) GN = SPCG_0703 PE = 4 SV = 1 - | |
| | | | | | [B2IND6_STRPS] | |
| B2IS98 | 58.85 | 9 | 209 | 53.7 | Probable nicotinate-nucleotide | Intracellular |
| | | | | | adenylyltransferase OS = | |
| | | | | | Streptococcus pneumoniae (strain | |
| | | | | | CGSP14) GN = nadD PE = 3 SV = 1 - | |
| | | | | | [B2IS98_STRPS] | |
| B2IPN8 | 43.01 | 8 | 272 | 53.21 | Cof family protein OS = -Streptococcus | Intracellular |
| | | | | | pneumoniae (strain CGSP14) | |
| | | | | | GN = SPCG_1058 PE = 4 SV = 1 - | |
| | | | | | [B2IPN8_STRPS] | |
| B2INN8 | 51.27 | 10 | 275 | 52.83 | Energy-coupling factor transporter | N- |
| | | | | | ATP-binding protein EcfA OS = | terminally |
| | | | | | Streptococcus pneumoniae (strain | anchored |
| | | | | | CGSP14) GN = ecfA PE = 3 SV = 1 - | (with CS) |
| | | | | | [B2INN8_STRPS] | |
| B2ISR0 | 23.55 | 8 | 535 | 52.43 | Glucan 1,6-alpha-glucosidase OS = | Intracellular |
| | | | | | Streptococcus pneumoniae (strain | |
| | | | | | CGSP14) GN = dexB PE = 4 SV = 1 - | |
| | | | | | [B2ISRO_STRPS] | |
| B2IRF6 | 52.19 | 8 | 228 | 52.22 | Potassium uptake protein, Trk | N- |
| | | | | | family OS = -Streptococcus pneumoniae | terminally |
| | | | | | (strain CGSP14) GN = trkA PE = 4 SV = 1 - | anchored |
| | | | | | [B2IRF6_STRPS] | (with CS) |
| B2IM43 | 17.16 | 7 | 443 | 51.63 | CBS domain protein OS = | Multi- |
| | | | | | Streptococcus pneumoniae (strain | transmembrane |
| | | | | | CGSP14) GN = SPCG_1928 PE = 4 SV = 1 - | |
| | | | | | [B2IM43_STRPS] | |
| B2ILX2 | 40 | 6 | 255 | 51.58 | Acetyl-coenzyme A carboxylase | Intracellular |
| | | | | | carboxyl transferase subunit alpha | |
| | | | | | OS = -Streptococcus pneumoniae (strain | |
| | | | | | CGSP14) GN = accA PE = 3 SV = 1 - | |
| | | | | | [ACCA_STRPS] | |
| B2ISA0 | 34.78 | 10 | 368 | 51.32 | GTP-binding protein OS = | Intracellular |
| | | | | | Streptococcus pneumoniae (strain | |
| | | | | | CGSP14) GN = SPCG_1723 PE = 4 SV = 1 - | |
| | | | | | [B2ISAO_STRPS] | |
| B2IPB9 | 23.19 | 10 | 677 | 50.7 | Endo-beta-N-acetylglucosaminidase OS =- | Secretory |
| | | | | | Streptococcus pneumoniae (strain | (released) |
| | | | | | CGSP14) GN = lytB PE = 4 SV = 1 - | (with CS) |
| | | | | | [B2IPB9_STRPS] | |
| B2IQW9 | 61.87 | 6 | 139 | 50.69 | ATP synthase epsilon chain OS = | Intracellular |
| | | | | | Streptococcus pneumoniae (strain | |
| | | | | | CGSP14) GN = atpC PE = 3 SV = 1 - | |
| | | | | | [ATPE_STRPS] | |
| B2IPN9 | 24.94 | 7 | 429 | 50.34 | Signal recognition particle receptor | Intracellular |
| | | | | | FtsY OS = -Streptococcus pneumoniae | |
| | | | | | (strain CGSP14) GN = ftsY PE = 3 SV = 1 - | |
| | | | | | [B2IPN9_STRPS] | |
| B2IRI7 | 17.95 | 9 | 713 | 49.57 | Uncharacterized protein OS = | Multi- |
| | | | | | Streptococcus pneumoniae (strain | transmembrane |
| | | | | | CGSP14) GN = SPCG_0106 PE = 4 SV = 1 - | |
| | | | | | [B2IRI7_STRPS] | |
| B2IQH3 | 24.39 | 7 | 488 | 49.39 | Chlorohydrolase OS = -Streptococcus | Intracellular |
| | | | | | pneumoniae (strain CGSP14) | |
| | | | | | GN = trzA PE = 4 SV = 1 - | |
| | | | | | [B2IQH3_STRPS] | |
| B2IRD2 | 21.59 | 7 | 264 | 48.68 | Acyltransferase family protein OS = | Multi- |
| | | | | | Streptococcus pneumoniae (strain | transmembrane |
| | | | | | CGSP14) GN = SPCG_1599 PE = 4 SV = 1 - | |
| | | | | | [B2IRD2_STRPS] | |
| B2IR63 | 31.14 | 7 | 395 | 48.62 | Aspartate aminotransferase OS = | Intracellular |
| | | | | | Streptococcus pneumoniae (strain | |
| | | | | | CGSP14) GN = aspB PE = 3 SV = 1 - | |
| | | | | | [B2IR63_STRPS] | |
| B2INK4 | 23.36 | 10 | 608 | 48.46 | Uncharacterized protein OS = | N- |
| | | | | | Streptococcus pneumoniae (strain | terminally |
| | | | | | CGSP14) GN = SPCG_2153 PE = 4 SV = 1 - | anchored |
| | | | | | [B2INK4_STRPS] | (with CS) |
| B2IN18 | 28.14 | 7 | 501 | 48.06 | Zinc ABC transporter, zinc-binding | Lipid |
| | | | | | lipoprotein OS = -Streptococcus | anchored |
| | | | | | pneumoniae (strain CGSP14) | |
| | | | | | GN = SPCG_2137 PE = 3 SV = 1 - | |
| | | | | | [B2IN18_STRPS] | |
| B2IPU4 | 40.71 | 7 | 253 | 47.82 | Lactose phosphotransferase system | Intracellular |
| | | | | | repressor OS = -Streptococcus | |
| | | | | | pneumoniae (strain CGSP14) | |
| | | | | | GN = lacR PE = 4 SV = 1 - | |
| | | | | | [B2IPU4_STRPS] | |
| B2INN7 | 41.94 | 8 | 279 | 47.56 | Energy-coupling factor transporter | N- |
| | | | | | ATP-binding protein EcfA OS = | terminally |
| | | | | | Streptococcus pneumoniae (strain | anchored |
| | | | | | CGSP14) GN = ecfA PE = 3 SV = 1 - | (with CS) |
| | | | | | [B2INN7_STRPS] | |
| B2IS50 | 52.46 | 7 | 122 | 47.46 | 50S ribosomal protein L14 OS = | Intracellular |
| | | | | | Streptococcus pneumoniae (strain | |
| | | | | | CGSP14) GN = rplN PE = 3 SV = 1 - | |
| | | | | | [RL14_STRPS] | |
| B2IRI6 | 32.63 | 3 | 95 | 47.46 | Bacteriocin, putative OS = | N- |
| | | | | | Streptococcus pneumoniae (strain | terminally |
| | | | | | CGSP14) GN = SPCG_0105 PE = 4 SV = 1 - | anchored |
| | | | | | [B2IRI6_STRPS] | (with CS) |
| B2IPJ1 | 50 | 9 | 264 | 47.22 | Iron-compound ABC transporter, | Intracellular |
| | | | | | ATP-binding protein OS = | |
| | | | | | Streptococcus pneumoniae (strain | |
| | | | | | CGSP14) GN = SPCG_1013 PE = 4 SV = 1 - | |
| | | | | | [B2IPJ1_STRPS] | |
| B2IPR3 | 40.89 | 7 | 247 | 47.21 | Uncharacterized protein OS = | N- |
| | | | | | Streptococcus pneumoniae (strain | terminally |
| | | | | | CGSP14) GN = SPCG_1083 PE = 4 SV = 1 - | anchored |
| | | | | | [B2IPR3_STRPS] | (with CS) |
| B2IR24 | 28.28 | 9 | 389 | 47.15 | Uncharacterized protein OS = | Intracellular |
| | | | | | Streptococcus pneumoniae (strain | |
| | | | | | CGSP14) GN = SPCG_0036 PE = 3 SV = 1 - | |
| | | | | | [B2IR24_STRPS] | |
| B2ISX7 | 60.12 | 8 | 173 | 47.03 | Phosphoesterase OS = -Streptococcus | Intracellular |
| | | | | | pneumoniae (strain CGSP14) | |
| | | | | | GN = SPCG_1852 PE = 3 SV = 1 - | |
| | | | | | [B2ISX7_STRPS] | |
| B2IM86 | 29.62 | 8 | 449 | 46.75 | Potassium uptake protein, Trk | N- |
| | | | | | family OS = -Streptococcus pneumoniae | terminally |
| | | | | | (strain CGSP14) GN = trkA PE = 4 SV = 1 - | anchored |
| | | | | | [B2IM86_STRPS] | (with CS) |
| B2IPT8 | 33.13 | 9 | 326 | 46.7 | Tagatose 1,6-diphosphate aldolase | Intracellular |
| | | | | | OS = -Streptococcus pneumoniae (strain | |
| | | | | | CGSP14) GN = lacD PE = 3 SV = 1 - | |
| | | | | | [B2IPT8_STRPS] | |
| B2ILZ5 | 39.81 | 10 | 540 | 46.59 | 60 kDa chaperonin OS = | Intracellular |
| | | | | | Streptococcus pneumoniae (strain | |
| | | | | | CGSP14) GN = groL PE = 3 SV = 1 - | |
| | | | | | [CH60_STRPS] | |
| B2ILX7 | 34.58 | 8 | 480 | 46.43 | Aspartyl/glutamyl-tRNA(Asn/Gln) | Intracellular |
| | | | | | amidotransferase subunit B OS = | |
| | | | | | Streptococcus pneumoniae (strain | |
| | | | | | CGSP14) GN = gatB PE = 3 SV = 1 - | |
| | | | | | [GATB_STRPS] | |
| B2IMQ9 | 16.96 | 8 | 737 | 45.59 | Polyribonucleotide | Intracellular |
| | | | | | nucleotidyltransferase OS = | |
| | | | | | Streptococcus pneumoniae (strain | |
| | | | | | CGSP14) GN = pnp PE = 3 SV = 2 - | |
| | | | | | [PNP_STRPS] | |
| B2IQN3 | 45.45 | 5 | 66 | 44.91 | 30S ribosomal protein S21 OS = | Intracellular |
| | | | | | Streptococcus pneumoniae (strain | |
| | | | | | CGSP14) GN = rpsU PE = 3 SV = 1 - | |
| | | | | | [B2IQN3_STRPS] | |
| B2IR90 | 38.37 | 2 | 172 | 44.68 | Non-heme iron-containing ferritin | Intracellular |
| | | | | | OS = -Streptococcus pneumoniae (strain | |
| | | | | | CGSP14) GN = SPCG_1557 PE = 3 SV = 1 - | |
| | | | | | [B2IR90_STRPS] | |
| B2IMS2 | 42.66 | 7 | 293 | 44.59 | Fructose-bisphosphate aldolase | Intracellular |
| | | | | | OS = -Streptococcus pneumoniae (strain | |
| | | | | | CGSP14) GN = fba PE = 3 SV = 1 - | |
| | | | | | [B2IMS2_STRPS] | |
| B2IRX2 | 10.53 | 5 | 399 | 44.06 | Uncharacterized protein OS = | Multi- |
| | | | | | Streptococcus pneumoniae (strain | transmembrane |
| | | | | | CGSP14) GN = SPCG_1689 PE = 4 SV = 1 - | |
| | | | | | [B2IRX2_STRPS] | |
| B2ISH7 | 20.98 | 10 | 815 | 43.34 | Formate acetyltransferase, putative | Intracellular |
| | | | | | OS = -Streptococcus pneumoniae (strain | |
| | | | | | CGSP14) GN = pfIF PE = 4 SV = 1 - | |
| | | | | | [B2ISH7_STRPS] | |
| B2ISG1 | 26.84 | 7 | 190 | 43.06 | Uncharacterized protein OS = | Multi- |
| | | | | | Streptococcus pneumoniae (strain | transmembrane |
| | | | | | CGSP14) GN = SPCG_1779 PE = 4 SV = 1 - | |
| | | | | | [B2ISG1_STRPS] | |
| B21MY2 | 73.21 | 7 | 56 | 42.86 | Uncharacterized protein OS = | Secretory |
| | | | | | Streptococcus pneumoniae (strain | (released) |
| | | | | | CGSP14) GN = SPCG_0626 PE = 4 SV = 1 - | (with CS) |
| | | | | | [B2IMY2_STRPS] | |
| B2IM36 | 15.5 | 8 | 671 | 42.81 | Uncharacterized protein OS = | Multi- |
| | | | | | Streptococcus pneumoniae (strain | transmembrane |
| | | | | | CGSP14) GN = SPCG_1921 PE = 4 SV = 1 | |
| | | | | | [B2IM36_STRPS] | |
| B2IM12 | 26.96 | 8 | 471 | 42.77 | Pneumolysin OS = -Streptococcus | Intracellular |
| | | | | | pneumoniae (strain CGSP14) GN = ply | |
| | | | | | PE = 4 SV = 1 - [B2IM12_STRPS] | |
| B2INM4 | 34.62 | 6 | 182 | 42.71 | Ribosomal subunit interface protein | Intracellular |
| | | | | | OS = -Streptococcus pneumoniae (strain | |
| | | | | | CGSP14) GN = SPCG_2173 PE = 4 SV = 1 | |
| | | | | | [B2INM4_STRPS] | |
| B2IPW4 | 27.34 | 9 | 567 | 42.61 | Dihydrolipoyl dehydrogenase OS = | Intracellular |
| | | | | | Streptococcus pneumoniae (strain | |
| | | | | | CGSP14) GN = acoL PE = 4 SV = 1 - | |
| | | | | | [B2IPW4_STRPS] | |
| B2IQZ9 | 38.89 | 6 | 180 | 42.56 | Hypoxanthine-guanine | Intracellular |
| | | | | | phosphoribosyltransferase OS = | |
| | | | | | Streptococcus pneumoniae (strain | |
| | | | | | CGSP14) GN = hgt PE = 4 SV = 1 - | |
| | | | | | [B2IQZ9_STRPS] | |
| B2IS21 | 19.23 | 6 | 234 | 42.44 | Uncharacterized protein OS = | Multi- |
| | | | | | Streptococcus pneumoniae (strain | transmembrane |
| | | | | | CGSP14) GN = SPCG_0199 PE = 4 SV = 1 - | |
| | | | | | [B2IS21_STRPS] | |
| B2IPB4 | 18.18 | 3 | 66 | 42.39 | 50S ribosomal protein L35 OS = | Intracellular |
| | | | | | Streptococcus pneumoniae (strain | |
| | | | | | CGSP14) GN = rpml PE = 3 SV = 1 - | |
| | | | | | [RL35_STRPS] | |
| B2IM29 | 23.68 | 5 | 418 | 42.24 | Putative competence-damage | Intracellular |
| | | | | | inducible protein OS = -Streptococcus | |
| | | | | | pneumoniae (strain CGSP14) | |
| | | | | | GN = cinA PE = 3 SV = 1 - [CINA_STRPS] | |
| B2IR86 | 66.5 | 11 | 197 | 41.89 | Probable GTP-binding protein EngB | Intracellular |
| | | | | | OS = -Streptococcus pneumoniae (strain | |
| | | | | | CGSP14) GN = engB PE = 3 SV = 1 - | |
| | | | | | [ENGB_STRPS] | |
| B2IPS2 | 46.7 | 4 | 212 | 41.29 | Uridine kinase OS = -Streptococcus | Intracellular |
| | | | | | pneumoniae (strain CGSP14) | |
| | | | | | GN = udk PE = 3 SV = 1 - [URK_STRPS] | |
| B2INP0 | 22.46 | 5 | 276 | 41.12 | Uncharacterized protein OS = | Intracellular/ |
| | | | | | Streptococcus pneumoniae (strain | TMH start |
| | | | | | CGSP14) GN = SPCG_2189 PE = 4 SV = 1 - | AFTER 60 |
| | | | | | [B2INP0_STRPS] | |
| B2ILX1 | 42.71 | 7 | 288 | 41.03 | Acetyl-coenzyme A carboxylase | Intracellular |
| | | | | | carboxyl transferase subunit beta | |
| | | | | | OS = -Streptococcus pneumoniae (strain | |
| | | | | | CGSP14) GN = accD PE = 3 SV = 1 - | |
| | | | | | [B2ILX1_STRPS] | |
| B2IMY9 | 31.48 | 10 | 486 | 40.7 | Glutamate--tRNA ligase OS = | Intracellular |
| | | | | | Streptococcus pneumoniae (strain | |
| | | | | | CGSP14) GN = gltX PE = 3 SV = 1 - | |
| | | | | | [SYE_STRPS] | |
| B2ISL4 | 51.11 | 9 | 270 | 40.64 | Cof family protein OS = -Streptococcus | Intracellular |
| | | | | | pneumoniae (strain CGSP14) | |
| | | | | | GN = SPCG_0299 PE = 4 SV = 1 - | |
| | | | | | [B2ISL4_STRPS] | |
| B2IQH7 | 42.21 | 5 | 289 | 40.55 | Homoserine kinase OS = | Intracellular |
| | | | | | Streptococcus pneumoniae (strain | |
| | | | | | CGSP14) GN = thrB PE = 3 SV = 1 - | |
| | | | | | [KHSE_STRPS] | |
| B2IS89 | 36.54 | 7 | 208 | 40.54 | Guanylate kinase OS = -Streptococcus | Intracellular |
| | | | | | pneumoniae (strain CGSP14) | |
| | | | | | GN = gmk PE = 3 SV = 1 - | |
| | | | | | [B2IS89_STRPS] | |
| B2IRQ3 | 18.92 | 7 | 650 | 40.16 | Endopeptidase O OS = -Streptococcus | Intracellular |
| | | | | | pneumoniae (strain CGSP14) | |
| | | | | | GN = pepO PE = 4 SV = 1 - | |
| | | | | | [B2IRQ3_STRPS] | |
| B2IR80 | 26.03 | 6 | 292 | 39.76 | Uncharacterized protein OS = | Multi- |
| | | | | | Streptococcus pneumoniae (strain | transmembrane |
| | | | | | CGSP14) GN = SPCG_1547 PE = 4 SV = 1 - | |
| | | | | | [B2IR80_STRPS] | |
| B2INZ2 | 20.82 | 6 | 413 | 39.4 | D-alanyl-D-alanine | N- |
| | | | | | carboxypeptidase OS = -Streptococcus | terminally |
| | | | | | pneumoniae (strain CGSP14) | anchored |
| | | | | | GN = dacA PE = 3 SV = 1 - | (with CS) |
| | | | | | [B2INZ2_STRPS] | |
| B2IR93 | 45.95 | 6 | 259 | 39.26 | Triosephosphate isomerase OS = | Intracellular |
| | | | | | Streptococcus pneumoniae (strain | |
| | | | | | CGSP14) GN = tpiA PE = 3 SV = 1 - | |
| | | | | | [B2IR93_STRPS] | |
| B2IP56 | 34.48 | 8 | 290 | 39.23 | Uncharacterized protein OS = | Lipid |
| | | | | | Streptococcus pneumoniae (strain | anchored |
| | | | | | CGSP14) GN = SPCG_0875 PE = 4 SV = 1 - | |
| | | | | | [B2IP56_STRPS] | |
| B2IPZ7 | 46.59 | 6 | 279 | 39.17 | DegV family protein OS = | Intracellular |
| | | | | | Streptococcus pneumoniae (strain | |
| | | | | | CGSP14) GN = SPCG_1169 PE = 4 SV = 1 - | |
| | | | | | [B2IPZ7_STRPS] | |
| B2INK5 | 19.52 | 8 | 502 | 39.03 | Glycerol kinase OS = -Streptococcus | Intracellular |
| | | | | | pneumoniae (strain CGSP14) | |
| | | | | | GN = glpK PE = 3 SV = 1 - [GLPK_STRPS] | |
| B2INM2 | 32.67 | 7 | 150 | 38.84 | 50S ribosomal protein L9 OS = | Intracellular |
| | | | | | Streptococcus pneumoniae (strain | |
| | | | | | CGSP14) GN = rplI PE = 3 SV = 1 - | |
| | | | | | [RL9_STRPS] | |
| B2IPH6 | 21.17 | 4 | 359 | 38.78 | Peptide chain release factor 1 OS = | Intracellular |
| | | | | | Streptococcus pneumoniae (strain | |
| | | | | | CGSP14) GN = prfA PE = 3 SV = 1 - | |
| | | | | | [RF1_STRPS] | |
| B2ING0 | 44.21 | 3 | 95 | 38.52 | 30S ribosomal protein S16 OS = | Intracellular |
| | | | | | Streptococcus pneumoniae (strain | |
| | | | | | CGSP14) GN = rpsP PE = 3 SV = 1 - | |
| | | | | | [RS16_STRPS] | |
| B2IQZ0 | 33.07 | 7 | 378 | 38.34 | DNA polymerase III subunit beta | Intracellular |
| | | | | | OS = -Streptococcus pneumoniae (strain | |
| | | | | | CGSP14) GN = dnaN PE = 4 SV = 1 - | |
| | | | | | [B2IQZO_STRPS] | |
| B2IR11 | 29.29 | 8 | 420 | 37.79 | DNA repair protein radA OS = | Intracellular |
| | | | | | Streptococcus pneumoniae (strain | |
| | | | | | CGSP14) GN = radA PE = 3 SV = 1 - | |
| | | | | | [B2IR11_STRPS] | |
| B2ISK0 | 7.86 | 8 | 1463 | 37.3 | DNA polymerase III PolC-type OS = | Intracellular |
| | | | | | Streptococcus pneumoniae (strain | |
| | | | | | CGSP14) GN = polC PE = 3 SV = 1 - | |
| | | | | | [B2ISKO_STRPS] | |
| B2IMD8 | 40.15 | 8 | 264 | 37.04 | Uncharacterized protein OS = | Intracellular |
| | | | | | Streptococcus pneumoniae (strain | |
| | | | | | CGSP14) GN = SPCG_0513 PE = 4 SV = 1 - | |
| | | | | | [B2IMD8_STRPS] | |
| B2IP06 | 33.66 | 7 | 303 | 36.91 | Tagatose-6-phosphate kinase OS | Intracellular |
| | | | | | Streptococcus pneumoniae (strain | |
| | | | | | CGSP14) GN = fruB PE = 3 SV = 1 - | |
| | | | | | [B2IP06_STRPS] | |
| B2IPT6 | 46.2 | 5 | 171 | 36.86 | Galactose-6-phosphate isomerase | Intracellular |
| | | | | | subunit LacB OS = -Streptococcus | |
| | | | | | pneumoniae (strain CGSP14) | |
| | | | | | GN = lacB PE = 3 SV = 1 - [LACB_STRPS] | |
| B2IMT1 | 28.33 | 7 | 406 | 36.74 | Beta-lactam resistance factor OS = | Intracellular |
| | | | | | Streptococcus pneumoniae (strain | |
| | | | | | CGSP14) GN = murM PE = 4 SV = 1 - | |
| | | | | | [B2IMT1_STRPS] | |
| B2IN52 | 24.87 | 8 | 567 | 36.72 | Dihydroxy-acid dehydratase OS = | Intracellular |
| | | | | | Streptococcus pneumoniae (strain | |
| | | | | | CGSP14) GN = ilvD PE = 3 SV = 1 - | |
| | | | | | [ILVD_STRPS] | |
| B2IRH4 | 23.13 | 5 | 320 | 36.59 | Uncharacterized protein OS = | N- |
| | | | | | Streptococcus pneumoniae (strain | terminally |
| | | | | | CGSP14) GN = SPCG_0093 PE = 4 SV = 1 - | anchored |
| | | | | | [B2IRH4_STRPS] | (No CS) |
| B2IME1 | 27.51 | 7 | 378 | 36.3 | Transcription | Intracellular |
| | | | | | termination/antitermination | |
| | | | | | protein NusA OS = -Streptococcus | |
| | | | | | pneumoniae (strain CGSP14) | |
| | | | | | GN = nusA PE = 3 SV = 1 - | |
| | | | | | [B2IME1 STRPS] | |
| B2ISX2 | 51.67 | 9 | 240 | 36.23 | Pseudouridine synthase OS = | Intracellular |
| | | | | | Streptococcus pneumoniae (strain | |
| | | | | | CGSP14) GN = rluB PE = 3 SV = 1 - | |
| | | | | | [B2ISX2_STRPS] | |
| B2IN61 | 77.55 | 6 | 49 | 35.89 | 50S ribosomal protein L33 OS = | Intracellular |
| | | | | | Streptococcus pneumoniae (strain | |
| | | | | | CGSP14) GN = rpmG PE = 3 SV = 1 - | |
| | | | | | [RL33_STRPS] | |
| B2IQ98 | 60.64 | 4 | 94 | 35.66 | 50S ribosomal protein L31 type B | Intracellular |
| | | | | | OS = -Streptococcus pneumoniae (strain | |
| | | | | | CGSP14) GN = rpmE PE = 3 SV = 1 - | |
| | | | | | [B2IQ98_STRPS] | |
| B2IPY5 | 32.19 | 7 | 379 | 35.53 | Glycogen biosynthesis protein GlgD | Intracellular |
| | | | | | OS = -Streptococcus pneumoniae (strain | |
| | | | | | CGSP14) GN = glgD PE = 4 SV = 1 - | |
| | | | | | [B2IPY5_STRPS] | |
| B2IRA3 | 21.23 | 4 | 438 | 35.35 | Pyridine nucleotide-disulfide | Intracellular |
| | | | | | oxidoreductase OS = -Streptococcus | |
| | | | | | pneumoniae (strain CGSP14) | |
| | | | | | GN = merA PE = 3 SV = 1 - | |
| | | | | | [B2IRA3_STRPS] | |
| B2IR65 | 48.8 | 6 | 209 | 35.25 | Uncharacterized protein OS = | Intracellular |
| | | | | | Streptococcus pneumoniae (strain | |
| | | | | | CGSP14) GN = SPCG_1532 PE = 4 SV = 1 | |
| | | | | | - [B2IR65_STRPS] | |
| B2INN1 | 36.99 | 7 | 346 | 34.94 | Elongation factor Ts OS = | Intracellular |
| | | | | | Streptococcus pneumoniae (strain | |
| | | | | | CGSP14) GN = tsf PE = 3 SV = 1 - | |
| | | | | | [EFTS_STRPS] | |
| B2IRV5 | 19.83 | 3 | 343 | 34.85 | Phospho-2-dehydro-3- | Intracellular |
| | | | | | deoxyheptonate aldolase OS = | |
| | | | | | Streptococcus pneumoniae (strain | |
| | | | | | CGSP14) GN = aroF PE = 3 SV = 1 - | |
| | | | | | [B2IRV5_STRPS] | |
| B2IN60 | 53.33 | 4 | 60 | 34.75 | 50S ribosomal protein L32 OS = | Intracellular |
| | | | | | Streptococcus pneumoniae (strain | |
| | | | | | CGSP14) GN = rpmF PE = 3 SV = 1 - | |
| | | | | | [RL32_STRPS] | |
| B2IQ13 | 32.08 | 6 | 371 | 34.73 | Aminotransferase, class-V OS = | Intracellular |
| | | | | | Streptococcus pneumoniae (strain | |
| | | | | | CGSP14) GN = SPCG_1185 PE = 3 SV = 1 - | |
| | | | | | [B2IQ13_STRPS] | |
| B2INQ1 | 36.11 | 5 | 180 | 34.11 | Transcriptional regulator, TetR | Intracellular |
| | | | | | family OS = -Streptococcus pneumoniae | |
| | | | | | (strain CGSP14) GN = SPCG_2200 | |
| | | | | | PE = 4 SV = 1 - [B2INQ1_STRPS] | |
| B2IP08 | 15.91 | 7 | 767 | 33.57 | SpoE family protein OS = | Multi- |
| | | | | | Streptococcus pneumoniae (strain | transmembrane |
| | | | | | CGSP14) GN = ftsK PE = 3 SV = 1 - | |
| | | | | | [B2IP08_STRPS] | |
| B2IRW8 | 41.24 | 6 | 177 | 33.56 | Transcriptional repressor NrdR OS = | Intracellular |
| | | | | | Streptococcus pneumoniae (strain | |
| | | | | | CGSP14) GN = nrdR PE = 3 SV = 1 - | |
| | | | | | [B2IRW8_STRPS] | |
| B2ISL9 | 57.61 | 6 | 184 | 33.49 | GTP cyclohydrolase 1 OS = | Intracellular |
| | | | | | Streptococcus pneumoniae (strain | |
| | | | | | CGSP14) GN = foIE PE = 3 SV = 1 - | |
| | | | | | [GCH1_STRPS] | |
| B2IQL2 | 25.35 | 4 | 217 | 32.6 | Phosphate-specific transport | Intracellular |
| | | | | | system accessory protein PhoU OS = | |
| | | | | | Streptococcus pneumoniae (strain | |
| | | | | | CGSP14) GN = SPCG_1384 PE = 3 SV = 1 - | |
| | | | | | [B2IQL2_STRPS] | |
| B2ILW1 | 39.58 | 4 | 144 | 32.47 | Transcriptional regulator, MarR | Intracellular |
| | | | | | family OS = -Streptococcus pneumoniae | |
| | | | | | (strain CGSP14) GN = SPCG_0414 | |
| | | | | | PE = 4 SV = 1 - [B2ILW1_STRPS] | |
| B2IS33 | 15.25 | 5 | 518 | 32.44 | Competence-induced protein Ccs4 | Multi- |
| | | | | | OS = -Streptococcus pneumoniae (strain | transmembrane |
| | | | | | CGSP14) GN = SPCG_0211 PE = 4 SV = 1 - | |
| | | | | | [B2IS33_STRPS] | |
| B2INX7 | 16.53 | 4 | 236 | 32.43 | Uncharacterized protein OS = | Multi- |
| | | | | | Streptococcus pneumoniae (strain | transmembrane |
| | | | | | CGSP14) GN = SPCG_0802 PE = 4 SV = 1 - | |
| | | | | | [B2INX7_STRPS] | |
| B2INM3 | 14.16 | 7 | 657 | 32.29 | DHH subfamily 1 protein OS = | Multi- |
| | | | | | Streptococcus pneumoniae (strain | transmembrane |
| | | | | | CGSP14) GN = SPCG_2172 PE = 4 SV = 1 - | |
| | | | | | [B2INM3_STRPS] | |
| B2INY8 | 22.62 | 6 | 420 | 32.18 | Uncharacterized protein OS = | Intracellular |
| | | | | | Streptococcus pneumoniae (strain | |
| | | | | | CGSP14) GN = SPCG_0813 PE = 4 SV = 1 | |
| | | | | | - [B2INY8_STRPS] | |
| B2IN02 | 20.77 | 5 | 443 | 32.03 | Sensor histidine kinase PnpS OS = | Multi- |
| | | | | | Streptococcus pneumoniae (strain | transmembrane |
| | | | | | CGSP14) GN = SPCG_2049 PE = 4 SV = 1 - | |
| | | | | | [B2IN02_STRPS] | |
| B2IMR8 | 13.73 | 5 | 459 | 31.87 | Transmembrane protein Vexp3 OS = | Multi- |
| | | | | | Streptococcus pneumoniae (strain | transmembrane |
| | | | | | CGSP14) GN = SPCG_0562 PE = 3 SV = 1 - | |
| | | | | | [B2IMR8_STRPS] | |
| B2IP11 | 34.65 | 7 | 404 | 31.83 | Probable tRNA sulfurtransferase | Intracellular |
| | | | | | OS = -Streptococcus pneumoniae (strain | |
| | | | | | CGSP14) GN = thiI PE = 3 SV = 1 - | |
| | | | | | [THII_STRPS] | |
| B2INZ1 | 26.38 | 8 | 470 | 31.82 | FeS assembly protein SufB OS = | Intracellular |
| | | | | | Streptococcus pneumoniae (strain | |
| | | | | | CGSP14) GN = SPCG_0816 PE = 4 SV = 1 - | |
| | | | | | [B2INZ1_STRPS] | |
| B2IQ37 | 41.27 | 5 | 252 | 31.78 | ABC transporter, ATP-binding | Intracellular |
| | | | | | protein OS = -Streptococcus | |
| | | | | | pneumoniae (strain CGSP14) | |
| | | | | | GN = SPCG_1209 PE = 4 SV = 1 - | |
| | | | | | [B2IQ37_STRPS] | |
| B2IN78 | 31.83 | 6 | 399 | 31.49 | Cell division protein DivIB OS = | Intracellular/ |
| | | | | | Streptococcus pneumoniae (strain | TMH start |
| | | | | | CGSP14) GN = divIB PE = 3 SV = 1 - | AFTER 60 |
| | | | | | [B2IN78_STRPS] | |
| B2IS71 | 21.69 | 5 | 249 | 31.41 | Phosphoglycerate mutase family | Intracellular |
| | | | | | protein OS = -Streptococcus | |
| | | | | | pneumoniae (strain CGSP14) | |
| | | | | | GN = gpmB PE = 4 SV = 1 - | |
| | | | | | [B2IS71_STRPS] | |
| B2IQJ2 | 23.71 | 5 | 388 | 31.39 | Chorismate synthase OS = = | Intracellular |
| | | | | | Streptococcus pneumoniae (strain | |
| | | | | | CGSP14) GN = aroC PE = 3 SV = 1 - | |
| | | | | | [AROC_STRPS] | |
| B2IST9 | 13.89 | 7 | 583 | 31.29 | ABC transporter, ATP- | Multi- |
| | | | | | binding/permease protein OS = | transmembrane |
| | | | | | Streptococcus pneumoniae (strain | |
| | | | | | CGSP14) GN = SPCG_1814 PE = 4 SV = 1 - | |
| | | | | | [B2IST9_STRPS] | |
| B2IPQ2 | 32.05 | 6 | 234 | 30.92 | Uncharacterized protein OS = | Intracellular |
| | | | | | Streptococcus pneumoniae (strain | |
| | | | | | CGSP14) GN = SPCG_1072 PE = 4 SV = 1 - | |
| | | | | | [B2IPQ2_STRPS] | |
| B2ISM0 | 18.89 | 3 | 270 | 30.69 | 7,8-dihydroneopterin aldolase OS = | Intracellular |
| | | | | | Streptococcus pneumoniae (strain | |
| | | | | | CGSP14) GN = suID PE = 3 SV = 1 - | |
| | | | | | [B2ISMO_STRPS] | |
| B2IMH1 | 16.74 | 6 | 472 | 30.67 | Cof family protein OS = -Streptococcus | Intracellular |
| | | | | | pneumoniae (strain CGSP14) | |
| | | | | | GN = SPCG_1962 PE = 4 SV = 1 - | |
| | | | | | [B2IMH1_STRPS] | |
| B2ISV2 | 17.09 | 5 | 392 | 30.34 | Galactokinase OS = -Streptococcus | Intracellular |
| | | | | | pneumoniae (strain CGSP14) | |
| | | | | | GN = gaIK PE = 3 SV = 1 - | |
| | | | | | [B2ISV2_STRPS] | |
| B2IR20 | 12.6 | 8 | 889 | 30.19 | DNA polymerase OS = -Streptococcus | Intracellular |
| | | | | | pneumoniae (strain CGSP14) | |
| | | | | | GN = poIA PE = 3 SV = 1 - | |
| | | | | | [B2IR20_STRPS] | |
| B2IR50 | 26.4 | 6 | 481 | 30 | UDP-N-acetylmuramoyl-L-alanyl-D- | Intracellular |
| | | | | | glutamate--L-lysine ligase OS = | |
| | | | | | Streptococcus pneumoniae (strain | |
| | | | | | CGSP14) GN = murE PE = 3 SV = 1 - | |
| | | | | | [B2IR50_STRPS] | |
| B2ISI6 | 20.93 | 6 | 258 | 29.94 | Isoprenyl transferase OS = | Intracellular |
| | | | | | Streptococcus pneumoniae (strain | |
| | | | | | CGSP14) GN = uppS PE = 3 SV = 1 - | |
| | | | | | [B2IS16_STRPS] | |
| B2IMQ1 | 31.47 | 7 | 375 | 29.85 | Phenylalanine--tRNA ligase alpha | Intracellular |
| | | | | | subunit OS = -Streptococcus | |
| | | | | | pneumoniae (strain CGSP14) | |
| | | | | | GN = pheS PE = 3 SV = 1 - | |
| | | | | | [B2IMQ1_STRPS] | |
| B2ISS1 | 20.39 | 6 | 407 | 29.77 | Tryptophan synthase beta chain | Intracellular |
| | | | | | OS = -Streptococcus pneumoniae (strain | |
| | | | | | CGSP14) GN = trpB PE = 3 SV = 1 - | |
| | | | | | [TRPB_STRPS] | |
| B2IMZ0 | 22.05 | 5 | 449 | 29.65 | Glucose-6-phosphate isomerase | Intracellular |
| | | | | | OS = -Streptococcus pneumoniae (strain | |
| | | | | | CGSP14) GN = pgi PE = 3 SV = 1 - | |
| | | | | | [G6PI_STRPS] | |
| B2IRD1 | 13.75 | 4 | 778 | 29.59 | Cation-transporting ATPase, E1-E2 | Multi- |
| | | | | | family OS = -Streptococcus pneumoniae | transmembrane |
| | | | | | (strain CGSP14) GN = ctpE PE = 3 SV = 1 - | |
| | | | | | [B2IRD1_STRPS] | |
| B2IQ17 | 51.09 | 5 | 229 | 29.49 | Glutamine amidotransferase, class I | Intracellular |
| | | | | | OS = -Streptococcus pneumoniae (strain | |
| | | | | | CGSP14) GN = SPCG_1189 PE = 4 SV = 1 - | |
| | | | | | [B2IQ17_STRPS] | |
| B2IPV5 | 30.5 | 5 | 200 | 29.44 | Uncharacterized protein OS = | Intracellular |
| | | | | | Streptococcus pneumoniae (strain | |
| | | | | | CGSP14) GN = SPCG_1127 PE = 4 SV = 1 - | |
| | | | | | [B2IPV5_STRPS] | |
| B2ILW5 | 27.78 | 6 | 306 | 29.34 | Malonyl CoA-acyl carrier protein | Intracellular |
| | | | | | transacylase OS = -Streptococcus | |
| | | | | | pneumoniae (strain CGSP14) | |
| | | | | | GN = fabD PE = 3 SV = 1 - | |
| | | | | | [B2ILW5_STRPS] | |
| B2IQ16 | 29.48 | 6 | 424 | 29.26 | Psr protein OS = -Streptococcus | Intracellular/ |
| | | | | | pneumoniae (strain CGSP14) | TMH start |
| | | | | | GN = SPCG_1358 PE = 4 SV = 1 - | AFTER 60 |
| | | | | | [B2IQ16_STRPS] | |
| B2IPB3 | 40.51 | 5 | 195 | 28.99 | Translation initiation factor IF-3 OS = | Intracellular |
| | | | | | Streptococcus pneumoniae (strain | |
| | | | | | CGSP14) GN = infC PE = 3 SV = 1 - | |
| | | | | | [B2IPB3_STRPS] | |
| B2IM63 | 18.52 | 7 | 432 | 28.92 | Competence-induced protein Ccs50 | N- |
| | | | | | OS = -Streptococcus pneumoniae (strain | terminally |
| | | | | | CGSP14) GN = SPCG_1944 PE = 4 SV = 1 - | anchored |
| | | | | | [B2IM63_STRPS] | (No CS) |
| B2IP99 | 44.53 | 6 | 247 | 28.66 | Uridylate kinase OS = -Streptococcus | Intracellular |
| | | | | | pneumoniae (strain CGSP14) | |
| | | | | | GN = pyrH PE = 3 SV = 1 - | |
| | | | | | [B2IP99_STRPS] | |
| B2IPF5 | 10.79 | 5 | 834 | 28.65 | Uncharacterized protein OS = | N- |
| | | | | | Streptococcus pneumoniae (strain | terminally |
| | | | | | CGSP14) GN = SPCG_0977 PE = 4 SV = 1 - | anchored |
| | | | | | [B2IPF5_STRPS] | (No CS) |
| B2IM26 | 26.1 | 6 | 318 | 28.61 | Autolysin OS = -Streptococcus | Intracellular |
| | | | | | pneumoniae (strain CGSP14) | |
| | | | | | GN = lytA PE = 4 SV = 1 - | |
| | | | | | [B2IM26_STRPS] | |
| B2INF6 | 43.1 | 5 | 239 | 28.52 | tRNA (guanine-N(1)-)- | Intracellular |
| | | | | | methyltransferase OS = -Streptococcus | |
| | | | | | pneumoniae (strain CGSP14) | |
| | | | | | GN = trmD PE = 3 SV = 1 - | |
| | | | | | [TRMD_STRPS] | |
| B2ISI0 | 15.25 | 6 | 833 | 28.41 | Leucine--tRNA ligase OS = | Intracellular |
| | | | | | Streptococcus pneumoniae (strain | |
| | | | | | CGSP14) GN = leuS PE = 3 SV = 1 - | |
| | | | | | [SYL_STRPS] | |
| B2IQH8 | 20.5 | 6 | 439 | 28.08 | Homoserine dehydrogenase OS = | Intracellular |
| | | | | | Streptococcus pneumoniae (strain | |
| | | | | | CGSP14) GN = hom PE = 3 SV = 1 - | |
| | | | | | [B2IQH8_STRPS] | |
| B2INT8 | 57.89 | 3 | 76 | 27.91 | Uncharacterized protein OS = | Intracellular |
| | | | | | Streptococcus pneumoniae (strain | |
| | | | | | CGSP14) GN = SPCG_0763 PE = 4 SV = 1 - | |
| | | | | | [B2INT8_STRPS] | |
| B2IMD9 | 15.64 | 2 | 211 | 27.64 | tRNA (guanine-N(7)-)- | Intracellular |
| | | | | | methyltransferase OS = -Streptococcus | |
| | | | | | pneumoniae (strain CGSP14) | |
| | | | | | GN = trmB PE = 3 SV = 1 - | |
| | | | | | [TRMB_STRPS] | |
| B2IQS2 | 26.88 | 4 | 253 | 27.63 | SpoU rRNA methylase family | Intracellular |
| | | | | | protein OS = -Streptococcus | |
| | | | | | pneumoniae (strain CGSP14) | |
| | | | | | GN = trmH PE = 3 SV = 1 - | |
| | | | | | [B2IQS2_STRPS] | |
| B2IR73 | 16.69 | 5 | 623 | 27.4 | ABC transporter, ATP-binding | Intracellular |
| | | | | | protein OS = -Streptococcus | |
| | | | | | pneumoniae (strain CGSP14) | |
| | | | | | GN = SPCG_1540 PE = 4 SV = 1 - | |
| | | | | | [B2IR73_STRPS] | |
| B2IPP4 | 10.12 | 6 | 662 | 27.29 | UvrABC system protein B OS = | Intracellular |
| | | | | | Streptococcus pneumoniae (strain | |
| | | | | | CGSP14) GN = uvrB PE = 3 SV = 1 - | |
| | | | | | [UVRB_STRPS] | |
| B2IQK6 | 26.91 | 4 | 301 | 27.27 | UDP-N- | Intracellular |
| | | | | | acetylenolpyruvoylglucosamine | |
| | | | | | reductase OS = -Streptococcus | |
| | | | | | pneumoniae (strain CGSP14) | |
| | | | | | GN = murB PE = 3 SV = 1 - | |
| | | | | | [MURB_STRPS] | |
| B2IP98 | 25.45 | 6 | 444 | 27.26 | Methylenetetrahydrofolate--tRNA- | Intracellular |
| | | | | | (uracil-5-)-methyltransferase | |
| | | | | | TrmFO OS = -Streptococcus | |
| | | | | | pneumoniae (strain CGSP14) | |
| | | | | | GN = trmFO PE = 3 SV = 2 - | |
| | | | | | [TRMFO _STRPS] | |
| B2IQK5 | 19.48 | 5 | 385 | 27.06 | Spermidine/putrescine import ATP- | Intracellular |
| | | | | | binding protein PotA OS = | |
| | | | | | Streptococcus pneumoniae (strain | |
| | | | | | CGSP14) GN = potA PE = 3 SV = 1 - | |
| | | | | | [B2IQK5_STRPS] | |
| B2INN3 | 30.61 | 7 | 392 | 26.86 | Secreted 45 kd protein OS = | Secretory |
| | | | | | Streptococcus pneumoniae (strain | (released) |
| | | | | | CGSP14) GN = SPCG_2182 PE = 4 SV = 1 - | (with CS) |
| | | | | | [B2INN3_STRPS] | |
| B2IN20 | 40.78 | 4 | 103 | 26.71 | Uncharacterized protein OS = | Intracellular |
| | | | | | Streptococcus pneumoniae (strain | |
| | | | | | CGSP14) GN = SPCG_2067 PE = 4 SV = 1 - | |
| | | | | | [B2IN20_STRPS] | |
| B2IPN6 | 8.14 | 6 | 1179 | 26.7 | Chromosome partition protein Smc | Intracellular |
| | | | | | OS = -Streptococcus pneumoniae (strain | |
| | | | | | CGSP14) GN = smc PE = 3 SV = 1 - | |
| | | | | | [B2IPN6_STRPS] | |
| B2IRW9 | 52.07 | 5 | 121 | 26.67 | Transcriptional regulator, GntR | Intracellular |
| | | | | | family OS = -Streptococcus pneumoniae | |
| | | | | | (strain CGSP14) GN = SPCG_1686 | |
| | | | | | PE = 4 SV = 1 - [B2IRW9_STRPS] | |
| B2IS84 | 13.47 | 2 | 438 | 26.55 | Ribosomal RNA small subunit | Intracellular |
| | | | | | methyltransferase B OS = | |
| | | | | | Streptococcus pneumoniae (strain | |
| | | | | | CGSP14) GN = sunL PE = 3 SV = 1 - | |
| | | | | | [B2IS84_STRPS] | |
| B2IMX9 | 19.88 | 7 | 332 | 26.49 | Pneumococcal surface protein, | Lipid |
| | | | | | putative OS = -Streptococcus | anchored |
| | | | | | pneumoniae (strain CGSP14) | |
| | | | | | GN = SPCG_0623 PE = 4 SV = 1 - | |
| | | | | | [B2IMX9_STRPS] | |
| B2IP15 | 8.69 | 4 | 541 | 26.36 | RNA methyltransferase, TrmA | Intracellular |
| | | | | | family OS = -Streptococcus pneumoniae | |
| | | | | | (strain CGSP14) GN = SPCG_1007 | |
| | | | | | PE = 3 SV = 1 - [B2IPI5_STRPS] | |
| B2IPS8 | 15.14 | 7 | 555 | 25.77 | DNA repair protein RecN OS = | Intracellular |
| | | | | | Streptococcus pneumoniae (strain | |
| | | | | | CGSP14) GN = recN PE = 3 SV = 1 - | |
| | | | | | [B2IPS8_STRPS] | |
| B2INY5 | 17.06 | 6 | 551 | 25.65 | DNA polymerase III subunits | Intracellular |
| | | | | | gamma and tau OS = -Streptococcus | |
| | | | | | pneumoniae (strain CGSP14) | |
| | | | | | GN = dnaX PE = 4 SV = 1 - | |
| | | | | | [B2INY5_STRPS] | |
| B2ISU0 | 10.1 | 4 | 594 | 25.61 | ABC transporter, ATP- | Multi- |
| | | | | | binding/permease protein OS = | transmembrane |
| | | | | | Streptococcus pneumoniae (strain | |
| | | | | | CGSP14) GN = SPCG_1815 PE = 4 SV = 1 - | |
| | | | | | [B2ISUO_STRPS] | |
| B2INU6 | 24.53 | 6 | 424 | 25.32 | Phosphopentomutase OS = | Intracellular |
| | | | | | Streptococcus pneumoniae (strain | |
| | | | | | CGSP14) GN = deoB PE = 3 SV = 1 - | |
| | | | | | [B2INU6_STRPS] | |
| B2IM77 | 17.44 | 2 | 281 | 25.26 | Undecaprenyl-diphosphatase OS = | Intracellular |
| | | | | | Streptococcus pneumoniae (strain | |
| | | | | | CGSP14) GN = uppP PE = 3 SV = 1 - | |
| | | | | | [UPPP_STRPS] | |
| B2IS76 | 21.11 | 5 | 398 | 25.26 | Hydroxymethylglutaryl-CoA | Intracellular |
| | | | | | synthase OS = -Streptococcus | |
| | | | | | pneumoniae (strain CGSP14) | |
| | | | | | GN = mvaS PE = 4 SV = 1 - | |
| | | | | | [B2IS76_STRPS] | |
| B2IPD9 | 20.3 | 4 | 266 | 24.91 | Uncharacterized protein OS = | N- |
| | | | | | Streptococcus pneumoniae (strain | terminally |
| | | | | | CGSP14) GN = SPCG_0961 PE = 4 SV = 1 - | anchored |
| | | | | | [B2IPD9_STRPS] | (No CS) |
| B2IP88 | 14.52 | 6 | 420 | 24.84 | Gamma-glutamyl phosphate | Intracellular |
| | | | | | reductase OS = -Streptococcus | |
| | | | | | pneumoniae (strain CGSP14) | |
| | | | | | GN = proA PE = 3 SV = 1 - | |
| | | | | | [PROA_STRPS] | |
| B2IQU2 | 18.36 | 4 | 305 | 24.77 | Glycine--tRNA ligase alpha subunit | Intracellular |
| | | | | | OS = -Streptococcus pneumoniae (strain | |
| | | | | | CGSP14) GN = glyQ PE = 3 SV = 1 - | |
| | | | | | [SYGA_STRPS] | |
| B2INW0 | 25 | 5 | 220 | 24.67 | Deoxyribose-phosphate aldolase | Intracellular |
| | | | | | OS = -Streptococcus pneumoniae (strain | |
| | | | | | CGSP14) GN = deoC PE = 3 SV = 1 - | |
| | | | | | [DEOC_STRPS] | |
| B2IRB6 | 28.25 | 6 | 223 | 24.63 | Cytidylate kinase OS = -Streptococcus | Intracellular |
| | | | | | pneumoniae (strain CGSP14) | |
| | | | | | GN = cmk PE = 3 SV = 1 - [KCY_STRPS] | |
| B2IP93 | 24.91 | 5 | 289 | 24.4 | Ribosomal RNA small subunit | Intracellular |
| | | | | | methyltransferase I OS = | |
| | | | | | Streptococcus pneumoniae (strain | |
| | | | | | CGSP14) GN = rsml PE = 3 SV = 1 - | |
| | | | | | [B2IP93_STRPS] | |
| B2IPN4 | 29.88 | 5 | 328 | 24.28 | GMP reductase OS = -Streptococcus | Intracellular |
| | | | | | pneumoniae (strain CGSP14) | |
| | | | | | GN = guaC PE = 3 SV = 1 - | |
| | | | | | [GUAC_STRPS] | |
| B2IN17 | 24.9 | 5 | 257 | 24.1 | Fucose operon repressor, putative | Intracellular |
| | | | | | OS = -Streptococcus pneumoniae (strain | |
| | | | | | CGSP14) GN = fcsR PE = 4 SV = 1 - | |
| | | | | | [B2IN17_STRPS] | |
| B21MP1 | 31.07 | 5 | 206 | 24.09 | Hydrolase, haloacid dehalogenase- | Intracellular |
| | | | | | like family OS = -Streptococcus | |
| | | | | | pneumoniae (strain CGSP14) | |
| | | | | | GN = SPCG_2031 PE = 4 SV = 1 - | |
| | | | | | [B2IMP1_STRPS] | |
| B2IRV6 | 17.78 | 3 | 343 | 23.78 | Phospho-2-dehydro-3- | Intracellular |
| | | | | | deoxyheptonate aldolase OS = | |
| | | | | | Streptococcus pneumoniae (strain | |
| | | | | | CGSP14) GN = aroG PE = 3 SV = 1 - | |
| | | | | | [B2IRV6_STRPS] | |
| B2IQ68 | 27.02 | 5 | 359 | 23.7 | Carbamoyl-phosphate synthase | Intracellular |
| | | | | | small chain OS = -Streptococcus | |
| | | | | | pneumoniae (strain CGSP14) | |
| | | | | | GN = carA PE = 3 SV = 1 - | |
| | | | | | [B2IQ68_STRPS] | |
| B2IMY1 | 17.2 | 3 | 279 | 23.45 | Thymidylate synthase OS = | Intracellular |
| | | | | | Streptococcus pneumoniae (strain | |
| | | | | | CGSP14) GN = thyA PE = 3 SV = 1 - | |
| | | | | | [B2IMY1_STRPS] | |
| B2IM44 | 17.88 | 3 | 274 | 23.39 | DNA-entry nuclease OS = | N- |
| | | | | | Streptococcus pneumoniae (strain | terminally |
| | | | | | CGSP14) GN = endA PE = 4 SV = 1 - | anchored |
| | | | | | [B2IM44_STRPS] | (No CS) |
| B2IR18 | 22.54 | 3 | 213 | 23.34 | Amino acid ABC transporter, ATP- | Intracellular |
| | | | | | binding protein, putative OS = | |
| | | | | | Streptococcus pneumoniae (strain | |
| | | | | | CGSP14) GN = SPCG_0107 PE = 4 SV = 1 - | |
| | | | | | [B2IRI8_STRPS] | |
| B2IRQ1 | 5.51 | 2 | 653 | 23.17 | PTS system IIABC components OS = | Multi- |
| | | | | | Streptococcus pneumoniae (strain | transmembrane |
| | | | | | CGSP14) GN = scrA PE = 4 SV = 1 - | |
| | | | | | [B2IRQ1_STRPS] | |
| B2IQR5 | 19.92 | 4 | 246 | 23.16 | CppA protein OS = -Streptococcus | Intracellular |
| | | | | | pneumoniae (strain CGSP14) | |
| | | | | | GN = cppA PE = 4 SV = 1 - | |
| | | | | | [B2IQR5_STRPS] | |
| B2ILR4 | 22.22 | 2 | 117 | 23.07 | Cell cycle protein GpsB OS = | Intracellular |
| | | | | | Streptococcus pneumoniae (strain | |
| | | | | | CGSP14) GN = gpsB PE = 3 SV = 1 - | |
| | | | | | [B2ILR4_STRPS] | |
| B2IQN2 | 20.25 | 4 | 316 | 22.86 | HPr kinase/phosphorylase OS = | Intracellular |
| | | | | | Streptococcus pneumoniae (strain | |
| | | | | | CGSP14) GN = hprK PE = 3 SV = 1 - | |
| | | | | | [B2IQN2_STRPS] | |
| B2IMA5 | 25 | 6 | 448 | 22.78 | Glutamine synthetase, type I OS = | Intracellular |
| | | | | | Streptococcus pneumoniae (strain | |
| | | | | | CGSP14) GN = glnA PE = 3 SV = 1 - | |
| | | | | | [B2IMA5_STRPS] | |
| B2IQ75 | 33.87 | 4 | 186 | 22.38 | LemA protein OS = -Streptococcus | N- |
| | | | | | pneumoniae (strain CGSP14) | terminally |
| | | | | | GN = lemA PE = 4 SV = 1 - | anchored |
| | | | | | [B2IQ75_STRPS] | (No CS) |
| B2INX5 | 19.41 | 5 | 340 | 21.91 | Branched-chain-amino-acid | Intracellular |
| | | | | | aminotransferase OS = -Streptococcus | |
| | | | | | pneumoniae (strain CGSP14) GN = ilvE | |
| | | | | | PE = 3 SV = 1 - [B2INX5_STRPS] | |
| B2IMY7 | 43.48 | 4 | 207 | 21.8 | Uncharacterized protein OS = | Intracellular |
| | | | | | Streptococcus pneumoniae (strain | |
| | | | | | CGSP14) GN = SPCG_0630 PE = 4 SV = 1 - | |
| | | | | | [B2IMY7_STRPS] | |
| B2IPY6 | 14.21 | 4 | 380 | 21.63 | Glucose-1-phosphate | Intracellular |
| | | | | | adenylyltransferase OS = | |
| | | | | | Streptococcus pneumoniae (strain | |
| | | | | | CGSP14) GN = glgC PE = 3 SV = 1 - | |
| | | | | | [GLGC_STRPS] | |
| B2IM69 | 20.91 | 4 | 416 | 21.42 | L-threonine dehydratase OS = | Intracellular |
| | | | | | Streptococcus pneumoniae (strain | |
| | | | | | CGSP14) GN = ilVA PE = 3 SV = 1 - | |
| | | | | | [B2IM69_STRPS] | |
| B2INA8 | 35.36 | 5 | 263 | 21.29 | Phosphomethylpyrimidine kinase | Intracellular |
| | | | | | OS = -Streptococcus pneumoniae (strain | |
| | | | | | CGSP14) GN = thiD PE = 4 SV = 1 - | |
| | | | | | [B2INA8_STRPS] | |
| B2ISK9 | 13.48 | 3 | 267 | 21.26 | PTS system, mannose-specific IIC | Multi- |
| | | | | | component OS = -Streptococcus | transmembrane(Lipid |
| | | | | | pneumoniae (strain CGSP14) | modified |
| | | | | | GN = manM PE = 4 SV = 1 - | N-termini) |
| | | | | | [B2ISK9_STRPS] | |
| B2IQ32 | 12.93 | 3 | 441 | 21.17 | Glycosyl transferase, group 1 OS = | Intracellular |
| | | | | | Streptococcus pneumoniae (strain | |
| | | | | | CGSP14) GN = SPCG_1204 PE = 4 SV = 1 - | |
| | | | | | [B2IQ32_STRPS] | |
| B21M58 | 8.12 | 3 | 308 | 20.91 | Membrane protein insertase YidC | Multi- |
| | | | | | OS = -Streptococcus pneumoniae (strain | transmembrane |
| | | | | | CGSP14) GN = yidC PE = 3 SV = 1 - | |
| | | | | | [B2IM58_STRPS] | |
| B2INR5 | 26.54 | 4 | 309 | 20.79 | Oxidoreductase, aldo/keto | Intracellular |
| | | | | | reductase family OS = -Streptococcus | |
| | | | | | pneumoniae (strain CGSP14) | |
| | | | | | GN = SPCG_0740 PE = 4 SV = 1 - | |
| | | | | | [B2INR5_STRPS] | |
| B2INC9 | 36.22 | 3 | 196 | 20.35 | ATP-dependent Clp protease | Intracellular |
| | | | | | proteolytic subunit OS = | |
| | | | | | CGSP14) GN = clpP PE = 3 SV = 1 - | |
| | | | | | [CLPP_STRPS] | |
| B2IQ35 | 25.75 | 4 | 369 | 20.25 | RNA polymerase sigma factor SigA | Intracellular |
| | | | | | OS = -Streptococcus pneumoniae (strain | |
| | | | | | CGSP14) GN = rpoD PE = 3 SV = 1 - | |
| | | | | | [B2IQ35_STRPS] | |
| B21MR7 | 20 | 3 | 215 | 20.15 | ABC transporter, ATP-binding | Intracellular |
| | | | | | protein Vexp2 OS = -Streptococcus | |
| | | | | | pneumoniae (strain CGSP14) | |
| | | | | | GN = SPCG_0561 PE = 4 SV = 1 - | |
| | | | | | [B2IMR7_STRPS] | |

TABLE 6
Proteins identified in MP from serotype 3. The accession numbers refer to
SwissProt on the date of filing.

| Accession # | Coverage | # Peptides | #AAs | Score | Description | Localization |
|---|---|---|---|---|---|---|
| B2IQV2 | 90.84 | 45 | 404 | 2064.28 | Elongation factor Tu OS = | Intracellular |
| | | | | | Streptococcus pneumoniae (strain | |
| | | | | | CGSP14) GN = tuf PE = 3 SV = 1 - | |
| | | | | | [B2IQV2_STRPS] | |
| B2IRT9 | 69.68 | 49 | 442 | 1423.4 | Sugar ABC transporter, sugar- | Lipid |
| | | | | | binding protein OS = -Streptococcus | anchored |
| | | | | | pneumoniae (strain CGSP14) | |
| | | | | | GN = SPCG_1656 PE = 4 SV = 1 - | |
| | | | | | [B2IRT9_STRPS] | |
| B2INY1 | 82.25 | 60 | 400 | 1396.37 | 30S ribosomal protein S1 OS = | Intracellular |
| | | | | | Streptococcus pneumoniae (strain | |
| | | | | | CGSP14) GN = rpsA PE = 4 SV = 1 - | |
| | | | | | [B2INY1_STRPS] | |
| B2ISJ9 | 86.15 | 57 | 693 | 1352.29 | Elongation factor G OS = | Intracellular |
| | | | | | Streptococcus pneumoniae (strain | |
| | | | | | CGSP14) GN = fusA PE = 3 SV = 1 - | |
| | | | | | [EFG_STRPS] | |
| B2IPX8 | 86.64 | 41 | 434 | 1214.93 | Enolase OS = -Streptococcus | Intracellular |
| | | | | | pneumoniae (strain CGSP14) | |
| | | | | | GN = eno PE = 3 SV = 1 - [ENO_STRPS] | |
| B2INB2 | 75.63 | 49 | 591 | 1080.64 | Pyruvate oxidase OS = Streptococcus | Intracellular |
| | | | | | pneumoniae (strain CGSP14) | |
| | | | | | GN = spxB PE = 3 SV = 1 - | |
| | | | | | [B2INB2_STRPS] | |
| B2IN25 | 76.44 | 47 | 450 | 1068.79 | Maltose/maltodextrin ABC | Lipid |
| | | | | | transporter, maltose/maltodextrin- | anchored |
| | | | | | binding protein OS = -Streptococcus | |
| | | | | | pneumoniae (strain CGSP14) | |
| | | | | | GN = SPCG_2072 PE = 4 SV = 1 - | |
| | | | | | [B2IN25_STRPS] | |
| B2INK4 | 76.15 | 51 | 608 | 829.85 | Uncharacterized protein OS = | N- |
| | | | | | Streptococcus pneumoniae (strain | terminally |
| | | | | | CGSP14) GN = SPCG_2153 PE = 4 | anchored |
| | | | | | SV = 1 - [B2INK4_STRPS] | (No CS) |
| B2ISK8 | 52.48 | 17 | 303 | 715.49 | PTS system, mannose-specific IID | Multi- |
| | | | | | component OS = -Streptococcus | transmembrane |
| | | | | | pneumoniae (strain CGSP14) | |
| | | | | | GN = manN PE = 4 SV = 1 - | |
| | | | | | [B2ISK8_STRPS] | |
| B2IR98 | 86.7 | 33 | 376 | 633.37 | Sugar ABC transporter, ATP-binding | Intracellular |
| | | | | | protein OS = -Streptococcus | |
| | | | | | pneumoniae (strain CGSP14) | |
| | | | | | GN = SPCG_1565 PE = 3 SV = 1 - | |
| | | | | | [B2IR98_STRPS] | |
| B2IMI7 | 75.21 | 29 | 359 | 616.73 | Glyceraldehyde-3-phosphate | Intracellular |
| | | | | | dehydrogenase OS = -Streptococcus | |
| | | | | | pneumoniae (strain CGSP14) | |
| | | | | | GN = gapA PE = 3 SV = 1 - | |
| | | | | | [B2IMI7_STRPS] | |
| B2ISL0 | 55.12 | 20 | 332 | 608.4 | PTS system, mannose-specific IIAB | Intracellular |
| | | | | | components OS = -Streptococcus | |
| | | | | | pneumoniae (strain CGSP14) | |
| | | | | | GN = manL PE = 4 SV = 1 - | |
| | | | | | [B2ISL0_STRPS] | |
| B2IRH0 | 78.91 | 39 | 512 | 573.11 | ABC transporter, substrate-binding | Lipid |
| | | | | | protein OS = -Streptococcus | anchored |
| | | | | | pneumoniae (strain CGSP14) | |
| | | | | | GN = SPCG_0089 PE = 4 SV = 1 - | |
| | | | | | [B2IRH0_STRPS] | |
| B2IS43 | 74.01 | 23 | 277 | 570.24 | 50S ribosomal protein L2 OS = | Intracellular |
| | | | | | Streptococcus pneumoniae (strain | |
| | | | | | CGSP14) GN = rpIB PE = 3 SV = 1 - | |
| | | | | | [RL2_STRPS] | |
| B2INK5 | 70.12 | 34 | 502 | 534.8 | Glycerol kinase OS = -Streptococcus | Intracellular |
| | | | | | pneumoniae (strain CGSP14) | |
| | | | | | GN = glpK PE = 3 SV = 1 - | |
| | | | | | [GLPK_STRPS] | |
| B2IRV7 | 73.6 | 55 | 837 | 530.96 | Protein translocase subunit SecA | Intracellular |
| | | | | | OS = -Streptococcus pneumoniae (strain | |
| | | | | | CGSP14) GN = secA PE = 3 SV = 1 - | |
| | | | | | [B2IRV7_STRPS] | |
| B2INW2 | 65.51 | 28 | 374 | 519.89 | Lipoprotein OS = -Streptococcus | Lipid |
| | | | | | pneumoniae (strain CGSP14) | anchored |
| | | | | | GN = SPCG_0787 PE = 4 SV = 1 - | |
| | | | | | [B2INW2_STRPS] | |
| B2IPD4 | 58.15 | 28 | 313 | 480.11 | Foldase protein PrsA OS = | Lipid |
| | | | | | Streptococcus pneumoniae (strain | anchored |
| | | | | | CGSP14) GN = prsA PE = 3 SV = 1 - | |
| | | | | | [PRSA_STRPS] | |
| B2IR00 | 59.97 | 38 | 652 | 462.58 | ATP-dependent zinc | Multi- |
| | | | | | metalloprotease FtsH OS = | transmembrane |
| | | | | | Streptococcus pneumoniae (strain | |
| | | | | | CGSP14) GN = ftsH PE = 3 SV = 1 - | |
| | | | | | [B2IR00_STRPS] | |
| B2IM39 | 54.12 | 57 | 1225 | 460.95 | DNA-directed RNA polymerase | Intracellular |
| | | | | | subunit beta′ OS = -Streptococcus | |
| | | | | | pneumoniae (strain CGSP14) | |
| | | | | | GN = rpoC PE = 3 SV = 1 - | |
| | | | | | [RPOC_STRPS] | |
| B2IM81 | 63.18 | 37 | 774 | 432.81 | Formate acetyltransferase OS = | Intracellular |
| | | | | | Streptococcus pneumoniae (strain | |
| | | | | | CGSP14) GN = pfl PE = 4 SV = 1 - | |
| | | | | | [B2IM81_STRPS] | |
| B2IM49 | 72.73 | 25 | 330 | 394.5 | Aspartate--ammonia ligase OS = | Intracellular |
| | | | | | Streptococcus pneumoniae (strain | |
| | | | | | CGSP14) GN = asnA PE = 3 SV = 1 - | |
| | | | | | [ASNA_STRPS] | |
| B2IN96 | 69.05 | 25 | 378 | 394.18 | Lactate oxidase OS = -Streptococcus | Intracellular |
| | | | | | pneumoniae (strain CGSP14) | |
| | | | | | GN = IctO PE = 4 SV = 1 - | |
| | | | | | [B2IN96_STRPS] | |
| B2IPT8 | 93.56 | 27 | 326 | 382.46 | Tagatose 1,6-diphosphate aldolase | Intracellular |
| | | | | | OS = -Streptococcus pneumoniae (strain | |
| | | | | | CGSP14) GN = lacD PE = 3 SV = 1 - | |
| | | | | | [B2IPT8_STRPS] | |
| B2INN2 | 73.36 | 26 | 259 | 380.63 | 30S ribosomal protein S2 OS = | Intracellular |
| | | | | | Streptococcus pneumoniae (strain | |
| | | | | | CGSP14) GN = rpsB PE = 3 SV = 2 - | |
| | | | | | [RS2_STRPS] | |
| B2IQX0 | 86.32 | 28 | 468 | 375.02 | ATP synthase subunit beta OS = | Intracellular |
| | | | | | Streptococcus pneumoniae (strain | |
| | | | | | CGSP14) GN = atpD PE = 3 SV = 1 - | |
| | | | | | [ATPB_STRPS] | |
| B2IMA2 | 85.18 | 25 | 398 | 374.17 | Phosphoglycerate kinase OS = | Intracellular |
| | | | | | Streptococcus pneumoniae (strain | |
| | | | | | CGSP14) GN = pgk PE = 3 SV = 1 - | |
| | | | | | [PGK_STRPS] | |
| B2IS46 | 81.11 | 26 | 217 | 368.3 | 30S ribosomal protein S3 OS = | Intracellular |
| | | | | | Streptococcus pneumoniae (strain | |
| | | | | | CGSP14) GN = rpsC PE = 3 SV = 1 - | |
| | | | | | [RS3_STRPS] | |
| B2ISZ0 | 64.49 | 31 | 659 | 336.33 | Oligopeptide ABC transporter, | Lipid |
| | | | | | oligopeptide-binding protein AmiA | anchored |
| | | | | | OS = -Streptococcus pneumoniae (strain | |
| | | | | | CGSP14) GN = amiA PE = 4 SV = 1 - | |
| | | | | | [B2ISZ0_STRPS] | |
| B2ISG3 | 77.23 | 21 | 202 | 332.38 | General stress protein 24, putative | Intracellular |
| | | | | | OS = -Streptococcus pneumoniae (strain | |
| | | | | | CGSP14) GN = SPCG_1781 PE = 4 SV = | |
| | | | | | 1 - [B2ISG3_STRPS] | |
| B21ML8 | 69.46 | 25 | 406 | 332.33 | Acetate kinase OS = -Streptococcus | Intracellular |
| | | | | | pneumoniae (strain CGSP14) | |
| | | | | | GN = ackA PE = 3 SV = 1 - | |
| | | | | | [B2IML8_STRPS] | |
| B2IP54 | 71.66 | 27 | 501 | 328.6 | Pyruvate kinase OS = -Streptococcus | Intracellular |
| | | | | | pneumoniae (strain CGSP14) | |
| | | | | | GN = SPCG_0873 PE = 3 SV = 1 - | |
| | | | | | [B2IP54_STRPS] | |
| B2IN10 | 57.69 | 12 | 338 | 324.09 | Glycerol-3-phosphate | Intracellular |
| | | | | | dehydrogenase [NAD(P)+] OS = | |
| | | | | | Streptococcus pneumoniae (strain | |
| | | | | | CGSP14) GN = gpsA PE = 3 SV = 1 - | |
| | | | | | [GPDA_STRPS] | |
| B2IRR1 | 66.09 | 12 | 230 | 318.07 | 2,3-bisphosphoglycerate- | Intracellular |
| | | | | | dependent phosphoglycerate | |
| | | | | | mutase OS = -Streptococcus | |
| | | | | | pneumoniae (strain CGSP14) | |
| | | | | | GN = gpmA PE = 3 SV = 1 - | |
| | | | | | [GPMA_STRPS] | |
| B2IRI6 | 36.84 | 5 | 95 | 310.42 | Bacteriocin, putative OS = | N- terminally |
| | | | | | Streptococcus pneumoniae (strain | anchored |
| | | | | | CGSP14) GN = SPCG_0105 PE = 4 | (with CS) |
| | | | | | SV = 1 - [B2IRI6_STRPS] | |
| B2IQY1 | 57.35 | 26 | 551 | 302.6 | Uncharacterized protein OS = | Intracellular/ |
| | | | | | Streptococcus pneumoniae (strain | TMH |
| | | | | | CGSP14) GN = SPCG_1503 PE = 4 SV = | start |
| | | | | | 1 - [B2IQY1_STRPS] | AFTER 60 |
| B2ISJ7 | 67.88 | 14 | 137 | 301.47 | 30S ribosomal protein S12 OS = | Intracellular |
| | | | | | Streptococcus pneumoniae (strain | |
| | | | | | CGSP14) GN = rpsL PE = 3 SV = 1 - | |
| | | | | | [RS12_STRPS] | |
| B2IQX2 | 38.92 | 21 | 501 | 285.3 | ATP synthase subunit alpha OS = | Intracellular |
| | | | | | Streptococcus pneumoniae (strain | |
| | | | | | CGSP14) GN = atpA PE = 3 SV = 1 - | |
| | | | | | [ATPA_STRPS] | |
| B2IP53 | 50.15 | 16 | 335 | 269.91 | ATP-dependent 6- | Intracellular |
| | | | | | phosphofructokinase OS = | |
| | | | | | Streptococcus pneumoniae (strain | |
| | | | | | CGSP14) GN = pfkA PE = 3 SV = 1 - | |
| | | | | | [PFKA_STRPS] | |
| B2IR47 | 51.69 | 27 | 652 | 263.77 | Oligopeptide ABC transporter, | Lipid |
| | | | | | oligopeptide-binding protein AliB | anchored |
| | | | | | OS = -Streptococcus pneumoniae (strain | |
| | | | | | CGSP14) GN = aliB PE = 4 SV = 1 - | |
| | | | | | [B2IR47_STRPS] | |
| B2ISZ6 | 50.36 | 17 | 419 | 260.6 | Sugar ABC transporter, sugar- | Lipid |
| | | | | | binding protein OS = -Streptococcus | anchored |
| | | | | | pneumoniae (strain CGSP14) | |
| | | | | | GN = SPCG_1871 PE = 4 SV = 1 - | |
| | | | | | [B2ISZ6_STRPS] | |
| B2IMB5 | 52.72 | 24 | 607 | 254.75 | Chaperone protein Dnak OS = | Intracellular |
| | | | | | Streptococcus pneumoniae (strain | |
| | | | | | CGSP14) GN = dnaK PE = 3 SV = 1 - | |
| | | | | | [DNAK_STRPS] | |
| B2INT1 | 42.12 | 26 | 584 | 253.89 | Septation ring formation regulator | N- |
| | | | | | EzrA OS = -Streptococcus pneumoniae | terminally |
| | | | | | (strain CGSP14) GN = ezrA PE = 3 SV = | anchored |
| | | | | | 1 - [B2INT1_STRPS] | (No CS) |
| B2IM12 | 66.03 | 26 | 471 | 237.78 | Pneumolysin OS = -Streptococcus | Intracellular |
| | | | | | pneumoniae (strain CGSP14) GN = ply | |
| | | | | | PE = 4 SV = 1 - [B2IM12_STRPS] | |
| B2IS47 | 55.47 | 11 | 137 | 228.66 | 50S ribosomal protein L16 OS = | Intracellular |
| | | | | | Streptococcus pneumoniae (strain | |
| | | | | | CGSP14) GN = rpIP PE = 3 SV = 1 - | |
| | | | | | [RL16_STRPS] | |
| B2IRM9 | 68.48 | 18 | 276 | 218.21 | ABC transporter, substrate-binding | Lipid |
| | | | | | protein OS = -Streptococcus | anchored |
| | | | | | pneumoniae (strain CGSP14) | |
| | | | | | GN = SPCG_0152 PE = 4 SV = 1 - | |
| | | | | | [B2IRM9_STRPS] | |
| B2IRG4 | 64.53 | 23 | 203 | 217.88 | 30S ribosomal protein S4 OS = | Intracellular |
| | | | | | Streptococcus pneumoniae (strain | |
| | | | | | CGSP14) GN = rpsD PE = 3 SV = 1 - | |
| | | | | | [RS4_STRPS] | |
| B2IS57 | 86.59 | 19 | 164 | 213.33 | 30S ribosomal protein S5 OS = | Intracellular |
| | | | | | Streptococcus pneumoniae (strain | |
| | | | | | CGSP14) GN = rpsE PE = 3 SV = 1 - | |
| | | | | | [B2IS57_STRPS] | |
| B2IM40 | 44.9 | 29.1 4 | 824 | 208.41 | DNA-directed RNA polymerase | Intracellular |
| | | | | | subunit beta OS = -Streptococcus | |
| | | | | | pneumoniae (strain CGSP14) | |
| | | | | | GN = rpoB PE = 3 SV = 1 - | |
| | | | | | [B2IM40_STRPS] | |
| B2ISM3 | 87.16 | 20 | 148 | 207.33 | 50S ribosomal protein L13 OS = | Intracellular |
| | | | | | Streptococcus pneumoniae (strain | |
| | | | | | CGSP14) GN = rpIM PE = 3 SV = 1 - | |
| | | | | | [B2ISM3_STRPS] | |
| B2IQ88 | 76.52 | 13 | 115 | 207.04 | 50S ribosomal protein L19 OS = | Intracellular |
| | | | | | Streptococcus pneumoniae (strain | |
| | | | | | CGSP14) GN = rpIS PE = 3 SV = 1 - | |
| | | | | | [RL19_STRPS] | |
| B2ISD1 | 80.77 | 12 | 104 | 201.22 | Thioredoxin OS = -Streptococcus | Intracellular |
| | | | | | pneumoniae (strain CGSP14) | |
| | | | | | GN = trxA PE = 3 SV = 1 - | |
| | | | | | [B2ISD1_STRPS] | |
| B2IS40 | 62.98 | 15 | 208 | 197.25 | 50S ribosomal protein L3 OS = | Intracellular |
| | | | | | Streptococcus pneumoniae (strain | |
| | | | | | CGSP14) GN = rpIC PE = 3 SV = 1 - | |
| | | | | | [RL3_STRPS] | |
| B2IMJ9 | 41.57 | 28 | 890 | 195.17 | Aldehyde-alcohol dehydrogenase | Intracellular |
| | | | | | OS = -Streptococcus pneumoniae (strain | |
| | | | | | CGSP14) GN = adhE PE = 3 SV = 1 - | |
| | | | | | [B2IMJ9_STRPS] | |
| B2IPW4 | 45.86 | 24 | 567 | 191.95 | Dihydrolipoyl dehydrogenase OS = | Intracellular |
| | | | | | Streptococcus pneumoniae (strain | |
| | | | | | CGSP14) GN = acoL PE = 4 SV = 1 - | |
| | | | | | [B2IPW4_STRPS] | |
| B2IP07 | 28.31 | 14 | 650 | 190.79 | PTS system, fructose specific IIABC | Multi- |
| | | | | | components OS = -Streptococcus | transmembrane |
| | | | | | pneumoniae (strain CGSP14) | |
| | | | | | GN = fruA PE = 4 SV = 1 - | |
| | | | | | [B2IP07_STRPS] | |
| B2IS55 | 84.27 | 13 | 178 | 180.96 | 50S ribosomal protein L6 OS = | Intracellular |
| | | | | | Streptococcus pneumoniae (strain | |
| | | | | | CGSP14) GN = rpIF PE = 3 SV = 1 - | |
| | | | | | [RL6_STRPS] | |
| B2IME4 | 33.44 | 20 | 930 | 180.14 | Translation initiation factor IF-2 OS = | Intracellular |
| | | | | | Streptococcus pneumoniae (strain | |
| | | | | | CGSP14) GN = infB PE = 3 SV = 1 - | |
| | | | | | [IF2_STRPS] | |
| B2ISW0 | 53.72 | 12 | 242 | 178.66 | Choline transporter OS = | Intracellular |
| | | | | | Streptococcus pneumoniae (strain | |
| | | | | | CGSP14) GN = SPCG_1835 PE = 4 SV = | |
| | | | | | 1 - [B2ISW0_STRPS] | |
| B2IS66 | 43.31 | 8 | 127 | 176.98 | 30S ribosomal protein S11 OS = | Intracellular |
| | | | | | Streptococcus pneumoniae (strain | |
| | | | | | CGSP14) GN = rpsK PE = 3 SV = 1 - | |
| | | | | | [RS11_STRPS] | |
| B2IPR1 | 43.98 | 13 | 332 | 173.61 | L-lactate dehydrogenase OS = | Intracellular |
| | | | | | Streptococcus pneumoniae (strain | |
| | | | | | CGSP14) GN = Idh PE = 3 SV = 1 - | |
| | | | | | [B2IPR1_STRPS] | |
| B2IRS1 | 44.15 | 15 | 419 | 169.63 | Cell division protein FtsZ OS = | Intracellular |
| | | | | | Streptococcus pneumoniae (strain | |
| | | | | | CGSP14) GN = ftsZ PE = 3 SV = 1 - | |
| | | | | | [B2IRS1_STRPS] | |
| B2IPP2 | 36.89 | 22 | 721 | 166.14 | Amino acid ABC transporter, amino | Multi- |
| | | | | | acid-binding protein/permease | transmembrane |
| | | | | | protein OS = -Streptococcus | |
| | | | | | pneumoniae (strain CGSP14) | |
| | | | | | GN = SPCG_1062 PE = 3 SV = 1 - | |
| | | | | | [B2IPP2_STRPS] | |
| B2ILZ5 | 43.89 | 21 | 540 | 165.77 | 60 kDa chaperonin OS = | Intracellular |
| | | | | | Streptococcus pneumoniae (strain | |
| | | | | | CGSP14) GN = groL PE = 3 SV = 1 - | |
| | | | | | [CH60_STRPS] | |
| B2IS41 | 65.22 | 16 | 207 | 162.59 | 50S ribosomal protein L4 OS = | Intracellular |
| | | | | | Streptococcus pneumoniae (strain | |
| | | | | | CGSP14) GN = rpID PE = 3 SV = 1 - | |
| | | | | | [RL4_STRPS] | |
| B2ILW6 | 75.31 | 15 | 243 | 162.56 | 3-ketoacyl-(Acyl-carrier-protein) | Intracellular |
| | | | | | reductase OS = -Streptococcus | |
| | | | | | pneumoniae (strain CGSP14) | |
| | | | | | GN = fabG PE = 4 SV = 1 - | |
| | | | | | [B2ILW6_STRPS] | |
| B2ISY9 | 34.54 | 14 | 498 | 161.94 | Oligopeptide ABC transporter, | Multi- |
| | | | | | permease protein Amic OS = | transmembrane |
| | | | | | Streptococcus pneumoniae (strain | |
| | | | | | CGSP14) GN = amiC PE = 3 SV = 1 - | |
| | | | | | [B2ISY9_STRPS] | |
| B2ISY6 | 58.31 | 18 | 331 | 161.56 | Oligopeptide ABC transporter, ATP- | Intracellular |
| | | | | | binding protein AmiF OS = | |
| | | | | | Streptococcus pneumoniae (strain | |
| | | | | | CGSP14) GN = amiF PE = 3 SV = 1 - | |
| | | | | | [B2ISY6_STRPS] | |
| B2IS52 | 77.22 | 13 | 180 | 159.07 | 50S ribosomal protein L5 OS = | Intracellular |
| | | | | | Streptococcus pneumoniae (strain | |
| | | | | | CGSP14) GN = rplE PE = 3 SV = 1 - | |
| | | | | | [RL5_STRPS] | |
| B2ILW7 | 50.24 | 10 | 414 | 153.03 | 3-oxoacyl-[acyl-carrier-protein] | Intracellular |
| | | | | | synthase 2 OS = -Streptococcus | |
| | | | | | pneumoniae (strain CGSP14) | |
| | | | | | GN = fabF PE = 3 SV = 1 - | |
| | | | | | [B2ILW7_STRPS] | |
| B2ISY7 | 46.76 | 16 | 355 | 151.21 | Oligopeptide ABC transporter, ATP- | Intracellular |
| | | | | | binding protein AmiE OS = | |
| | | | | | Streptococcus pneumoniae (strain | |
| | | | | | CGSP14) GN = amiE PE = 3 SV = 1 - | |
| | | | | | [B2ISY7_STRPS] | |
| B2ILU5 | 34.89 | 13 | 427 | 150.71 | Trigger factor OS = -Streptococcus | Intracellular |
| | | | | | pneumoniae (strain CGSP14) GN = tig | |
| | | | | | PE = 3 SV = 1 - [TIG_STRPS] | |
| B2IMU8 | 55.46 | 15 | 229 | 148.33 | 50S ribosomal protein L1 OS = | Intracellular |
| | | | | | Streptococcus pneumoniae (strain | |
| | | | | | CGSP14) GN = rpIA PE = 3 SV = 1 - | |
| | | | | | [RL1_STRPS] | |
| B2IS44 | 56.99 | 10 | 93 | 148.01 | 30S ribosomal protein S19 OS = | Intracellular |
| | | | | | Streptococcus pneumoniae (strain | |
| | | | | | CGSP14) GN = rpsS PE = 3 SV = 1 - | |
| | | | | | [RS19_STRPS] | |
| B2IQT6 | 47.49 | 18 | 459 | 146.83 | NADH oxidase OS = -Streptococcus | Intracellular |
| | | | | | pneumoniae (strain CGSP14) | |
| | | | | | GN = nox PE = 4 SV = 1 - | |
| | | | | | [B2IQT6_STRPS] | |
| B2INP5 | 40.65 | 15 | 492 | 145.57 | Inosine-5′-monophosphate | Intracellular |
| | | | | | dehydrogenase OS = -Streptococcus | |
| | | | | | pneumoniae (strain CGSP14) | |
| | | | | | GN = imdH PE = 3 SV = 1 - | |
| | | | | | [B2INP5_STRPS] | |
| B2INF2 | 51.27 | 20 | 513 | 140.36 | ABC transporter, ATP-binding | Intracellular |
| | | | | | protein OS = -Streptococcus | |
| | | | | | pneumoniae (strain CGSP14) | |
| | | | | | GN = SPCG_0719 PE = 4 SV = 1 - | |
| | | | | | [B2INF2_STRPS] | |
| B2IRS2 | 50.33 | 12 | 457 | 140.22 | Cell division protein FtsA OS = | Intracellular |
| | | | | | Streptococcus pneumoniae (strain | |
| | | | | | CGSP14) GN = ftsZ PE = 3 SV = 1 - | |
| | | | | | [B2IRS2_STRPS] | |
| B2IPY5 | 51.98 | 15 | 379 | 138.63 | Glycogen biosynthesis protein GlgD | Intracellular |
| | | | | | OS = -Streptococcus pneumoniae (strain | |
| | | | | | CGSP14) GN = glgD PE = 4 SV = 1 - | |
| | | | | | [B2IPY5_STRPS] | |
| B2IQH2 | 47.59 | 12 | 187 | 137.62 | 50S ribosomal protein L10 OS = | Intracellular |
| | | | | | Streptococcus pneumoniae (strain | |
| | | | | | CGSP14) GN = rpIJ PE = 3 SV = 1 - | |
| | | | | | [B2IQH2_STRPS] | |
| B2IPM7 | 29.64 | 14 | 577 | 137.31 | Phosphoenolpyruvate-protein | Intracellular |
| | | | | | phosphotransferase OS = | |
| | | | | | Streptococcus pneumoniae (strain | |
| | | | | | CGSP14) GN = SPCG_1121 PE = 3 SV = | |
| | | | | | 1 - [B2IPM7_STRPS] | |
| B2ISJ8 | 57.69 | 11 | 156 | 133.5 | 30S ribosomal protein S7 OS = | Intracellular |
| | | | | | Streptococcus pneumoniae (strain | |
| | | | | | CGSP14) GN = rpsG PE = 3 SV = 1 - | |
| | | | | | [RS7_STRPS] | |
| B2IS54 | 74.24 | 10 | 132 | 133.28 | 30S ribosomal protein S8 OS = | Intracellular |
| | | | | | Streptococcus pneumoniae (strain | |
| | | | | | CGSP14) GN = rpsH PE = 3 SV = 1 - | |
| | | | | | [RS8_STRPS] | |
| B2INZ1 | 46.38 | 17 | 470 | 132.61 | FeS assembly protein SufB OS = | Intracellu |
| | | | | | Streptococcus pneumoniae (strain | lar |
| | | | | | CGSP14) GN = SPCG_0816 PE = 4 SV = | |
| | | | | | 1 - [B2INZ1_STRPS] | |
| B2IS53 | 62.92 | 10 | 89 | 131.91 | 30S ribosomal protein S14 OS = | Intracellular |
| | | | | | Streptococcus pneumoniae (strain | |
| | | | | | CGSP14) GN = rpsN PE = 3 SV = 1 - | |
| | | | | | [RS14_STRPS] | |
| B2IS59 | 63.7 | 9 | 146 | 131.45 | 50S ribosomal protein L15 OS = | Intracellular |
| | | | | | Streptococcus pneumoniae (strain | |
| | | | | | CGSP14) GN = rpIO PE = 3 SV = 1 - | |
| | | | | | [RL15_STRPS] | |
| B2INH5 | 50.5 | 17 | 299 | 127.85 | SPFH domain/Band 7 family OS = | N- |
| | | | | | Streptococcus pneumoniae (strain | terminally |
| | | | | | CGSP14) GN = SPCG_2124 PE = 4 SV = | anchored |
| | | | | | 1 - [B2INH5_STRPS] | (No CS) |
| B2IRE3 | 73.42 | 12 | 158 | 127.54 | PTS system, IIB component OS = | Intracellular |
| | | | | | Streptococcus pneumoniae (strain | |
| | | | | | CGSP14) GN = SPCG_0062 PE = 4 SV = | |
| | | | | | 1 - [B2IRE3_STRPS] | |
| B2IN11 | 64.88 | 14 | 299 | 125.53 | UTP--glucose-1-phosphate | Secretory |
| | | | | | uridylyltransferase OS = | (released) |
| | | | | | Streptococcus pneumoniae (strain | (with CS) |
| | | | | | CGSP14) GN = galU PE = 3 SV = 1 - | |
| | | | | | [B2IN11_STRPS] | |
| B2IS19 | 43.76 | 23 | 617 | 125.33 | Proline--tRNA ligase OS = | Intracellular |
| | | | | | Streptococcus pneumoniae (strain | |
| | | | | | CGSP14) GN = proS PE = 3 SV = 1 - | |
| | | | | | [SYP_STRPS] | |
| B2IPB5 | 60.5 | 12 | 119 | 125.17 | 50S ribosomal protein L20 OS = | Intracellular |
| | | | | | Streptococcus pneumoniae (strain | |
| | | | | | CGSP14) GN = rpIT PE = 3 SV = 1 - | |
| | | | | | [RL20_STRPS] | |
| B2IMN9 | 40.47 | 11 | 425 | 125.01 | Uncharacterized protein OS = | Intracellular |
| | | | | | Streptococcus pneumoniae (strain | |
| | | | | | CGSP14) GN = SPCG_2034 PE = 4 SV = | |
| | | | | | 1 - [B2IMN9_STRPS] | |
| B2IS90 | 47.86 | 20 | 537 | 123.3 | Ribonuclease Y OS = -Streptococcus | Intracellular |
| | | | | | pneumoniae (strain CGSP14) GN = rny | |
| | | | | | PE = 3 SV = 1 - [B2IS90_STRPS] | |
| B2IPB4 | 36.36 | 6 | 66 | 122.81 | 50S ribosomal protein L35 OS = | Intracellular |
| | | | | | Streptococcus pneumoniae (strain | |
| | | | | | CGSP14) GN = rpml PE = 3 SV = 1 - | |
| | | | | | [RL35_STRPS] | |
| B2IQW2 | 62.61 | 5 | 115 | 121.34 | Bacterocin transport accessory | Intracellular |
| | | | | | protein OS = -Streptococcus | |
| | | | | | pneumoniae (strain CGSP14) GN = bta | |
| | | | | | PE = 4 SV = 1 - [B2IQW2_STRPS] | |
| B2INS1 | 30.9 | 21 | 848 | 120 | Aminopeptidase N OS = | Intracellular |
| | | | | | Streptococcus pneumoniae (strain | |
| | | | | | CGSP14) GN = pepN PE = 4 SV = 1 - | |
| | | | | | [B2INS1_STRPS] | |
| B2IPP1 | 53.66 | 9 | 246 | 116.21 | Amino acid ABC transporter, ATP- | Intracellular |
| | | | | | binding protein OS = -Streptococcus | |
| | | | | | pneumoniae (strain CGSP14) | |
| | | | | | GN = SPCG_1061 PE = 4 SV = 1 - | |
| | | | | | [B2IPP1_STRPS] | |
| B2IMN0 | 34.94 | 8 | 352 | 116.08 | Alcohol dehydrogenase, zinc- | Intracellular |
| | | | | | containing OS = -Streptococcus | |
| | | | | | pneumoniae (strain CGSP14) | |
| | | | | | GN = adh PE = 3 SV = 1 - | |
| | | | | | [B2IMN0_STRPS] | |
| B2IRN0 | 53.52 | 14 | 284 | 114.19 | Lipoprotein OS = -Streptococcus | Lipid |
| | | | | | pneumoniae (strain CGSP14) | anchored |
| | | | | | GN = SPCG_0153 PE = 3 SV = 1 - | |
| | | | | | [B2IRN0_STRPS] | |
| B2IQZ2 | 48.66 | 14 | 374 | 110.45 | Ribosome-binding ATPase YchF OS = | Intracellular |
| | | | | | Streptococcus pneumoniae (strain | |
| | | | | | CGSP14) GN = ychF PE = 3 SV = 1 - | |
| | | | | | [B2IQZ2_STRPS] | |
| B2IQ10 | 41.57 | 13 | 344 | 105.33 | Uncharacterized protein OS = | Multi- |
| | | | | | Streptococcus pneumoniae (strain | transmembrane |
| | | | | | CGSP14) GN = SPCG_1352 PE = 4 SV = | |
| | | | | | 1 - [B2IQ10_STRPS] | |
| B2IS49 | 60.47 | 7 | 86 | 105.08 | 30S ribosomal protein S17 OS = | Intracellular |
| | | | | | Streptococcus pneumoniae (strain | |
| | | | | | CGSP14) GN = rpsQ PE = 3 SV = 1 - | |
| | | | | | [RS17_STRPS] | |
| B2IR07 | 35.97 | 15 | 442 | 104.04 | Adenylosuccinate synthetase OS = | Intracellular |
| | | | | | Streptococcus pneumoniae (strain | |
| | | | | | CGSP14) GN = purA PE = 3 SV = 1 - | |
| | | | | | [B2IR07_STRPS] | |
| B2IMU7 | 42.55 | 6 | 141 | 103.8 | 50S ribosomal protein L11 OS = | Intracellular |
| | | | | | Streptococcus pneumoniae (strain | |
| | | | | | CGSP14) GN = rpIK PE = 3 SV = 1 - | |
| | | | | | [RL11_STRPS] | |
| B2ISV9 | 21.53 | 9 | 511 | 103.52 | Choline transporter OS = | Multi- |
| | | | | | Streptococcus pneumoniae (strain | transmembrane |
| | | | | | CGSP14) GN = SPCG_1834 PE = 3 SV = | |
| | | | | | 1 - [B2ISV9_STRPS] | |
| B2IQV9 | 40.56 | 16 | 572 | 103.11 | Phosphoglucomutase OS = | Intracellular |
| | | | | | Streptococcus pneumoniae (strain | |
| | | | | | CGSP14) GN = pgm PE = 3 SV = 1 - | |
| | | | | | [B2IQV9_STRPS] | |
| B2IR17 | 52.51 | 12 | 339 | 101.9 | Ribose-phosphate | Intracellular |
| | | | | | pyrophosphokinase OS = | |
| | | | | | Streptococcus pneumoniae (strain | |
| | | | | | CGSP14) GN = prsA PE = 3 SV = 1 - | |
| | | | | | [B2IR17_STRPS] | |
| B2IRQ3 | 37.38 | 18 | 650 | 101.35 | Endopeptidase O OS = -Streptococcus | Intracellular |
| | | | | | pneumoniae (strain CGSP14) | |
| | | | | | GN = pepO PE = 4 SV = 1 - | |
| | | | | | [B2IRQ3_STRPS] | |
| B2IS63 | 54.72 | 6 | 212 | 100 | Adenylate kinase OS = -Streptococcus | Intracellular |
| | | | | | pneumoniae (strain CGSP14) GN = adk | |
| | | | | | PE = 3 SV = 1 - [KAD_STRPS] | |
| B2IMK3 | 38.15 | 13 | 658 | 99.61 | Transketolase OS = -Streptococcus | Intracellular |
| | | | | | pneumoniae (strain CGSP14) | |
| | | | | | GN = tktA PE = 3 SV = 1 - | |
| | | | | | [B2IMK3_STRPS] | |
| B2IMS2 | 55.97 | 12 | 293 | 98.56 | Fructose-bisphosphate aldolase | Intracellular |
| | | | | | OS = -Streptococcus pneumoniae (strain | |
| | | | | | CGSP14) GN = fba PE = 3 SV = 1 - | |
| | | | | | [B2IMS2_STRPS] | |
| B2IMH3 | 55.78 | 12 | 346 | 96.72 | Catabolite control protein A OS = | Intracellular |
| | | | | | Streptococcus pneumoniae (strain | |
| | | | | | CGSP14) GN = ccpA PE = 4 SV = 1 - | |
| | | | | | [B2IMH3_STRPS] | |
| B2IPY8 | 48.31 | 15 | 474 | 96.35 | Glyceraldehyde-3-phosphate | Intracellular |
| | | | | | dehydrogenase, NADP-dependent | |
| | | | | | OS = -Streptococcus pneumoniae (strain | |
| | | | | | CGSP14) GN = gapN PE = 3 SV = 1 - | |
| | | | | | [B2IPY8_STRPS] | |
| B2ILY5 | 30.81 | 10 | 555 | 96.12 | Uncharacterized protein OS = | Intracellular |
| | | | | | Streptococcus pneumoniae (strain | |
| | | | | | CGSP14) GN = SPCG_0438 PE = 4 SV = | |
| | | | | | 1 - [B2ILY5_STRPS] | |
| B2IS42 | 95.92 | 11 | 98 | 95.91 | 50S ribosomal protein L23 OS = | Intracellular |
| | | | | | Streptococcus pneumoniae (strain | |
| | | | | | CGSP14) GN = rpIW PE = 3 SV = 1 - | |
| | | | | | [RL23_STRPS] | |
| B2IM28 | 43.56 | 13 | 388 | 95.69 | Protein RecA OS = -Streptococcus | Intracellular |
| | | | | | pneumoniae (strain CGSP14) GN = recA | |
| | | | | | PE = 3 SV = 1 - [RECA_STRPS] | |
| B2IRR6 | 27.44 | 10 | 266 | 95.52 | Cell division protein DivIVA OS = | Intracellular |
| | | | | | Streptococcus pneumoniae (strain | |
| | | | | | CGSP14) GN = SPCG_1633 PE = 4 SV = | |
| | | | | | 1 - [B2IRR6_STRPS] | |
| B2IS68 | 64.06 | 8 | 128 | 93.87 | 50S ribosomal protein L17 OS = | Intracellular |
| | | | | | Streptococcus pneumoniae (strain | |
| | | | | | CGSP14) GN = rpIQ PE = 3 SV = 1 - | |
| | | | | | [RL17_STRPS] | |
| B2IPR2 | 22.47 | 13 | 841 | 93.61 | DNA gyrase subunit A OS = | Intracellular |
| | | | | | Streptococcus pneumoniae (strain | |
| | | | | | CGSP14) GN = gyrA PE = 3 SV = 1 - | |
| | | | | | [B2IPR2_STRPS] | |
| B2IM73 | 29.94 | 12 | 521 | 93.46 | Amino acid ABC transporter, amino | Multi- |
| | | | | | acid-binding protein/permease | transmembrane |
| | | | | | protein OS = -Streptococcus | |
| | | | | | pneumoniae (strain CGSP14) | |
| | | | | | GN = gInH PE = 3 SV = 1 - | |
| | | | | | [B2IM73_STRPS] | |
| B2INV5 | 59.04 | 7 | 83 | 93.23 | 30S ribosomal protein S20 OS = | Intracellular |
| | | | | | Streptococcus pneumoniae (strain | |
| | | | | | CGSP14) GN = rpsT PE = 3 SV = 1 - | |
| | | | | | [B2INV5_STRPS] | |
| B2IRH9 | 39.61 | 21 | 616 | 93.09 | Capsular polysaccharide | Multi- |
| | | | | | biosynthesis protein, putative OS = | transmembrane |
| | | | | | Streptococcus pneumoniae (strain | |
| | | | | | CGSP14) GN = SPCG_0098 PE = 4 SV = | |
| | | | | | 1 - [B2IRH9_STRPS] | |
| B2INY7 | 55.13 | 9 | 263 | 92.69 | ABC transporter, ATP-binding | Intracellular |
| | | | | | protein OS = -Streptococcus | |
| | | | | | pneumoniae (strain CGSP14) | |
| | | | | | GN = SPCG_0812 PE = 4 SV = 1 - | |
| | | | | | [B2INY7_STRPS] | |
| B2INW3 | 45.21 | 14 | 511 | 92.64 | Sugar ABC transporter, ATP-binding | Intracellular |
| | | | | | protein OS = -Streptococcus | |
| | | | | | pneumoniae (strain CGSP14) | |
| | | | | | GN = SPCG_0788 PE = 4 SV = 1 - | |
| | | | | | [B2INW3_STRPS] | |
| B2IQU4 | 57.5 | 9 | 280 | 89.47 | Oxidoreductase, aldo/keto | Intracellular |
| | | | | | reductase family OS = -Streptococcus | |
| | | | | | pneumoniae (strain CGSP14) | |
| | | | | | GN = SPCG_1466 PE = 4 SV = 1 - | |
| | | | | | [B2IQU4_STRPS] | |
| B2ILR7 | 36.8 | 13 | 481 | 87.34 | 6-phosphogluconate | Intracellular |
| | | | | | dehydrogenase, decarboxylating | |
| | | | | | OS = -Streptococcus pneumoniae (strain | |
| | | | | | CGSP14) GN = gnd PE = 3 SV = 1 - | |
| | | | | | [B2ILR7_STRPS] | |
| B2IRW4 | 23.85 | 14 | 436 | 87.08 | GTPase Der OS = -Streptococcus | Intracellular |
| | | | | | pneumoniae (strain CGSP14) GN = der | |
| | | | | | PE = 3 SV = 1 - [DER_STRPS] | |
| B2IQW3 | 46.76 | 10 | 278 | 86.59 | Amino acid ABC transporter, amino | Lipid |
| | | | | | acid-binding protein OS = | anchored |
| | | | | | Streptococcus pneumoniae (strain | |
| | | | | | CGSP14) GN = SPCG_1485 PE = 4 SV = | |
| | | | | | 1 - [B2IQW3_STRPS] | |
| B2IPI7 | 33.14 | 8 | 341 | 85.78 | Iron-compound ABC transporter, | Lipid |
| | | | | | iron compound-binding protein OS = | anchored |
| | | | | | Streptococcus pneumoniae (strain | |
| | | | | | CGSP14) GN = SPCG_1009 PE = 4 SV = | |
| | | | | | 1 - [B2IPI7_STRPS] | |
| B2IS65 | 58.68 | 12 | 121 | 85.63 | 30S ribosomal protein S13 OS = | Intracellular |
| | | | | | Streptococcus pneumoniae (strain | |
| | | | | | CGSP14) GN = rpsM PE = 3 SV = 1 - | |
| | | | | | [RS13_STRPS] | |
| B2ISM4 | 59.23 | 8 | 130 | 85.44 | 30S ribosomal protein S9 OS = | Intracellular |
| | | | | | Streptococcus pneumoniae (strain | |
| | | | | | CGSP14) GN = rpsI PE = 3 SV = 1 - | |
| | | | | | [RS9_STRPS] | |
| B2INT7 | 37.9 | 17 | 752 | 85.35 | ATP-dependent Clp protease, ATP- | Intracellular |
| | | | | | binding subunit ClpE OS = | |
| | | | | | Streptococcus pneumoniae (strain | |
| | | | | | CGSP14) GN = clpE PE = 3 SV = 1 - | |
| | | | | | [B2INT7_STRPS] | |
| B2IMU6 | 41.18 | 8 | 238 | 85.34 | Uncharacterized protein OS = | Lipid |
| | | | | | Streptococcus pneumoniae (strain | anchored |
| | | | | | CGSP14) GN = SPCG_0590 PE = 4 SV = | |
| | | | | | 1 - [B2IMU6_STRPS] | |
| B2IQR1 | 39.42 | 15 | 520 | 84.59 | GMP synthase [glutamine- | Intracellular |
| | | | | | hydrolyzing] OS = -Streptococcus | |
| | | | | | pneumoniae (strain CGSP14) | |
| | | | | | GN = guaA PE = 3 SV = 1 - | |
| | | | | | [GUAA_STRPS] | |
| B2IPT6 | 68.42 | 8 | 171 | 84.01 | Galactose-6-phosphate isomerase | Intracellular |
| | | | | | subunit LacB OS = -Streptococcus | |
| | | | | | pneumoniae (strain CGSP14) GN = lacB | |
| | | | | | PE = 3 SV = 1 - [LACB_STRPS] | |
| B2INL3 | 30.25 | 17 | 810 | 83.74 | ATP-dependent Clp protease, ATP- | Intracellular |
| | | | | | binding subunit OS = -Streptococcus | |
| | | | | | pneumoniae (strain CGSP14) | |
| | | | | | GN = SPCG_2162 PE = 3 SV = 1 - | |
| | | | | | [B2INL3_STRPS] | |
| B2IS51 | 78.22 | 10 | 101 | 83.43 | 50S ribosomal protein L24 OS = | Intracellular |
| | | | | | Streptococcus pneumoniae (strain | |
| | | | | | CGSP14) GN = rpIX PE = 3 SV = 1 - | |
| | | | | | [RL24_STRPS] | |
| B2INN1 | 42.77 | 10 | 346 | 83.3 | Elongation factor Ts OS = | Intracellular |
| | | | | | Streptococcus pneumoniae (strain | |
| | | | | | CGSP14) GN = tsf PE = 3 SV = 1 - | |
| | | | | | [EFTS_STRPS] | |
| B2IS56 | 67.18 | 10 | 131 | 82.77 | 50S ribosomal protein L18 OS = | Intracellular |
| | | | | | Streptococcus pneumoniae (strain | |
| | | | | | CGSP14) GN = rpIR PE = 3 SV = 1 - | |
| | | | | | [B2IS56_STRPS] | |
| B2ISX8 | 27.68 | 8 | 336 | 82.74 | Non-canonical purine NTP | Intracellular |
| | | | | | pyrophosphatase OS = -Streptococcus | |
| | | | | | pneumoniae (strain CGSP14) | |
| | | | | | GN = SPCG_1853 PE = 3 SV = 1 - | |
| | | | | | [B2ISX8_STRPS] | |
| B2INT0 | 24.07 | 12 | 648 | 81.85 | DNA gyrase subunit B OS = | Intracellular |
| | | | | | Streptococcus pneumoniae (strain | |
| | | | | | CGSP14) GN = gyrB PE = 3 SV = 1 - | |
| | | | | | [B2INT0_STRPS] | |
| B2IN71 | 31.13 | 15 | 620 | 81.8 | Elongation factor Tu family protein | Intracellular |
| | | | | | OS = -Streptococcus pneumoniae (strain | |
| | | | | | CGSP14) GN = SPCG_0638 PE = 4 SV = | |
| | | | | | 1 - [B2IN71_STRPS] | |
| B2IPU8 | 19.19 | 9 | 719 | 79.81 | Ribonucleoside-diphosphate | Intracellular |
| | | | | | reductase OS = -Streptococcus | |
| | | | | | pneumoniae (strain CGSP14) | |
| | | | | | GN = nrdE PE = 3 SV = 1 - | |
| | | | | | [B2IPU8_STRPS] | |
| B2INM4 | 49.45 | 9 | 182 | 79.8 | Ribosomal subunit interface protein | Intracellular |
| | | | | | OS = -Streptococcus pneumoniae (strain | |
| | | | | | CGSP14) GN = SPCG_2173 PE = 4 SV = | |
| | | | | | 1 - [B2INM4_STRPS] | |
| B2ISV2 | 28.57 | 7 | 392 | 79.49 | Galactokinase OS = -Streptococcus | Intracellular |
| | | | | | pneumoniae (strain CGSP14) | |
| | | | | | GN = gaIK PE = 3 SV = 1 - | |
| | | | | | [B2ISV2_STRPS] | |
| B2IMZO | 45.21 | 9 | 449 | 77.7 | Glucose-6-phosphate isomerase | Intracellular |
| | | | | | OS = -Streptococcus pneumoniae (strain | |
| | | | | | CGSP14) GN = pgi PE = 3 SV = 1 - | |
| | | | | | [G6PI_STRPS] | |
| B2IRD4 | 61.8 | 6 | 89 | 77.46 | 30S ribosomal protein S15 OS = | Intracellular |
| | | | | | Streptococcus pneumoniae (strain | |
| | | | | | CGSP14) GN = rpsO PE = 3 SV = 1 - | |
| | | | | | [RS15_STRPS] | |
| B2IR65 | 69.38 | 11 | 209 | 76.49 | Uncharacterized protein OS = | Intracellular |
| | | | | | Streptococcus pneumoniae (strain | |
| | | | | | CGSP14) GN = SPCG_1532 PE = 4 SV = | |
| | | | | | 1 - [B2IR65_STRPS] | |
| B2IR90 | 43.02 | 4 | 172 | 76.3 | Non-heme iron-containing ferritin | Intracellular |
| | | | | | OS = -Streptococcus pneumoniae (strain | |
| | | | | | CGSP14) GN = SPCG_1557 PE = 3 SV = | |
| | | | | | 1 - [B2IR90_STRPS] | |
| B2IPY6 | 28.16 | 9 | 380 | 75.91 | Glucose-1-phosphate | Intracellular |
| | | | | | adenylyltransferase OS = | |
| | | | | | Streptococcus pneumoniae (strain | |
| | | | | | CGSP14) GN = glgC PE = 3 SV = 1 - | |
| | | | | | [GLGC_STRPS] | |
| B2IN68 | 38.1 | 7 | 126 | 75.25 | Uncharacterized protein OS = | N-terminally |
| | | | | | Streptococcus pneumoniae (strain | anchored |
| | | | | | CGSP14) GN = SPCG_0635 PE = 4 SV = | (No CS) |
| | | | | | 1 - [B2IN68_STRPS] | |
| B2IR60 | 97.92 | 8 | 96 | 74.99 | 30S ribosomal protein S6 OS = | Intracellular |
| | | | | | Streptococcus pneumoniae (strain | |
| | | | | | CGSP14) GN = rpsF PE = 3 SV = 1 - | |
| | | | | | [RS6_STRPS] | |
| B2IQ02 | 48.45 | 7 | 97 | 74.43 | 50S ribosomal protein L27 OS = | Intracellular |
| | | | | | Streptococcus pneumoniae (strain | |
| | | | | | CGSP14) GN = rpmA PE = 3 SV = 1 - | |
| | | | | | [RL27_STRPS] | |
| B2IM41 | 40.05 | 10 | 377 | 74.15 | Uncharacterized protein OS = | Intracellular |
| | | | | | Streptococcus pneumoniae (strain | |
| | | | | | CGSP14) GN = SPCG_1926 PE = 3 SV = | |
| | | | | | 1 - [B2IM41_STRPS] | |
| B2IR71 | 16.41 | 12 | 914 | 73.99 | Cation-transporting ATPase, E1-E2 | Multi- |
| | | | | | family OS = -Streptococcus pneumoniae | transmembrane |
| | | | | | (strain CGSP14) GN = pacL PE = 3 | |
| | | | | | SV = 1 - [B2IR71_STRPS] | |
| B2IQV3 | 35.99 | 6 | 289 | 72.94 | Glycerol uptake facilitator protein, | Multi- |
| | | | | | putative OS = -Streptococcus | transmembrane |
| | | | | | pneumoniae (strain CGSP14) | |
| | | | | | GN = glpF PE = 3 SV = 1 - | |
| | | | | | [B2IQV3_STRPS] | |
| B2ILW5 | 35.95 | 9 | 306 | 72.85 | Malonyl CoA-acyl carrier protein | Intracellular |
| | | | | | transacylase OS = -Streptococcus | |
| | | | | | pneumoniae (strain CGSP14) | |
| | | | | | GN = fabD PE = 3 SV = 1 - | |
| | | | | | [B2ILW5_STRPS] | |
| B2IRQ6 | 42.81 | 10 | 313 | 71.49 | Manganese ABC transporter, | Lipid |
| | | | | | manganese-binding adhesion | anchored |
| | | | | | liprotein OS = -Streptococcus | |
| | | | | | pneumoniae (strain CGSP14) | |
| | | | | | GN = SPCG_1623 PE = 3 SV = 1 - | |
| | | | | | [B2IRQ6_STRPS] | |
| B2IQX4 | 60.98 | 9 | 164 | 71.29 | ATP synthase subunit b OS = | N- |
| | | | | | Streptococcus pneumoniae (strain | terminally |
| | | | | | CGSP14) GN = atpF PE = 3 SV = 1 - | anchored |
| | | | | | [ATPF_STRPS] | (No CS) |
| B2IPT0 | 26.59 | 10 | 628 | 71.03 | Elongation factor 4 OS = | Intracellular |
| | | | | | Streptococcus pneumoniae (strain | |
| | | | | | CGSP14) GN = lepA PE = 3 SV = 1 - | |
| | | | | | [B2IPT0_STRPS] | |
| B2ISJ1 | 27.74 | 11 | 602 | 69.46 | Glutamine--fructose-6-phosphate | Intracellular |
| | | | | | aminotransferase [isomerizing] OS = | |
| | | | | | Streptococcus pneumoniae (strain | |
| | | | | | CGSP14) GN = glmS PE = 3 SV = 1 - | |
| | | | | | [B2ISJ1_STRPS] | |
| B2IQU1 | 23.75 | 13 | 678 | 68.47 | Glycine--tRNA ligase beta subunit | Intracellular |
| | | | | | OS = -Streptococcus pneumoniae (strain | |
| | | | | | CGSP14) GN = glyS PE = 3 SV = 1 - | |
| | | | | | [SYGB_STRPS] | |
| B2INH0 | 41.42 | 11 | 338 | 68.3 | Ornithine carbamoyltransferase | Intracellular |
| | | | | | OS = -Streptococcus pneumoniae (strain | |
| | | | | | CGSP14) GN = arcB PE = 3 SV = 1 - | |
| | | | | | [OTC_STRPS] | |
| B2IQL4 | 61.42 | 12 | 267 | 68.04 | Phosphate import ATP-binding | Intracellular |
| | | | | | protein PstB OS = -Streptococcus | |
| | | | | | pneumoniae (strain CGSP14) | |
| | | | | | GN = pstB PE = 3 SV = 1 - | |
| | | | | | [B2IQL4_STRPS] | |
| B2ING9 | 37.9 | 10 | 409 | 65.94 | Arginine deiminase OS = | Intracellular |
| | | | | | Streptococcus pneumoniae (strain | |
| | | | | | CGSP14) GN = arcA PE = 3 SV = 1 - | |
| | | | | | [ARCA_STRPS] | |
| B2IR87 | 27.8 | 9 | 410 | 65.69 | ATP-dependent Clp protease ATP- | Intracellular |
| | | | | | binding subunit ClpX OS = | |
| | | | | | Streptococcus pneumoniae (strain | |
| | | | | | CGSP14) GN = clpX PE = 3 SV = 1 - | |
| | | | | | [CLPX_STRPS] | |
| B2IPY4 | 19.29 | 7 | 477 | 64.75 | Glycogen synthase OS = | Intracellular |
| | | | | | Streptococcus pneumoniae (strain | |
| | | | | | CGSP14) GN = glgA PE = 3 SV = 1 - | |
| | | | | | [GLGA_STRPS] | |
| B2ISY0 | 36 | 4 | 100 | 63.86 | UPF0154 protein SPCG_1855 OS = | N- |
| | | | | | Streptococcus pneumoniae (strain | terminally |
| | | | | | CGSP14) GN = SPCG_1855 PE = 3 SV = | anchored |
| | | | | | 1 - [B2ISY0_STRPS] | (No CS) |
| B2IQL1 | 28.04 | 6 | 271 | 63.33 | Amino acid ABC transporter, amino | Lipid |
| | | | | | acid-binding protein OS = | anchored |
| | | | | | Streptococcus pneumoniae (strain | |
| | | | | | CGSP14) GN = SPCG_1383 PE = 4 SV = | |
| | | | | | 1 - [B2IQL1_STRPS] | |
| B2IN60 | 55 | 5 | 60 | 63.27 | 50S ribosomal protein L32 OS = | Intracellular |
| | | | | | Streptococcus pneumoniae (strain | |
| | | | | | CGSP14) GN = rpmF PE = 3 SV = 1 - | |
| | | | | | [RL32_STRPS] | |
| B2IQK0 | 22.48 | 15 | 872 | 63.2 | Alanine -- tRNA ligase OS = | Intracellular |
| | | | | | Streptococcus pneumoniae (strain | |
| | | | | | CGSP14) GN = alaS PE = 3 SV = 1 - | |
| | | | | | [SYA_STRPS] | |
| B2INE4 | 30.43 | 8 | 447 | 62.91 | DEAD-box ATP-dependent RNA | Intracellular |
| | | | | | helicase CshB OS = -Streptococcus | |
| | | | | | pneumoniae (strain CGSP14) | |
| | | | | | GN = rheB PE = 3 SV = 1 - | |
| | | | | | [B2INE4_STRPS] | |
| B2IS50 | 59.84 | 8 | 122 | 62.57 | 50S ribosomal protein L14 OS = | Intracellular |
| | | | | | Streptococcus pneumoniae (strain | |
| | | | | | CGSP14) GN = rpIN PE = 3 SV = 1 - | |
| | | | | | [RL14_STRPS] | |
| B2IND2 | 34.2 | 8 | 386 | 62.51 | Branched-chain amino acid ABC | Lipid |
| | | | | | transporter, amino acid-binding | anchored |
| | | | | | protein OS = -Streptococcus | |
| | | | | | pneumoniae strain CGSP14) | |
| | | | | | GN = SPCG_0699 PE = 4 SV = 1 - | |
| | | | | | [B2IND2_STRPS] | |
| B2IRC1 | 40.12 | 8 | 339 | 61.9 | UDP-glucose 4-epimerase OS = | Intracellular |
| | | | | | Streptococcus pneumoniae (strain | |
| | | | | | CGSP14) GN = galE PE = 4 SV = 1 - | |
| | | | | | [B2IRC1_STRPS] | |
| B2IR96 | 77.27 | 7 | 176 | 61.33 | Adenine phosphoribosyltransferase | Intracellular |
| | | | | | OS = -Streptococcus pneumoniae (strain | |
| | | | | | CGSP14) GN = apt PE = 3 SV = 1 - | |
| | | | | | [B2IR96_STRPS] | |
| B2IM46 | 42.61 | 9 | 345 | 61.11 | Uncharacterized protein OS = | N- |
| | | | | | Streptococcus pneumoniae (strain | terminally |
| | | | | | CGSP14) GN = SPCG_1931 PE = 4 SV = | anchored |
| | | | | | 1 - [B2IM46_STRPS] | (No CS) |
| B21MG8 | 28.22 | 8 | 404 | 61 | Aspartate aminotransferase OS = | Intracellular |
| | | | | | Streptococcus pneumoniae (strain | |
| | | | | | CGSP14) GN = aspC PE = 4 SV = 1 - | |
| | | | | | [B2IMG8_STRPS] | |
| B2IQN0 | 59.06 | 5 | 127 | 60.7 | Uncharacterized protein OS = | N- |
| | | | | | Streptococcus pneumoniae (strain | terminally |
| | | | | | CGSP14) GN = SPCG_1399 PE = 4 SV = | anchored |
| | | | | | 1 - [B2IQN0_STRPS] | (No CS) |
| B2IMQ9 | 22.52 | 11 | 737 | 60.06 | Polyribonucleotide | Intracellular |
| | | | | | nucleotidyltransferase OS = | |
| | | | | | Streptococcus pneumoniae (strain | |
| | | | | | CGSP14) GN = pnp PE = 3 SV = 2 - | |
| | | | | | [PNP_STRPS] | |
| B2IQ40 | 24.39 | 14 | 898 | 59.69 | Phosphoenolpyruvate carboxylase | Intracellular |
| | | | | | OS = -Streptococcus pneumoniae (strain | |
| | | | | | CGSP14) GN = ppc PE = 3 SV = 1 - | |
| | | | | | [B2IQ40_STRPS] | |
| B2IND9 | 60.43 | 10 | 230 | 59.42 | Cell division ABC transporter, ATP- | Intracellular |
| | | | | | binding protein FtsE OS = | |
| | | | | | Streptococcus pneumoniae (strain | |
| | | | | | CGSP14) GN = ftsE PE = 4 SV = 1 - | |
| | | | | | [B2IND9_STRPS] | |
| B2IQU5 | 27.92 | 10 | 480 | 59.4 | Peptidoglycan N-acetylglucosamine | N- |
| | | | | | deacetylase A OS = -Streptococcus | terminally |
| | | | | | pneumoniae (strain CGSP14) | anchored |
| | | | | | GN = pgdA PE = 4 SV = 1 - | (No CS) |
| | | | | | [B2IQU5_STRPS] | |
| B2IQ23 | 39.06 | 8 | 425 | 59.39 | Uncharacterized protein OS = | Intracellular |
| | | | | | Streptococcus pneumoniae (strain | |
| | | | | | CGSP14) GN = SPCG_1195 PE = 4 SV = | |
| | | | | | 1 - [B2IQ23_STRPS] | |
| B2INH1 | 22.54 | 6 | 315 | 59 | Carbamate kinase OS = -Streptococcus | Secretory |
| | | | | | pneumoniae (strain CGSP14) | (released) |
| | | | | | GN = arcC PE = 3 SV = 1 - | (with CS) |
| | | | | | [B2INH1_STRPS] | |
| B2IRI7 | 20.76 | 11 | 713 | 58.73 | Uncharacterized protein OS = | Multi- |
| | | | | | Streptococcus pneumoniae (strain | transmembrane |
| | | | | | CGSP14) GN = SPCG_0106 PE = 4 SV = | |
| | | | | | 1 - [B2IRI7_STRPS] | |
| B2IMP1 | 37.86 | 6 | 206 | 56.51 | Hydrolase, haloacid dehalogenase- | Intracellular |
| | | | | | like family OS = -Streptococcus | |
| | | | | | pneumoniae (strain CGSP14) | |
| | | | | | GN = SPCG_2031 PE = 4 SV = 1 - | |
| | | | | | [B2IMP1_STRPS] | |
| B2IQH6 | 45.83 | 13 | 312 | 56.05 | Bifunctional methionine sulfoxide | N- |
| | | | | | reductase A/B protein OS = | terminally |
| | | | | | Streptococcus pneumoniae (strain | anchored |
| | | | | | CGSP14) GN = msrA PE = 3 SV = 1 - | (No CS) |
| | | | | | [B2IQH6_STRPS] | |
| B2IMB6 | 28.84 | 8 | 378 | 56.02 | Chaperone protein DnaJ OS = | Intracellular |
| | | | | | Streptococcus pneumoniae (strain | |
| | | | | | CGSP14) GN = dnaJ PE = 3 SV = 1 - | |
| | | | | | [B2IMB6_STRPS] | |
| B2IS18 | 32.46 | 7 | 419 | 55.7 | Zinc metalloprotease OS = | Multi- |
| | | | | | Streptococcus pneumoniae (strain | transmembrane |
| | | | | | CGSP14) GN = eep PE = 3 SV = 1 - | |
| | | | | | [B2IS18_STRPS] | |
| B2IR93 | 26.64 | 4 | 259 | 55.23 | Triosephosphate isomerase OS = | Intracellular |
| | | | | | Streptococcus pneumoniae (strain | |
| | | | | | CGSP14) GN = tpiA PE = 3 SV = 1 - | |
| | | | | | [B2IR93_STRPS] | |
| B2ILX0 | 37.58 | 11 | 455 | 54.82 | Acetyl-CoA carboxylase OS = | Intracellular |
| | | | | | Streptococcus pneumoniae (strain | |
| | | | | | CGSP14) GN = accC PE = 4 SV = 1 - | |
| | | | | | [B2ILX0_STRPS] | |
| B2IS39 | 65.69 | 6 | 102 | 54.76 | 30S ribosomal protein S10 OS = | Intracellular |
| | | | | | Streptococcus pneumoniae (strain | |
| | | | | | CGSP14) GN = rpsJ PE = 3 SV = 1 - | |
| | | | | | [RS10_STRPS] | |
| B2IQZ9 | 53.89 | 9 | 180 | 54.16 | Hypoxanthine-guanine | Intracellular |
| | | | | | phosphoribosyltransferase OS = | |
| | | | | | Streptococcus pneumoniae (strain | |
| | | | | | CGSP14) GN = hgt PE = 4 SV = 1 - | |
| | | | | | [B2IQZ9_STRPS] | |
| B2IMZ6 | 14.35 | 10 | 857 | 54.16 | DNA mismatch repair protein MutS | Intracellular |
| | | | | | OS = -Streptococcus pneumoniae (strain | |
| | | | | | CGSP14) GN = hexA PE = 3 SV = 1 - | |
| | | | | | [B2IMZ6_STRPS] | |
| B2IR84 | 36.49 | 7 | 296 | 53.72 | Nucleotide-binding protein | Intracellular |
| | | | | | SPCG_1551 OS = -Streptococcus | |
| | | | | | pneumoniae (strain CGSP14) | |
| | | | | | GN = SPCG_1551 PE = 3 SV = 1 - | |
| | | | | | [Y1551_STRPS] | |
| B2ISX0 | 31.78 | 8 | 321 | 53.7 | Iron-compound ABC transporter, | Lipid |
| | | | | | iron-compound-binding protein | anchored |
| | | | | | OS = -Streptococcus pneumoniae (strain | |
| | | | | | CGSP14) GN = SPCG_1845 PE = 4 SV = | |
| | | | | | 1 - [B2ISX0_STRPS] | |
| B2ILX6 | 56.45 | 6 | 186 | 52.99 | Elongation factor P OS = | Intracellular |
| | | | | | Streptococcus pneumoniae (strain | |
| | | | | | CGSP14) GN = efp PE = 3 SV = 1 - | |
| | | | | | [EFP_STRPS] | |
| B2IMZ8 | 23.45 | 9 | 563 | 52.53 | Arginine--tRNA ligase OS = | Intracellular |
| | | | | | Streptococcus pneumoniae (strain | |
| | | | | | CGSP14) GN = argS PE = 3 SV = 1 - | |
| | | | | | [SYR_STRPS] | |
| B2INY8 | 20.48 | 9 | 420 | 52.28 | Uncharacterized protein OS = | Intracellular |
| | | | | | Streptococcus pneumoniae (strain | |
| | | | | | CGSP14) GN = SPCG_0813 PE = 4 SV = | |
| | | | | | 1 - [B2INY8_STRPS] | |
| B2IRA3 | 23.52 | 7 | 438 | 52.2 | Pyridine nucleotide-disulfide | Intracellular |
| | | | | | oxidoreductase OS = -Streptococcus | |
| | | | | | pneumoniae (strain CGSP14) | |
| | | | | | GN = merA PE = 3 SV = 1 - | |
| | | | | | [B2IRA3_STRPS] | |
| B2IPW2 | 16.67 | 6 | 330 | 51.71 | Acetoin dehydrogenase, E1 | Intracellular |
| | | | | | component, beta subunit, putative | |
| | | | | | OS = -Streptococcus pneumoniae (strain | |
| | | | | | CGSP14) GN = acoB PE = 4 SV = 1 - | |
| | | | | | [B2IPW2_STRPS] | |
| B2IRU1 | 40.52 | 9 | 232 | 51.05 | Putative N-acetylmannosamine-6- | Intracellular |
| | | | | | phosphate 2-epimerase OS = | |
| | | | | | Streptococcus pneumoniae (strain | |
| | | | | | CGSP14) GN = nanE PE = 3 SV = 1 - | |
| | | | | | [B2IRU1_STRPS] | |
| B2ISY2 | 5.67 | 4 | 705 | 50.76 | Trehalose PTS system, IIABC | Multi- |
| | | | | | components OS = -Streptococcus | transmembrane |
| | | | | | pneumoniae (strain CGSP14) | |
| | | | | | GN = treP PE = 4 SV = 1 - | |
| | | | | | [B2ISY2_STRPS] | |
| B2IRX3 | 29.29 | 10 | 297 | 50.56 | ABC transporter, ATP-binding | Intracellular |
| | | | | | protein OS = -Streptococcus | |
| | | | | | pneumoniae (strain CGSP14) | |
| | | | | | GN = SPCG_1690 PE = 4 SV = 1 - | |
| | | | | | [B2IRX3_STRPS] | |
| B2IQY9 | 24.72 | 10 | 453 | 50.5 | Chromosomal replication initiator | Intracellular |
| | | | | | protein DnaA OS = -Streptococcus | |
| | | | | | pneumoniae (strain CGSP14) | |
| | | | | | GN = dnaA PE = 3 SV = 1 - | |
| | | | | | [DNAA_STRPS] | |
| B2IMI1 | 36.61 | 4 | 183 | 50.13 | Transcription | Intracellular |
| | | | | | termination/antitermination | |
| | | | | | protein NusG OS = -Streptococcus | |
| | | | | | pneumoniae (strain CGSP14) | |
| | | | | | GN = nusG PE = 3 SV = 1 - | |
| | | | | | [B2IMI1_STRPS] | |
| B2ING0 | 66.32 | 5 | 95 | 49.96 | 30S ribosomal protein S16 OS = | Intracellular |
| | | | | | Streptococcus pneumoniae (strain | |
| | | | | | CGSP14) GN = rpsP PE = 3 SV = 1 - | |
| | | | | | [RS16_STRPS] | |
| B2INE1 | 11.85 | 6 | 726 | 48.69 | PTS system, IIABC components OS = | Multi- |
| | | | | | Streptococcus pneumoniae (strain | transmembrane |
| | | | | | CGSP14) GN = ptsG PE = 4 SV = 1 - | |
| | | | | | [B2INE1_STRPS] | |
| B2IMN6 | 33 | 9 | 494 | 48.52 | Threonine synthase OS = | Intracellular |
| | | | | | Streptococcus pneumoniae (strain | |
| | | | | | CGSP14) GN = thrC PE = 4 SV = 1 - | |
| | | | | | [B2IMN6_STRPS] | |
| B2ILR1 | 12.8 | 8 | 719 | 47.81 | Penicillin-binding protein 1A OS = | N- |
| | | | | | Streptococcus pneumoniae (strain | terminally |
| | | | | | CGSP14) GN = pbp1A PE = 4 SV = 1 - | anchored |
| | | | | | [B2ILR1_STRPS] | (No CS) |
| B2IR57 | 22.53 | 8 | 466 | 47.14 | Cof family protein/peptidyl-prolyl | Intracellular |
| | | | | | cis-trans isomerase, cyclophilin | |
| | | | | | type OS = -Streptococcus pneumoniae | |
| | | | | | (strain CGSP14) GN = SPCG_1524 | |
| | | | | | PE = 4 SV = 1 - [B2IR57_STRPS] | |
| B2IRQ1 | 10.57 | 5 | 653 | 46.37 | PTS system IIABC components OS = | Multi- |
| | | | | | Streptococcus pneumoniae (strain | transmembrane |
| | | | | | CGSP14) GN = scrA PE = 4 SV = 1 - | |
| | | | | | [B2IRQ1_STRPS] | |
| B2IRE5 | 23.91 | 5 | 276 | 46.36 | PTS system, IID component OS = | Multi- |
| | | | | | Streptococcus pneumoniae (strain | transmembrane |
| | | | | | CGSP14) GN = SPCG_0064 PE = 4 SV = | |
| | | | | | 1 - [B2IRE5_STRPS] | |
| B2IRX0 | 47.08 | 8 | 240 | 46.21 | ABC transporter, ATP-binding | Intracellular |
| | | | | | protein OS = -Streptococcus | |
| | | | | | pneumoniae (strain CGSP14) | |
| | | | | | GN = SPCG_1687 PE = 4 SV = 1 - | |
| | | | | | [B2IRX0_STRPS] | |
| B2IR19 | 40.73 | 7 | 302 | 45.81 | Amino acid ABC transporter, | Lipid |
| | | | | | periplasmic amino acid-binding | anchored |
| | | | | | protein, putative OS = -Streptococcus | |
| | | | | | pneumoniae (strain CGSP14) | |
| | | | | | GN = SPCG_0108 PE = 4 SV = 1 - | |
| | | | | | [B2IR19_STRPS] | |
| B2IQL3 | 29.76 | 6 | 252 | 45.8 | Phosphate import ATP-binding | Intracellular |
| | | | | | protein PstB OS = -Streptococcus | |
| | | | | | pneumoniae (strain CGSP14) | |
| | | | | | GN = pstB PE = 3 SV = 1 - | |
| | | | | | [B2IQL3_STRPS] | |
| B2IQ98 | 60.64 | 5 | 94 | 45.67 | 50S ribosomal protein L31 type B | Intracellular |
| | | | | | OS = -Streptococcus pneumoniae (strain | |
| | | | | | CGSP14) GN = rpmE PE = 3 SV = 1 - | |
| | | | | | [B2IQ98_STRPS] | |
| B2IRE7 | 15.8 | 6 | 386 | 44.99 | Sugar isomerase domain protein | Intracellular |
| | | | | | AgaS OS = -Streptococcus pneumoniae | |
| | | | | | (strain CGSP14) GN = agaS PE = 4 | |
| | | | | | SV = 1 - [B2IRE7_STRPS] | |
| B2IMZ5 | 21.99 | 9 | 564 | 44.92 | ABC transporter, ATP- | Multi- |
| | | | | | binding/permease protein OS = | transmembrane |
| | | | | | Streptococcus pneumoniae (strain | |
| | | | | | CGSP14) GN = SPCG_2042 PE = 4 SV = | |
| | | | | | 1 - [B2IMZ5_STRPS] | |
| B2IRQ7 | 56.4 | 7 | 172 | 44.31 | Probable thiol peroxidase OS = | Intracellular |
| | | | | | Streptococcus pneumoniae (strain | |
| | | | | | CGSP14) GN = tpx PE = 3 SV = 1 - | |
| | | | | | [B2IRQ7_STRPS] | |
| B2ILX8 | 33.61 | 8 | 488 | 43.22 | Glutamyl-tRNA(Gln) | Intracellular |
| | | | | | amidotransferase subunit A OS = | |
| | | | | | Streptococcus pneumoniae (strain | |
| | | | | | CGSP14) GN = gatA PE = 3 SV = 1 - | |
| | | | | | [GATA_STRPS] | |
| B2IM45 | 29.04 | 8 | 427 | 43.07 | UDP-N-acetylglucosamine 1- | Intracellular |
| | | | | | carboxyvinyltransferase OS = | |
| | | | | | Streptococcus pneumoniae (strain | |
| | | | | | CGSP14) GN = murA PE = 3 SV = 1 - | |
| | | | | | [B2IM45_STRPS] | |
| B2IM60 | 25 | 6 | 416 | 43.06 | Diaminopimelate decarboxylase | Intracellular |
| | | | | | OS = -Streptococcus pneumoniae (strain | |
| | | | | | CGSP14) GN = lysA PE = 3 SV = 1 - | |
| | | | | | [B2IM60_STRPS] | |
| B2IRA1 | 21.95 | 9 | 524 | 42.94 | DEAD-box ATP-dependent RNA | Intracellular |
| | | | | | helicase CshA OS = -Streptococcus | |
| | | | | | pneumoniae (strain CGSP14) | |
| | | | | | GN = cshA PE = 3 SV = 1 - | |
| | | | | | [B2IRA1_STRPS] | |
| B2IR78 | 17.33 | 6 | 450 | 42.72 | Phosphoglucosamine mutase OS = | Intracellular |
| | | | | | Streptococcus pneumoniae (strain | |
| | | | | | CGSP14) GN = glmM PE = 3 SV = 1 - | |
| | | | | | [GLMM_STRPS] | |
| B2IST7 | 22.06 | 7 | 417 | 42.6 | Capsular polysaccharide | Intracellular |
| | | | | | biosynthesis protein, putative OS = | |
| | | | | | Streptococcus pneumoniae (strain | |
| | | | | | CGSP14) GN = SPCG_1812 PE = 3 SV = | |
| | | | | | 1 - [B2IST7_STRPS] | |
| B2INT8 | 57.89 | 2 | 76 | 41.48 | Uncharacterized protein OS = | Intracellular |
| | | | | | Streptococcus pneumoniae (strain | |
| | | | | | CGSP14) GN = SPCG_0763 PE = 4 SV = | |
| | | | | | 1 - [B2INT8_STRPS] | |
| B2IQ61 | 25.95 | 6 | 289 | 41.38 | Choline kinase OS = -Streptococcus | Intracellular |
| | | | | | pneumoniae (strain CGSP14) GN = pck | |
| | | | | | PE = 4 SV = 1 - [B2IQ61_STRPS] | |
| B2ILR6 | 14.66 | 5 | 464 | 41.32 | Mid-cell-anchored protein Z OS = | Multi- |
| | | | | | Streptococcus pneumoniae (strain | transmembrane |
| | | | | | CGSP14) GN = mapZ PE = 3 SV = 1 - | |
| | | | | | [B2ILR6_STRPS] | |
| B2ILR4 | 29.91 | 3 | 117 | 40.9 | Cell cycle protein GpsB OS = | Intracellular |
| | | | | | Streptococcus pneumoniae (strain | |
| | | | | | CGSP14) GN = gpsB PE = 3 SV = 1 - | |
| | | | | | [B2ILR4_STRPS] | |
| B2IQ25 | 28.88 | 8 | 419 | 40.5 | UDP-N-acetylglucosamine 1- | Intracellular |
| | | | | | carboxyvinyltransferase OS = | |
| | | | | | Streptococcus pneumoniae (strain | |
| | | | | | CGSP14) GN = murZ PE = 3 SV = 1 - | |
| | | | | | [B2IQ25_STRPS] | |
| B2IQH8 | 27.11 | 8 | 439 | 40.48 | Homoserine dehydrogenase OS = | Intracellular |
| | | | | | Streptococcus pneumoniae (strain | |
| | | | | | CGSP14) GN = hom PE = 3 SV = 1 - | |
| | | | | | [B2IQH8_STRPS] | |
| B2IN58 | 28.96 | 6 | 335 | 40.08 | Uncharacterized protein OS = | Multi- |
| | | | | | Streptococcus pneumoniae (strain | transmembrane |
| | | | | | CGSP14) GN = SPCG_2101 PE = 4 SV = | |
| | | | | | 1 - [B2IN58_STRPS] | |
| B2IRK5 | 20.21 | 7 | 559 | 40.01 | Ribonuclease J OS = -Streptococcus | Intracellular |
| | | | | | pneumoniae (strain CGSP14) GN = rnj | |
| | | | | | PE = 3 SV = 1 - [B2IRK5_STRPS] | |
| B2INR2 | 26.51 | 10 | 679 | 39.94 | Methionine--tRNA ligase OS = | Intracellular |
| | | | | | Streptococcus pneumoniae (strain | |
| | | | | | CGSP14) GN = metG PE = 3 SV = 1 - | |
| | | | | | [B2INR2_STRPS] | |
| B2IQ29 | 30.5 | 8 | 436 | 39.93 | GTPase Obg OS = -Streptococcus | Intracellular |
| | | | | | pneumoniae (strain CGSP14) | |
| | | | | | GN = obg PE = 3 SV = 1 - [OBG_STRPS] | |
| B2IMY2 | 73.21 | 7 | 56 | 39.82 | Uncharacterized protein OS = | Secretory |
| | | | | | Streptococcus pneumoniae (strain | (released) |
| | | | | | CGSP14) GN = SPCG_0626 PE = 4 SV = | (with CS) |
| | | | | | 1 - [B2IMY2_STRPS] | |
| B2IPW3 | 33.6 | 8 | 375 | 39.59 | Dihydrolipoamide acetyltransferase | Intracellular |
| | | | | | OS = -Streptococcus pneumoniae (strain | |
| | | | | | CGSP14) GN = acoC PE = 3 SV = 1 - | |
| | | | | | [B2IPW3_STRPS] | |
| B2IR18 | 38.5 | 6 | 213 | 39.57 | Amino acid ABC transporter, ATP- | Intracellular |
| | | | | | binding protein, putative OS = | |
| | | | | | Streptococcus pneumoniae (strain | |
| | | | | | CGSP14) GN = SPCG_0107 PE = 4 SV = | |
| | | | | | 1 - [B2IRI8_STRPS] | |
| B2ISR0 | 13.08 | 6 | 535 | 39.53 | Glucan 1,6-alpha-glucosidase OS = | Intracellular |
| | | | | | Streptococcus pneumoniae (strain | |
| | | | | | CGSP14) GN = dexB PE = 4 SV = 1 - | |
| | | | | | [B2ISR0_STRPS] | |
| B2IM72 | 56.91 | 6 | 246 | 39.3 | Amino acid ABC transporter, ATP- | Intracellular |
| | | | | | binding protein OS = -Streptococcus | |
| | | | | | pneumoniae (strain CGSP14) | |
| | | | | | GN = glnQ PE = 4 SV = 1 - | |
| | | | | | [B2IM72_STRPS] | |
| B2IS45 | 48.25 | 3 | 114 | 39.27 | 50S ribosomal protein L22 OS = | Intracellular |
| | | | | | Streptococcus pneumoniae (strain | |
| | | | | | CGSP14) GN = rpIV PE = 3 SV = 1 - | |
| | | | | | [RL22_STRPS] | |
| B2IM26 | 26.1 | 5 | 318 | 39.16 | Autolysin OS = -Streptococcus | Intracellular |
| | | | | | pneumoniae (strain CGSP14) | |
| | | | | | GN = lytA PE = 4 SV = 1 - | |
| | | | | | [B2IM26_STRPS] | |
| B2IM71 | 46.48 | 4 | 71 | 38.4 | Uncharacterized protein OS = | Intracellular |
| | | | | | Streptococcus pneumoniae (strain | |
| | | | | | CGSP14) GN = SPCG_0446 PE = 4 SV = | |
| | | | | | 1 - [B2IM71_STRPS] | |
| B2INE0 | 30.09 | 8 | 329 | 38.01 | Cell division protein FtsX OS = | Multi- |
| | | | | | Streptococcus pneumoniae (strain | transmembrane |
| | | | | | CGSP14) GN = ftsX PE = 3 SV = 1 - | |
| | | | | | [B2INE0_STRPS] | |
| B2IQ19 | 15.99 | 8 | 763 | 37.52 | DNA helicase OS = -Streptococcus | Intracellular |
| | | | | | pneumoniae (strain CGSP14) | |
| | | | | | GN = pcrA PE = 3 SV = 1 - | |
| | | | | | [B2IQ19_STRPS] | |
| B2IRN7 | 21.52 | 6 | 660 | 37.25 | Threonine--tRNA ligase OS = | Intracellular |
| | | | | | Streptococcus pneumoniae (strain | |
| | | | | | CGSP14) GN = thrS PE = 3 SV = 1 - | |
| | | | | | [B2IRN7_STRPS] | |
| B2IS67 | 24.44 | 7 | 311 | 36.89 | DNA-directed RNA polymerase | Intracellular |
| | | | | | subunit alpha OS = -Streptococcus | |
| | | | | | pneumoniae (strain CGSP14) | |
| | | | | | GN = rpoA PE = 3 SV = 1 - | |
| | | | | | [B2IS67_STRPS] | |
| B2IQA4 | 21.65 | 8 | 448 | 36.16 | Glutamate dehydrogenase OS = | Intracellular |
| | | | | | Streptococcus pneumoniae (strain | |
| | | | | | CGSP14) GN = gdhA PE = 3 SV = 1 - | |
| | | | | | [B2IQA4_STRPS] | |
| B2INE5 | 20.96 | 6 | 396 | 36.08 | S-adenosylmethionine synthase | Intracellular |
| | | | | | OS = -Streptococcus pneumoniae (strain | |
| | | | | | CGSP14) GN = metK PE = 3 SV = 1 - | |
| | | | | | [METK_STRPS] | |
| B2IQN3 | 51.52 | 4 | 66 | 35.91 | 30S ribosomal protein S21 OS = | Intracellular |
| | | | | | Streptococcus pneumoniae (strain | |
| | | | | | CGSP14) GN = rpsU PE = 3 SV = 1 - | |
| | | | | | [B2IQN3_STRPS] | |
| B2IRK4 | 18.21 | 8 | 637 | 35.62 | tRNA uridine 5- | Intracellular |
| | | | | | carboxymethylaminomethyl | |
| | | | | | modification enzyme MnmG OS = | |
| | | | | | Streptococcus pneumoniae (strain | |
| | | | | | CGSP14) GN = mnmG PE = 3 SV = 1 - | |
| | | | | | [B2IRK4_STRPS] | |
| B2IR76 | 14.89 | 4 | 282 | 35.33 | DegV family protein OS = | Intracellular |
| | | | | | Streptococcus pneumoniae (strain | |
| | | | | | CGSP14) GN = SPCG_1543 PE = 4 SV = | |
| | | | | | 1 - [B2IR76_STRPS] | |
| B2IRT0 | 25.85 | 4 | 294 | 35.15 | ROK family protein OS = | Intracellular |
| | | | | | Streptococcus pneumoniae (strain | |
| | | | | | CGSP14) GN = SPCG_1647 PE = 4 SV = | |
| | | | | | 1 - [B2IRT0_STRPS] | |
| B2IR21 | 58.62 | 7 | 145 | 35.06 | Uncharacterized protein OS = | Intracellular |
| | | | | | Streptococcus pneumoniae (strain | |
| | | | | | CGSP14) GN = SPCG_0033 PE = 4 SV = | |
| | | | | | 1 - [B2IR21_STRPS] | |
| B2IPF4 | 25.4 | 8 | 311 | 35.02 | Adhesion lipoprotein OS = | Lipid |
| | | | | | Streptococcus pneumoniae (strain | anchored |
| | | | | | CGSP14) GN = Imb PE = 3 SV = 1 - | |
| | | | | | [B2IPF4_STRPS] | |
| B2ILV9 | 26.21 | 6 | 454 | 34.9 | Aspartokinase OS = -Streptococcus | Intracellular |
| | | | | | pneumoniae (strain CGSP14) | |
| | | | | | GN = lysC PE = 3 SV = 1 - | |
| | | | | | [B2ILV9_STRPS] | |
| B2IPP0 | 17.37 | 7 | 495 | 34.88 | Glucose-6-phosphate 1- | Intracellular |
| | | | | | dehydrogenase OS = -Streptococcus | |
| | | | | | pneumoniae (strain CGSP14) GN = zwf | |
| | | | | | PE = 3 SV = 1 - [B2IPP0_STRPS] | |
| B2IRU0 | 8.43 | 5 | 510 | 34.78 | PTS system, IIBC components OS = | Multi- |
| | | | | | Streptococcus pneumoniae (strain | transmembrane |
| | | | | | CGSP14) GN = SPCG_1657 PE = 4 SV = | |
| | | | | | 1 - [B2IRU0_STRPS] | |
| B2IQX1 | 34.93 | 7 | 292 | 34.34 | ATP synthase gamma chain OS = | Intracellular |
| | | | | | Streptococcus pneumoniae (strain | |
| | | | | | CGSP14) GN = atpG PE = 3 SV = 1 - | |
| | | | | | [ATPG_STRPS] | |
| B2ISZ2 | 16.94 | 5 | 490 | 34.05 | Sucrose phosphorylase OS = | Intracellular |
| | | | | | Streptococcus pneumoniae (strain | |
| | | | | | CGSP14) GN = SPCG_1867 PE = 4 SV = | |
| | | | | | 1 - [B2ISZ2_STRPS] | |
| B2IP00 | 28.21 | 6 | 397 | 33.73 | Serine protease OS = -Streptococcus | N- |
| | | | | | pneumoniae (strain CGSP14) | terminally |
| | | | | | GN = sphtra PE = 4 SV = 1 - | anchored |
| | | | | | [B2IP00_STRPS] | (No CS) |
| B2INW0 | 31.82 | 5 | 220 | 33.64 | Deoxyribose-phosphate aldolase | Intracellular |
| | | | | | OS = -Streptococcus pneumoniae (strain | |
| | | | | | CGSP14) GN = deoC PE = 3 SV = 1 - | |
| | | | | | [DEOC_STRPS] | |
| B2IM98 | 15.7 | 6 | 535 | 33.16 | CTP synthase OS = -Streptococcus | Intracellular |
| | | | | | pneumoniae (strain CGSP14) | |
| | | | | | GN = pyrG PE = 3 SV = 1 - | |
| | | | | | [B2IM98_STRPS] | |
| B2IMU0 | 11.8 | 4 | 466 | 33.1 | Dipeptidase OS = -Streptococcus | Intracellular |
| | | | | | pneumoniae (strain CGSP14) | |
| | | | | | GN = pepV PE = 4 SV = 1 - | |
| | | | | | [B2IMU0_STRPS] | |
| B2IPU4 | 37.15 | 5 | 253 | 33.02 | Lactose phosphotransferase system | Intracellular |
| | | | | | repressor OS = -Streptococcus | |
| | | | | | pneumoniae (strain CGSP14) | |
| | | | | | GN = lacR PE = 4 SV = 1 - | |
| | | | | | [B2IPU4_STRPS] | |
| B2IRR9 | 38.55 | 5 | 179 | 32.57 | Cell division protein SepF OS = | Intracellular |
| | | | | | Streptococcus pneumoniae (strain | |
| | | | | | CGSP14) GN = sepF PE = 3 SV = 1 - | |
| | | | | | [SEPF_STRPS] | |
| B2IQJ5 | 27.56 | 5 | 225 | 32.43 | 3-dehydroquinate dehydratase OS = | Intracellular |
| | | | | | Streptococcus pneumoniae (strain | |
| | | | | | CGSP14) GN = aroD PE = 3 SV = 1 - | |
| | | | | | [AROD_STRPS] | |
| B2INC8 | 38.43 | 7 | 216 | 32.37 | Uracil phosphoribosyltransferase | Intracellular |
| | | | | | OS = -Streptococcus pneumoniae (strain | |
| | | | | | CGSP14) GN = upp PE = 3 SV = 1 - | |
| | | | | | [B2INC8_STRPS] | |
| B2INU6 | 33.73 | 8 | 424 | 32.09 | Phosphopentomutase OS = | Intracellular |
| | | | | | Streptococcus pneumoniae (strain | |
| | | | | | CGSP14) GN = deoB PE = 3 SV = 1 - | |
| | | | | | [B2INU6_STRPS] | |
| B2IME1 | 24.87 | 6 | 378 | 32.06 | Transcription | Intracellular |
| | | | | | termination/antitermination | |
| | | | | | protein NusA OS = -Streptococcus | |
| | | | | | pneumoniae (strain CGSP14) | |
| | | | | | GN = nusA PE = 3 SV = 1 - | |
| | | | | | [B2IME1_STRPS] | |
| B2INN5 | 22.79 | 4 | 272 | 31.93 | Cell shape-determining protein | N- |
| | | | | | MreC OS = -Streptococcus pneumoniae | terminally |
| | | | | | (strain CGSP14) GN = mreC PE = 3 | anchored |
| | | | | | SV = 1 - [B2INN5_STRPS] | (No CS) |
| B2IN76 | 12.22 | 6 | 450 | 31.55 | UDP-N-acetylmuramoylalanine--D- | Intracellular |
| | | | | | glutamate ligase OS = -Streptococcus | |
| | | | | | pneumoniae (strain CGSP14) | |
| | | | | | GN = murD PE = 3 SV = 1 - | |
| | | | | | [MURD_STRPS] | |
| B2INF3 | 16.1 | 4 | 267 | 31.17 | Peptidyl-prolyl cis-trans isomerase | Lipid |
| | | | | | OS = -Streptococcus pneumoniae (strain | anchored |
| | | | | | CGSP14) GN = ppiA PE = 3 SV = 1 - | |
| | | | | | [B2INF3_STRPS] | |
| B2IQ22 | 31.82 | 6 | 286 | 30.97 | Methionine aminopeptidase OS = | Intracellular |
| | | | | | Streptococcus pneumoniae (strain | |
| | | | | | CGSP14) GN = map PE = 1 SV = 1 - | |
| | | | | | [B2IQ22_STRPS] | |
| B2IS48 | 70.59 | 5 | 68 | 30.8 | 50S ribosomal protein L29 OS = | Intracellular |
| | | | | | Streptococcus pneumoniae (strain | |
| | | | | | CGSP14) GN = rpmC PE = 3 SV = 1 - | |
| | | | | | [RL29_STRPS] | |
| B2IS10 | 19.57 | 6 | 649 | 30.36 | DNA mismatch repair protein MutL | Intracellular |
| | | | | | OS = -Streptococcus pneumoniae (strain | |
| | | | | | CGSP14) GN = mutL PE = 3 SV = 1 - | |
| | | | | | [MUTL_STRPS] | |
| B2IM44 | 29.93 | 6 | 274 | 30.16 | DNA-entry nuclease OS = | N- |
| | | | | | Streptococcus pneumoniae (strain | terminally |
| | | | | | CGSP14) GN = endA PE = 4 SV = 1 - | anchored |
| | | | | | [B2IM44_STRPS] | (No CS) |
| B2INF7 | 54.46 | 5 | 112 | 29.66 | ATP cone domain-containing | Intracellular |
| | | | | | protein OS = -Streptococcus | |
| | | | | | pneumoniae (strain CGSP14) | |
| | | | | | GN = SPCG_0728 PE = 4 SV = 1 - | |
| | | | | | [B2INF7_STRPS] | |
| B2IS34 | 13.3 | 7 | 737 | 29.66 | Anaerobic ribonucleoside | Intracellular |
| | | | | | triphosphate reductase OS = | |
| | | | | | Streptococcus pneumoniae (strain | |
| | | | | | CGSP14) GN = SPCG_0212 PE = 4 SV = | |
| | | | | | 1 - [B2IS34_STRPS] | |
| B2IN20 | 40.78 | 3 | 103 | 29.58 | Uncharacterized protein OS = | Intracellular |
| | | | | | Streptococcus pneumoniae (strain | |
| | | | | | CGSP14) GN = SPCG_2067 PE = 4 SV = | |
| | | | | | 1 - [B2IN20_STRPS] | |
| B2IMB7 | 38.95 | 4 | 95 | 29.51 | Uncharacterized protein OS = | Intracellular |
| | | | | | Streptococcus pneumoniae (strain | |
| | | | | | CGSP14) GN = SPCG_0492 PE = 4 SV = | |
| | | | | | 1 - [B2IMB7_STRPS] | |
| B2IMY9 | 20.58 | 6 | 486 | 29.3 | Glutamate--tRNA ligase OS = | Intracellular |
| | | | | | Streptococcus pneumoniae (strain | |
| | | | | | CGSP14) GN = gltX PE = 3 SV = 1 - | |
| | | | | | [SYE_STRPS] | |
| B2IQ75 | 46.77 | 5 | 186 | 29.27 | LemA protein OS = -Streptococcus | N- |
| | | | | | pneumoniae (strain CGSP14) | terminally |
| | | | | | GN = lemA PE = 4 SV = 1 - | anchored |
| | | | | | [B2IQ75_STRPS] | (No CS) |
| B21PN9 | 20.75 | 6 | 429 | 29.19 | Signal recognition particle receptor | Intracellular |
| | | | | | FtsY OS = -Streptococcus pneumoniae | |
| | | | | | (strain CGSP14) GN = ftsY PE = 3 SV = | |
| | | | | | 1 - [B2IPN9_STRPS] | |
| B2INM2 | 45.33 | 7 | 150 | 28.94 | 50S ribosomal protein L9 OS = | Intracellular |
| | | | | | Streptococcus pneumoniae (strain | |
| | | | | | CGSP14) GN = rplI PE = 3 SV = 1 - | |
| | | | | | [RL9_STRPS] | |
| B2IR80 | 20.89 | 5 | 292 | 28.84 | Uncharacterized protein OS = | Multi- |
| | | | | | Streptococcus pneumoniae (strain | transmembrane |
| | | | | | CGSP14) GN = SPCG_1547 PE = 4 SV = | |
| | | | | | 1 - [B2IR80_STRPS] | |
| B2IPW9 | 32.16 | 8 | 283 | 28.64 | Ribosome biogenesis GTPase A OS = | Intracellular |
| | | | | | Streptococcus pneumoniae (strain | |
| | | | | | CGSP14) GN = SPCG_1141 PE = 3 SV = | |
| | | | | | 1 - [B2IPW9_STRPS] | |
| B2IN61 | 75.51 | 4 | 49 | 28.27 | 50S ribosomal protein L33 OS = | Intracellular |
| | | | | | Streptococcus pneumoniae (strain | |
| | | | | | CGSP14) GN = rpmG PE = 3 SV = 1 - | |
| | | | | | [RL33_STRPS] | |
| B2IM15 | 25.95 | 5 | 158 | 28.26 | Uncharacterized protein OS = | Multi- |
| | | | | | Streptococcus pneumoniae (strain | transmembrane |
| | | | | | CGSP14) GN = SPCG_1901 PE = 4 SV = | |
| | | | | | 1 - [B2IM15_STRPS] | |
| B2IPZ5 | 13.9 | 7 | 633 | 28.19 | ABC transporter, ATP-binding | Intracellular |
| | | | | | protein OS = -Streptococcus | |
| | | | | | pneumoniae (strain CGSP14) | |
| | | | | | GN = SPCG_1167 PE = 4 SV = 1 - | |
| | | | | | [B2IPZ5_STRPS] | |
| B2IMA9 | 20.94 | 6 | 487 | 28.12 | Type I restriction-modification | Intracellular |
| | | | | | system, M subunit OS = | |
| | | | | | Streptococcus pneumoniae (strain | |
| | | | | | CGSP14) GN = hsdM PE = 4 SV = 1 - | |
| | | | | | [B2IMA9_STRPS] | |
| B2IQ67 | 11.63 | 7 | 1058 | 28.04 | Carbamoyl-phosphate synthase | Intracellular |
| | | | | | large chain OS = -Streptococcus | |
| | | | | | pneumoniae (strain CGSP14) GN = carB | |
| | | | | | PE = 3 SV = 1 - [CARB_STRPS] | |
| B2IMV8 | 16.5 | 4 | 491 | 28.03 | PTS system, IIC component, | Multi- |
| | | | | | putative OS = -Streptococcus | transmembrane |
| | | | | | pneumoniae (strain CGSP14) | |
| | | | | | GN = SPCG_0602 PE = 4 SV = 1 - | |
| | | | | | [B2IMV8_STRPS] | |
| B2INP6 | 15.54 | 4 | 341 | 28 | Tryptophan--tRNA ligase OS = | Intracellular |
| | | | | | Streptococcus pneumoniae (strain | |
| | | | | | CGSP14) GN = trpS PE = 3 SV = 1 - | |
| | | | | | [B2INP6_STRPS] | |
| B2INU9 | 26.02 | 4 | 269 | 27.83 | Purine nucleoside phosphorylase | Intracellular |
| | | | | | OS = -Streptococcus pneumoniae (strain | |
| | | | | | CGSP14) GN = pnp PE = 3 SV = 1 - | |
| | | | | | [B2INU9_STRPS] | |
| B2IMS9 | 20.8 | 7 | 553 | 27.26 | Ribonuclease J OS = -Streptococcus | Intracellular |
| | | | | | pneumoniae (strain CGSP14) GN = rnj | |
| | | | | | PE = 3 SV = 1 - [B2IMS9_STRPS] | |
| B2ILY3 | 41.94 | 4 | 62 | 27.17 | 50S ribosomal protein L28 OS = | Intracellular |
| | | | | | Streptococcus pneumoniae (strain | |
| | | | | | CGSP14) GN = rpmB PE = 3 SV = 1 - | |
| | | | | | [RL28_STRPS] | |
| B2IRW8 | 34.46 | 5 | 177 | 26.92 | Transcriptional repressor NrdR OS = | Intracellular |
| | | | | | Streptococcus pneumoniae (strain | |
| | | | | | CGSP14) GN = nrdR PE = 3 SV = 1 - | |
| | | | | | [B2IRW8_STRPS] | |
| B2IMH1 | 20.97 | 7 | 472 | 26.92 | Cof family protein OS = -Streptococcus | Intracellular |
| | | | | | pneumoniae (strain CGSP14) | |
| | | | | | GN = SPCG_1962 PE = 4 SV = 1 - | |
| | | | | | [B2IMH1_STRPS] | |
| B21PM 6 | 13.79 | 2 | 87 | 26.87 | Phosphocarrier protein HPr OS = | Intracellular |
| | | | | | Streptococcus pneumoniae (strain | |
| | | | | | CGSP14) GN = SPCG_1120 PE = 4 SV = | |
| | | | | | 1 [B2IPM6_STRPS] | |
| B2IRB7 | 27.22 | 2 | 158 | 26.84 | Uncharacterized protein OS = | N- terminally |
| | | | | | Streptococcus pneumoniae (strain | anchored |
| | | | | | CGSP14) GN = SPCG_1584 PE = 4 SV = | (No CS) |
| | | | | | 1 - [B2IRB7_STRPS] | |
| B2ILW4 | 23.46 | 5 | 324 | 26.69 | Enoyl-(Acyl-carrier-protein) | Intracellular |
| | | | | | reductase OS = -Streptococcus | |
| | | | | | pneumoniae (strain CGSP14) | |
| | | | | | GN = fabK PE = 4 SV = 1 - | |
| | | | | | [B2ILW4_STRPS] | |
| B2IN41 | 16.32 | 7 | 429 | 26.52 | Histidine -- tRNA ligase OS = | Intracellular |
| | | | | | Streptococcus pneumoniae (strain | |
| | | | | | CGSP14) GN = hisS PE = 3 SV = 1 - | |
| | | | | | [SYH_STRPS] | |
| B2IQU2 | 16.07 | 3 | 305 | 26.2 | Glycine -- tRNA ligase alpha subunit | Intracellular |
| | | | | | OS = -Streptococcus pneumoniae (strain | |
| | | | | | CGSP14) GN = glyQ PE = 3 SV = 1 - | |
| | | | | | [SYGA_STRPS] | |
| B2IPW1 | 23.91 | 4 | 322 | 26.19 | Acetoin dehydrogenase, E1 | Intracellular |
| | | | | | component, alpha subunit, putative | |
| | | | | | OS = -Streptococcus pneumoniae (strain | |
| | | | | | CGSP14) GN = acoA PE = 4 SV = 1 - | |
| | | | | | [B2IPW1_STRPS] | |
| B2ISG1 | 25.79 | 5 | 190 | 26.04 | Uncharacterized protein OS = | Multi- |
| | | | | | Streptococcus pneumoniae (strain | transmembrane |
| | | | | | CGSP14) GN = SPCG_1779 PE = 4 SV = | |
| | | | | | 1 - [B2ISG1_STRPS] | |
| B2IPZ7 | 19.35 | 4 | 279 | 25.42 | DegV family protein OS = | Intracellular |
| | | | | | Streptococcus pneumoniae (strain | |
| | | | | | CGSP14) GN = SPCG_1169 PE = 4 SV = | |
| | | | | | 1 - [B2IPZ7_STRPS] | |
| B2ISK9 | 13.48 | 4 | 267 | 25.25 | PTS system, mannose-specific IIC | Multi- |
| | | | | | component OS = -Streptococcus | transmembrane |
| | | | | | pneumoniae (strain CGSP14) | (Lipid |
| | | | | | GN = manM PE = 4 SV = 1 - | modified |
| | | | | | [B2ISK9_STRPS] | N-termini) |
| B2IS70 | 20.32 | 5 | 433 | 25.16 | UPF0210 protein SPCG_0246 OS = | Intracellular |
| | | | | | Streptococcus pneumoniae (strain | |
| | | | | | CGSP14) GN = SPCG_0246 PE = 3 SV = | |
| | | | | | 1 - [B2IS70_STRPS] | |
| B2INL6 | 15.22 | 4 | 335 | 24.97 | ABC transporter, substrate-binding | Lipid |
| | | | | | protein, putative OS = -Streptococcus | anchored |
| | | | | | pneumoniae (strain CGSP14) | |
| | | | | | GN = SPCG_2165 PE = 4 SV = 1 - | |
| | | | | | [B2INL6_STRPS] | |
| B2IPF2 | 46.6 | 4 | 191 | 24.92 | Thioredoxin family protein OS = | Lipid |
| | | | | | Streptococcus pneumoniae (strain | anchored |
| | | | | | CGSP14) GN = SPCG_0974 PE = 4 SV = | |
| | | | | | 1 - [B2IPF2_STRPS] | |
| B2INJ2 | 24.59 | 7 | 427 | 24.82 | DItD protein OS = -Streptococcus | N- |
| | | | | | pneumoniae (strain CGSP14) | terminally |
| | | | | | GN = dltD PE = 4 SV = 1 - | anchored |
| | | | | | [B2INJ2_STRPS] | (No CS) |
| B2IPH6 | 13.65 | 3 | 359 | 24.36 | Peptide chain release factor 1 OS = | Intracellular |
| | | | | | Streptococcus pneumoniae (strain | |
| | | | | | CGSP14) GN = prfA PE = 3 SV = 1 - | |
| | | | | | [RF1_STRPS] | |
| B2ILY0 | 20.43 | 4 | 514 | 24.35 | Peptide chain release factor 3 OS = | Intracellular |
| | | | | | Streptococcus pneumoniae (strain | |
| | | | | | CGSP14) GN = prfC PE = 3 SV = 1 - | |
| | | | | | [RF3_STRPS] | |
| B2ILV5 | 20.88 | 4 | 182 | 24.04 | Alkyl hydroperoxide reductase | Intracellular |
| | | | | | AhpD OS = -Streptococcus pneumoniae | |
| | | | | | (strain CGSP14) GN = mip PE = 3 SV = | |
| | | | | | 1 - [B2ILV5_STRPS] | |
| B2IRX2 | 21.8 | 4 | 399 | 23.82 | Uncharacterized protein OS = | Multi- |
| | | | | | Streptococcus pneumoniae (strain | transmembrane |
| | | | | | CGSP14) GN = SPCG_1689 PE = 4 SV = | |
| | | | | | 1 - [B2IRX2_STRPS] | |
| B2IM58 | 11.36 | 4 | 308 | 23.79 | Membrane protein insertase YidC | Multi- |
| | | | | | OS = -Streptococcus pneumoniae (strain | transmembrane |
| | | | | | CGSP14) GN = yidC PE = 3 SV = 1 - | |
| | | | | | [B2IM58_STRPS] | |
| B2IR27 | 23.03 | 4 | 330 | 23.79 | Phosphate acyltransferase OS = | Intracellular |
| | | | | | Streptococcus pneumoniae (strain | |
| | | | | | CGSP14) GN = plsX PE = 3 SV = 1 - | |
| | | | | | [PLSX_STRPS] | |
| B2IQL7 | 22.95 | 4 | 292 | 23.74 | Phosphate ABC transporter, | Lipid |
| | | | | | phosphate-binding protein, | anchored |
| | | | | | putative OS = -Streptococcus | |
| | | | | | pneumoniae (strain CGSP14) | |
| | | | | | GN = SPCG_1389 PE = 4 SV = 1 - | |
| | | | | | [B2IQL7_STRPS] | |
| B2IR86 | 41.62 | 7 | 197 | 23.54 | Probable GTP-binding protein EngB | Intracellular |
| | | | | | OS = -Streptococcus pneumoniae (strain | |
| | | | | | CGSP14) GN = engB PE = 3 SV = 1 - | |
| | | | | | [ENGB_STRPS] | |
| B2IP77 | 21 | 5 | 419 | 23.53 | Uncharacterized protein OS = | Intracellular |
| | | | | | Streptococcus pneumoniae (strain | |
| | | | | | CGSP14) GN = SPCG_0896 PE = 4 SV = | |
| | | | | | 1 - [B2IP77_STRPS] | |
| B2ISL2 | 23.6 | 5 | 339 | 23.5 | Alcohol dehydrogenase OS = | Intracellular |
| | | | | | Streptococcus pneumoniae (strain | |
| | | | | | CGSP14) GN = adhP PE = 3 SV = 1 - | |
| | | | | | [B2ISL2_STRPS] | |
| B2IPR3 | 21.05 | 5 | 247 | 23.35 | Uncharacterized protein OS = | N- |
| | | | | | Streptococcus pneumoniae (strain | terminally |
| | | | | | CGSP14) GN = SPCG_1083 PE = 4 SV = | anchored |
| | | | | | 1 - [B2IPR3_STRPS] | (with CS) |
| B2IR97 | 25 | 6 | 252 | 23.12 | Methyltransferase, putative OS = | Intracellular |
| | | | | | Streptococcus pneumoniae (strain | |
| | | | | | CGSP14) GN = SPCG_1564 PE = 4 SV = | |
| | | | | | 1 - [B2IR97_STRPS] | |
| B2IS98 | 24.4 | 4 | 209 | 22.81 | Probable nicotinate-nucleotide | Intracellular |
| | | | | | adenylyltransferase OS = | |
| | | | | | Streptococcus pneumoniae (strain | |
| | | | | | CGSP14) GN = nadD PE = 3 SV = 1 - | |
| | | | | | [B2IS98_STRPS] | |
| B2IQS3 | 22.11 | 4 | 303 | 22.58 | Thioredoxin reductase OS = | Intracellular |
| | | | | | Streptococcus pneumoniae (strain | |
| | | | | | CGSP14) GN = trxB PE = 3 SV = 1 - | |
| | | | | | [B2IQS3_STRPS] | |
| B21PW5 | 31.61 | 6 | 329 | 22.56 | Lipoate-protein ligase, putative OS = | Intracellular |
| | | | | | Streptococcus pneumoniae (strain | |
| | | | | | CGSP14) GN = SPCG_1137 PE = 4 SV = | |
| | | | | | 1 - [B2IPW5_STRPS] | |
| B2IMB4 | 30.77 | 5 | 182 | 22.24 | Protein GrpE OS = -Streptococcus | Intracellular |
| | | | | | pneumoniae (strain CGSP14) | |
| | | | | | GN = grpE PE = 3 SV = 1 - | |
| | | | | | [B2IMB4_STRPS] | |
| B2ILY4 | 43.8 | 3 | 121 | 22.23 | Uncharacterized protein OS = | Intracellular |
| | | | | | Streptococcus pneumoniae (strain | |
| | | | | | CGSP14) GN = asp23 PE = 4 SV = 1 - | |
| | | | | | [B2ILY4_STRPS] | |
| B2IPH3 | 18.37 | 7 | 479 | 22.18 | tRNA modification GTPase MnmE | Intracellular |
| | | | | | OS = -Streptococcus pneumoniae (strain | |
| | | | | | CGSP14) GN = thdF PE = 3 SV = 1 - | |
| | | | | | [B2IPH3_STRPS] | |
| B2INP7 | 17.59 | 6 | 540 | 22.16 | ABC transporter, ATP-binding | Intracellular |
| | | | | | protein OS = -Streptococcus | |
| | | | | | pneumoniae (strain CGSP14) | |
| | | | | | GN = SPCG_2196 PE = 4 SV = 1 - | |
| | | | | | [B2INP7_STRPS] | |
| B2IP74 | 14.66 | 5 | 491 | 21.9 | Lysine decarboxylase OS = | Intracellular |
| | | | | | Streptococcus pneumoniae (strain | |
| | | | | | CGSP14) GN = cad PE = 4 SV = 1 - | |
| | | | | | [B2IP74_STRPS] | |
| B2ISG4 | 52.24 | 2 | 67 | 21.88 | Uncharacterized protein OS = | Intracellular |
| | | | | | Streptococcus pneumoniae (strain | |
| | | | | | CGSP14) GN = SPCG_1782 PE = 3 SV = | |
| | | | | | 1 - [B2ISG4_STRPS] | |
| B2ISY1 | 17.74 | 7 | 541 | 21.79 | Dextran glucosidase DexS, putative | Intracellular |
| | | | | | OS = -Streptococcus pneumoniae (strain | |
| | | | | | CGSP14) GN = dexS PE = 4 SV = 1 - | |
| | | | | | [B2ISY1_STRPS] | |
| B2IMA5 | 15.4 | 5 | 448 | 21.67 | Glutamine synthetase, type I OS = | Intracellular |
| | | | | | Streptococcus pneumoniae (strain | |
| | | | | | CGSP14) GN = gInA PE = 3 SV = 1 - | |
| | | | | | [B2IMA5_STRPS] | |
| B2IN77 | 24.72 | 6 | 352 | 21.61 | UDP-N-acetylglucosamine -- N- | Intracellular |
| | | | | | acetylmuramyl-(pentapeptide) | |
| | | | | | pyrophosphoryl-undecaprenol N- | |
| | | | | | acetylglucosamine transferase OS = | |
| | | | | | Streptococcus pneumoniae (strain | |
| | | | | | CGSP14) GN = murG PE = 3 SV = 1 - | |
| | | | | | [MURG_STRPS] | |
| B2IQ12 | 25.39 | 5 | 319 | 21.48 | Ribose-phosphate | Intracellular |
| | | | | | pyrophosphokinase OS = | |
| | | | | | Streptococcus pneumoniae (strain | |
| | | | | | CGSP14) GN = prs PE = 3 SV = 1 - | |
| | | | | | [B2IQ12_STRPS] | |
| B2ISL4 | 16.3 | 4 | 270 | 21.32 | Cof family protein OS = -Streptococcus | Intracellular |
| | | | | | pneumoniae (strain CGSP14) | |
| | | | | | GN = SPCG_0299 PE = 4 SV = 1 - | |
| | | | | | [B2ISL4_STRPS] | |
| B2IQ78 | 13.38 | 5 | 523 | 21.2 | Signal recognition particle protein | Intracellular |
| | | | | | OS = -Streptococcus pneumoniae (strain | |
| | | | | | CGSP14) GN = ffh PE = 3 SV = 1 - | |
| | | | | | [B2IQ78_STRPS] | |
| B2IS71 | 21.69 | 5 | 249 | 21.06 | Phosphoglycerate mutase family | Intracellular |
| | | | | | protein OS = -Streptococcus | |
| | | | | | pneumoniae (strain CGSP14) | |
| | | | | | GN = gpmB PE = 4 SV = 1 - | |
| | | | | | [B2IS71_STRPS] | |
| B2IM30 | 15.38 | 5 | 338 | 20.64 | Transcriptional regulator, putative | Secretory) |
| | | | | | OS = -Streptococcus pneumoniae | (released |
| | | | | | (strain CGSP14) GN = lytR PE = 4 SV = | (with CS) |
| | | | | | 1 - [B2IM30_STRPS] | |
| B2IQN9 | 10.91 | 4 | 486 | 20.01 | Nicotinate | Intracellular |
| | | | | | phosphoribosyltransferase OS = | |
| | | | | | Streptococcus pneumoniae (strain | |
| | | | | | CGSP14) GN = SPCG_1408 PE = 3 SV = | |
| | | | | | 1 - [B2IQN9_STRPS] | |

### Example 2: Microparticles (MPs) Confer Serotype-Independent Protective Immunity

For each immunization experiment male C57BL/6 wild-type mice ca 5 weeks old were used. Before immunization, mice were anesthetized by inhalation of isofluorane (Abbott) and then intranasally administered with 50 μl/mouse of microparticles (MP) combined with the adjuvant aluminium hydroxide (Sigma Aldrich, 10 mg/ml in PBS) or the adjuvant alone for the control groups. Immunization was repeated again after two weeks from the first immunization following the same conditions described above. After 4 weeks of immunization, mice were infected by intranasal administration of 50 μl/mouse of 5×10⁶ CFU, for S. pneumoniae type 1 infection experiments (FIG. 7), or 10⁶ CFU, for S. pneumoniae type 3 infection experiments (FIG. 11).

All mice were anesthetized by inhalation of isofluorane prior to challenge with bacteria. After the infection, clinical symptoms of the mice were monitored multiple times per day (in accordance with the ethical permit). Blood samples (5 μl/mouse) were taken every day of infection and level of bacteremia was assessed by plating serial dilutions of blood samples onto blood-agar plates. Mice that reached humane end-points were anesthetized by inhalation of isofluorane and intranasal administered with 100 μl/mouse of the fluorescent marker Bacterisense 645 (Perkin Elmer) 30 minutes before the sacrifice. Prior to sacrifice, mice were anesthetized again (isofluorane inhalation). After sacrifice, mice were placed in the IVIS Spectrum Imaging System to detect the fluorescent signal of the bacterial infection. After the IVIS imaging, lungs and spleens were collected for further analysis. Bacterial amount in the lungs was assessed by CFU count of bacteria after plating serial dilutions of lung homogenates onto blood-agar plates.

Intranasal immunization with microparticles (MP) from a serotype 4 S. pneumoniae increased survival after a challenge with serotype 1 S. pneumoniae (FIG. 7A). The increase in survival was significant also in comparison to immunization with MV_{L}. The immunization greatly reduced the bacterial load in the lungs of the mice surviving the challenge with serotype 1 S. pneumoniae (FIG. 7B). Furthermore, mice immunized with serotype 3 MP and then challenged with serotype 3 bacteria showed 100% protection in the pneumonia model, higher than immunization with the currently available vaccine PCV13, which only conferred a protection of 75% in the model. Moreover, protection against pneumococcal infection was absent in mice lacking B cells, suggesting the involvement of an adaptive immune response with anti-pneumococcal antibodies (FIG. 11).

### Example 3: Serotype-Independent Binding and Opsonophagocitic Activity of Antibodies Raised Against Pneumococcal Membrane Particles

Antibodies raised against the inventive MP from serotype 4 as antigen were reactive against Streptococcus pneumoniae of other serotypes, such as 1, 6B and 3 (FIG. 9). In other words, the MP were able to elicit production of serotype-independent antibodies, much more so than MV_{L} (FIG. 9B).

The production of anti-MP antibodies in mice was more efficient (in particular at time point 2 weeks) when the antigen was administered together with an adjuvant. However, satisfactory results were obtainable even without an adjuvant (FIG. 8). Moreover, antibodies in sera were able to bind both the encapsulated T4 (TIGR4) and the unencapsulated isogenic mutant T4R to the same extent, indicating that the antibody response was capsule-independent.

Importantly, the anti-MP antibodies showed opsonophagocitic activity, increasing adhesion of bacteria and killing of internalized bacteria by RAW murine macrophages (FIG. 10).

### Materials and Methods

Bacterial strains and growth conditions. In this study six strains of Streptococcus pneumoniae were used: TIGR4 or T4 (serotype 4) (22), its isogenic mutants lacking the cytotoxin pneumolysin (T4Δply) (23), the autolysin LytA (T4ΔlytA) (3), or the capsule (T4R) (24), ATCC 6301 (serotype 1) and 1-33 (serotype 3). Bacteria were grown in C+Y medium pH 7.9-8.0 (for serotype 4) or THY medium (for serotype 1 and 3) at 37° C. A spectrophotometer (Genesys 20, Thermo Spectronic) was used to follow the growth by monitoring the optical density (OD) at 600 nm.

Isolation and purification of membrane vesicles (MV) and microparticles (MP). For isolation of MV_{L} from a liquid culture, pneumococcal strains (TIGR4, T4Δply or T4ΔlytA) were grown at 37° C. in C+Y medium, pH 7.9-8.0 until OD_{600 nm}=0.9. The culture was then centrifuged (17,000×g for 30 minutes at 4° C.) to remove bacterial cells from the supernatant. The cell-free supernatant was filtered through a 0.22 μm filter (Sarstedt) and centrifuged (120,000×g for 2 hours at 4° C.) to sediment the vesicles. Pellets were washed twice in phosphate-buffered saline (PBS) and resuspended in PBS.

For isolation of microparticles (MP) from bacteria grown on plates, the pneumococcal strains were streaked on blood agar plates and incubated overnight at 37° C. with 5% CO₂. Bacteria were harvested from plates, resuspended in PBS and MP were pelleted following the same procedure as mentioned above.

Crude MV_{L}/MP preparations were further purified by density gradient centrifugation using Optiprep™ Density Gradient Medium (Sigma). Pelleted particle fractions were adjusted to 50% (w/v) Optiprep™ in a total volume of 2 ml and overlayed with one fraction of 30% (w/v) Optiprep™ (9 ml) followed by a fraction of 5% (w/v) Optiprep™ (3 ml). Gradients were centrifuged at 250,000×g for 3 hours at 4° C. and the first 4 ml on top, containing the particles, were collected. After 3 washes with PBS (250,000×g for 2 hours at 4° C.), pellets were recovered in PBS and stored at −80° C.

Electron microscopy. To visualize MV_{L} and MP on bacteria, S. pneumoniae T4R was grown in C+Y medium until OD₆₀₀=0.4 at 37° C. Bacteria were harvested by centrifugation for 10 min at 4,000×g, 4° C. and pellets were suspended in 100 μl PBS. Glow discharged carbon coated grids (Oxford Instruments, UK) were incubated for 1 min with a drop of bacterial solution or purified MV_{L}/MP preparation and negatively stained with 2% uranyl acetate in water (7 times for 10 sec). Specimens were examined on a FEI CM120 microscope operated at 80 kV. Images were collected with a side mounted camera MegaView III (Olympus Soft Imaging solutions).

Atomic force microscopy. 5 μl of MP samples isolated from S. pneumoniae were placed onto freshly cleaved mica (Goodfellow Cambridge Ltd., Cambridge, United Kingdom). The specimens on the mica were blot dried and placed into a desiccator for at least 2 h. Imaging was performed on a Nanoscope IIIa (Digital Instruments, Santa Barbara) Atomic Force Microscope using Tapping Mode with standard silicon cantilevers oscillating at resonant frequency (270 to 305 kHz). Images were collected at a scan rate of 0.8-1.5 Hz, depending on sample number and the size of the scan. The final images were fitted in both axes and presented in a surface plot of the height mode.

SDS-PAGE and western blotting. The total amount of proteins in purified MV_{L} or MP, lysates of A549 cells or dendritic cells stimulated with MV_{L} or MP were quantified with Pierce™ BCA Protein Assay Kit (Life Technologies). Samples containing equal amounts of total protein were resolved by SDS-PAGE using 4-12% Bis-Tris gels (Life Technologies) and transferred to PVDF membranes. Membranes were then blocked with 5% skim milk in PBS containing 0.1% Tween-20 and incubated with antibodies as indicated. For detection of pneumolysin a mouse monoclonal antibody (Abcam, final dilution 1:500) was used. Polyclonal GAPDH (1:2,000) and LytA (1:2,000) (3) antisera raised in rabbits were used. Rabbit polyclonal antibodies against PspC (1:1,000), RrgB (1:1,000), PsaA (1:25,000), PhtD (1:25,000) and mouse polyclonal antibodies against SrtA (1:500) were kindly provided by Novartis Vaccines and Diagnostics. As loading control for A549 cells and dendritic cell lysates, a mouse monoclonal β-actin antibody (Santa Cruz) was used as primary antibody. Anti-mouse IgG or anti-rabbit IgG conjugated to horseradish peroxidase (GE Healhcare) were used as secondary antibodies (final dilution 1:10,000). Blots were developed with Amersham™ ECL Plus Western blotting detection system (GE Healthcare Life Sciences), using a ChemiDoc™ XRS+ (Bio-Rad Laboratories).

Tandem mass spectrometry. Proteins in MV_{L} or MP samples were reduced, alkylated and in-solution digested by trypsin according to a standard operating procedure. Thereafter the samples were purified by Pierce C18 Spin Columns (Thermo Scientific), dried and resolved in 0.1% formic acid. The resulting peptides were separated in reversed-phase on a C18-column and electrosprayed on-line to a Q Exactive Plus mass spectrometer (Thermo Finnigan). Tandem mass spectrometry was performed applying HCD.

Database searches were made using the Sequest algorithm towards a FASTA database including proteins from Streptococcus pneumoniae TIGR4. The search criteria for protein identification were set to at least two matching peptides of 95% confidence level per protein. Only proteins with a Sequest score above 20 were considered for analysis, to avoid the possibility of false positives.

Subcellular localizations of proteins were predicted using the algorithm of website Locate P located at the following URL: www.cmbi.ru.nl/locatep-db/cgi-bin/locatepdb.py (11).

Hemolysis assay. Purified MV_{L} or MP were incubated in 96-well plates with blood from buffy coats (diluted 1:50 in PBS containing 1 mM dithiothreitol (DTT)) for 1 hour at 37° C. After 50 minutes 1% Triton X-100 in PBS was added to the positive control wells in order to lyse all the erythrocytes. Plates were then spun at 400×g for 15 minutes at 4° C.; supernatant was transferred to an optical plate and the optical density was measured at 540 nm.

A549 cell toxicity assay. A459 lung epithelial cells were grown and maintained at 37° C., with 5% CO₂ in RPMI medium (Gibco) supplemented with 10% (v/v) Fetal bovine serum (FBS) (HyClone). To assess MV_{L} or MP associated cytotoxic effects, 0.6×10⁶ A549 cells were seeded in 6-well plates and incubated overnight at 37° C. Cells were then washed with PBS and incubated for 24 hours with medium containing MV_{L} or MP at indicated concentrations. Washed cells were labelled with Fixable Viability Dye eFluor® 780 (1:50,000, eBioscience) for 30 minutes at 4° C. in the dark, in presence or absence of 0.02% NP40 (Sigma) as positive control, and fixed with 4% paraformaldehyde (PFA) for 30 minutes at room temperature. Next, cells were gently scraped into PBS containing 1% FBS and analyzed in a Gallios™ Flow Cytometer (Beckman Coulter).

A549 cell uptake assay. To assess MV_{L} and MP uptake by A549 cells via immunoblotting, 0.6×10⁶ A549 cells were seeded in 6-well plates and incubated overnight at 37° C. Cells were then washed with PBS and incubated for 24 hours with medium containing the particles at indicated concentrations. After washing, cells were lysed in RIPA buffer containing 1× protease inhibitor cocktail (Roche). Cell lysates were analyzed by SDS-PAGE and western blotting.

Immunofluorescence microscopy. A549 cells (6.25×10⁴) were seeded in 24-well plates with coverslips on the bottom of the wells, and incubated at 37° C. overnight. Cells were then washed, fixed with 4% PFA for 30 minutes and permeabilized with 1% Triton X-100 in PBS for 5 minutes. MV_{L} or MP were detected with mouse monoclonal anti-Ply (1:200) and polyclonal rabbit antibodies to LytA (1:200). Respective antigens were visualized with Alexa Fluor® 488-conjugated goat-anti mouse IgG antibody (Life Technologies) and Alexa Fluor® 350-conjugated goat-anti rabbit IgG (Life Technologies) (1:1,000).

Actin cytoskeleton was stained with Alexa Fluor® 594 Phalloidin (Life Technologies, 1:40 dilution) for 1 hour and coverslips were mounted with Vectashield (Vector Laboratories, Inc.) on microscope slides. Images were acquired with a DeltaVision microscope equipped with a 60x-objective. Quick projection images of approximately 20 z-stacks were taken. Orthogonal views were used to visualize the cell monolayer from the x, y, and z axes.

After growth, pneumococci (serotypes 1, 3 and 6B) have been stained using the sera from the immunized mice as primary antibody (dilution 1:100 in PBS 1% BSA) and Alexa Fluor 488 goat anti mouse (dilution 1:500 in PBS 1% BSA) as secondary antibody.

Imaging has been performed with high-resolution Delta Vision Elite System using 100×objective (1000× total magnification). FITC Laser intensity 50%, exposure time 0,025 ms (FIGS. 13-15).

Quantification of the Signal Detected on the Bacteria after Immunofluorescence Staining

Using the functions Image>Adjust>Threshold and Analyze>Measure of ImageJ, the area covered by the bacteria and the area covered by the signal detected on the bacteria after staining with sera were selected, defined and measured. The final signal ratio was calculated by dividing the area of the signal (detected using the sera) by the total area of the bacteria.

Isolation and differentiation of human monocyte-derived dendritic cells. Dendritic cells were isolated using RosetteSep™ Human Monocyte Enrichment Cocktail (Stemcell Technologies) according to the manufacturer's instructions. In brief, blood from buffy coats from healthy donors was incubated for 20 minutes with RosetteSep™ Human Monocyte Enrichment Cocktail (Stemcell Technologies), layered on top of Ficoll-Paque™ Plus (GE Healthcare) and centrifuged at 1200×g for 20 minutes without acceleration or brake. The monocyte containing layer was recovered, cells washed 7 times with PBS and passed through a 100 μm cell strainer. Monocytes were then differentiated for 6 days in RPMI containing 10% FBS supplemented with 37.5 ng/ml of Granulocyte macrophage colony-stimulating factor (GM-CSF) (Peprotech) and 37.5 ng/ml of Interleukin 4 (IL-4) (Peprotech), changing the medium after 4 days. For experiments, cells were resuspended in RPMI containing 10% FBS.

Toxicity and apoptosis assay of dendritic cells. 6×10⁵ cells were seeded in 96-well plates and incubated with RPMI containing 10% FBS and MV_{L} or MP at indicated concentrations, or with T4R in a multiplicity of infection (MOI) of 20, as positive control, for 24 hours. Gentamicin (100 μg/ml, Sigma) was added after 1 hour of incubation to stop bacteria from growing in samples stimulated with T4R. Before staining, cells were washed once in PBS and once in Annexin V Binding Buffer (BD Pharmingen). Staining was performed with Fixable Viability Dye eFluor® 780 (1:50,000, eBioscience) and FITC Annexin V (1:20, BD Pharmingen) for 30 minutes at 4° C., followed by two washes with Annexin buffer. Labelled cells were fixed in 4% PFA for 30 minutes, resuspended in PBS containing 1% FBS and analyzed in a Gallios™ Flow Cytometer (Beckman Coulter).

Activation assay of dendritic cells. 6×10⁵ cells were seeded in 96-well plates and incubated in RPMI containing 10% FBS with MV_{L} or MP at indicated concentrations, or with 1 μg/ml lipopolysaccharide (LPS) (Sigma) as positive control, for 24 hours. Cells were stained with Phycoerythrin (PE) Mouse Anti-Human CD86 (BD Pharmingen) and PE-Cy™5 Mouse Anti-Human HLA-DR (BD Pharmingen) for 20 minutes at 4° C., washed twice with PBS and resuspended in PBS 1% FBS. Labelled cells were analyzed in a Gallios™ Flow Cytometer (Beckman Coulter).

Uptake assay with dendritic cells. 10⁶ cells were seeded in 96-well plates and incubated with RPMI containing 10% FBS, in presence or absence of the inhibitors cytochalasin D (0.5 μg/ml, Sigma) and wortmannin (0.5 μg/ml, Sigma) (C/W), or methyl-β-cyclodextrin (MβCD) (10 μM, Sigma), for 30 minutes. Cells were then incubated with particles at indicated concentration for 1 hour and lysed in RIPA buffer containing 1× protease inhibitor cocktail (Roche). Cell lysates were analyzed by SDS-PAGE and western blotting.

Quantification of cytokines. Different cytokines were assessed (IL-6, IL-8, IL-10 and TNF) in cell-free supernatants of 10⁵ dendritic cells by Enzyme-Linked Immunosorbent Assay (ELISA), using commercially available BD OptEIA™ kits from BD Biosciences. Cells were incubated with particles at indicated concentrations, or with T4R or LPS (as previously described), for 24 hours.

Opsonophagocytosis assay with RAW cells. RAW 264.7 murine macrophages were grown and maintained at 37° C., with 5% CO₂ in RPMI medium (Gibco) supplemented with 10% (v/v) Fetal bovine serum (FBS) (HyClone). To assess the opsonophagocytosis activity of antibodies in immunized mice sera 2×10⁵ RAW 264.7 cells were seeded in 24-well plates and incubated overnight at 37° C. Serotype 1 bacteria were incubated for 30 minutes at 37° C. with 5% CO₂ with 20% serum from mice immunized with MP+adjuvant, or with adjuvant alone as negative control. RAW cells were then washed with PBS and incubated for 1.5 hours with 2.5×10⁷/well of pre-treated bacteria. Cells were washed three times with PBS to remove unattached bacteria. To measure total uptake of bacteria, cells were incubated with a 50/50 solution of 2% saponin (Sigma) and trypsin-EDTA (Gibco) for 15 minutes at 37° C., to lyse eukaryotic cells, and total bacteria were plated for enumeration. To evaluate phagocytosis 300 μg/ml of Gentamicin (Sigma) and 0.12 mg/ml of Penicillin G (Sigma) were added to separate wells and incubated 15 minutes at 37° C., to kill extracellular bacteria. Cells were then washed three times with PBS and incubated with a 50/50 solution of 2% saponin and trypsin-EDTA for 15 minutes at 37° C. to lyse eukaryotic cells. To evaluate killing of bacteria inside macrophages separate wells were treated with antibiotics (as for phagocytosis), washed three times with PBS and then incubated for 1 hour at 37° C. with medium. Cells were then washed three times with PBS and incubated with a 50/50 solution of 2% saponin and trypsin-EDTA for 15 minutes at 37° C. to lyse eukaryotic cells.

Mouse IgG ELISA assay. To detect MV_{L}- and MP-specific mouse IgG in sera of immunized mice optical plates (Sarstedt) were coated with 1 μg/ml of MP in 0.1 M Sodium Carbonate buffer pH 9.5, overnight at 4° C. Wells were then washed three times with PBS containing 0.05% Tween-20 and incubated with PBS with 10% FBS, for 1 hours at room temperature. After three washes wells were incubated with mice sera diluted 1:500 in PBS with 10% FBS, for 2 hours at room temperature. Wells were then washed three times and incubated with anti-mouse IgG-HRP (GE Healthcare) diluted 1:500 in PBS 10% FBS, for 2 hours at room temperature. After three washes wells were then incubated with TMB substrate (BD Bioscience) for 10 minutes and the reaction was stopped with 1 M H₃PO₄. Absorbance at 450 nm was then measured with a plate reader.

To detect pneumo-specific mouse IgG in sera of immunized mice, bacteria were grown on blood agar plates overnight at 37° C., resuspended in PBS and heat-inactivated for 2 hours at 60° C. After diluting the bacteria to OD₆₀₀ 0.6, optical plates (Sarstedt) were coated with 100 μl of bacteria in 0.1 M Sodium Carbonate buffer pH 9.5, overnight at 4° C. Wells were then washed three times with PBS and incubated with PBS with 2.5% skim milk, for 2 hours at room temperature. After three washes wells were incubated with mice sera diluted 1:500 in PBS, for 1 hour at room temperature. Wells were then washed three times and incubated with anti-mouse IgG-HRP (GE Healthcare) diluted 1:500 in PBS, for 1 hour at room temperature. After three washes well were then incubated with TMB substrate (BD Bioscience) for 10 minutes and the reaction was stopped with 1 M H₃PO₄. Absorbance at 450 nm was then measured with a plate reader.

Statistical analysis. For multiple comparisons the nonparametric ANOVA test was used to assess the presence of the differences between the groups, then the Dunn's test was used to make pairwise comparisons; for two groups comparison the non-parametric two tailed Wilcoxon's rank sum test (also known as Mann-Whitney test) was used. Statistically significant data was defined as * P<0.05, ** P<0.01, *** P<0.001, ****=P<0.0001.

### REFERENCES

1. Olaya-Abril A, Prados-Rosales R, McConnell M J, Martin-Pena R, Gonzalez-Reyes J A, Jimenez-Munguia I, et al. Characterization of protective extracellular membrane-derived vesicles produced by Streptococcus pneumoniae. Journal of proteomics. 2014; 106:46-60.
2. Mitchell T J, Dalziel C E. The biology of pneumolysin. Subcell Biochem. 2014; 80:145-60.
3. Mellroth P, Daniels R, Eberhardt A, Ronnlund D, Blom H, Widengren J, et al. LytA, major autolysin of Streptococcus pneumoniae, requires access to nascent peptidoglycan. J Biol Chem. 2012; 287 (14): 11018-29.
4. Bergmann S, Hammerschmidt S. Versatility of pneumococcal surface proteins. Microbiology. 2006; 152(Pt 2):295-303.
5. Iannelli F, Oggioni M R, Pozzi G. Allelic variation in the highly polymorphic locus pspC of Streptococcus pneumoniae. Gene. 2002; 284 (1-2): 63-71.
6. Barocchi M A, Ries J, Zogaj X, Hemsley C, Albiger B, Kanth A, et al. A pneumococcal pilus influences virulence and host inflammatory responses. Proc Natl Acad Sci USA. 2006; 103(8):2857-62.
7. Nelson A L, Ries J, Bagnoli F, Dahlberg S, Falker S, Rounioja S, et al. RrgA is a pilus-associated adhesin in Streptococcus pneumoniae. Mol Microbiol. 2007; 66 (2): 329-40.
8. lovino F, Hammarlof D L, Garriss G, Brovall S, Nannapaneni P, Henriques-Normark B. Pneumococcal meningitis is promoted by single cocci expressing pilus adhesin RrgA. J Clin Invest. 2016; 126 (8): 2821-6.
9. Bersch B, Bougault C, Roux L, Favier A, Vernet T, Durmort C. New insights into histidine triad proteins: solution structure of a Streptococcus pneumoniae PhtD domain and zinc transfer to AdcAll. PloS one. 2013; 8 (11):e81168.
10. Kilian M, Mestecky J, Schrohenloher R E. Pathogenic species of the genus Haemophilus and Streptococcus pneumoniae produce immunoglobulin A1 protease. Infection and immunity. 1979; 26 (1):143-9.
11. Zhou M, Boekhorst J, Francke C, Siezen R J. LocateP: genome-scale subcellular-location predictor for bacterial proteins. BMC Bioinformatics. 2008; 9:173.
12. Gosink K K, Mann E R, Guglielmo C, Tuomanen E I, Masure H R. Role of novel choline binding proteins in virulence of Streptococcus pneumoniae. Infection and immunity. 2000; 68(10):5690-5.
13. Singh A K, Pluvinage B, Higgins M A, Dalia A B, Woodiga S A, Flynn M, et al. Unravelling the multiple functions of the architecturally intricate Streptococcus pneumoniae beta-galactosidase, BgaA. PLOS Pathog. 2014; 10(9):e1004364.
14. Terrasse R, Amoroso A, Vernet T, Di Guilmi A M. Streptococcus pneumoniae GAPDH Is Released by Cell Lysis and Interacts with Peptidoglycan. PloS one. 2015; 10(4):e0125377.
15. Johnston J W, Myers L E, Ochs M M, Benjamin W H, Jr., Briles D E, Hollingshead S K. Lipoprotein PsaA in virulence of Streptococcus pneumoniae: surface accessibility and role in protection from superoxide. Infection and immunity. 2004; 72(10):5858-67.
16 Paterson G K, Mitchell T J. The role of Streptococcus pneumoniae sortase A in colonisation and pathogenesis. Microbes Infect. 2006; 8(1):145-53.
17. Brooks-Walter A, Briles D E, Hollingshead S K. The pspC gene of Streptococcus pneumoniae encodes a polymorphic protein, PspC, which elicits cross-reactive antibodies to PspA and provides immunity to pneumococcal bacteremia. Infection and immunity. 1999; 67(12):6533-42.
18. Thay B, Wai S N, Oscarsson J. Staphylococcus aureus alpha-toxin-dependent induction of host cell death by membrane-derived vesicles. PloS one. 2013; 8(1):e54661.
19. Gurung M, Moon D C, Choi C W, Lee J H, Bae Y C, Kim J, et al. Staphylococcus aureus produces membrane-derived vesicles that induce host cell death. Plos one. 2011; 6(11):e27958.
20. Ellis T N, Leiman S A, Kuehn M J. Naturally produced outer membrane vesicles from Pseudomonas aeruginosa elicit a potent innate immune response via combined sensing of both lipopolysaccharide and protein components. Infection and immunity. 2010; 78(9):3822-31.
21. Bielig H, Rompikuntal P K, Dongre M, Zurek B, Lindmark B, Ramstedt M, et al. NOD-like receptor activation by outer membrane vesicles from Vibrio cholerae non-O1 non-O139 strains is modulated by the quorum-sensing regulator HapR. Infection and immunity. 2011; 79(4):1418-27.
22. Tettelin H, Nelson K E, Paulsen I T, Eisen J A, Read T D, Peterson S, et al. Complete genome sequence of a virulent isolate of Streptococcus pneumoniae. Science. 2001; 293(5529):498-506.
23. Littmann M, Albiger B, Frentzen A, Normark S, Henriques-Normark B, Plant L. Streptococcus pneumoniae evades human dendritic cell surveillance by pneumolysin expression. EMBO Mol Med. 2009; 1(4):211-22.
24. Fernebro J, Andersson I, Sublett J, Morfeldt E, Novak R, Tuomanen E, et al. Capsular expression in Streptococcus pneumoniae negatively affects spontaneous and antibiotic-induced lysis and contributes to antibiotic tolerance. The Journal of infectious diseases. 2004; 189(2):328-38.

What is claimed is:

1. A method for inducing protective immunity against Streptococcus pneumoniae in a subject, comprising administering to the subject a composition comprising a Streptococcus pneumoniae membrane vesicle microparticle (MP), wherein said MP comprises:
i. the protein pneumolysin (Ply) at the level of ≥0.070 μg/mg total protein in the MP;
ii. the protein autolysin (LytA) at the level of ≥0.070 μg/mg total protein in the MP;
iii. the protein Pneumococcal surface protein C (PspC) at the level of ≥0.130 μg/mg total protein in the MP; or
iv. the protein pilus-1 backbone protein (RrgB) at the level of ≥0.020 μg/mg total protein in the MP.

2. The method according to claim 1, wherein the immunity is protective against a condition selected from pneumococcal sinusitis, pneumococcal otitis, pneumococcal pneumonia, and invasive pneumococcal disease including but not limited to pneumococcal sepsis and pneumococcal meningitis.

3. The method according to claim 1, wherein the composition is a liquid and comprises MPs in an amount of 1 μg/ml.

4. The method according to claim 1, wherein said MP comprises:
i. the protein pneumolysin (Ply) at the level of ≥0.070 μg/mg total protein in the MP;
ii. the protein autolysin (LytA) at the level of ≥0.070 μg/mg total protein in the MP;
iii. the protein Pneumococcal surface protein C (PspC) at the level of ≥0.130 μg/mg total protein in the MP; and
iv. the protein pilus-1 backbone protein (RrgB) at the level of ≥0.020 μg/mg total protein in the MP.

5. The method according to claim 1, wherein the MP is 5-300 nm in diameter.

6. The method according to claim 1, wherein the MP is 10-125 nm in diameter.

7. The method according to claim 1, wherein the composition elicits antibodies against Streptococcus pneumoniae serotype 3 when administered to a mammalian host.

8. The method according to claim 1, wherein the composition elicits serotype independent antibodies against Streptococcus pneumoniae when administered to a mammalian host.

9. The method according to claim 1, wherein the composition is administered intranasally.

10. The method according to claim 1, wherein the composition further comprises an adjuvant.

11. The method according to claim 10, wherein the adjuvant comprises aluminium hydroxide.

12. The method according to claim 2, wherein the immunity is protective against invasive pneumococcal disease.

13. The method according to claim 1, wherein the MP comprises a capsular polysaccharide of a capsular serotype of Streptococcus pneumoniae at a level of ≥0.001 μg/mg total protein in the MP.

14. The method according to claim 1, wherein said MP comprises the protein Ply at the level of ≥0.070 μg/mg total protein in the MP.

15. The method according to claim 1, wherein said MP comprises the protein LytA at the level of ≥0.070 μg/mg total protein in the MP.

16. The method according to claim 1, wherein said MP comprises the protein PspC at the level of ≥0.130 μg/mg total protein in the MP.

17. The method according to claim 1, wherein said MP comprises the protein RrgB at the level of ≥0.020 μg/mg total protein in the MP.

18. The method according to claim 1, wherein said MP comprises the protein Ply at the level of ≥0.35 μg/mg total protein in the MP.

19. The method according to claim 1, wherein said MP comprises the protein LytA at the level of ≥0.20 μg/mg total protein in the MP.

20. The method according to claim 1, wherein said MP comprises the protein PspC at the level of >0.3 μg/mg total protein in the MP.

21. The method according to claim 1, wherein said MP comprises the protein RrgB at the level of ≥0.028 μg/mg total protein in the MP.
